(19) United States
(12) Patent Application Publication
(10) Pub. No.: US 2024/0199714 A1
(43) Pub. Date: Jun. 20, 2024

(54) IL-2 BASED CONSTRUCTS

(71) Applicants: Orionis Biosciences, Inc., Waltham, MA (US); Orionis Biosciences BV, Ghent (BE)

(72) Inventors: Nikolai KLEY, Waltham, MA (US); Erik DEPLA, Ghent (BE); Lennart ZABEAU, Ghent (BE); Anje CAUWELS, Ghent (BV)

(21) Appl. No.: 18/286,147

(22) PCT Filed: Apr. 16, 2022
(86) PCT No.: PCT/US22/25143

§ 371 Date: Oct. 9, 2023

Related U.S. Application Data
(60) Provisional application No. 63/297,330, filed on Jan. 7, 2022.
(60) Provisional application No. 63/175,827, filed on Apr. 16, 2021.

(51) Int. Cl. C07K 14/55; A61K 38/00; A61P 35/00

(52) U.S. Cl. CPC C07K 14/55; A61P 35/00; A61K 38/00; C07K 2319/01; C07K 2319/30; C07K 2319/33

(57) ABSTRACT

The present invention relates, in part, to chimeric proteins or chimeric protein complexes, such as Fc-based chimeric protein complexes, comprising interleukin 2, or a variant thereof, and their use as therapeutic agents.

### CROSS-REFERENCE TO RELATED APPLICATIONS

This application claims the benefit of U.S. Provisional Patent Application Nos. 63/175,827, filed Apr. 16, 2021, and 63/297,330, filed Jan. 7, 2022, the entire contents of all of which are hereby incorporated by reference in their entirety.

### FIELD

The present invention relates, in part, to chimeric proteins, or chimeric protein complexes (including Fc-based chimeric protein complexes) comprising interleukin 2 (IL-2) or variants thereof and their use as therapeutic agents.

### SEQUENCE LISTING

The contents of the text file submitted electronically herewith are incorporated herein by reference in their entirety. A computer readable format copy of the Sequence Listing (filename: ORN-080PC_ST25.txt, date created: Apr. 14, 2022; file size: 1,151,315 bytes).

### BACKGROUND

Interleukin 2 (IL-2) is a member of a cytokine family, each member of which has a four alpha helix bundle. IL-2 signals through the IL-2 receptor, a complex consisting of two or three polypeptide chains, i.e., the alpha and/or beta and gamma chains.

IL-2 has been approved for cancer treatment with a high-dose regimen. Illustrative cancers treated with IL-2 include renal cell (kidney) and melanoma, a skin cancer.

IL-2 stimulates immune cell proliferation and activation through receptor-signaling complexes containing alpha (IL-2Rα, CD25) and/or beta (IL-2Rβ, CD122), and common gamma receptor polypeptide chains (γc, CD132), but the effects are pleiotropic and contextual. At high doses, IL-2 binds to heterodimeric IL-2Rβγ receptor leading to desired expansion of tumor killing CD8⁺ effector, memory T cells, and NK cells. IL-2 also binds to its heterotrimeric receptor IL-2Rαβγ with greater affinity, which expands immunosuppressive CD4⁺CD25⁺ regulatory T cells (Tregs) expressing high constitutive levels of IL-2Rα. Expansion of Tregs represents an undesirable effect of IL-2 for cancer immunotherapy. Furthermore, activation of IL-2Rαβγ on endothelial cells can cause vascular leak syndrome (VLS).

Conversely, stimulation of CD8⁺ T cells represents an undesirable effect of IL-2 in autoimmune disease therapy, where stimulation of Tregs by IL-2 is desired to suppress autoreactive CD8⁺ T cells. Due to its overall pleiotropic effects on multiple cell types, IL-2 therapies have been plagued by significant side effects and systemic toxicities, including vascular leakage syndrome (VLS), limiting exploitation of its therapeutic potential.

Constructs for receptor selective IL-2 have been made (e.g., IL-2Rα and IL-2Rβ interaction deficient or attenuated constructs). However, these constructs still have side effect and toxicity issues. Some of the issues could be ameliorated with reducing dose (e.g., via extension of half-life), but the dose reduction alone does not solve cell-selectivity issues. As such, there remains a need for IL-2-based agents that have reduced cellular side effects (off-target effects) and lower systemic toxicity for use in IL-2Rβγ targeting (e.g., for CD8⁺ T cells activation in the treatment of cancer, for Thelp cells activation in the treatment of cancer, and for NK cells activation in the treatment of cancer) and IL-2Rαβγ targeting (e.g., for Treg stimulation in autoimmune diseases).

Accordingly, there remains a need for safe and effective IL-2-based therapeutics with improved target selectivity, pharmacokinetic and therapeutic properties and minimal toxicity profiles.

### SUMMARY

Accordingly, in some aspects, the present invention relates to chimeric proteins and chimeric protein complexes, including Fc-based chimeric protein complexes, comprising a modified IL-2 as a signaling agent. In some embodiments, the modified IL-2 is a modified wild type IL-2, wherein the wild type IL-2 has the amino acid sequence of SEQ ID NO: 1. In some embodiments, the modified IL-2 is a modified neoleukin, wherein the neoleukin has the amino acid sequence of SEQ ID NO: 2.

In some embodiments, the modified IL-2 signaling agent comprises: (i) a mutation selected from D20E, D20F, D20G, D20H, D20I, D20K, D20L, and D20V; or (ii) a mutation selected from N88G, N88E, N88K, N88Q, and N88V; or (iii) a mutation selected from Q126G, Q126A, Q126E, Q126F, Q126H, Q126I, Q126K, Q126L, Q126N, Q126P, Q126R, Q126S, Q126T, Q126V, Q126W, and Q126Y; or (iv) a mutation selected from (i) and (iii) or a mutation selected from (ii) and (iii); or (v) one or more mutations selected from: R38A/F42K/N88G/C125A, F42K/C125A, D20E/C125A, D20F/C125A, D20G/C125A, D20H/C125A, D20I/C125A, D20K/C125A, D20L/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, N88V/C125A, D20E/R38A/F42K, D20E/R38A/F42K/C125A, D20V/R38A/F42K, D20V/R38A/F42K/C125A, R38A/F42Y/E62A/C125A, R38A/F42Y/Y45A/E62A, F42Y/Y45A/L72G, R38A/F42Y/Y45A/E62A/C125A, F42Y/Y45A/L72G/C125A, R38A/F42K/N88G, R38A/F42K/Q126G, R38A/F42K/Q126I, R38A/F42K/Q126Y, R38A/F42K/Q126G/C125A, R38A/F42K/Q126I/C125A, R38A/F42K/Q126Y/C125A, R38A/F42K/E62A/N88G, and R38A/F42K/E62A/N88G/C125A.

In some embodiments, the modified IL-2 signaling agent comprises one or more mutations at an amino acid residue selected from R38, F42, E62, C125, Y45, L72, D20, N88, and Q126 relative to SEQ ID NO: 1. In such embodiments, the modified IL-2 signaling agent comprises one or more mutations selected from R38A, F42K, F42Y, E62A, C125A, Y45A, L72G, D20E, D20F, D20G, D20H, D20I, D20K, D20L, D20N, D20V, N88G, N88E, N88I, N88K, N88Q, N88R, N88V, R38A/F42Y/E62A/C125A, F42Y/Y45A/L72G/C125A, F42K/C125A, R38A/F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K/C125A, D20V/R38A/F42K/C125A, R38A/F42K/N88A/C125A, R38A/F42K/N88G/C125A, Q126A, Q126E, Q126F, Q126G, Q126H, Q126I, Q126K, Q126L, Q126N, Q126P, Q126R, Q126S, Q126T, Q126V, Q126W, and Q126Y, relative to SEQ ID NO: 1.

In various embodiments, the modified IL-2 signaling agent comprises one or more mutations selected from D20V, D20E, N88G, F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, R38A/F42K/E62A/N88G/C125A, Q126A, Q126E, Q126F, Q126G, Q126H, Q126I, Q126K, Q126L, Q126N, Q126P, Q126R, Q126S, Q126T, Q126V, Q126W, and Q126Y, relative to SEQ ID NO: 1.

In particular embodiments, the modified IL-2 signaling agent comprises one or more mutations selected from R38A/F42Y/E62A/C125A, F42Y/Y45A/L72G/C125A, F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K/C125A, D20V/R38A/F42K/C125A, and R38A/F42K/N88G/C125A, relative to SEQ ID NO: 1.

In some embodiments, the modified IL-2 signaling agent is modified neoleukin, which comprises the amino acid sequence of SEQ ID NO: 2. In such embodiments, the modified IL-2 signaling agent comprising SEQ ID NO: 2 has one or more mutations at amino acid residues D15 and/or N40. For example, in some embodiments, the modified IL-2 signaling agent comprising SEQ ID NO: 2 comprises D15T, D15H, N401, N40G, or N40R.

In some embodiments, the one or more mutations or modifications improve the safety profile of the modified IL-2, relative to, for example, wild type IL-2. In some embodiments, the one or more mutations or modifications reduce the biological activity of the modified IL-2, relative to, for example, wild type IL-2. For example, the one or more mutations or modifications may reduce the affinity of the modified IL-2 for a target receptor. In some embodiments, the target receptor is the heterotrimeric receptor IL-2Rαβγ, which is composed of the IL-2Rα, IL-2Rβ, and IL-2Ry subunits. In some embodiments, the target receptor is the heterodimeric receptor IL-2Rβγ, which is composed of the IL-2RB and IL-2Ry subunits. In some embodiments, the modified IL-2 comprises one or more mutations or modifications that reduce its affinity for IL-2Rα subunit. In another embodiment, the modified IL-2 comprises one or more mutations or modifications that reduce its affinity for IL-2Rβ. In yet another embodiment, the modified IL-2 comprises one or more mutations or modifications that reduce its affinity for IL-2Ry. In an embodiment, the modified IL-2 comprises one or more mutations or modifications that reduce its affinity for any combination of IL-2Rα, IL-2Rβ, and IL-2Ry (e.g., one or more mutations or modifications that reduce the modified IL-2's affinity for IL-2Rα, IL-2Rβ, and IL-2Ry or for IL-2Rα and IL-2RB or for IL-2Ry and IL-2RB or for IL-2Rα and IL-2Ry).

In some embodiments, the modified IL-2 exhibits reduced affinity and/or activity for IL-2Rα and IL2Rβ, optionally wherein the modified IL-2 comprises one or more mutations selected from D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, and R38A/F42K/E62A/N88G/C125A.

In some embodiments, the modified IL-2 exhibits reduced affinity and/or activity for IL-2Rα and IL-2Rγ, optionally wherein the modified IL-2 comprises one or more mutations selected from R38A/F42K/Q126G, R38A/F42K/Q126I, and R38A/F42K/Q126Y.

In some embodiments, the modified IL-2 exhibits reduced affinity and/or activity for IL-2Rβ, optionally wherein the modified IL-2 comprises one or more mutations selected from D20E, D20F, D20G, D20H, D20I, D20K, D20L, D20V, N88G, N88E, N88K, N88Q, N88V, D20E/C125A, D20F/C125A, D20G/C125A, D20H/C125A, D20I/C125A, D20L/C125A, D20K/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, and N88V/C125A.

In some embodiments, the modified IL-2 exhibits reduced affinity and/or activity for IL-2Rγ, optionally wherein the modified IL-2 comprises one or more mutations selected from Q126A, Q126E, Q126F, Q126G, Q126H, Q126I, Q126K, Q126L, Q126N, Q126P, Q126R, Q126S, Q126T, Q126V, Q126W, and Q126Y.

In some embodiments, the modified IL-2 exhibits reduced affinity and/or activity for IL-2Rβγ, optionally wherein the modified IL-2 comprises one or more mutations selected from D20E/Q126A, D20E/Q126D, D20E/Q126E, D20E/Q126F, D20E/Q126G, D20E/Q126H, D20E/Q126I, D20E/Q126K, D20E/Q126L, D20E/Q126M, D20E/Q126N, D20E/Q126P, D20E/Q126R, D20E/Q126S, D20E/Q126T, D20E/Q126V, D20E/Q126W, D20E/Q126Y, D20V/Q126A, D20V/Q126D, D20V/Q126E, D20V/Q126F, D20V/Q126G, D20V/Q126H, D20V/Q126I, D20V/Q126K, D20V/Q126L, D20V/Q126M, D20V/Q126N, D20V/Q126P, D20V/Q126R, D20V/Q126S, D20V/Q126T, D20V/Q126V, D20V/Q126W, D20V/Q126Y, N88A/Q126A, N88A/Q126D, N88A/Q126E, N88A/Q126F, N88A/Q126G, N88A/Q126H, N88A/Q126I, N88A/Q126K, N88A/Q126L, N88A/Q126M, N88A/Q126N, N88A/Q126P, N88A/Q126R, N88A/Q126S, N88A/Q126T, N88A/Q126V, N88A/Q126W, N88A/Q126Y, N88G/Q126A, N88G/Q126D, N88G/Q126E, N88G/Q126F, N88G/Q126G, N88G/Q126H, N88G/Q126I, N88G/Q126K, N88G/Q126L, N88G/Q126M, N88G/Q126N, N88G/Q126P, N88G/Q126R, N88G/Q126S, N88G/Q126T, N88G/Q126V, N88G/Q126W, and N88G/Q126Y. In further embodiments, the modified IL-2 exhibits reduced affinity and/or activity for IL-2Rβγ and exhibits increased selectivity toward activated CD8 cells.

In another aspect, the modified IL-2 exhibits reduced affinity and/or activity for IL-2Rβ and comprises a single mutation relative to a wild type signaling agent having SEQ ID NO: 1 selected from D20E, D20F, D20G, D20H, D20I, D20K, D20L, D20N, D20V, N88G, N88E, N88I, N88K, N88Q, N88R and N88V, D20E/C125A, D20F/C125A, D20G/C125A, D20H/C125A, D20I/C125A, D20K/C125A, D20L/C125A, D20N/C125A, D20S/C125A, D20T/C125A, D20V/C125A, N88A/C125A, N88D/C125A, N88G/C125A, N88H/C125A, N88Q/C125A, and N88T/C125A, and includes no mutation that confers reduced affinity or bioactivity for the IL-2R alpha subunit (IL-2Rα). In further embodiments, the modified IL-2 exhibits reduced affinity and/or activity for IL-2RB and exhibits increased selectivity toward activated CD8 cells and decreased selectivity toward Tregs.

In some embodiments, the target receptor is the heterotrimeric receptor IL-2Rαβγ, which is composed of the IL-2Rα, IL-2Rβ, and IL-2Ry subunits. In some embodiments, the target receptor is the heterodimeric receptor IL-2Rβγ, which is composed of the IL-2Rβ and IL-2Ry subunits.

In some embodiments, the IL-2 has one or more mutations or modifications: one or more mutations or modifications that reduce or ablate the activity or affinity of the modified IL-2 for an IL-2Rα chain; and/or one or more mutations or modifications that reduce the activity or affinity of the modified IL-2 for an IL-2R lacking an a chain. In some embodiments, the mutations or modifications that reduce the activity or affinity of the modified IL-2 and/or attenuate the modified IL-2 are restorable at a target cell or site of therapeutic action through a targeting moiety that is linked to the modified IL-2.

In some embodiments, the IL-2 has one or more mutations or modifications: one or more mutations or modifications that reduces the activity or affinity of the modified IL-2 for an IL-2R lacking an a chain and/or one or more mutations or modifications that reduce the activity or affinity of the modified IL-2 for an IL-2R comprising an a chain, such mutation or modification being an attenuation mutation for which the activity or affinity is restorable by induced proximity at/to a target or a site of therapeutic action (e.g., a cell or the extracellular matrix) through a targeting moiety.

In various embodiments, the present invention contemplates a chimeric protein, a chimeric protein complex, or a Fc-based chimeric protein complex comprising a modified IL-2-signaling agent that comprises a glycosylation mutation. In some embodiments, the glycosylation mutation decreases or eliminates glycosylation of the modified IL-2 signaling agent. In some embodiments, the glycosylation mutation is a T3 substitution, wherein the mutation is one of T3A, T3F, T3H, T3L, T3V, and T3Y. In some embodiments, the glysosylation mutation is a deletion of the first 3, 4, 5, 6, 7, or 8 residues of the modified IL-2.

In some embodiments, the chimeric protein comprises one or more additional signaling agents, e.g., without limitation, an interferon, an interleukin, and a tumor necrosis factor, that may be modified.

In some embodiments, the chimeric protein comprises one or more targeting moieties which allow for a restoration of the weakened or attenuated activity described herein (e.g. on IL-2). In some embodiments, the chimeric protein comprises one or more targeting moieties which have recognition domains (e.g. antigen recognition domains, including without limitation various antibody formats, inclusive of single-domain antibodies) that specifically bind to a target (e.g. antigen, receptor) of interest. In various embodiments, the targeting moieties have recognition domains that specifically bind to a target (e.g. antigen, receptor) of interest, including those found on one or more immune cells, which can include, without limitation, T cells, Tregs, cytotoxic T lymphocytes, T helper cells, natural killer (NK) cells, natural killer T (NKT) cells, anti-tumor or tumor macrophages (e.g. M1 or M2 macrophages, respectively), B cells, and dendritic cells. In some embodiments, the immune cell is a T cell and the targeting moiety targets CD8. In embodiments, the immune cell is a Treg and the targeting moiety targets CTLA4. In embodiments, the immune cell is an NK cell and the targeting moiety is NKp46.

In some embodiments, the recognition domains specifically bind to a target (e.g. antigen, receptor) of interest and effectively recruit one of more immune cells. In some embodiments, the targets (e.g. antigens, receptors) of interest can be found on one or more tumor cells. In some embodiments, the present chimeric proteins may recruit an immune cell, e.g. an immune cell that can kill and/or suppress a tumor cell, to a site of action (such as, by way of non-limiting example, the tumor microenvironment). In some embodiments, the recognition domain binds to a target of interest found in a normal tissue of interest. In some embodiments the present chimeric proteins may bind to a target cell to direct localization of the chimeric protein to a tissue cell of interest while also capable of binding to a second, different cell type (e.g., an immune cell) to impart a tissue localized biological effect mediated by the modified IL-2. In some embodiments, the recognition domains specifically bind to a target (e.g. antigen, receptor) of interest which is part of a non-cellular structure.

In various embodiments, the chimeric protein of the present technology provides improved cell target selectivity, safety and/or therapeutic activity and/or pharmacokinetic profiles (e.g., increased serum half-life) compared to an untargeted modified IL-2.

In some embodiments, the chimeric protein of the present technology is a single chain polypeptide.

In some embodiments, the chimeric protein of the present technology is a protein complex comprising two or more polypeptide chains. In some embodiments, the targeting moiety (or moieties) and the signaling agent(s) (e.g., modified IL-2) are on the same polypeptide chain in the protein complex. In some embodiments, the targeting moiety (or moieties) and the signaling agent(s) (e.g., modified IL-2) are on different polypeptide chains in the protein complex.

In various embodiments, the present chimeric proteins find use in the treatment of various diseases or disorders such as cancer, infections, immune disorders, autoimmune diseases, cardiovascular diseases, wound healing, ischemia-related diseases, neurodegenerative diseases, metabolic diseases and many other diseases and disorders, and the present invention encompasses various methods of treatment.

In another aspect, the present invention relates to Fc-based chimeric protein complexes comprising a modified IL-2 as a signaling agent. In some embodiments, the Fc-based chimeric protein complex of the present invention comprises a polypeptide having an amino acid sequence having at least 90%, or at least 95%, or at least 96%, or at least 97%, or at least 98%, or at least 99% identity with any one of SEQ ID NOs: 291-296, 298-335. In particular embodiments, the Fc-based chimeric protein complex of the present invention comprises a polypeptide having an amino acid sequence having at least 90%, or at least 95%, or at least 96%, or at least 97%, or at least 98%, or at least 99% identity with any one of SEQ ID NOs: 292, 293, 294, 299, 301, 310, 311, 312, 315, 316, 319, 320, 325, 328, 332, 333, and 335.

In some embodiments, the present invention relates to a chimeric protein or an Fc-based chimeric protein complex comprising a polypeptide having an amino acid sequence having at least 90%, or at least 95%, or at least 96%, or at least 97%, or at least 98%, or at least 99% identity with any one of SEQ ID NOs: 290-449, 478-495, or 501-531.

### BRIEF DESCRIPTION OF THE DRAWINGS

FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F show various non-limiting illustrative schematics of the Fc-based chimeric protein complexes of the present invention. In embodiments, each schematic is a composition of the present invention. Where applicable in the figures, “TM” refers to a “targeting moiety” as described herein, “SA” refers to a “signaling agent” as described herein, [symbol] is an optional “linker” as described herein, the two long parallel rectangles are human Fc domains, e.g. from IgG1, from IgG2, or from IgG4, as described herein and optionally with effector knock-out and/or stabilization mutations as also described herein, and the two long parallel rectangles with one having a protrusion and the other having an indentation are human Fc domains, e.g. from IgG1, from IgG2, or from IgG4 as described herein, with knob-in-hole and/or ionic pair (a/k/a charged pairs, ionic bond, or charged residue pair) mutations as described herein and optionally with effector knock-out and/or stabilization mutations as also described herein. At least one signaling agent is IL-2, or modified IL-2, or neoleukin, or modified neoleukin.

FIGS. 1A-F show illustrative homodimeric 2-chain complexes. These figures show illustrative configurations for the homodimeric 2-chain complexes.

FIGS. 2A-H show illustrative homodimeric 2-chain complexes with two targeting moieties TM (as described herein, more targeting moieties may be present in some embodiments). In embodiments, the position of TM1 and TM2 are interchangeable. In embodiments, the constructs shown in the box (i.e., FIGS. 2G and 2H) have signaling agent (SA) between TM1 and TM2 or between TM1 and Fc.

FIGS. 3A-H show illustrative homodimeric 2-chain complexes with two signaling agents (as described herein, more signaling agents may be present in some embodiments). In embodiments, the position of SA1 and SA2 are interchangeable. In embodiments, the constructs shown in the box (i.e., FIGS. 3G and 3H) have a TM between Fc and SA1 and/or SA2, N- or C-terminal of the Fc chains.

FIGS. 4A-D show illustrative heterodimeric 2-chain complexes with split TM and SA chains, namely the TM on the knob chain of the Fc and the SA on hole chain of the Fc.

FIGS. 5A-F show illustrative heterodimeric 2-chain complexes with split TM and SA chains, namely with both TMs on the knob chain of the Fc and with SA on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 6A-J show illustrative heterodimeric 2-chain complexes, namely with TM on the knob chain of the Fc and with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 7A-D show illustrative heterodimeric 2-chain complexes with split TM and SA chains, namely the SA on the knob chain of the Fc and the TM on hole chain of the Fc.

FIGS. 8A-F show illustrative heterodimeric 2-chain complexes with split TM and SA chains, namely with SA on the knob chain of the Fc and both TMs on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 9A-J show illustrative heterodimeric 2-chain complexes, namely with a TM on hole chain of the Fc and with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 10A-F show illustrative heterodimeric 2-chain complexes with TM and SA on the same chain, namely the SA and TM both on the knob chain of the Fc.

FIGS. 11A-L show illustrative heterodimeric 2-chain complexes with a TM and a SA on the same chain, namely with SA and with TM both on the knob chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 12A-L show illustrative heterodimeric 2-chain complexes with a TM and a SA on the same chain, namely with SA and with TM both on the knob chain of the Fc, with two signaling agents (as described herein, more signaling agents may be present in some embodiments). In embodiments, the position of SA1 and SA2 are interchangeable.

FIGS. 13A-F show illustrative heterodimeric 2-chain complexes with TM and SA on the same chain, namely the SA and TM both on the hole chain of the Fc.

FIGS. 14A-L show illustrative heterodimeric 2-chain complexes with a TM and a SA on the same chain, namely with SA and with TM both on the hole chain of the Fc, with two targeting moieties (as described herein, more targeting moieties are present in some embodiments). In embodiments, the position of TM1 and TM2 are interchangeable. In embodiments, TM1 and TM2 can be identical.

FIGS. 15A-L show illustrative heterodimeric 2-chain complexes with a TM and a SA on the same chain, namely with SA and with TM both on the hole chain of the Fc, with two signaling agents (as described herein, more signaling agents may be present in some embodiments). In embodiments, the position of SA1 and SA2 are interchangeable.

FIGS. 16A-J show illustrative heterodimeric 2-chain complexes with two targeting moieties (as described herein, more targeting moieties may be present in some embodiments) and with SA on knob Fc and TM on each chain. In embodiments, TM1 and TM2 can be identical.

FIGS. 17A-J show illustrative heterodimeric 2-chain complexes with two targeting moieties (as described herein, more targeting moieties may be present in some embodiments) and with SA on hole Fc and TM on each chain. In embodiments, TM1 and TM2 can be identical.

FIGS. 18A-F show illustrative heterodimeric 2-chain complexes with two signaling agents (as described herein, more signaling agents may be present in some embodiments) and with split SA and TM chains: SA on knob and TM on hole Fc.

FIGS. 19A-F show illustrative heterodimeric 2-chain complexes with two signaling agents (as described herein, more signaling agents may be present in some embodiments) and with split SA and TM chains: TM on knob and SA on hole Fc.

FIG. 20 depicts IL-2 and Fc-IL-2 driven STAT5 phosphorylation in PBMC lymphocyte populations.

FIGS. 21A-G show the effect of CD25 knock-out mutations and CD8 targeting on STAT5 phosphorylation in lymphocyte populations.

FIGS. 22A-G depict sensorgrams of binding of IL-2 and Fc-IL-2 fusion-proteins to biotinylated CD25 in bio-layer interferometry.

FIGS. 23A-R depict screening of D20 mutants for pSTAT5 in CD8⁺, CD4⁺CD25, and CD4⁺CD25⁺ PMBCs.

FIGS. 24A-R depict screening of N88 mutants for pSTAT5 in CD8⁺, CD4⁺CD25, and CD4⁺CD25⁺ PMBCs

FIGS. 25A-D show the effect of beta mutations (here N88G) and CD8 targeting on STAT5 phosphorylation in PBMC sub-populations.

FIGS. 26A-J show various combinations of CD25 and IL-2RB mutations and their effect on pSTAT5 in PMBCs populations.

FIGS. 27A-B depict binding of CD25 scFv Fc-fusions to HEK-Blue IL-2 and HEK-Blue IL-1 cells in FACS.

FIG. 28 shows the effect of CD25 scFv Fc-fusions on wild type IL-2 signaling in HEK-Blue IL-2 cells.

FIGS. 29A-M depict the effect of CD25 targeting of ALN2 variants with CD25 or beta mutations on STAT5 phosphorylation in certain PBMC sub-populations.

FIGS. 30A-H show STAT5 phosphorylation in PBMC subpopulations by IL-2 and Fc-IL-2 mimic/neoleukin variants.

FIG. 31 depicts different bi-valent (if VHH1 and VHH2 are identical) or bi-specific (if VHH1 and VHH2 represent different targeting domains) knob-in-hole ALN2 formats.

FIG. 32 depicts various ALN2 configurations.

FIGS. 33A-G depicts a STAT5 comparison of STAT5 phosphorylation by bi-valent and bi-specific CD8 VHH targeted ALN2's compared to the mono-valent counterparts.

FIGS. 34A-E depicts STAT5 phosphorylation ALN2's in different formats in CD8⁺ and Treg PBMC subpopulations.

FIGS. 35A-D show that single-peptide ALN2 induced pSTAT5 in CD8+ compared to Treg PBMC subpopulations.

FIGS. 36A-M depict STAT5 phosphorylation in CD8⁺ and CD8− PBMC lymphocyte populations by CD8 VHH-Fc-IL-2 and T3 O-glycosylation variants thereof.

FIGS. 37A-D show STAT5 phosphorylation by mouse CD8 targeted alpha:beta ALN2 variants in CD8⁺ and Treg primary mouse splenocytes.

FIG. 38 depicts tumor growth (in mm³) of animals treated with buffer, Fc-ALN2, or CD8-Fc-ALN2 in the MC38 and CT26 syngeneic mouse models for colon carcinoma.

FIG. 39 shows the effect of CD8-Fc-ALN2 and anti-PD-1 co-treatment in the MC38 and CT26 mouse models.

FIGS. 40A-D show STAT5 phosphorylation by CD8 targeted Fc gamma common Q126 mutants in CD8 positive PBMCs.

FIGS. 41A-S depict sensorgrams of binding of CD8-Fc-IL-2 and Q126 mutants thereof fusion proteins to biotinylated CD25 in BLI.

FIGS. 42A-D show pSTAT5 phosphorylation by alpha:gamma mutants in CD8⁺ and Treg PBMC subpopulations of three different donors.

FIGS. 43A-D depict beta:gamma ALN2 STAT5 phosphorylation of ‘resting’ or TransAct activated CD8⁺ or Treg PBMC subpopulations.

FIG. 44 shows the effect of CD8-Fc-ALN2 (12.5 μg), Fc-ALN2 (10.7 μg) or Fc-IL2 (10.7 μg) on T cell activation 1 day after single dose.

FIG. 45 depicts the effect of CD8-Fc-ALN2 (12.5 μg), Fc-ALN2 (10.7 μg) or Fc-IL2 (10.7 μg) with and without anti-PD-1 Ab co-treatment on T cell activation 7 days after two doses.

FIG. 46 shows the effect of CD8-Fc-ALN2 (25 μg), Fc-ALN2 (21.4 μg) or Fc-IL2 (21.4 μg) on T cell activation 3 days after a single dose.

FIG. 47 depicts the effect of three injections of 12.5 μg CD8-Fc-ALN2 and anti-PD-1 Ab, compared to wild type Fc-IL2, 1 in the A20 mouse model.

FIG. 48 shows the effect of three doses of 12.5 μg CD8-Fc-ALN2, compared to equimolar Fc-ALN2 treatments in the B16F10 mouse model

FIG. 49 depicts the effect of two injections of 12.5 μg CD8-Fc-ALN2, compared to equimolar Fc-ALN2 treatment in the Panc02 mouse model.

FIG. 50 depicts STAT5 phosphorylation in mouse splenocytes (singlet population) comparing wild type IL2, PD-L1 targeted, and untargeted ALN2.

FIG. 51 shows the effect of three doses of 12.5 μg CD8-Fc-ALN2 and anti-PD-1 Ab, compared to equimolar Fc-ALN2 treatments in the MC38 mouse model.

FIG. 52 depicts the effect of three doses of 12.5 μg TNCA1-Fc-ALN2 or equivalent dose of untargeted ALN2 without or with anti-PD-1 Ab co-treatment in the MC38 mouse model.

FIG. 53 shows STAT5 phosphorylation by mouse CD8, or NKp46, or bispecific targeted alpha:beta ALN2 variants in CD8 and NK primary mouse splenocytes compared to untargeted or wild type IL-2.

FIG. 54 depicts CD25 (left) and PD-1 (right) up-regulation by ALN2 variants R38A_F42K_D20E (upper) and R38A_F42K_N88G (lower) in three PBMC donors. Data are plotted as means±SEM.

FIG. 55 shows the effect of hCD8-Fc-ALN2 on human T cell activation in human immune system (HIS) mice 45 min after 1 treatment (5 μg).

FIG. 56 depicts CD25 up-regulation in primary mouse splenocytes by mouse CD8 targeted or untargeted ALN2 variants with different loss-of-function beta mutations.

FIG. 57 shows the effect of treatment on days 6 and 9 with 12.5 μg N88G- or D20E-based CD8-Fc-ALN2 or equimolar untargeted ALN2s in the MC38 mouse model.

FIG. 58 depicts STAT5 phosphorylation in primary mouse splenocytes by ALN2 variants with different loss-of-function gamma mutations.

FIG. 59 shows the effect of three doses of 12.5 or 4.17 μg CD8-Fc-ALN2 alpha:gamma mutation combination in the MC38 mouse model and compared to equimolar dosing of untargeted Fc-ALN.

FIG. 60 shows the effect of three doses of 4.17 or 1.4 μg CD8-Fc-ALN2 beta:gamma mutation combination in the MC38 mouse model and compared to equimolar dosing of untargeted Fc-ALN.

FIG. 61 depicts signaling of D20 ALN2 variants in HEK-Blue CD8 vs HEK-Blue NKp46 cells.

FIG. 62 depicts signaling of N88 ALN2 variants in HEK-Blue CD8 vs HEK-Blue NKp46 cells.

### DETAILED DESCRIPTION

The present technology is based, in part, on the discovery that targeted chimeric proteins or protein complexes, including Fc-based chimeric protein complexes, that include a modified IL-2 exhibit beneficial therapeutic properties and reduced side effects. For example, the chimeric proteins of the present technology are highly active and/or long-acting while eliciting minimal side effects. The present technology provides pharmaceutical compositions comprising the chimeric proteins and their use in the treatment of various diseases.

In some embodiments, the chimeric protein or protein complexes having modified IL-2 may favor attenuated activation of CD8⁺ T cells, Thelp cells, or NK cells (which can provide an anti-tumor effect), which primarily express IL-2 receptors β and γ, and disfavor T_{regs} (which can provide an immune suppressive, pro-tumor effect), which have IL-2 receptors α, β, and γ. Further, in some embodiments, the preferences for IL-2RB and/or IL-2Rγ over IL-2Rα avoid IL-2 side effects such as pulmonary edema and/or vascular leak syndrome (VLS). Also, IL-2-based chimeras are useful for the treatment of diseases (e.g., autoimmune disease), for instance when the modified IL-2 is antagonistic (e.g. natural antagonistic activity or antagonistic activity that is the result of one or more mutations, see, e.g., WO 2015/007520, the entire contents of which are hereby incorporated by reference) at IL-2Rβ and/or IL-2Rγ. For instance, the present constructs may favor attenuated suppression of CD8⁺ T cells (and therefore dampen the immune response), which have IL-2 receptors β and γ and disfavor T_{regs} which have IL-2 receptors α, β, and γ. Alternatively, in some embodiments, the chimeras bearing modified IL-2 favor the agonistic activation of T_{regs}, and therefore immune suppression, and disfavor activation of CD8⁺ T cells. For instance, these constructs find use in the treatment of diseases or diseases that would benefit from immune suppression, e.g., autoimmune disorders.

In some embodiments, the chimeric protein has targeting moieties as described herein directed to CD8⁺ T cells as well as a modified IL-2 having reduced affinity and/or activity for IL-2RB and/or IL-2Rγ and/or substantially reduced or ablated affinity and/or activity for IL-2Rα. In some embodiments, these constructs provide targeted CD8⁺ T cell activity and are generally inactive (or have substantially reduced activity) towards Treg cells. In some embodiments, such constructs have enhanced immune stimulatory effect compared to wild type IL-2 (e.g., without wishing to be bound by theory, by not stimulating T_{regs}), whilst eliminating or reducing the systemic side effects and/or toxicity associated with wild type IL-2 and modified IL-2 proteins constructs that preferentially target IL-2Rβγ but not in a cell-selective, targeted and conditional manner, such as described in this invention.

In some embodiments, the present invention relates to a chimeric protein or an Fc-based chimeric protein complex comprising a polypeptide having an amino acid sequence having at least 90%, or at least 95%, or at least 96%, or at least 97%, or at least 98%, or at least 99% identity with any one of SEQ ID NOs: 290-449, 478-495, or 501-531. In further embodiments, the chimeric protein or Fc-based chimeric protein complex comprises a polypeptide having an amino acid sequence constructed in one or more of Examples 1-25.

### Modified IL-2

In one aspect, the present technology provides a chimeric protein that includes a modified IL-2. In various embodiments, the chimeric protein of the present technology comprises a modified IL-2 as a signaling agent. In various embodiments, the modified IL-2 signaling agent encompasses functional derivatives, analogs, precursors, isoforms, splice variants, or fragments of the modified IL-2. In some embodiments, the modified IL-2 encompasses mutations and/or modifications to SEQ ID NO: 1, which is the amino acid sequence of wild type IL-2. In some embodiments, the modified IL-2 has a reduced binding affinity for its target receptor.

In some embodiments, the modified IL-2 signaling agent has reduced affinity and/or activity for IL-2Rα and/or IL-2Rβ and/or IL-2Rγ. In some embodiments, the modified IL-2 has reduced affinity and/or activity for IL-2Rβ and/or IL-2Rγ. In some embodiments, the modified IL-2 has substantially reduced or ablated affinity and/or activity for IL-2Rα. Such embodiments may be relevant for treatment of cancer, for instance when the modified IL-2 is agonistic at IL-2Rβγ.

In embodiments, the IL-2 has one or more mutations or modifications that biases it away from an IL-2R having an a chain and one or more attenuating mutations that reduces the activity or affinity of the IL-2 in a restorable manner at an IL-2R having only the βγ chain.

In embodiments, the IL-2 has one or more mutations or modifications that biases it away from an IL-2R having only the βγ chain and an attenuating mutation that reduces the activity or affinity of the IL-2 in a restorable manner at an IL-2R having an a chain.

In some embodiments, the IL-2 has one or more mutations or modifications: one or more mutations or modifications that reduce or ablate the activity or affinity of the modified IL-2 for an IL-2Rα chain and/or one or more mutations or modifications that reduce the activity or affinity of the modified IL-2 for an IL-2Rβγ complex. In some embodiments, the reduced activity or affinity of the modified IL-2 for the IL-2Rα chain is restorable by induced proximity at/to a target (e.g., a cell) through a targeting moiety disclosed herein. In some embodiments, the mutation or modification that ablates the activity or affinity of the modified IL-2 for the IL-2Rα chain is not restorable by induced proximity at/to a target (e.g., a cell) through a targeting moiety. In some embodiments, the reduced activity or affinity of the modified IL-2 for the IL-2Rβγ complex is restorable by induced proximity at/to a target (e.g., a cell) through a targeting moiety disclosed herein. In some embodiments, the one or more mutations in reduce the activity or affinity of the modified IL-2 for an IL-2Rα chain and IL-2Rβγ complex, respectively, are both restorable by induced proximity at/to a target (e.g., a cell) through a targeting moiety disclosed herein.

In embodiments, the IL-2 has one or more mutations or modifications: one or more mutations or modifications that substantially ablate the activity or affinity of the modified IL-2 for an IL-2R α chain and/or one or more mutations or modifications that reduce the activity or affinity of the modified IL-2 for IL-2R lacking an a chain, such reduced activity or affinity being restorable by induced proximity at/to a target (e.g., a cell) through a targeting moiety and such IL-2 being substantially inactive at an IL-2R on cells that do not express a target for the targeting moiety or substantially inactive, compared to wild type IL-2, at IL-2R comprising the alpha chain when contacting cells that express the target for the targeting moiety, while being active at IL-2R lacking an a chain and/or cells that express the target for the targeting moiety. In embodiments, the activity or affinity from mutation or modification is about 3- or about 5-, or about 10-, or about 30-, or about 100-, or about 300-, or about 500-, or about 1000-fold reduced as compared to the activity or affinity from a different mutation or modification.

In embodiments, the IL-2 has one or more mutations or modifications: one or more mutations or modifications that reduce the activity or affinity of the modified IL-2 for IL-2Rαβγ and/or one or more mutations or modifications that reduce the activity or affinity of the modified IL-2 for IL-2Rα, such reduced activity or affinity being restorable by induced proximity at/to a target (e.g., a cell) through a targeting moiety.

In embodiments, the IL-2 has one or more mutations or modifications: one or more mutations or modifications that reduce the activity or affinity of the modified IL-2 for IL-2Rαβγ and/or one or more mutations or modifications that reduce the activity or affinity of the modified IL-2 for IL-2Rβγ, such reduced activity or affinity being restorable by induced proximity at/to a target (e.g., a cell) through a targeting moiety and such IL-2 being substantially inactive at an IL-2R on cells that do not express a target for the targeting moiety or substantially inactive at IL-2Rαβγ, compared to wild type IL-2, when contacting cells that express the target for the targeting moiety, while being active at IL-2Rβγ and/or cells that express the target for the targeting moiety.

In embodiments, the IL-2 has selective (e.g., biased for and/or strongly favored) action at IL-2Rβγ (and not IL-2Rαβγ) but the activity at IL-2Rβγ is attenuated by further mutations or modifications to render the IL-2 safe for systemic use (e.g. the IL-2 is active at IL-2Rβγ predominantly at the site of therapeutic action, to which it is directed via the targeting moiety).

In embodiments, the IL-2 has selective (e.g., biased for and/or strongly favored) action at NK cells and/or CD8⁺ cells and not substantially at Treg cells and the selective (e.g., biased for and/or strongly favored) action at NK cells and/or CD8⁺ cells is controlled by targeting to reduce or eliminate systemic side effects. For instance, the present chimeric protein is, in some embodiments, not only directed to cells that have an immune stimulation effect but also done so in a controllable way that focuses IL-2 action at the site of desired therapy. As described herein, such chimeric proteins find use, in embodiments, in safely stimulating the immune system for an anti-cancer effect. In some embodiments, the IL-2 has one or more mutations or modifications: one or more mutations or modifications that reduce the activity or affinity of the modified IL-2 for an IL-2R lacking an a chain and/or one or more mutations or modifications reduce the activity or affinity of the modified IL-2 for an IL-2R comprising an a chain, such mutation or modification being an attenuation mutation for which the activity or affinity is restorable by induced proximity at/to a target (e.g., a cell) through a targeting moiety.

In embodiments, the IL-2 has one or more mutations or modifications: one or more mutations or modifications that reduce the activity or affinity of the modified IL-2 for an IL-2R lacking an a chain and/or one or more mutations or modifications that reduce the activity or affinity of the modified IL-2 for IL-2R comprising an a chain, such reduced activity or affinity being restorable by induced proximity at/to a target (e.g., a cell) through a targeting moiety and such IL-2 being substantially inactive at an IL-2R on cells that do not express a target for the targeting moiety or substantially inactive, compared to wild type IL-2, at IL-2R lacking an a chain when contacting cells that express the target for the targeting moiety, while being active at an IL-2R comprising an a chain and/or cells that express the target for the targeting moiety.

In some embodiments, the IL-2 has one or more mutations or modifications: one or more mutations or modifications that reduce the activity or affinity of the modified IL-2 for IL-2Rβγ and/or one or more mutations or modifications that reduce the activity or affinity of the modified IL-2 for IL-2Rα such mutation or modification being an attenuation mutation for which the activity or affinity is restorable by induced proximity at/to a target (e.g., a cell) through a targeting moiety.

In embodiments, the IL-2 has one or more mutations or modifications: one or more mutations or modifications that reduce the activity or affinity of the modified IL-2 for IL-2Rβγ and/or one or more mutations or modifications that reduce the activity or affinity of the modified IL-2 for IL-2Rαβγ, such reduced activity or affinity being restorable by induced proximity at/to a target (e.g., a cell) through a targeting moiety.

In embodiments, the IL-2 has one or more mutations or modifications: one or more mutations or modifications that reduce the activity or affinity of the modified IL-2 for IL-2Rβγ and/or one or more mutations or modifications that reduce the activity or affinity of the modified IL-2 for IL-2Rαβγ, such reduced activity or affinity being restorable by induced proximity at/to a target (e.g., a cell) through a targeting moiety and such IL-2 being substantially inactive at an IL-2R on cells that do not express a target for the targeting moiety or substantially inactive at IL-2Rβγ, compared to wild type IL-2, when contacting cells that express the target for the targeting moiety, while being active at IL-2Rαβγ and/or cells that express the target for the targeting moiety.

In embodiments, the modified IL-2 has selective (e.g., biased for and/or strongly favored) action at IL-2Rαβγ (and not IL-2Rβγ) but the activity at IL-2Rαβγ is attenuated by further mutations or modifications to render the IL-2 safe for systemic use (e.g. the IL-2 is active at IL-2Rαβγ predominantly at the site of therapeutic action, to which it is directed via the targeting moiety).

In some embodiments, the IL-2 has one or more mutations or modifications that reduce the activity or affinity of the modified IL-2 for an IL-2Rβγ chain and normal activity or affinity to an IL-2Rα chain, wherein the reduced activity or affinity of the modified IL-2 for the IL-2Rβγ chain is restorable by induced proximity at/to a target (e.g., a cell) through a targeting moiety.

In embodiments, the IL-2 has one or more mutations or modifications that reduce the activity or affinity of the modified IL-2 for IL-2Rα and normal activity or affinity to an IL-2Rβγ chain, wherein the reduced activity or affinity of the modified IL-2 for the IL-2Rα chain is restorable by induced proximity at/to a target (e.g., a cell) through a targeting moiety.

In some embodiments, the IL-2 has one or more mutations or modifications: one or more mutations or modifications that ablate the activity or affinity of the modified IL-2 for an IL-2Rα chain and/or one or more mutations or modifications that reduce the activity or affinity of the modified IL-2 for an IL-2Rβγ chain, wherein the reduced activity or affinity of the modified IL-2 for the IL-2Rβγ chain is restorable by induced proximity at/to a target (e.g., a cell) through a targeting moiety (e.g., a CD8, CD3, or PD-1 targeting moiety) and is useful in mediating a T-cell mediated immune response (e.g., a CD8 T-cell mediated immune response).

In some embodiments, the IL-2 has one or more mutations or modifications: one or more mutations or modifications that reduce the activity or affinity of the modified IL-2 for an IL-2Rα chain and/or one or more mutations or modifications that reduce the activity or affinity of the modified IL-2 for an IL-2Rβγ chain, wherein the reduced activity or affinity of the modified IL-2 for the IL-2Rα and/or the IL-2Rβγ chain is restorable by induced proximity at/to a target (e.g., a cell) through a targeting moiety (e.g., a CD8, CD3, or PD-1 targeting moiety) and is useful in mediating a T-cell mediated immune response (e.g., a CD8 T-cell mediated immune response).

In some embodiments, the IL-2 has one or more mutations or modifications: one or more mutations or modifications that reduce the activity or affinity of the modified IL-2 for an IL-2Rα chain and/or one or more mutations or modifications that reduce the activity or affinity of the modified IL-2 for an IL-2Rβγ chain, wherein the reduced activity or affinity of the modified IL-2 for the IL-2Rα chain and/or IL-2Rβγ chain is restorable by induced proximity at/to a target (e.g., a cell) through a targeting moiety and is useful in mediating a Treg response.

In some embodiments, the IL-2 has one or more mutations or modifications: one or more mutations or modifications that ablate the activity or affinity of the modified IL-2 for an IL-2Rα chain and/or one or more mutations or modifications that reduce the activity or affinity of the modified IL-2 for an IL-2Rβγ chain, wherein the reduced activity or affinity of the modified IL-2 for the IL-2Rβγ chain is restorable by induced proximity at/to a target (e.g., a cell) through a targeting moiety and is useful in mediating a Treg response.

In some embodiments, the IL-2 has one or more mutations or modifications that reduce the activity or affinity of the modified IL-2 for an IL-2Rβγ chain and normal activity or affinity to an IL-2Rα chain, wherein the reduced activity or affinity of the modified IL-2 for the IL-2Rβγ chain is restorable by induced proximity at/to a target (e.g., a cell) through a targeting moiety and is useful in mediating a Treg response.

In embodiments, the IL-2 has selective (e.g., biased for and/or strongly favored) action at Treg cells and not substantially at NK cells and/or CD8⁺ cells and the selective (e.g., biased for and/or strongly favored) action at Treg cells is controlled by targeting to reduce or eliminate systemic side effects. For instance, the present chimeric protein is, in some embodiments, not only directed to cells that have an immune inhibition effect but also done so in a controllable way that focuses IL-2 action at the site of desired therapy. As described herein, such chimeric proteins find use, in embodiments, in safely stimulating the immune system for an anti-autoimmune effect.

In some embodiments, the modified IL-2 signaling agent is modified wild type IL-2. Wild type IL-2 has the amino acid sequence of:

(SEQ ID NO: 1)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT.

In some embodiments, the modified IL-2 signaling agent comprises: (i) a mutation selected from D20E, D20F, D20G, D20H, D20I, D20K, D20L, and D20V; or (ii) a mutation selected from N88G, N88E, N88K, N88Q, and N88V; or (iii) a mutation selected from Q126G, Q126A, Q126E, Q126F, Q126H, Q126I, Q126K, Q126L, Q126N, Q126P, Q126R, Q126S, Q126T, Q126V, Q126W, and Q126Y; or (iv) a mutation selected from (i) and (iii) or a mutation selected from (ii) and (iii); or (v) one or more mutations selected from: R38A/F42K/N88G/C125A, F42K/C125A, D20E/C125A, D20F/C125A, D20G/C125A, D20H/C125A, D20I/C125A, D20K/C125A, D20L/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, N88V/C125A, D20E/R38A/F42K, D20E/R38A/F42K/C125A, D20V/R38A/F42K, D20V/R38A/F42K/C125A, R38A/F42Y/E62A/C125A, R38A/F42Y/Y45A/E62A, F42Y/Y45A/L72G, R38A/F42Y/Y45A/E62A/C125A, F42Y/Y45A/L72G/C125A, R38A/F42K/N88G, R38A/F42K/Q126G, R38A/F42K/Q126I, R38A/F42K/Q126Y, R38A/F42K/Q126G/C125A, R38A/F42K/Q126I/C125A, R38A/F42K/Q126Y/C125A, R38A/F42K/E62A/N88G, and R38A/F42K/E62A/N88G/C125A.

In some embodiments, the modified IL-2 signaling agent comprises one or more mutations at an amino acid residue selected from R38, F42, E62, C125, Y45, L72, D20, N88, and Q126 relative to SEQ ID NO: 1. In such embodiments, the modified IL-2 signaling agent comprises one or more mutations selected from R38A, F42K, F42Y, E62A, C125A, Y45A, L72G, D20E, D20F, D20G, D20H, D20I, D20K, D20L, D20N, D20V, N88G, N88E, N88I, N88K, N88Q, N88R, N88V, R38A/F42Y/E62A/C125A, F42Y/Y45A/L72G/C125A, F42K/C125A, R38A/F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K/C125A, D20V/R38A/F42K/C125A, R38A/F42K/N88A/C125A, R38A/F42K/N88G/C125A, Q126A, Q126E, Q126F, Q126G, Q126H, Q126I, Q126K, Q126L, Q126N, Q126P, Q126R, Q126S, Q126T, Q126V, Q126W, and Q126Y, relative to SEQ ID NO: 1.

In various embodiments, the modified IL-2 signaling agent comprises one or more mutations selected from D20V, D20E, N88G, F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, R38A/F42K/E62A/N88G/C125A, Q126A, Q126E, Q126F, Q126G, Q126H, Q126I, Q126K, Q126L, Q126N, Q126P, Q126R, Q126S, Q126T, Q126V, Q126W, and Q126Y, relative to SEQ ID NO: 1.

In particular embodiments, the modified IL-2 signaling agent comprises one or more mutations selected from R38A/F42Y/E62A/C125A, F42Y/Y45A/L72G/C125A, F42K/C125A, D20E/C125A, D20G/C125A, D20S/C125A, D20T/C125A, D20V/C125A, N88A/C125A, N88D/C125A, N88G/C125A, N88H/C125A, N88Q/C125A, N88T/C125A, D20E/R38A/F42K/C125A, D20V/R38A/F42K/C125A, and R38A/F42K/N88G/C125A, relative to SEQ ID NO: 1.

In some embodiments, the modified IL-2 signaling agent comprises one or more of the mutation combinations selected from those listed in Table 8.

Without wishing to be bound by theory, it is believed that modified IL-2 agents have reduced affinity for a high-affinity IL-2 receptor (i.e., IL-2Rαβγ) and preserves affinity to the intermediate-affinity IL-2 receptor (i.e., IL-2Rβγ), as compared to the wild-type IL-2.

Without wishing to be bound by theory, it is further believed that these modified IL-2 agents have reduced affinity for an IL-2 receptor (e.g., IL-2Rβγ), as compared to the wild-type IL-2.

In some embodiments, the modified IL-2 signaling agent with mutations at R38, F42, Y45, and/or E62 is able to induce an expansion of effector cells including CD8⁺ T cells and NK cells but not Treg cells and/or endothelial cells. In some embodiments, the modified IL-2 signaling agent with mutations at R38, F42, Y45, and/or E62 is less toxic than wildtype IL-2 agents. A chimeric protein comprising the modified IL-2 agent with substantially reduced affinity and/or activity for IL-2Rα may find application in oncology, for example.

In some embodiments, the modified IL-2 agent has a mutation at amino acid residue D20. For example, the modified IL-2 agent may comprise D20E, D20G, D20S, D20T, or D20V. In some embodiments, the modified IL-2 agent with a mutation at D20 has decreased toxicity as compared to wild type IL-2 agents. In some embodiments, a D20 mutation decreases toxicity by inhibiting binding to and activation of endothelial cells.

In some embodiments, the modified IL-2 agent has a mutation at amino acids C125. For example, the modified IL-2 agent may comprise the mutation C125A. In some embodiments, the modified IL-2 agent with a mutation at C125 optimize production of such a modified IL-2 agent. In some embodiments, a mutation at C125 promotes stability of such a modified IL-2 agent by removing an unpaired cysteine residue. In such embodiments, the modified IL-2 agent may have substantially reduced affinity and/or activity for IL-2Rα, as described in, for example, Carmenate et al. (2013) The Journal of Immunology, 190:6230-6238, the entire disclosure of which is hereby incorporated by reference.

In other embodiments, the modified IL-2 agent may have substantially reduced affinity and/or activity for IL-2Rβ, as described in, for example, WO 2016/025385, the entire disclosure of which is hereby incorporated by reference. In such embodiments, the modified IL-2 agent may induce an expansion of Treg cells but not effector cells such as CD8⁺ T cells and NK cells. A chimeric protein comprising the modified IL-2 agent with substantially reduced affinity and/or activity for IL-2Rβ may find application in the treatment of autoimmune disease for example. In some embodiments, the modified IL-2 agent may comprise a mutation at amino acid residue N88. For example, the modified IL-2 agent may comprise N88A, N88D, N88G, N88H, N88Q, or N88T.

In some embodiments, the modified IL-2 signaling agent comprises a deletion of the Ala at the N-terminus of SEQ ID NO: 1. In some embodiments, the modified IL-2 agent comprises the substitution of a Serine or Alanine for the Cysteine at position 125 of SEQ ID NO: 1. In some embodiments, the modified IL-2 agent comprises a deletion of the Ala at the N-terminus and the substitution of a Serine or Alanine for the Cysteine at position 125 of SEQ ID NO: 1.

In some embodiments, the modified IL-2 exhibits reduced affinity and/or activity for IL-2Rα and IL2Rβ, optionally wherein the modified IL-2 comprises one or more mutations selected from D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, and R38A/F42K/E62A/N88G/C125A.

In some embodiments, the modified IL-2 exhibits reduced affinity and/or activity for IL-2Rα and IL-2Rγ, optionally wherein the modified IL-2 comprises one or more mutations selected from R38A/F42K/Q126G, R38A/F42K/Q126I, and R38A/F42K/Q126Y.

In some embodiments, the modified IL-2 exhibits reduced affinity and/or activity for IL-2Rβ, optionally wherein the modified IL-2 comprises one or more mutations selected from D20E, D20F, D20G, D20H, D20I, D20K, D20L, D20V, N88G, N88E, N88K, N88Q, N88V, D20E/C125A, D20F/C125A, D20G/C125A, D20H/C125A, D20I/C125A, D20L/C125A, D20K/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, and N88V/C125A.

In some embodiments, the modified IL-2 exhibits reduced affinity and/or activity for IL-2Rγ, optionally wherein the modified IL-2 comprises one or more mutations selected from Q126A, Q126E, Q126F, Q126G, Q126H, Q126I, Q126K, Q126L, Q126N, Q126P, Q126R, Q126S, Q126T, Q126V, Q126W, and Q126Y.

In some embodiments, the modified IL-2 exhibits reduced affinity and/or activity for IL-2Rβγ, optionally wherein the modified IL-2 comprises one or more mutations selected from D20E/Q126A, D20E/Q126D, D20E/Q126E, D20E/Q126F, D20E/Q126G, D20E/Q126H, D20E/Q126I, D20E/Q126K, D20E/Q126L, D20E/Q126M, D20E/Q126N, D20E/Q126P, D20E/Q126R, D20E/Q126S, D20E/Q126T, D20E/Q126V, D20E/Q126W, D20E/Q126Y, D20V/Q126A, D20V/Q126D, D20V/Q126E, D20V/Q126F, D20V/Q126G, D20V/Q126H, D20V/Q126I, D20V/Q126K, D20V/Q126L, D20V/Q126M, D20V/Q126N, D20V/Q126P, D20V/Q126R, D20V/Q126S, D20V/Q126T, D20V/Q126V, D20V/Q126W, D20V/Q126Y, N88A/Q126A, N88A/Q126D, N88A/Q126E, N88A/Q126F, N88A/Q126G, N88A/Q126H, N88A/Q126I, N88A/Q126K, N88A/Q126L, N88A/Q126M, N88A/Q126N, N88A/Q126P, N88A/Q126R, N88A/Q126S, N88A/Q126T, N88A/Q126V, N88A/Q126W, N88A/Q126Y, N88G/Q126A, N88G/Q126D, N88G/Q126E, N88G/Q126F, N88G/Q126G, N88G/Q126H, N88G/Q126I, N88G/Q126K, N88G/Q126L, N88G/Q126M, N88G/Q126N, N88G/Q126P, N88G/Q126R, N88G/Q126S, N88G/Q126T, N88G/Q126V, N88G/Q126W, and N88G/Q126Y. In further embodiments, the modified IL-2 exhibits reduced affinity and/or activity for IL-2Rβγ and exhibits increased selectivity toward activated CD8 cells.

### Neoleukin

In some embodiments, the modified IL-2 signaling agent is modified neoleukin, which comprises the amino acid sequence of:

(SEQ ID NO: 2)
PKKKIQLHAEHALYDALMILNIVKTNSPPAEEKLEDYAFNFELILEEIAR
LFESGDQKDEAEKAKRMKEWMKRIKTTASEDEQEEMANAIITILQSWIF
S.

In such embodiments, the modified IL-2 signaling agent comprising SEQ ID NO: 2 has one or more mutations at amino acid residues D15 and/or N40. For example, in some embodiments, the modified IL-2 signaling agent comprising SEQ ID NO: 2 comprises D15T, D15H, N401, N40G, or N40R.

In some embodiments, the modified IL-2 signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NOs: 1 or 2 (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments, the modified IL-2 is modified to have one or more mutations. In some embodiments, the modified IL-2 is modified to have one or more mutations and one or more modifications (e.g., glycosylation). IL-2 contains a threonine O-glycosylation site on position 3 (T3). For example, in some embodiments the modified IL-2-signaling agent comprises a T3 O-glycosylation mutation, wherein the mutation is one of T3A, T3F, T3H, T3L,

T3V, and T3Y. In some embodiments, the modified IL-2-signaling agent comprises a T3 O-glycosylation deletion mutation, wherein the first 3, 4, 5, 6, 7, or 8 residues of the modified IL-2 sequence are deleted.

In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In some embodiments, the mutations allow for the modified IL-2 to have one or more of attenuated activity, such as one or more of reduced binding affinity, reduced endogenous activity, and reduced specific bioactivity, relative to unmutated IL-2, e.g., the wild type form of IL-2 (e.g., SEQ ID NO: 1). For instance, the one or more of attenuated activity, such as reduced binding affinity, reduced endogenous activity, and reduced specific bioactivity, relative to unmutated IL-2, e.g., the wild type form of IL-2 (e.g., SEQ ID NO: 1), may be at a receptor such as IL-2Rαβγ. Consequentially, in various embodiments, the mutations allow for the modified IL-2 to have reduced systemic toxicity, reduced side effects, and reduced off-target effects relative to unmutated IL-2, e.g., the wild type form of IL-2.

In some embodiments, the modified IL-2 is modified to have one or more mutations that reduces its binding affinity or activity at a therapeutic or target receptor. In some embodiments, the activity provided by the modified IL-2 is agonism at the therapeutic or target receptor (e.g. activation of a cellular effect at a site of therapy) or antagonism at the therapeutic or target receptor (e.g., reduced or ablated activation of a cellular effect at a site of therapy). For example, the modified IL-2 may activate the therapeutic or target receptor. In such embodiments, the mutation results in the modified IL-2 to have reduced activating activity at the therapeutic or target receptor.

In some embodiments, the reduced affinity or activity at the therapeutic or target receptor is restorable by attachment with a targeting moiety. In other embodiments, the reduced affinity or activity at the therapeutic or target receptor is not substantially restorable by attachment with the targeting moiety. In various embodiments, the chimeric proteins of the present technology reduce off-target effects because the modified IL-2 has mutations that weaken binding affinity or activity at a therapeutic or target receptor. In various embodiments, this reduces side effects observed with, for example, the wild type IL-2. In various embodiments, the modified IL-2 is substantially inactive en route to the site of therapeutic activity and has its effect substantially on specifically targeted cell types which greatly reduces undesired side effects.

In various embodiments, the modified IL-2 has one or more mutations that cause the modified IL-2 to have attenuated or reduced affinity, e.g. binding (e.g. K_{D}) and/or activation (measurable as, for example, KA and/or EC50) for one or more therapeutic receptors. In various embodiments, the reduced affinity at the therapeutic receptor allows for attenuation of activity and/or signaling from the therapeutic receptor.

In various embodiments, the modified IL-2 has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the therapeutic or target receptor (e.g., IL-2Rαβγ or IL-2Rβγ or any one of their subunits IL-2Rα, IL-2Rβ, and/or IL-2Rγ) relative to the wild type IL-2. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type IL-2.

Receptor binding activity may be measured using methods known in the art. For example, affinity and/or binding activity may be assessed by Scatchard plot analysis and computer-fitting of binding data (e.g. Scatchard, The attractions of proteins for small molecules and ions. Ann NY Acad Sci 51: 660-672, 1949) or by reflectometric interference spectroscopy under flow through conditions, as described by Brecht et al. Biosens Bioelectron 1993; 8:387-392, the entire contents of all of which are hereby incorporated by reference. In some embodiments, receptor binding activity is measured by bio-layer interferometry (BLI).

In various embodiments, the attenuated activity at the therapeutic or target receptor, the weakened affinity at the therapeutic or target receptor is restorable by attachment with a targeting moiety, having high affinity for an antigen at the site of therapeutic activity (e.g. an antibody or antibody format described herein). The targeting is realized by linking the modified IL-2 to a targeting moiety. In an embodiment, the modified IL-2 is linked to a targeting moiety through its amino-terminus. In another embodiment, the modified IL-2 is linked to a targeting moiety through its carboxy-terminus. In this way, the present chimeric proteins provide, in some embodiments, localized, on-target, and controlled therapeutic action at the therapeutic or target receptor.

In various embodiments, the activity of the modified IL-2 at a therapeutic or target receptor, is enhanced by attachment with a targeting moiety, having high affinity for an antigen at the site of therapeutic activity (e.g. an antibody or antibody format described herein). The targeting is realized by linking the modified IL-2 to a targeting moiety. In an embodiment, the modified IL-2 is linked to a targeting moiety through its amino-terminus. In another embodiment, the modified IL-2 is linked to a targeting moiety through its carboxy-terminus. In this way, the present chimeric proteins provide, in some embodiments, localized, on-target, and controlled therapeutic action at the therapeutic or target receptor.

### Therapeutic Agents Comprising the Interleukin or a Variant Thereof

In embodiments, the present invention provides chimeric proteins or protein complexes comprising one or more targeting moieties that bind to one or more of the following targets: CD8, CTLA4, CD3, CD4, DNAM-1, Nrp1 (neurophilin), TNFR1, TNFR2, GITR, ICOS, CD20, CD70, Clec9a, NKp46, PD-1, PD-L1, PD-L2, SIRP1a, FAP, XCR1, tenascin, and ECM proteins.

### Targeting Moiety Cellular Recruitment

In various embodiments, the chimeric proteins or chimeric protein complexes, such as Fc-based chimeric protein complexes of the present invention additionally comprise one or more targeting moieties having recognition domains which specifically bind to a target (e.g. antigen, receptor) of interest. In some embodiments, the chimeric protein or chimeric protein complexes, such as Fc-based chimeric protein complexes may comprise two, three, four, five, six, seven, eight, nine, ten or more targeting moieties. In illustrative embodiments, the chimeric proteins or chimeric protein complexes, such as Fc-based chimeric protein complexes of the invention comprise two or more targeting moieties. In such embodiments, the chimeric proteins or chimeric protein complexes, such as Fc-based chimeric protein complexes can target two different cells (e.g. to make a synapse) or the same cell (e.g. to get a more concentrated signaling agent effect).

In some embodiments, the target (e.g. antigen, receptor) of interest is directed against an immune cell and/or organ cells, and/or tissue cells.

In various embodiments, the target (e.g. antigen, receptor) of interest can be found on one or more immune cells, which can include, without limitation, T cells, cytotoxic T lymphocytes, T helper cells, natural killer (NK) cells, natural killer T (NKT) cells, anti-tumor or tumor-associated macrophages (e.g. M1 or M2 macrophages), B cells, Breg cells, dendritic cells, or subsets thereof. In some embodiments, the recognition domains specifically bind to a target (e.g. antigen, receptor) of interest and effectively, directly or indirectly, recruit one of more immune cells. In some embodiments, the target (e.g. antigen, receptor) of interest can be found on one or more tumor cells. In some embodiments, the target (e.g. antigen, receptor) of interest can be found on or in the tumor neovasculature. In some embodiments, the present chimeric proteins or chimeric protein complexes, such as Fc-based chimeric protein complexes may directly or indirectly recruit an immune cell, e.g., in some embodiments, to a therapeutic site (e.g. a locus with one or more disease cell or cell to be modulated for a therapeutic effect). In some embodiments, the present chimeric proteins or chimeric protein complexes such as Fc-based chimeric protein complexes may directly or indirectly recruit an immune cell, e.g. an immune cell that can kill and/or suppress a tumor cell, to a site of action (such as, by way of non-limiting example, the tumor microenvironment).

In various embodiments, the chimeric proteins or chimeric protein complexes such as Fc-based chimeric protein complexes have targeting moieties having recognition domains which specifically bind to a target (e.g. antigen, receptor) which is part of a non-cellular structure. In some embodiments, the antigen or receptor is not an integral component of an intact cell or cellular structure. In some embodiments, the antigen or receptor is an extracellular antigen or receptor. In some embodiments, the target is a non-proteinaceous, non-cellular marker, including, without limitation, nucleic acids, inclusive of DNA or RNA, such as, for example, DNA released from necrotic tumor cells or extracellular deposits such as cholesterol.

In some embodiments, the target (e.g. antigen, receptor) of interest is part of the non-cellular component of the stroma or the extracellular matrix (ECM) or the markers associated therewith. As used herein, stroma refers to the connective and supportive framework of a tissue or organ. Stroma may include a compilation of cells such as fibroblasts/myofibroblasts, glial, epithelia, fat, immune, vascular, smooth muscle, and immune cells along with the extracellular matrix (ECM) and extracellular molecules. In various embodiments, the target (e.g. antigen, receptor) of interest is part of the non-cellular component of the stroma such as the extracellular matrix and extracellular molecules. As used herein, the ECM refers to the non-cellular components present within all tissues and organs. The ECM is composed of a large collection of biochemically distinct components including, without limitation, proteins, glycoproteins, proteoglycans, and polysaccharides. These components of the ECM are usually produced by adjacent cells and secreted into the ECM via exocytosis. Once secreted, the ECM components often aggregate to form a complex network of macromolecules. In various embodiments, the chimeric proteins or chimeric protein complexes such as Fc-based chimeric protein chimeric proteins of the invention comprise a targeting moiety that recognizes a target (e.g., an antigen or receptor or non-proteinaceous molecule) located on any component of the ECM. Illustrative components of the ECM include, without limitation, the proteoglycans, the non-proteoglycan polysaccharides, fibers, and other ECM proteins or ECM non-proteins, e.g. polysaccharides and/or lipids, or ECM associated molecules (e.g. proteins or non-proteins, e.g. polysaccharides, nucleic acids and/or lipids).

In some embodiments, the targeting moiety recognizes a target (e.g. antigen, receptor) on ECM proteoglycans. Proteoglycans are glycosylated proteins. The basic proteoglycan unit includes a core protein with one or more covalently attached glycosaminoglycan (GAG) chains. Proteoglycans have a net negative charge that attracts positively charged sodium ions (Na+), which attracts water molecules via osmosis, keeping the ECM and resident cells hydrated. Proteoglycans may also help to trap and store growth factors within the ECM. Illustrative proteoglycans that may be targeted by the chimeric proteins or chimeric protein complexes such as Fc-based chimeric protein complexes of the invention include, but are not limited to, heparan sulfate, chondroitin sulfate, and keratan sulfate. In an embodiment, the targeting moiety recognizes a target (e.g. antigen, receptor) on non-proteoglycan polysaccharides such as hyaluronic acid.

In some embodiments, the targeting moiety recognizes a target (e.g. antigen, receptor) on ECM fibers. ECM fibers include collagen fibers and elastin fibers. In some embodiments, the targeting moiety recognizes one or more epitopes on collagens or collagen fibers. Collagens are the most abundant proteins in the ECM. Collagens are present in the ECM as fibrillar proteins and provide structural support to resident cells. In one or more embodiments, the targeting moiety recognizes and binds to various types of collagens present within the ECM including, without limitation, fibrilar collagens (types I, II, III, V, XI), facit collagens (types IX, XII, XIV), short chain collagens (types VIII, X), basement membrane collagens (type IV), and/or collagen types VI, VII, or XIII. Elastin fibers provide elasticity to tissues, allowing them to stretch when needed and then return to their original state. In some embodiments, the target moiety recognizes one or more epitopes on elastins or elastin fibers.

In some embodiments, the targeting moiety recognizes one or more ECM proteins including, but not limited to, a tenascin, a fibronectin, a fibrin, a laminin, or a nidogen/entactin.

In an embodiment, the targeting moiety recognizes and binds to tenascin. The tenascin (TN) family of glycoproteins includes at least four members, tenascin-C, tenascin-R, tenascin-X, and tenascin W. The primary structures of tenascin proteins include several common motifs ordered in the same consecutive sequence: amino-terminal heptad repeats, epidermal growth factor (EGF)-like repeats, fibronectin type III domain repeats, and a carboxyl-terminal fibrinogen-like globular domain. Each protein member is associated with typical variations in the number and nature of EGF-like and fibronectin type III repeats. Isoform variants also exist particularly with respect to tenascin-C. Over 27 splice variants and/or isoforms of tenascin-C are known. In a particular embodiment, the targeting moiety recognizes and binds to tenascin-CA1. Similarly, tenascin-R also has various splice variants and isoforms. Tenascin-R usually exists as dimers or trimers. Tenascin-X is the largest member of the tenascin family and is known to exist as trimers. Tenascin-W exists as trimers. In some embodiments, the targeting moiety recognizes one or more epitopes on a tenascin protein. In some embodiments, the targeting moiety recognizes the monomeric and/or the dimeric and/or the trimeric and/or the hexameric forms of a tenascin protein.

In an embodiment, the targeting moieties recognize and bind to fibronectin. Fibronectins are glycoproteins that connect cells with collagen fibers in the ECM, allowing cells to move through the ECM. Upon binding to integrins, fibronectins unfold to form functional dimers. In some embodiments, the targeting moiety recognizes the monomeric and/or the dimeric forms of fibronectin. In some embodiments, the targeting moiety recognizes one or more epitopes on fibronectin. In illustrative embodiments, the targeting moiety recognizes fibronectin extracellular domain A (EDA) or fibronectin extracellular domain B (EDB). Elevated levels of EDA are associated with various diseases and disorders including psoriasis, rheumatoid arthritis, diabetes, and cancer. In some embodiments, the targeting moiety recognizes fibronectin that contains the EDA isoform and may be utilized to target the chimeric proteins or chimeric protein complexes such as Fc-based chimeric protein complexes to diseased cells including cancer cells. In some embodiments, the targeting moiety recognizes fibronectin that contains the EDB isoform. In various embodiments, such targeting moieties may be utilized to target the chimeric proteins or chimeric protein complexes such as Fc-based chimeric protein complexes to tumor cells including the tumor neovasculature.

In an embodiment, the targeting moiety recognizes and binds to fibrin. Fibrin is another protein substance often found in the matrix network of the ECM. Fibrin is formed by the action of the protease thrombin on fibrinogen which causes the fibrin to polymerize. In some embodiments, the targeting moiety recognizes one or more epitopes on fibrin. In some embodiments, the targeting moiety recognizes the monomeric as well as the polymerized forms of fibrin.

In an embodiment, the targeting moiety recognizes and binds to laminin. Laminin is a major component of the basal lamina, which is a protein network foundation for cells and organs. Laminins are heterotrimeric proteins that contain an α-chain, a β-chain, and a γ-chain. In some embodiments, the targeting moiety recognizes one or more epitopes on laminin. In some embodiments, the targeting moiety recognizes the monomeric, the dimeric as well as the trimeric forms of laminin.

In an embodiment, the targeting moiety recognizes and binds to a nidogen or entactin. Nidogens/entactins are a family of highly conserved, sulfated glycoproteins. They make up the major structural component of the basement membranes and function to link laminin and collagen IV networks in basement membranes. Members of this family include nidogen-1 and nidogen-2. In various embodiments, the targeting moiety recognizes an epitope on nidogen-1 and/or nidogen-2.

In various embodiments, the targeting moiety comprises an antigen recognition domain that recognizes an epitope present on any of the targets described herein. In an embodiment, the antigen-recognition domain recognizes one or more linear epitopes present on the protein. As used herein, a linear epitope refers to any continuous sequence of amino acids present on the protein. In another embodiment, the antigen-recognition domain recognizes one or more conformational epitopes present on the protein. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the targeting moiety may bind to the full-length and/or mature forms and/or isoforms and/or splice variants and/or fragments and/or any other naturally occurring or synthetic analogs, variants, or mutants of any of the targets described herein. In various embodiments, the targeting moiety may bind to any forms of the proteins described herein, including monomeric, dimeric, trimeric, tetrameric, heterodimeric, multimeric and associated forms. In various embodiments, the targeting moiety may bind to any post-translationally modified forms of the proteins described herein, such as glycosylated and/or phosphorylated forms.

In various embodiments, the targeting moiety comprises an antigen recognition domain that recognizes extracellular molecules such as DNA. In some embodiments, the targeting moiety comprises an antigen recognition domain that recognizes DNA. In an embodiment, the DNA is shed into the extracellular space from necrotic or apoptotic tumor cells or other diseased cells.

In various embodiments, the targeting moiety comprises an antigen recognition domain that recognizes one or more non-cellular structures associated with atherosclerotic plaques. Two types of atherosclerotic plaques are known. The fibro-lipid (fibro-fatty) plaque is characterized by an accumulation of lipid-laden cells underneath the intima of the arteries. Beneath the endothelium there is a fibrous cap covering the atheromatous core of the plaque. The core includes lipid-laden cells (macrophages and smooth muscle cells) with elevated tissue cholesterol and cholesterol ester content, fibrin, proteoglycans, collagen, elastin, and cellular debris. In advanced plaques, the central core of the plaque usually contains extracellular cholesterol deposits (released from dead cells), which form areas of cholesterol crystals with empty, needle-like clefts. At the periphery of the plaque are younger foamy cells and capillaries. A fibrous plaque is also localized under the intima, within the wall of the artery resulting in thickening and expansion of the wall and, sometimes, spotty localized narrowing of the lumen with some atrophy of the muscular layer. The fibrous plaque contains collagen fibers (eosinophilic), precipitates of calcium (hematoxylinophilic) and lipid-laden cells. In some embodiments, the targeting moiety recognizes and binds to one or more of the non-cellular components of these plaques such as the fibrin, proteoglycans, collagen, elastin, cellular debris, and calcium or other mineral deposits or precipitates. In some embodiments, the cellular debris is a nucleic acid, e.g. DNA or RNA, released from dead cells.

In various embodiments, the targeting moiety comprises an antigen recognition domain that recognizes one or more non-cellular structures found in the brain plaques associated with neurodegenerative diseases. In some embodiments, the targeting moiety recognizes and binds to one or more non-cellular structures located in the amyloid plaques found in the brains of patients with Alzheimer's disease. For example, the targeting moiety may recognize and bind to the peptide amyloid beta, which is a major component of the amyloid plaques. In some embodiments, the targeting moiety recognizes and binds to one or more non-cellular structures located in the brains plaques found in patients with Huntington's disease. In various embodiments, the targeting moiety recognizes and binds to one or more non-cellular structures found in plaques associated with other neurodegenerative or musculoskeletal diseases such as Lewy body dementia and inclusion body myositis.

In some embodiments, the chimeric proteins or chimeric protein complexes such as Fc-based chimeric protein complexes of the invention may have two or more targeting moieties that bind to non-cellular structures. In some embodiments, there are two targeting moieties and one targets a cell while the other targets a non-cellular structure. In various embodiments, the targeting moieties can directly or indirectly recruit cells, such as disease cells and/or effector cells. In some embodiments, the present chimeric proteins or chimeric protein complexes such as Fc-based chimeric protein complexes are capable of, or find use in methods involving, shifting the balance of immune cells in favor of immune attack of a tumor. For instance, the present chimeric proteins or chimeric protein complexes such as Fc-based chimeric protein complexes can shift the ratio of immune cells at a site of clinical importance in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, the present chimeric proteins or chimeric protein complexes such as Fc-based chimeric protein complexes are capable of increasing a ratio of effector T cells to regulatory T cells.

For example, in some embodiments, the recognition domains specifically bind to a target (e.g. antigen, receptor) associated with T cells. In some embodiments, the recognition domains directly or indirectly recruit T cells. In an embodiment, the recognition domains specifically bind to effector T cells. In some embodiments, the recognition domain directly or indirectly recruits effector T cells, e.g., in some embodiments, to a therapeutic site (e.g. a locus with one or more disease cell or cell to be modulated for a therapeutic effect). Illustrative effector T cells include cytotoxic T cells (e.g. αβ TCR, CD3⁺, CD8⁺, CD45RO⁺); CD4⁺ effector T cells (e.g. αβ TCR, CD3⁺, CD4⁺, CCR7⁺, CD62Lhi, IL:7R/CD127⁺); CD8⁺ effector T cells (e.g. αβ TCR, CD3⁺, CD8⁺, CCR7⁺, CD62Lhi, IL-7R/CD127⁺); effector memory T cells (e.g. CD62Llow, CD44⁺, TCR, CD3⁺, IL-7R/CD127⁺, IL-15R⁺, CCR7low); central memory T cells (e.g. CCR7⁺, CD62L+, CD27+; or CCR7hi, CD44⁺, CD62Lhi, TCR, CD3⁺, IL-7R/CD127⁺, IL-15R⁺); CD62L+ effector T cells; CD8⁺ effector memory T cells (TEM) including early effector memory T cells (CD27⁺CD62L⁻) and late effector memory T cells (CD27⁻CD62L⁻) (TemE and TemL, respectively); CD127(+)CD25(low/−) effector T cells; CD127(−)CD25(−) effector T cells; CD8⁺ stem cell memory effector cells (TSCM) (e.g. CD44(low)CD62L(high)CD122(high)sca(+)); TH1 effector T-cells (e.g. CXCR3⁺, CXCR6⁺ and CCR5⁺; or αβ TCR, CD3⁺, CD4⁺, IL-12R⁺, IFNγR⁺, CXCR3⁺), TH2 effector T cells (e.g. CCR3⁺, CCR4⁺ and CCR8+; or αβ TCR, CD3⁺, CD4⁺, IL-4R⁺, IL-33R⁺, CCR4⁺, IL-17R8⁺, CRTH2⁺); TH9 effector T cells (e.g. αβ TCR, CD3⁺, CD4⁺); TH17 effector T cells (e.g. αβ TCR, CD3⁺, CD4⁺, IL-23R⁺, CCR6⁺, IL-1R⁺); CD4⁺CD45RO⁺CCR7⁺ effector T cells, ICOS⁺ effector T cells; CD4⁺CD45RO⁺CCR7(−) effector T cells; and effector T cells secreting IL-2, IL-4 and/or IFN-γ.

Illustrative T cell antigens of interest include, for example (and inclusive of the extracellular domains, where applicable): CD8, CD3, SLAMF4, IL-2Rα, 4-1BB/TNFRSF9, IL-2 R β, ALCAM, B7-1, IL-4 R, B7-H3, BLAME/SLAMFS, CEACAM1, IL-6 R, CCR3, IL-7 Rα, CCR4, CXCRI/IL-S RA, CCR5, CCR6, IL-10R α, CCR 7, IL-I 0 R β, CCRS, IL-12 R β1, CCR9, IL-12 R β2, CD2, IL-13 R a 1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R Y, Osteopontin, CRACC/SLAMF7, PD-1, CRTAM, PSGL-1, CTLA-4, RANK/TNFRSF11A, CX3CR1, CX3CL1, L-Selectin, CXCR3, SIRP B 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R. In various embodiments, a targeting moiety of the chimeric proteins or chimeric protein complexes such as Fc-based chimeric protein complexes binds one or more of these illustrative T cell antigens.

By way of non-limiting example, in various embodiments, the present chimeric proteins or chimeric protein complexes, such as Fc-based chimeric protein complexes, comprise a targeting moiety directed against a checkpoint marker expressed on a T cell, e.g. one or more of PD-1, PD-L1, CD28, CTLA4, ICOS, BTLA, KIR, LAG3, CD137, OX40, CD27, CD40L, TIM3, and A2aR.

For example, in some embodiments, the recognition domains specifically bind to a target (e.g. antigen, receptor) associated with B cells. In some embodiments, the recognition domains directly or indirectly recruit B cells, e.g., in some embodiments, to a therapeutic site (e.g. a locus with one or more disease cell or cell to be modulated for a therapeutic effect). Illustrative B cell antigens of interest include, for example, CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, CDw150, CS1, and B-cell maturation antigen (BCMA). In various embodiments, a targeting moiety of the chimeric proteins or chimeric protein complexes, such as Fc-based chimeric protein complexes, bind one or more of these illustrative B cell antigens.

By way of further example, in some embodiments, the recognition domains specifically bind to a target (e.g. antigen, receptor) associated with Natural Killer cells. In some embodiments, the recognition domains directly or indirectly recruit Natural Killer cells, e.g., in some embodiments, to a therapeutic site (e.g. a locus with one or more disease cell or cell to be modulated for a therapeutic effect). Illustrative Natural Killer cell antigens of interest include, for example TIGIT, 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAMF6, Rae-1, Rae-1 α, Rae-1 β, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d and ULBP-3. In various embodiments, a targeting moiety of the chimeric proteins or chimeric protein complexes, such as Fc-based chimeric protein complexes, bind one or more of these illustrative NK cell antigens.

Also, in some embodiments, the recognition domains specifically bind to a target (e.g. antigen, receptor) associated with macrophages/monocytes. In some embodiments, the recognition domains directly or indirectly recruit macrophages/monocytes, e.g., in some embodiments, to a therapeutic site (e.g. a locus with one or more disease cell or cell to be modulated for a therapeutic effect). Illustrative macrophages/monocyte antigens of interest include, for example SIRP1a, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-γ R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, 20) LMIR1/CD300A, CD97, LMIR2/CD300c, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-I, IL-6 R, TREM-2, CXCRI/IL-8 RA, TREM-3 and TREMLI/TLT-1. In various embodiments, a targeting moiety of the chimeric proteins or chimeric protein complexes, such as Fc-based chimeric protein complexes, bind one or more of these illustrative macrophage/monocyte antigens.

Also, in some embodiments, the recognition domains specifically bind to a target (e.g. antigen, receptor) associated with dendritic cells. In some embodiments, the recognition domains directly or indirectly recruit dendritic cells, e.g., in some embodiments, to a therapeutic site (e.g. a locus with one or more disease cell or cell to be modulated for a therapeutic effect). Illustrative dendritic cell antigens of interest include, for example, CLEC9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, Integrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, CLEC-8, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC/CLEC4C, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102 and Vanilloid R1. In various embodiments, a targeting moiety of the chimeric proteins or chimeric protein complexes, such as Fc-based chimeric protein complexes, bind one or more of these illustrative DC antigens.

In some embodiments, the recognition domains specifically bind to a target (e.g. antigen, receptor) on immune cells selected from, but not limited to, megakaryocytes, thrombocytes, erythrocytes, mast cells, basophils, neutrophils, myeloid cells, monocytes, eosinophils, or subsets thereof. In some embodiments, the recognition domains directly or indirectly recruit megakaryocytes, thrombocytes, erythrocytes, mast cells, basophils, neutrophils, myeloid cells, monocytes, eosinophils, or subsets thereof, e.g., in some embodiments, to a therapeutic site (e.g. a locus with one or more disease cell or cell to be modulated for a therapeutic effect). In some embodiments, the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof. In some embodiments, the immune cell is a T cell and the targeting moiety targets CD8. In some embodiments, the immune cell is a Treg and the targeting moiety targets CTLA4. In some embodiments, the immune cell is an NK cell and the targeting moiety is NKp46.

In some embodiments, the recognition domains specifically bind to a target (e.g. antigen, receptor) associated with megakaryocytes and/or thrombocytes. Illustrative megakaryocyte and/or thrombocyte antigens of interest include, for example, GP IIb/IIIa, GPIb, vWF, PF4, and TSP. In various embodiments, a targeting moiety of the chimeric proteins or chimeric protein complexes, such as Fc-based chimeric protein complexes, bind one or more of these illustrative megakaryocyte and/or thrombocyte antigens.

In some embodiments, the recognition domains specifically bind to a target (e.g. antigen, receptor) associated with erythrocytes. Illustrative erythrocyte antigens of interest include, for example, CD34, CD36, CD38, CD41a (platelet glycoprotein IIb/IIIa), CD41b (GPIIb), CD71 (transferrin receptor), CD105, glycophorin A, glycophorin C, c-kit, HLA-DR, H2 (MHC-II), and Rhesus antigens. In various embodiments, a targeting moiety of the chimeric proteins or chimeric protein complexes, such as Fc-based chimeric protein complexes, bind one or more of these illustrative erythrocyte antigens.

In some embodiments, the recognition domains specifically bind to a target (e.g. antigen, receptor) associated with mast cells. Illustrative mast cells antigens of interest include, for example, SCFR/CD117, Fc_{ε}RI, CD2, CD25, CD35, CD88, CD203c, C5R1, CMAI, FCERIA, FCER2, TPSABI. In various embodiments, a targeting moiety of the chimeric proteins or chimeric protein complexes. such as Fc-based chimeric protein complexes, bind one or more of these mast cell antigens.

In some embodiments, the recognition domains specifically bind to a target (e.g. antigen, receptor) associated with basophils. Illustrative basophils antigens of interest include, for example, Fc_{ε}RI, CD203c, CD123, CD13, CD107a, CD107b, and CD164. In various embodiments, a targeting moiety of the chimeric proteins or chimeric protein complexes, such as Fc-based chimeric protein complexes, bind one or more of these basophil antigens.

In some embodiments, the recognition domains specifically bind to a target (e.g. antigen, receptor) associated with neutrophils. Illustrative neutrophils antigens of interest include, for example, 7D5, CD10/CALLA, CD13, CD16 (FcRIII), CD18 proteins (LFA-1, CR3, and p150, 95), CD45, CD67, and CD177. In various embodiments, a targeting moiety of the chimeric proteins or chimeric protein complexes, such as Fc-based chimeric protein complexes, bind one or more of these neutrophil antigens.

In some embodiments, the recognition domains specifically bind to a target (e.g. antigen, receptor) associated with eosinophils. Illustrative eosinophils antigens of interest include, for example, CD35, CD44 and CD69. In various embodiments, a targeting moiety of the chimeric proteins or chimeric protein complexes, such as Fc-based chimeric protein complexes, bind one or more of these eosinophil antigens.

In various embodiments, the recognition domain may bind to any appropriate target, antigen, receptor, or cell surface markers known by the skilled artisan. In some embodiments, the antigen or cell surface marker is a tissue-specific marker. Illustrative tissue-specific markers include, but are not limited to, endothelial cell surface markers such as ACE, CD14, CD34, CDH5, ENG, ICAM2, MCAM, NOS3, PECAMI, PROCR, SELE, SELP, TEK, THBD, VCAMI, VWF; smooth muscle cell surface markers such as ACTA2, MYHIO, MYHI 1, MYH9, MYOCD; fibroblast (stromal) cell surface markers such as ALCAM, CD34, COLIAI, COL1A2, COL3A1, FAP, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (αᵥβ₃), E-selectin; and adipocyte surface markers such as ADIPOQ, FABP4, and RETN. In various embodiments, a targeting moiety of the chimeric proteins or chimeric protein complexes such as Fc-based chimeric protein complexes binds one or more of these antigens. In various embodiments, a targeting moiety of the chimeric proteins or chimeric protein complexes, such as Fc-based chimeric protein complexes, bind one or more of cells having these antigens.

In some embodiments, the recognition domains specifically bind to a target (e.g. antigen, receptor) associated with tumor cells. In some embodiments, the recognition domains directly or indirectly recruit tumor cells. For instance, in some embodiments, the direct or indirect recruitment of the tumor cell is to one or more effector cell (e.g. an immune cell as described herein) that can kill and/or suppress the tumor cell.

Tumor cells, or cancer cells refer to an uncontrolled growth of cells or tissues and/or an abnormal increase in cell survival and/or inhibition of apoptosis which interferes with the normal functioning of bodily organs and systems. For example, tumor cells include benign and malignant cancers, polyps, hyperplasia, as well as dormant tumors or micrometastases. Illustrative tumor cells include, but are not limited to cells of: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Tumor cells, or cancer cells also include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative tumor antigens include, but are not limited to, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD47, CS1, CD38, ASGPR, CD56, CD70, CD74, CD138, AGS 16, MUC1, GPNMB, Ep-CAM, PD-L1, PD-L2, PMSA, and BCMA (TNFRSF17). In various embodiments, a targeting moiety of the chimeric proteins or chimeric protein complexes, such as Fc-based chimeric protein complexes, bind one or more of these tumor antigens. In an embodiment, the chimeric proteins or chimeric protein complexes, such as Fc-based chimeric protein complexes, bind to HER2. In another embodiment, the chimeric proteins or chimeric protein complexes, such as Fc-based chimeric protein complexes, bind to PD-L2.

In some embodiments, the present chimeric proteins or chimeric protein complexes, such as Fc-based chimeric protein complexes, have one or more of the targeting moieties which is directed against an immune cell selected from a T cell, a B cell, a dendritic cell, a neutrophil, a myeloid derived suppressor cell, a macrophage, a NK cell, or subsets thereof, along with any of the signaling agents (e.g., modified IL-2) described herein. In one embodiment, the present chimeric proteins or chimeric protein complexes such as Fc-based chimeric protein complexes have a targeting moiety directed against a T cell (including, without limitation an effector T cell), along with any of the signaling agents described herein. In one embodiment, the present chimeric proteins or chimeric protein complexes such as Fc-based chimeric protein complexes have a targeting moiety directed against a B cell, along with any of the signaling agents described herein. In one embodiment, the present chimeric proteins or chimeric protein complexes such as Fc-based chimeric protein complexes have a targeting moiety directed against a dendritic cell, along with any of the signaling agents described herein. In one embodiment, the present chimeric proteins or chimeric protein complexes such as Fc-based chimeric protein complexes have a targeting moiety directed against a macrophage, along with any of the signaling agents described herein. In one embodiment, the present chimeric proteins or chimeric protein complexes such as Fc-based chimeric protein complexes have a targeting moiety directed against a NK cell, along with any of the signaling agents described herein.

By way of non-limiting example, in various embodiments, the present chimeric proteins or chimeric protein complexes such as Fc-based chimeric protein complexes have a targeting moiety directed against a T cell, for example, mediated by targeting to CD8, SLAMF4, IL-2 R α, 4-1BB/TNFRSF9, IL-2 R β, ALCAM, B7-1, IL-4 R, B7-H3, BLAME/SLAMFS, CEACAM1, IL-6 R, CCR3, IL-7 Rα, CCR4, CXCRI/IL-S RA, CCR5, CCR6, IL-10R α, CCR 7, IL-10 R β, CCRS, IL-12 R β1, CCR9, IL-12 R β2, CD2, IL-13 R a 1, IL-13, CD3, CD4, ILT2/CDS5j, ILT3/CDS5k, ILT4/CDS5d, ILT5/CDS5a, Integrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R Y, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1, or TSLP R; along with any of the signaling agents (e.g., a modified IL-2) described herein.

By way of non-limiting example, in various embodiments, the present chimeric proteins or chimeric protein complexes such as Fc-based chimeric protein complexes have a targeting moiety directed against a checkpoint marker expressed on a T cell, e.g. one or more of PD-1, CD28, CTLA4, ICOS, BTLA, KIR, LAG3, CD137, OX40, CD27, CD40L, TIM3, and A2aR, along with any of the signaling agents described herein.

In various embodiments, the present chimeric proteins or chimeric protein complexes such as Fc-based chimeric protein complexes have one or more targeting moieties directed against PD-1. In some embodiments, the chimeric proteins or chimeric protein complexes such as Fc-based chimeric protein complexes have one or more targeting moieties which selectively bind a PD-1 polypeptide. In some embodiments, the chimeric proteins or chimeric protein complexes, such as Fc-based chimeric protein complexes, comprise one or more antibodies, antibody derivatives or formats, peptides or polypeptides, or fusion proteins that selectively bind a PD-1 polypeptide.

In an embodiment, the targeting moiety comprises the anti-PD-1 antibody pembrolizumab (aka MK-3475, KEYTRUDA), or fragments thereof. Pembrolizumab and other humanized anti-PD-1 antibodies are disclosed in Hamid, et al. (2013) New England Journal of Medicine 369 (2): 134-44, U.S. Pat. No. 8,354,509, and WO 2009/114335, the entire disclosures of which are hereby incorporated by reference. In illustrative embodiments, pembrolizumab or an antigen-binding fragment thereof for use in the methods provided herein comprises a heavy chain comprising the amino acid sequence of SEQ ID NO: 7 and/or a light chain comprising the amino acid sequence of SEQ ID NO: 8.

In an embodiment, the targeting moiety comprises the anti-PD-1 antibody, nivolumab (aka BMS-936558, MDX-1106, ONO-4538, OPDIVO), or fragments thereof. Nivolumab (clone 5C4) and other human monoclonal antibodies that specifically bind to PD-1 are disclosed in U.S. Pat. No. 8,008,449 and WO 2006/121168, the entire disclosures of which are hereby incorporated by reference. In illustrative embodiments, nivolumab or an antigen-binding fragment thereof comprises a heavy chain comprising the amino acid sequence of SEQ ID NO: 9 and/or a light chain comprising the amino acid sequence of SEQ ID NO: 10.

In an embodiment, the targeting moiety comprises the anti-PD-1 antibody pidilizumab (aka CT-011, hBAT or hBAT-1), or fragments thereof. Pidilizumab and other humanized anti-PD-I monoclonal antibodies are disclosed in US 2008/0025980 and WO 2009/101611, the entire disclosures of which are hereby incorporated by reference. In illustrative embodiments, the anti-PD-1 antibody or an antigen-binding fragment thereof for use in the methods provided herein comprises a light chain variable regions comprising an amino acid sequence selected from SEQ ID NOs: 15-18 of US 2008/0025980: SEQ ID NO: 15 of US 2008/0025980 (SEQ ID NO: 11); SEQ ID NO: 16 of US 2008/0025980 (SEQ ID NO: 12); SEQ ID NO: 17 of US 2008/0025980 (SEQ ID NO: 13); and SEQ ID NO: 18 of US 2008/0025980 (SEQ ID NO: 14); and/or a heavy chain comprising an amino acid sequence selected from SEQ ID NOs: 20-24 of US 2008/0025980: SEQ ID NO: 20 of US 2008/0025980 (SEQ ID NO: 15); SEQ ID NO: 21 of US 2008/0025980 (SEQ ID NO: 16); SEQ ID NO: 22 of US 2008/0025980 (SEQ ID NO: 17); SEQ ID NO: 23 of US 2008/0025980 (SEQ ID NO: 18); and SEQ ID NO: 24 of US 2008/0025980 (SEQ ID NO: 19).

In an embodiment, the targeting moiety comprises a light chain comprising SEQ ID NO: 18 of US 2008/0025980 (SEQ ID NO: 14) and a heavy chain comprising SEQ ID NO: 22 of US 2008/0025980 (SEQ ID NO: 17).

In an embodiment, the targeting moiety comprises AMP-514 (aka MEDI-0680).

In an embodiment, the targeting moiety comprises the PD-L2-Fc fusion protein AMP-224, which is disclosed in WO2010/027827 and WO 2011/066342, the entire disclosures of which are hereby incorporated by reference. In such an embodiment, the targeting moiety may include a targeting domain which comprises SEQ ID NO:4 of WO2010/027827 (SEQ ID NO: 20) and/or the B7-DC fusion protein which comprises SEQ ID NO:83 of WO2010/027827 (SEQ ID NO: 21).

In an embodiment, the targeting moiety comprises the PD-L1-Fc fusion protein and/or the PD-1-Fc fusion protein, as described in WO 2019/191519.

In an embodiment, the targeting moiety comprises the peptide AUNP 12 or any of the other peptides disclosed in US 2011/0318373 or 8,907,053. For example, the targeting moiety may comprise AUNP 12 (i.e., Compound 8 or SEQ ID NO:49 of US 2011/0318373) which has the sequence of:

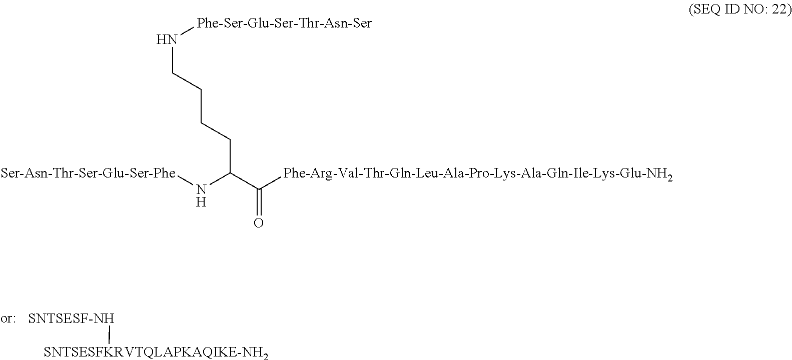

In an embodiment, the targeting moiety comprises the anti-PD-1 antibody 1E3, or fragments thereof, as disclosed in US 2014/0044738, the entire disclosures of which are hereby incorporated by reference. In illustrative embodiments, 1E3 or an antigen-binding fragment thereof for use in the methods provided herein comprises a heavy chain variable region comprising the amino acid sequence of SEQ ID NO: 23; and/or a light chain variable region comprising the amino acid sequence of SEQ ID NO: 24.

In an embodiment, the targeting moiety comprises the anti-PD-1 antibody 1E8, or fragments thereof, as disclosed in US 2014/0044738, the entire disclosures of which are hereby incorporated by reference. In illustrative embodiments, 1E8 or an antigen-binding fragment thereof for use in the methods provided herein comprises a heavy chain variable region comprising the amino acid sequence of SEQ ID NO: 25 and/or a light chain variable region comprising the amino acid sequence of SEQ ID NO: 26.

In an embodiment, the targeting moiety comprises the anti-PD-1 antibody 1H3, or fragments thereof, as disclosed in US 2014/0044738, the entire disclosures of which are hereby incorporated by reference. In illustrative embodiments, 1H3 or an antigen-binding fragment thereof for use in the methods provided herein comprises a heavy chain variable region comprising the amino acid sequence of SEQ ID NO: 27 and/or light chain variable region comprising the amino acid sequence of SEQ ID NO: 28.

In an embodiment, the targeting moiety comprises a VHH directed against PD-1 as disclosed, for example, in U.S. Pat. No. 8,907,065 and WO 2008/071447, the entire disclosures of which are hereby incorporated by reference. In illustrative embodiments, the VHHs against PD-1 comprise SEQ ID NOs: 347-351 of U.S. Pat. No. 8,907,065 (SEQ ID NO: 347 of U.S. Pat. No. 8,907,065 (SEQ ID NO: 29); SEQ ID NO: 348 of U.S. Pat. No. 8,907,065 (SEQ ID NO:30); SEQ ID NO: 349 of U.S. Pat. No. 8,907,065 (SEQ ID NO:31); SEQ ID NO: 350 of U.S. Pat. No. 8,907,065 (SEQ ID NO: 32); and SEQ ID NO: 351 of U.S. Pat. No. 8,907,065 (SEQ ID NO:33)).

In an embodiment, the targeting moiety comprises any one of the anti-PD-1 antibodies, or fragments thereof, as disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID NOs: 25-29 of US2011/0271358 (SEQ ID NO: 25 of US2011/0271358 (SEQ ID NO:34); SEQ ID NO: 26 of US2011/0271358 (SEQ ID NO:35); SEQ ID NO: 27 of US2011/0271358 (SEQ ID NO:36); SEQ ID NO: 28 of US2011/0271358 (SEQ ID NO:37); and SEQ ID NO: 29 of US2011/0271358 (SEQ ID NO:38); and/or a light chain comprising an amino acid sequence selected from SEQ ID NOs: 30-33 of US2011/0271358 (SEQ ID NO: 30 of US2011/0271358 (SEQ ID NO:39); SEQ ID NO: 31 of US2011/0271358 (SEQ ID NO:40); SEQ ID NO: 32 of US2011/0271358 (SEQ ID NO:41); and SEQ ID NO: 33 of US2011/0271358 (SEQ ID NO:42)).

In various embodiments, the present chimeric proteins or chimeric protein complexes, such as Fc-based chimeric protein complexes, comprise one or more antibodies directed against PD-1, or antibody fragments thereof, selected from TSR-042 (Tesaro, Inc.), REGN2810 (Regeneron Pharmaceuticals, Inc.), PDR001 (Novartis Pharmaceuticals), and BGB-A317 (BeiGene Ltd.).

In various embodiments, the present chimeric proteins or chimeric protein complexes, such as Fc-based chimeric protein complexes, have one or more targeting moieties directed against PD-L1. In some embodiments, the chimeric proteins or chimeric protein complexes, such as Fc-based chimeric protein complexes, have one or more targeting moieties which selectively bind a PD-L1 polypeptide. In some embodiments, the chimeric proteins or chimeric protein complexes, such as Fc-based chimeric protein complexes, comprise one or more antibodies, antibody derivatives or formats, peptides or polypeptides, or fusion proteins that selectively bind a PD-L1 polypeptide.

In an embodiment, the targeting moiety comprises the anti-PD-L1 antibody MEDI4736 (aka durvalumab), or fragments thereof. MEDI4736 is selective for PD-L1 and blocks the binding of PD-L1 to the PD-1 and CD80 receptors. MEDI4736 and antigen-binding fragments thereof for use in the methods provided herein comprises a heavy chain and a light chain or a heavy chain variable region and a light chain variable region. The sequence of MEDI4736 is disclosed in WO/2016/06272, the entire contents of which are hereby incorporated by reference. In illustrative embodiments, MEDI4736 or an antigen-binding fragment thereof for use in the methods provided herein comprises a heavy chain comprising the amino acid sequence of SEQ ID NO:43; and/or a light chain comprising the amino acid sequence of SEQ ID NO:44.

In illustrative embodiments, the MEDI4736 or an antigen-binding fragment thereof for use in the methods provided herein comprises a heavy chain variable region comprising the amino acid sequence of SEQ ID NO:4 of WO/2016/06272 (SEQ ID NO:45); and/or a light chain variable region comprising the amino acid sequence of SEQ ID NO:3 of WO/2016/06272 (SEQ ID NO:46).

In an embodiment, the targeting moiety comprises the anti-PD-L1 antibody atezolizumab (aka MPDL3280A, RG7446), or fragments thereof. In illustrative embodiments, atezolizumab or an antigen-binding fragment thereof for use in the methods provided herein comprises a heavy chain comprising the amino acid sequence of SEQ ID NO:47; and/or a light chain comprising the amino acid sequence of SEQ ID NO:48.

In an embodiment, the targeting moiety comprises the anti-PD-L1 antibody avelumab (aka MSB0010718C), or fragments thereof. In illustrative embodiments, avelumab or an antigen-binding fragment thereof for use in the methods provided herein comprises a heavy chain comprising the amino acid sequence of SEQ ID NO:49; and/or a light chain comprising the amino acid sequence of SEQ ID NO:50.

In an embodiment, the targeting moiety comprises the anti-PD-L1 antibody BMS-936559 (aka 12A4, MDX-1105), or fragments thereof, as disclosed in US 2013/0309250 and WO2007/005874, the entire disclosures of which are hereby incorporated by reference. In illustrative embodiments, BMS-936559 or an antigen-binding fragment thereof for use in the methods provided herein comprises a heavy chain variable region comprising the amino acid sequence of: (SEQ ID NO:51); and/or a light chain variable region comprising the amino acid sequence of (SEQ ID NO:52).

In an embodiment, the targeting moiety comprises the anti-PD-L1 antibody 3G10, or fragments thereof, as disclosed in US 2013/0309250 and WO2007/005874, the entire disclosures of which are hereby incorporated by reference. In illustrative embodiments, 3G10 or an antigen-binding fragment thereof for use in the methods provided herein comprises a heavy chain variable region comprising the amino acid sequence of (SEQ ID NO: 53); and/or a light chain variable region comprising the amino acid sequence of (SEQ ID NO: 54).

In an embodiment, the targeting moiety comprises the anti-PD-L1 antibody 10A5, or fragments thereof, as disclosed in US 2013/0309250 and WO2007/005874, the entire disclosures of which are hereby incorporated by reference. In illustrative embodiments, 10A5 or an antigen-binding fragment thereof for use in the methods provided herein comprises a heavy chain variable region comprising the amino acid sequence of (SEQ ID NO: 55); and/or a light chain variable region comprising the amino acid sequence of (SEQ ID NO: 56).

In an embodiment, the targeting moiety comprises the anti-PD-L1 antibody 5F8, or fragments thereof, as disclosed in US 2013/0309250 and WO2007/005874, the entire disclosures of which are hereby incorporated by reference. In illustrative embodiments, 5F8 or an antigen-binding fragment thereof for use in the methods provided herein comprises a heavy chain variable region comprising the amino acid sequence of (SEQ ID NO: 57); and/or a light chain variable region comprising the amino acid sequence of (SEQ ID NO: 58).

In an embodiment, the targeting moiety comprises the anti-PD-L1 antibody 10H10, or fragments thereof, as disclosed in US 2013/0309250 and WO2007/005874, the entire disclosures of which are hereby incorporated by reference. In illustrative embodiments, 10H10 or an antigen-binding fragment thereof for use in the methods provided herein comprises a heavy chain variable region comprising the amino acid sequence of (SEQ ID NO: 59); and/or a light chain variable region comprising the amino acid sequence of (SEQ ID NO: 60).

In an embodiment, the targeting moiety comprises the anti-PD-L1 antibody 1B12, or fragments thereof, as disclosed in US 2013/0309250 and WO2007/005874, the entire disclosures of which are hereby incorporated by reference. In illustrative embodiments, 1B12 or an antigen-binding fragment thereof for use in the methods provided herein comprises a heavy chain variable region comprising the amino acid sequence of (SEQ ID NO: 61); and/or a light chain variable region comprising the amino acid sequence of (SEQ ID NO: 62).

In an embodiment, the targeting moiety comprises the anti-PD-L1 antibody 7H1, or fragments thereof, as disclosed in US 2013/0309250 and WO2007/005874, the entire disclosures of which are hereby incorporated by reference.

In illustrative embodiments, 7H1 or an antigen-binding fragment thereof for use in the methods provided herein comprises a heavy chain variable region comprising the amino acid sequence of (SEQ ID NO: 63); and/or a light chain variable region comprising the amino acid sequence of (SEQ ID NO: 64).

In an embodiment, the targeting moiety comprises the anti-PD-L1 antibody 11E6, or fragments thereof, as disclosed in US 2013/0309250 and WO2007/005874, the entire disclosures of which are hereby incorporated by reference. In illustrative embodiments, 11E6 or an antigen-binding fragment thereof for use in the methods provided herein comprises a heavy chain variable region comprising the amino acid sequence of (SEQ ID NO: 65); and/or a light chain variable region comprising the amino acid sequence of (SEQ ID NO: 66).

In an embodiment, the targeting moiety comprises the anti-PD-L1 antibody 12B7, or fragments thereof, as disclosed in US 2013/0309250 and WO2007/005874, the entire disclosures of which are hereby incorporated by reference. In illustrative embodiments, 12B7 or an antigen-binding fragment thereof for use in the methods provided herein comprises a heavy chain variable region comprising the amino acid sequence of (SEQ ID NO: 67); and/or a light chain variable region comprising the amino acid sequence of (SEQ ID NO: 68).

In an embodiment, the targeting moiety comprises the anti-PD-L1 antibody 13G4, or fragments thereof, as disclosed in US 2013/0309250 and WO2007/005874, the entire disclosures of which are hereby incorporated by reference. In illustrative embodiments, 13G4 or an antigen-binding fragment thereof for use in the methods provided herein comprises a heavy chain variable region comprising the amino acid sequence of (SEQ ID NO: 69); and/or a light chain variable region comprising the amino acid sequence of (SEQ ID NO: 70).

In an embodiment, the targeting moiety comprises the anti-PD-L1 antibody 1E12, or fragments thereof, as disclosed in US 2014/0044738, the entire disclosures of which are hereby incorporated by reference. In illustrative embodiments, 1E12 or an antigen-binding fragment thereof for use in the methods provided herein comprises a heavy chain variable region comprising the amino acid sequence of (SEQ ID NO: 71); and/or a light chain variable region comprising the amino acid sequence of (SEQ ID NO: 72).

In an embodiment, the targeting moiety comprises the anti-PD-L1 antibody 1F4, or fragments thereof, as disclosed in US 2014/0044738, the entire disclosures of which are hereby incorporated by reference. In illustrative embodiments, 1F4 or an antigen-binding fragment thereof for use in the methods provided herein comprises a heavy chain variable region comprising the amino acid sequence of (SEQ ID NO: 73); and/or a light chain variable region comprising the amino acid sequence of (SEQ ID NO: 74).

In an embodiment, the targeting moiety comprises the anti-PD-L1 antibody 2G11, or fragments thereof, as disclosed in US 2014/0044738, the entire disclosures of which are hereby incorporated by reference. In illustrative embodiments, 2G11 or an antigen-binding fragment thereof for use in the methods provided herein comprises a heavy chain variable region comprising the amino acid sequence of (SEQ ID NO: 75); and/or a light chain variable region comprising the amino acid sequence of (SEQ ID NO: 76).

In an embodiment, the targeting moiety comprises the anti-PD-L1 antibody 3B6, or fragments thereof, as disclosed in US 2014/0044738, the entire disclosures of which are hereby incorporated by reference. In illustrative embodiments, 3B6 or an antigen-binding fragment thereof for use in the methods provided herein comprises a heavy chain variable region comprising the amino acid sequence of (SEQ ID NO: 77); and/or a light chain variable region comprising the amino acid sequence of (SEQ ID NO: 78).

In an embodiment, the targeting moiety comprises the anti-PD-L1 antibody 3D10, or fragments thereof, as disclosed in US 2014/0044738 and WO2012/145493, the entire disclosures of which are hereby incorporated by reference. In illustrative embodiments, 3D10 or an antigen-binding fragment thereof for use in the methods provided herein comprises a heavy chain variable region comprising the amino acid sequence of (SEQ ID NO: 79); and/or a light chain variable region comprising the amino acid sequence of (SEQ ID NO: 80).

In an embodiment, the targeting moiety comprises any one of the anti-PD-L1 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 34-38 of US2011/0271358 (SEQ ID No: 34 of US2011/0271358 (SEQ ID NO: 81); SEQ ID No: 35 of US2011/0271358 (SEQ ID NO: 82); SEQ ID No: 36 of US2011/0271358 (SEQ ID NO: 83); SEQ ID No: 37 of US2011/0271358 (SEQ ID NO: 84); and SEQ ID No: 38 of US2011/0271358 (SEQ ID NO: 85); and/or a light chain comprising an amino acid sequence selected from SEQ ID Nos: 39-42 of US2011/0271358 (SEQ ID No: 39 of US2011/0271358 (SEQ ID NO: 86); SEQ ID No: 40 of US2011/0271358 (SEQ ID NO: 87); SEQ ID No: 41 of US2011/0271358 (SEQ ID NO: 88); and SEQ ID No: 42 of US2011/0271358(SEQ ID NO: 89).

In an embodiment, the targeting moiety comprises the anti-PD-L1 antibody 2.7A4, or fragments thereof, as disclosed in WO 2011/066389, U.S. Pat. No. 8,779,108, and US2014/0356353, the entire disclosures of which are hereby incorporated by reference. In illustrative embodiments, 2.7A4 or an antigen-binding fragment thereof for use in the methods provided herein comprises a heavy chain variable region comprising the amino acid sequence of SEQ ID No: 2 of WO 2011/066389 (SEQ ID NO: 90); and/or a light chain variable region comprising the amino acid sequence of SEQ ID No: 7 of WO 2011/066389 (SEQ ID NO: 91).

In an embodiment, the targeting moiety comprises the anti-PD-L1 antibody 2.9D10, or fragments thereof, as disclosed in WO 2011/066389, U.S. Pat. No. 8,779,108, and US2014/0356353, the entire disclosures of which are hereby incorporated by reference. In illustrative embodiments, 2.9D10 or an antigen-binding fragment thereof for use in the methods provided herein comprises a heavy chain variable region comprising the amino acid sequence of SEQ ID No: 12 of WO 2011/066389 (SEQ ID NO: 92); and/or a light chain variable region comprising the amino acid sequence of SEQ ID No: 17 of WO 2011/066389 (SEQ ID NO: 93).

In an embodiment, the targeting moiety comprises the anti-PD-L1 antibody 2.14H9, or fragments thereof, as disclosed in WO 2011/066389, U.S. Pat. No. 8,779,108, and US2014/0356353, the entire disclosures of which are hereby incorporated by reference. In illustrative embodiments, 2.14H9 or an antigen-binding fragment thereof for use in the methods provided herein comprises a heavy chain variable region comprising the amino acid sequence of SEQ ID No: 22 of WO 2011/066389 (SEQ ID NO: 94); and/or a light chain variable region comprising the amino acid sequence of SEQ ID No: 27 of WO 2011/066389 (SEQ ID NO: 95).

In an embodiment, the targeting moiety comprises the anti-PD-L1 antibody 2.20A8, or fragments thereof, as disclosed in WO 2011/066389, U.S. Pat. No. 8,779,108, and US2014/0356353, the entire disclosures of which are hereby incorporated by reference. In illustrative embodiments, 2.20A8 or an antigen-binding fragment thereof for use in the methods provided herein comprises a heavy chain variable region comprising the amino acid sequence of SEQ ID No: 32 of WO 2011/066389 (SEQ ID NO: 96); and/or a light chain variable region comprising the amino acid sequence of SEQ ID No: 37 of WO 2011/066389 (SEQ ID NO: 97).

In an embodiment, the targeting moiety comprises the anti-PD-L1 antibody 3.15G8, or fragments thereof, as disclosed in WO 2011/066389, U.S. Pat. No. 8,779,108, and US2014/0356353, the entire disclosures of which are hereby incorporated by reference. In illustrative embodiments, 3.15G8 or an antigen-binding fragment thereof for use in the methods provided herein comprises a heavy chain variable region comprising the amino acid sequence of SEQ ID No: 42 of WO 2011/066389 (SEQ ID NO: 98); and/or a light chain variable region comprising the amino acid sequence of SEQ ID No: 47 of WO 2011/066389 (SEQ ID NO: 99).

In an embodiment, the targeting moiety comprises the anti-PD-L1 antibody 3.18G1, or fragments thereof, as disclosed in WO 2011/066389, U.S. Pat. No. 8,779,108, and US2014/0356353, the entire disclosures of which are hereby incorporated by reference. In illustrative embodiments, 3.18G1 or an antigen-binding fragment thereof for use in the methods provided herein comprises a heavy chain variable region comprising the amino acid sequence of SEQ ID No: 52 of WO 2011/066389 (SEQ ID NO: 100); and/or a light chain variable region comprising the amino acid sequence of SEQ ID No: 57 of WO 2011/066389 (SEQ ID NO: 101).

In an embodiment, the targeting moiety comprises the anti-PD-L1 antibody 2.7A4OPT, or fragments thereof, as disclosed in WO 2011/066389, U.S. Pat. No. 8,779,108, and US2014/0356353, and US2014/0356353, the entire disclosures of which are hereby incorporated by reference. In illustrative embodiments, 2.7A4OPT or an antigen-binding fragment thereof for use in the methods provided herein comprises a heavy chain variable region comprising the amino acid sequence of SEQ ID No: 62 of WO 2011/066389 (SEQ ID NO:102); and/or a light chain variable region comprising the amino acid sequence of SEQ ID No: 67 of WO 2011/066389 (SEQ ID NO:103).

In an embodiment, the targeting moiety comprises the anti-PD-L1 antibody 2.14H9OPT, or fragments thereof, as disclosed in WO 2011/066389, U.S. Pat. No. 8,779,108, and US2014/0356353, the entire disclosures of which are hereby incorporated by reference. In illustrative embodiments, 2.14H9OPT or an antigen-binding fragment thereof for use in the methods provided herein comprises a heavy chain variable region comprising the amino acid sequence of SEQ ID No: 72 of WO 2011/066389 (SEQ ID NO:104); and/or a light chain variable region comprising the amino acid sequence of SEQ ID No: 77 of WO 2011/066389 (SEQ ID NO:105).

In an embodiment, the targeting moiety comprises any one of the anti-PD-L1 antibodies disclosed in WO2016/061142, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 18, 30, 38, 46, 50, 54, 62, 70, and 78 of WO2016/061142 (SEQ ID No: 18 of WO2016/061142 (SEQ ID NO:106); SEQ ID No: 30 of WO2016/061142 (SEQ ID NO: 107); SEQ ID No: 38 of WO2016/061142 (SEQ ID NO: 108); SEQ ID No: 46 of WO2016/061142 (SEQ ID NO:109); SEQ ID No: 50 of WO2016/061142 (SEQ ID NO:110); SEQ ID No: 54 of WO2016/061142 (SEQ ID NO:111); SEQ ID No: 62 of WO2016/061142 (SEQ ID NO:112); SEQ ID No: 70 of WO2016/061142 (SEQ ID NO: 113); and SEQ ID No: 78 of WO2016/061142 (SEQ ID NO:114)); and/or a light chain comprising an amino acid sequence selected from SEQ ID Nos: 22, 26, 34, 42, 58, 66, 74, 82, and 86 of WO2016/061142 (SEQ ID No: 22 of WO2016/061142 (SEQ ID NO:115); SEQ ID No: 26 of WO2016/061142 (SEQ ID NO:116); SEQ ID No: 34 of WO2016/061142 (SEQ ID NO:117); SEQ ID No: 42 of WO2016/061142 (SEQ ID NO:118); SEQ ID No: 58 of WO2016/061142 (SEQ ID NO:119); SEQ ID No: 66 of WO2016/061142(SEQ ID NO:120); SEQ ID No: 74 of WO2016/061142 (SEQ ID NO:121); SEQ ID No: 82 of WO2016/061142 (SEQ ID NO:122); and SEQ ID No: 86 of WO2016/061142 (SEQ ID NO:123).

In an embodiment, the targeting moiety comprises any one of the anti-PD-L1 antibodies disclosed in WO2016/022630, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 6, 10, 14, 18, 22, 26, 30, 34, 38, 42, and 46 of WO2016/022630 (SEQ ID No: 2 of WO2016/022630 (SEQ ID NO:124); SEQ ID No: 6 of WO2016/022630 (SEQ ID NO:125); SEQ ID No: 10 of WO2016/022630 (SEQ ID NO: 126); SEQ ID No: 14 of WO2016/022630 (SEQ ID NO: 127); SEQ ID No: 18 of WO2016/022630 (SEQ ID NO:128); SEQ ID No: 22 of WO2016/022630 (SEQ ID NO:129); SEQ ID No: 26 of WO2016/022630 (SEQ ID NO: 130); SEQ ID No: 30 of WO2016/022630 (SEQ ID NO:131); SEQ ID No: 34 of WO2016/022630 (SEQ ID NO:132); SEQ ID No: 38 of WO2016/022630 (SEQ ID NO:133); SEQ ID No: 42 of WO2016/022630 (SEQ ID NO: 134); and SEQ ID No: 46 of WO2016/022630 (SEQ ID NO: 135); and/or a light chain comprising an amino acid sequence selected from SEQ ID Nos: 4, 8, 12, 16, 20, 24, 28, 32, 36, 40, 44, and 48 of WO2016/022630 (SEQ ID No: 4 of WO2016/022630 (SEQ ID NO:136); SEQ ID No: 8 of WO2016/022630 (SEQ ID NO:137); SEQ ID No: 12 of WO2016/022630 (SEQ ID NO:138); SEQ ID No: 16 of

WO2016/022630 (SEQ ID NO:139); SEQ ID No: 20 of WO2016/022630 (SEQ ID NO:140); SEQ ID No: 24 of WO2016/022630 (SEQ ID NO:141); SEQ ID No: 28 of WO2016/022630 (SEQ ID NO:142); SEQ ID No: 32 of WO2016/022630 (SEQ ID NO:143); SEQ ID No: 36 of WO2016/022630 (SEQ ID NO:144); SEQ ID No: 40 of WO2016/022630 (SEQ ID NO:145); SEQ ID No: 44 of WO2016/022630 (SEQ ID NO:146); and SEQ ID No: 48 of WO2016/022630 (SEQ ID NO:147)).

In an embodiment, the targeting moiety comprises any one of the anti-PD-L1 antibodies disclosed in WO2015/112900, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 38, 50, 82, and 86 of WO 2015/112900 (SEQ ID No: 38 of WO2015/112900 (SEQ ID NO:148); SEQ ID No: 50 of WO 2015/112900 (SEQ ID NO:149); SEQ ID No: 82 of WO 2015/112900 (SEQ ID NO:150); and SEQ ID No: 86 of WO 2015/112900 (SEQ ID NO:151)); 20) and/or a light chain comprising an amino acid sequence selected from SEQ ID Nos: 42, 46, 54, 58, 62, 66, 70, 74, and 78 of WO 2015/112900 (SEQ ID No: 42 of WO2015/112900 (SEQ ID NO:152); SEQ ID No: 46 of WO 2015/112900: (SEQ ID NO:153); SEQ ID No: 54 of WO 2015/112900 (SEQ ID NO:154); SEQ ID No: 58 of WO 2015/112900 (SEQ ID NO:155); SEQ ID No: 62 of WO 2015/112900 (SEQ ID NO:156); SEQ ID No: 66 of WO 2015/112900 (SEQ ID NO:157); SEQ ID No: 70 of WO 2015/112900 (SEQ ID NO:158); SEQ ID No: 74 of WO 2015/112900 (SEQ ID NO:159); and SEQ ID No: 78 of WO 2015/112900 (SEQ ID NO:160)).

In an embodiment, the targeting moiety comprises any one of the anti-PD-L1 antibodies disclosed in WO 2010/077634 and U.S. Pat. No. 8,217,149, the entire disclosures of which are hereby incorporated by reference. In illustrative embodiments, the anti-PD-L1 antibody or an antigen-binding fragment thereof for use in the methods provided herein comprises a heavy chain region comprising the amino acid sequence of SEQ ID No: 20 of WO 2010/077634 (SEQ ID NO: 161); and/or a light chain variable region comprising the amino acid sequence of SEQ ID No: 21 of WO 2010/077634 (SEQ ID NO: 162).

In an embodiment, the targeting moiety comprises any one of the anti-PD-L1 antibodies obtainable from the hybridoma accessible under CNCM deposit numbers CNCM I-4122, CNCM I-4080 and CNCM I-4081 as disclosed in US 20120039906, the entire disclosures of which are hereby incorporated by reference.

In an embodiment, the targeting moiety comprises a VHH directed against PD-L1 as disclosed, for example, in U.S. Pat. No. 8,907,065 and WO 2008/071447, the entire disclosures of which are hereby incorporated by reference. In illustrative embodiments, the VHHs against PD-L1 comprise SEQ ID NOS: 394-399 of U.S. Pat. No. 8,907,065 (SEQ ID No: 394 of U.S. Pat. No. 8,907,065 (SEQ ID NO:163); SEQ ID No: 395 of U.S. Pat. No. 8,907,065 (SEQ ID NO:164); SEQ ID No: 396 of U.S. Pat. No. 8,907,065 (SEQ ID NO:165); SEQ ID No: 397 of U.S. Pat. No. 8,907,065 (SEQ ID NO:166); SEQ ID No: 398 of U.S. Pat. No. 8,907,065 (SEQ ID NO:167); and SEQ ID No: 399 of U.S. Pat. No. 8,907,065 (SEQ ID NO:168).

In various embodiments, the present chimeric proteins or chimeric protein complexes such as Fc-based chimeric protein complexes have one or more targeting moieties directed against PD-L2. In some embodiments, the chimeric proteins or chimeric protein complexes such as Fc-based chimeric protein complexes have one or more targeting moieties which selectively bind a PD-L2 polypeptide. In some embodiments, the chimeric proteins or chimeric protein complexes such as Fc-based chimeric protein complexes comprise one or more antibodies, antibody derivatives or formats, peptides or polypeptides, or fusion proteins that selectively bind a PD-L2 polypeptide.

In an embodiment, the targeting moiety comprises a VHH directed against PD-L2 as disclosed, for example, in U.S. Pat. No. 8,907,065 and WO 2008/071447, the entire disclosures of which are hereby incorporated by reference. In illustrative embodiments, the VHHs against PD-L2 comprise SEQ ID Nos: 449-455 of U.S. Pat. No. 8,907,065 (SEQ ID No: 449 of U.S. Pat. No. 8,907,065 (SEQ ID NO:169); SEQ ID No: 450 of U.S. Pat. No. 8,907,065 (SEQ ID NO:170); SEQ ID No: 451 of U.S. Pat. No. 8,907,065 (SEQ ID NO:171); SEQ ID No: 452 of U.S. Pat. No. 8,907,065 (SEQ ID NO:172); SEQ ID No: 453 of U.S. Pat. No. 8,907,065 (SEQ ID NO:173); SEQ ID No: 454 of U.S. Pat. No. 8,907,065 (SEQ ID NO:174); and SEQ ID No: 455 of U.S. Pat. No. 8,907,065 (SEQ ID NO:175)).

In an embodiment, the targeting moiety comprises any one of the anti-PD-L2 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 43-47 of US2011/0271358 (SEQ ID No: 43 of US2011/0271358 (SEQ ID NO:176); SEQ ID No: 44 of US2011/0271358 (SEQ ID NO:177); SEQ ID No: 45 of US2011/0271358 (SEQ ID NO:178); SEQ ID No: 46 of US2011/0271358 (SEQ ID NO:179); and SEQ ID No: 47 of US2011/0271358 (SEQ ID NO:180)); and/or a light chain comprising an amino acid sequence selected from SEQ ID Nos: 48-51 of US2011/0271358 (SEQ ID No: 48 of US2011/0271358 (SEQ ID NO:181); SEQ ID No: 49 of US2011/0271358 (SEQ ID NO:182); SEQ ID No: 50 of US2011/0271358 (SEQ ID NO:183); and SEQ ID No: 51 of US2011/0271358 (SEQ ID NO:184)).

In various embodiments, the targeting moieties of the invention may comprise a sequence that targets PD-1, PD-L1, and/or PD-L2 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein.

In various embodiments, the targeting moieties of the invention may comprise any combination of heavy chain, light chain, heavy chain variable region, light chain variable region, complementarity determining region (CDR), and framework region sequences that target PD-1, PD-L1, and/or PD-L2 as disclosed herein.

Additional antibodies, antibody derivatives or formats, peptides or polypeptides, or fusion proteins that selectively bind or target PD-1, PD-L1 and/or PD-L2 are disclosed in WO 2011/066389, US 2008/0025980, US 2013/0034559, U.S. Pat. No. 8,779,108, US 2014/0356353, U.S. Pat. No. 8,609,089, US 2010/028330, US 2012/0114649, WO 2010/027827, WO 2011,/066342, U.S. Pat. No. 8,907,065, WO 2016/062722, WO 2009/101611, WO2010/027827, WO 2011/066342, WO 2007/005874, WO 2001/014556, US2011/0271358, WO 2010/036959, WO 2010/077634, U.S. Pat. No. 8,217,149, US 2012/0039906, WO 2012/145493, US 2011/0318373, U.S. Pat. No. 8,779,108, US 20140044738, WO 2009/089149, WO 2007/00587, WO 2016061142, WO 2016,02263, WO 2010/077634, and WO 2015/112900, the entire disclosures of which are hereby incorporated by reference.

In one embodiment, the present chimeric proteins or chimeric protein complexes, such as Fc-based chimeric protein complexes, have a targeting moiety directed against a T cell, for example, mediated by targeting to CD8, along with any of the signaling agents (e.g., a modified IL-2) described herein. In an embodiment, the present chimeric proteins or chimeric protein complexes, such as Fc-based chimeric protein complexes, have a targeting moiety directed against CD8 on T cells.

In an embodiment, the targeting moiety directed against CD8 comprises a VHH comprising the amino acid sequence of:

(SEQ ID NO: 454)
EVQLVESGGGLVQPGGSLRLSCAASGFTFEDYAIGWFRQAPGKGREGVAC
IRIFDRHTYYADSVKGRFTISSDNSKNTVYLQMNSLRAEDTATYYCAAGS
FFGCTRPEGDMDYFGQGTLVQVQSA.

In an embodiment, the targeting moiety directed against CD8 comprises a VHH comprising the amino acid sequence of:

(SEQ ID NO: 455)
EVQLVESGGGLVQPGGSLRLSCAASGFTFDDYVIGWFRQAPGKGREGVAC
IRIFDRHTYYADSVKGRFTISSDNSKNTVYLQMNSLRAEDTATYYCAAGS
FFGCTRPEGDMDYFGQGTLVQVQSA.

In an embodiment, the targeting moiety directed against CD8 comprises a VHH comprising the amino acid sequence of:

(SEQ ID NO: 456)
EVQLVESGGGLVQPGGSLRLSCAASGFTFDDYAIGWFRQAPGKGREGVAC
IRIFDRHTYYADSVKGRFTISSDNSKNTVYLQMNSLRAEDTATYYCAAGS
FWGCTRPEGDMDYFGQGTLVQVQSA.

In various embodiments, the targeting moieties of the invention may comprise a sequence that targets CD8 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein).

In one embodiment, the present chimeric proteins or chimeric protein complexes such as Fc-based chimeric protein complexes have a targeting moiety directed against a T cell, for example, mediated by targeting to CD4, along with any of the signaling agents (e.g., a modified IL-2) described herein. In an embodiment, the present chimeric proteins or chimeric protein complexes, such as Fc-based chimeric protein complexes, have a targeting moiety directed against CD4 on T cells.

In one embodiment, the present chimeric proteins or chimeric protein complexes, such as Fc-based chimeric protein complexes, have a targeting moiety directed against a T cell, for example, mediated by targeting to CD3, CXCR3, CCR4, CCR9, CD70, CD103, or one or more immune checkpoint markers, along with any of the signaling agents (e.g., modified IL-2) described herein. In an embodiment, the present chimeric proteins or chimeric protein complexes such as Fc-based chimeric protein complexes have a targeting moiety directed against CD3 on T cells and a second targeting moiety directed against PD-L1 or PD-L2 on tumor cells.

In some embodiments, the present chimeric proteins or chimeric protein complexes such as Fc-based chimeric protein complexes have one or more targeting moieties directed against CD3 expressed on T cells. In some embodiments, the chimeric proteins or chimeric protein complexes such as Fc-based chimeric protein complexes have one or more targeting moieties which selectively bind a CD3 polypeptide. In some embodiments, the chimeric proteins or chimeric protein complexes such as Fc-based chimeric protein complexes comprise one or more antibodies, antibody derivatives or formats, peptides or polypeptides, or fusion proteins that selectively bind a CD3 polypeptide.

In an embodiment, the targeting moiety comprises the anti-CD3 antibody muromonab-CD3 (aka Orthoclone OKT3), or fragments thereof. Muromonab-CD3 is disclosed in U.S. Pat. No. 4,361,549 and Wilde et al. (1996) 51:865-894, the entire disclosures of which are hereby incorporated by reference. In illustrative embodiments, muromonab-CD3 or an antigen-binding fragment thereof for use in the methods provided herein comprises a heavy chain comprising the amino acid sequence of (SEQ ID NO:185); and/or a light chain comprising the amino acid sequence of (SEQ ID NO:186).

In an embodiment, the targeting moiety comprises the anti-CD3 antibody otelixizumab, or fragments thereof. Otelixizumab is disclosed in U.S. Patent Publication No. 20160000916 and Chatenoud et al. (2012) 9:372-381, the entire disclosures of which are hereby incorporated by reference. In illustrative embodiments, otelixizumab or an antigen-binding fragment thereof for use in the methods provided herein comprises a heavy chain comprising the amino acid sequence of: SEQ ID NO: 187; and/or a light chain comprising the amino acid sequence of SEQ ID NO:188.

In an embodiment, the targeting moiety comprises the anti-CD3 antibody teplizumab (AKA MGA031 and hOKT3γ1(Ala-Ala), or fragments thereof. Teplizumab is disclosed in Chatenoud et al. (2012) 9:372-381, the entire disclosures of which are hereby incorporated by reference. In illustrative embodiments, teplizumab or an antigen-binding fragment thereof for use in the methods provided herein comprises a heavy chain comprising the amino acid sequence of SEQ ID NO: 189; and/or a light chain comprising the amino acid sequence of SEQ ID NO: 190.

In an embodiment, the targeting moiety comprises the anti-CD3 antibody visilizumab (AKA Nuvion®; HuM291), or fragments thereof. Visilizumab is disclosed in U.S. Pat. No. 5,834,597 and WO2004052397, and Cole et al., Transplantation (1999) 68:563-571, the entire disclosures of which are hereby incorporated by reference. In illustrative embodiments, visilizumab or an antigen-binding fragment thereof for use in the methods provided herein comprises a heavy chain variable region comprising the amino acid sequence of SEQ ID NO: 191; and/or a light chain variable region comprising the amino acid sequence of SEQ ID NO: 192.

In an embodiment, the targeting moiety comprises the anti-CD3 antibody foralumab (aka NI-0401), or fragments thereof. In various embodiments, the targeting moiety comprises any one of the anti-CD3 antibodies disclosed in US20140193399, U.S. Pat. No. 7,728,114, US20100183554, and U.S. Pat. No. 8,551,478, the entire disclosures of which are hereby incorporated by reference.

In illustrative embodiments, the anti-CD3 antibody or an antigen-binding fragment thereof for use in the methods provided herein comprises a heavy chain variable region comprising the amino acid sequence of SEQ ID Nos: 2 and 6 of U.S. Pat. No. 7,728,114 (SEQ ID No: 2 of U.S. Pat. No. 7,728,114 (SEQ ID NO:193) and SEQ ID No: 6 of U.S. Pat. No. 7,728,114 (SEQ ID NO: 194); and/or a light chain variable region comprising the amino acid sequence of SEQ ID NOs 4 and 8 of U.S. Pat. No. 7,728,114 (SEQ ID No: 4 of U.S. Pat. No. 7,728,114 (SEQ ID NO:195) and SEQ ID No: 8 of U.S. Pat. No. 7,728,114 (SEQ ID NO:196).

In an embodiment, the targeting moiety comprises a heavy chain variable region comprising the amino acid sequence of SEQ ID NO:2 of U.S. Pat. No. 7,728,114 and a light chain variable region comprising the amino acid sequence of SEQ ID NO:4 of U.S. Pat. No. 7,728,114. In an embodiment, the targeting moiety comprises any one of the anti-CD3 antibodies disclosed in US2016/0168247, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0168247 (SEQ ID No: 6 of US2016/0168247 (SEQ ID NO:197); SEQ ID No: 7 of US2016/0168247 (SEQ ID NO: 198); SEQ ID No: 8 of US2016/0168247 (SEQ ID NO: 199); and SEQ ID No: 9 of US2016/0168247 (SEQ ID NO:200)); and/or a light chain comprising an amino acid sequence selected from SEQ ID Nos: 10-12 of US2016/0168247 (SEQ ID No: 10 of US2016/0168247 (SEQ ID NO:201); SEQ ID No: 11 of US2016/0168247 (SEQ ID NO:202); and SEQ ID No: 12 of US2016/0168247 (SEQ ID NO:203).

In an embodiment, the targeting moiety comprises any one of the anti-CD3 antibodies disclosed in US2015/0175699, the entire contents of which are hereby incorporated by reference. In illustrative embodiments, the antibody or an antigen-binding fragment thereof for use in the methods provided herein comprises a heavy chain comprising an amino acid sequence selected from SEQ ID No: 9 of US2015/0175699 (SEQ ID NO:204); and/or a light chain comprising an amino acid sequence selected from SEQ ID No: 10 of US2015/0175699 (SEQ ID NO:205).

In an embodiment, the targeting moiety comprises any one of the anti-CD3 antibodies disclosed in U.S. Pat. No. 8,784,821, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 18, 34, 50, 66, 82, 98 and 114 of U.S. Pat. No. 8,784,821 (SEQ ID No: 2 of U.S. Pat. No. 8,784,821 (SEQ ID NO:206); SEQ ID No: 18 of U.S. Pat. No. 8,784,821 (SEQ ID NO:207); SEQ ID No: 34 of U.S. Pat. No. 8,784,821 (SEQ ID NO:208); SEQ ID No: 50 of U.S. Pat. No. 8,784,821 (SEQ ID NO:209); SEQ ID No: 66 of U.S. Pat. No. 8,784,821 (SEQ ID NO:210); SEQ ID No: 82 of U.S. Pat. No. 8,784,821 (SEQ ID NO:211); SEQ ID No: 98 of U.S. Pat. No. 8,784,821 (SEQ ID NO:212); and SEQ ID No: 114 of U.S. Pat. No. 8,784,821 (SEQ ID NO:213)); and/or a light chain comprising an amino acid sequence selected from SEQ ID Nos: 10, 26, 42, 58, 74, 90, 106 and 122 of U.S. Pat. No. 8,784,821 (SEQ ID No: 10 of U.S. Pat. No. 8,784,821 (SEQ ID NO:214); SEQ ID No: 26 of U.S. Pat. No. 8,784,821 (SEQ ID NO:215); SEQ ID No: 42 of U.S. Pat. No. 8,784,821 (SEQ ID NO:216); SEQ ID No: 58 of U.S. Pat. No. 8,784,821 (SEQ ID NO:217); SEQ ID No: 74 of U.S. Pat. No. 8,784,821 (SEQ ID NO:218); SEQ ID No: 90 of U.S. Pat. No. 8,784,821 (SEQ ID NO:219); SEQ ID No: 106 of U.S. Pat. No. 8,784,821 (SEQ ID NO:220); and SEQ ID No: 122 of U.S. Pat. No. 8,784,821 (SEQ ID NO:221).

In an embodiment, the targeting moiety comprises any one of the anti-CD3 binding constructs disclosed in US20150118252, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6 and 86 of US20150118252 (SEQ ID No: 6 of US20150118252 (SEQ ID NO:222) and SEQ ID No: 86 of US20150118252 (SEQ ID NO:223)) and/or a light chain comprising an amino acid sequence selected from SEQ ID No: 3 of US2015/0175699 (SEQ ID No: 3 of US20150118252 (SEQ ID NO:224)).

In an embodiment, the targeting moiety comprises any one of the anti-CD3 binding proteins disclosed in US2016/0039934, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0039934 (SEQ ID No: 6 of US2016/0039934 (SEQ ID NO:225); SEQ ID No: 7 of US2016/0039934 (SEQ ID NO:226); SEQ ID No: 8 of US2016/0039934 (SEQ ID NO:227); and SEQ ID No: 9 of US2016/0039934 (SEQ ID NO:228)); and/or a light chain comprising an amino acid sequence selected from SEQ ID Nos: 1-4 of US2016/0039934 (SEQ ID No: 1 of US2016/0039934 (SEQ ID NO:229); SEQ ID No: 2 of US2016/0039934 (SEQ ID NO:230); SEQ ID No: 3 of US2016/0039934 (SEQ ID NO:231); and SEQ ID No: 4 of US2016/0039934 (SEQ ID NO:232).

In various embodiments, the targeting moieties of the invention may comprise a sequence that targets CD3 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein).

In various embodiments, the targeting moieties of the invention may comprise any combination of heavy chain, light chain, heavy chain variable region, light chain variable region, complementarity determining region (CDR), and framework region sequences that target CD3 as disclosed herein. In various embodiments, the targeting moieties of the invention may comprise any heavy chain, light chain, heavy chain variable region, light chain variable region, complementarity determining region (CDR), and framework region sequences of the CD3-specific antibodies including, but not limited to, X35-3, VIT3, BMA030 (BW264/56), CLB-T3/3, CRIS7, YTH12.5, FI 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, WT31 and F101.01. These CD3-specific antibodies are well known in the art and, inter alia, described in Tunnacliffe (1989), Int. Immunol. 1, 546-550, the entire disclosures of which are hereby incorporated by reference.

Additional antibodies, antibody derivatives or formats, peptides or polypeptides, or fusion proteins that selectively bind or target CD3 are disclosed in US Patent Publication No. 2016/0000916, U.S. Pat. Nos. 4,361,549, 5,834,597, 6,491,916, 6,406,696, 6,143,297, 6,750,325 and International Publication No. WO 2004/052397, the entire disclosures of which are hereby incorporated by reference.

In one embodiment, the present chimeric proteins or chimeric protein complexes such as Fc-based chimeric protein complexes have (a targeting moiety directed against a T cell, for example, mediated by targeting to PD-1, along with any of the signaling agents (e.g., a modified IL-2) described herein.

By way of non-limiting example, in various embodiments, the present chimeric proteins or chimeric protein complexes such as Fc-based chimeric protein complexes have a targeting moiety directed against a B cell, for example, mediated by targeting to CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, or CDw150; along with any of the signaling agents (e.g., a modified IL-2) described herein. In an embodiment, the present chimeric proteins or chimeric protein complexes such as Fc-based chimeric protein complexes have a targeting moiety directed against CD20.

In one embodiment, the present chimeric proteins or chimeric protein complexes such as Fc-based chimeric protein complexes have a targeting moiety directed against a B cell, for example, mediated by targeting to CD19, CD20 or CD70, along with any of the signaling agents (e.g., a modified IL-2) described herein.

In one embodiment, the present chimeric proteins or chimeric protein complexes such as Fc-based chimeric protein complexes have a targeting moiety directed against a B cell, for example, mediated by targeting to CD20, along with any of the signaling agents (e.g., a modified IL-2) described herein. In an embodiment, the present chimeric proteins or chimeric protein complexes such as Fc-based chimeric protein complexes have a targeting moiety directed against CD20 on B cells and a second targeting moiety directed against PD-L1 or PD-L2 on tumor cells. By way of example, in some embodiments, the CD20 targeting moiety is a recombinant heavy-chain-only antibody (VHH) having the sequence of:

(SEQ ID NO: 288)
QVQLQESGGGLAQAGGSLRLSCAASGRTFSMGWFRQAPGKEREFVAAITY
SGGSPYYASSVRGRFTISRDNAKNTVYLQMNSLKPEDTAVYYCAANPTYG
SDWNAENWGQGTQVTVSS.

By way of non-limiting example, in various embodiments, the present chimeric proteins or chimeric protein complexes such as Fc-based chimeric protein complexes have a targeting moiety directed against a NK cell, for example, mediated by targeting to 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAMF6, Rae-1, Rae-1 a, Rae-1 B, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d, or ULBP-3; along with any of the signaling agents (e.g., a modified IL-2) described herein.

In one embodiment, the present chimeric proteins or chimeric protein complexes such as Fc-based chimeric protein complexes have a targeting moiety directed against a NK cell, for example, mediated by targeting to Kir1alpha, DNAM-1 or CD64, along with any of the signaling agents (e.g., a modified IL-2) described herein.

In one embodiment, the present chimeric proteins or chimeric protein complexes such as Fc-based chimeric protein complexes have a targeting moiety directed against a NK cell, for example, mediated by targeting to TIGIT or KIR1, along with any of the signaling agents (e.g., a modified IL-2) described herein. In an embodiment, the present chimeric proteins or chimeric protein complexes such as Fc-based chimeric protein complexes have a targeting moiety directed against TIGIT on NK cells and a second targeting moiety directed against PD-L1 or PD-L2 on tumor cells.

By way of non-limiting example, in various embodiments, the present chimeric proteins or chimeric protein complexes such as Fc-based chimeric protein complexes have a targeting moiety directed against a dendritic cell, for example, mediated by targeting to CLEC-9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, Integrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102, or Vanilloid R1; along with any of the signaling agents (e.g., a modified IL-2) described herein.

In one embodiment, the present chimeric proteins or chimeric protein complexes such as Fc-based chimeric protein complexes have a targeting moiety directed against a dendritic cell, for example, mediated by targeting to CLEC-9A, DC-SIGN, CD64, CLEC4A, or DEC205, along with any of the signaling agents (e.g., a modified IL-2) described herein. In an embodiment, the present chimeric proteins or chimeric protein complexes such as Fc-based chimeric protein complexes have a targeting moiety directed against CLEC9A on dendritic cells and a second targeting moiety directed against PD-L1 or PD-L2 on tumor cells.

In one embodiment, the present chimeric proteins or chimeric protein complexes such as Fc-based chimeric protein complexes have a targeting moiety directed against a dendritic cell, for example, mediated by targeting to CLEC9A, along with any of the signaling agents (e.g., a modified IL-2) described herein. In an embodiment, the present chimeric proteins or chimeric protein complexes such as Fc-based chimeric protein complexes have a targeting moiety directed against CLEC9A on dendritic cells and a second targeting moiety directed against PD-L1 or PD-L2 on tumor cells.

In one embodiment, the present chimeric proteins or chimeric protein complexes such as Fc-based chimeric protein complexes have a targeting moiety directed against a dendritic cell, for example, mediated by targeting to XCR1, along with any of the signaling agents (e.g., a modified IL-2) described herein. In an embodiment, the present chimeric proteins or chimeric protein complexes such as Fc-based chimeric protein complexes have a targeting moiety directed against XCR1 on dendritic cells and a second targeting moiety directed against PD-L1 or PD-L2 on tumor cells.

In one embodiment, the present chimeric proteins or chimeric protein complexes such as Fc-based chimeric protein complexes have a targeting moiety directed against a dendritic cell, for example, mediated by targeting to RANK, along with any of the signaling agents (e.g., a modified IL-2) described herein. In an embodiment, the present chimeric proteins or chimeric protein complexes such as Fc-based chimeric protein complexes have a targeting moiety directed against RANK on dendritic cells and a second targeting moiety directed against PD-L1 or PD-L2 on tumor cells.

By way of non-limiting example, in various embodiments, the present chimeric proteins or chimeric protein complexes such as Fc-based chimeric protein complexes have a targeting moiety directed against a monocyte/macrophage, for example, mediated by targeting to SIRP1a, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-Y R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, CD206, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, LMIR1/CD300A, CD97, LMIR2/CD300c, CD163, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-I, IL-6 R, TREM-2, CXCRI/IL-8 RA, TREM-3, or TREMLI/TLT-1; along with any of the signaling agents (e.g., a modified IL-2) described herein.

In one embodiment, the present chimeric proteins or chimeric protein complexes such as Fc-based chimeric protein complexes have a targeting moiety directed against a monocyte/macrophage, for example, mediated by targeting to B7-H1, CD31/PECAM-1, CD163, CCR2, or Macrophage Mannose Receptor CD206, along with any of the signaling agents (e.g., a modified IL-2) described herein.

In one embodiment, the present chimeric proteins or chimeric protein complexes such as Fc-based chimeric protein complex has a targeting moiety directed against a monocyte/macrophage, for example, mediated by targeting to SIRP1a, along with any of the signaling agents (e.g., a modified IL-2) described herein. In an embodiment, the present chimeric proteins or chimeric protein complexes such as Fc-based chimeric protein complex has a targeting moiety directed against SIRP1a on macrophage cells and a second targeting moiety directed against PD-L1 or PD-L2 on tumor cells.

In various embodiments, the present chimeric proteins or chimeric protein complexes such as Fc-based chimeric protein complex has one or more targeting moieties directed against a checkpoint marker, e.g. one or more of PD-1/PD-L1 or PD-L2, CD28/CD80 or CD86, CTLA4/CD80 or CD86, ICOS/ICOSL or B7RP1, BTLA/HVEM, KIR, LAG3, CD137/CD137L, OX40/OX40L, CD27, CD40L, TIM3/Gal9, CD47, CD70, and A2aR, along with any of the signaling agents (e.g., a modified IL-2) described herein.

In some embodiments, the present chimeric proteins or chimeric protein complexes such as Fc-based chimeric protein complex comprises one or more targeting moieties directed to the same or different immune cells. In some embodiments, the present chimeric proteins or chimeric protein complexes such as Fc-based chimeric protein complex has one or more targeting moieties directed against an immune cell selected from a T cell, a B cell, a dendritic cell, a macrophage, a NK cell, or subsets thereof, along with any of the signaling agents (e.g., a modified IL-2) described herein.

In one embodiment, the present chimeric proteins or chimeric protein complexes such as Fc-based chimeric protein complex comprises a targeting moiety directed against a tumor cell. In such embodiments, the targeting moiety may bind to any of the tumor antigens described herein.

In some embodiments, the chimeric proteins or chimeric protein complexes, such as Fc-based chimeric protein complexes, of the invention comprise one or more targeting moieties having recognition domains that bind to a target (e.g. antigen, receptor) of interest including those found on one or more cells selected from adipocytes (e.g., white fat cell, brown fat cell), liver lipocytes, hepatic cells, kidney cells (e.g., kidney parietal cell, kidney salivary gland, mammary gland, etc.), duct cells (of seminal vesicle, prostate gland, etc.), intestinal brush border cells (with microvilli), exocrine gland striated duct cells, gall bladder epithelial cells, ductulus efferens nonciliated cells, epididymal principal cells, epididymal basal cells, endothelial cells, ameloblast epithelial cells (tooth enamel secretion), planum semilunatum epithelial cells of vestibular system of ear (proteoglycan secretion), organ of Corti interdental epithelial cells (secreting tectorial membrane covering hair cells), loose connective tissue fibroblasts, corneal fibroblasts (corneal keratocytes), tendon fibroblasts, bone marrow reticular tissue fibroblasts, nonepithelial fibroblasts, pericytes, nucleus pulposus cells of intervertebral disc, cementoblasts/cementocytes (tooth root bonelike ewan cell secretion), odontoblasts/odontocytes (tooth dentin secretion), hyaline cartilage chondrocytes, fibrocartilage chondrocytes, elastic cartilage chondrocytes, osteoblasts/osteocytes, osteoprogenitor cells (stem cell of osteoblasts), hyalocytes of vitreous body of eye, stellate cells of perilymphatic space of ear, hepatic stellate cells (Ito cell), pancreatic stelle cells, skeletal muscle cells, satellite cells, heart muscle cells, smooth muscle cells, myoepithelial cells of iris, myoepithelial cells of exocrine glands, exocrine secretory epithelial cells (e.g., salivary gland cells, mammary gland cells, lacrimal gland cells, sweat gland cells, sebaceious gland cells, prostate gland cells, gastric glad cells, pancreatic acinar cells, pneumocytes), a hormone secreting cells (e.g., pituitary cells, neurosecretory cells, gut and respiratory tract cells, thyroid gland cells, parathyroid glad cells, adrenal gland cells, Leydig cells of testes, pancreatic islet cells), keratinizing epithelial cells, wet stratified barrier epithelial cells, neuronal cells (e.g., sensory transducer cells, autonomic neuron cells, sense organ and peripheral neuron supporting cells, and central nervous system neurons and glial cells such as interneurons, principal cells, astrocytes, oligodendrocytes, and ependymal cells).

### Targeting Moiety Formats

In various embodiments, the targeting moiety of the present chimeric proteins or chimeric protein complexes, such as Fc-based chimeric protein complexes, are protein-based agents capable of specific binding, such as an antibodies or derivatives thereof. In an embodiment, the targeting moiety comprises an antibody. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the targeting moiety comprises antibody derivatives or formats. In some embodiments, the targeting moiety of the present chimeric proteins or chimeric protein complexes such as Fc-based chimeric protein complex is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterases; a plastic antibodies; a phylomer; a stradobodies; a maxibodies; an evibody; a fynomer, an armadillo repeat protein, a Kunitz domain, an avimer, an atrimer, a probody, an immunobody, a triomab, a troybody; a pepbody; a vaccibody, a UniBody; affimers, a DuoBody, a Fv, a Fab, a Fab′, a F(ab′)2, a peptide mimetic molecule, or a synthetic molecule, as described in US patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In one embodiment, the targeting moiety comprises a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3). VHHs are commercially available under the trademark of NANOBODY or NANOBODIES.

In an embodiment, the targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. a HUMABODIES are described in, for example, WO 2016/113555 and WO2016/113557, the entire disclosure of which is incorporated by reference.

In various embodiments, the targeting moiety of the present chimeric proteins or chimeric protein complexes such as Fc-based chimeric protein complex is a protein-based agent capable of specific binding to a cell receptor, such as a natural ligand for the cell receptor. In various embodiments, the cell receptor is found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In some embodiments, the cell receptor is found on megakaryocytes, thrombocytes, erythrocytes, mast cells, basophils, neutrophils, eosinophils, or subsets thereof.

In some embodiments, the targeting moiety is a natural ligand such as a chemokine. Illustrative chemokines that may be included in the chimeric proteins or chimeric protein complexes such as Fc-based chimeric protein complex of the invention include, but are not limited to, CCL1, CCL2, CCL4, CCL5, CCL6, CCL7, CCL8, CCL9, CCL10, CCL11, CCL12, CCL13, CCL14, CCL15, CCL16, CL17, CCL18, CCL19, CCL20, CCL21, CCL22, CCL23, CCL24, CLL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, HCC-4, and LDGF-PBP. In an illustrative embodiment, the targeting moiety may be XCL1 which is a chemokine that recognizes and binds to the dendritic cell receptor XCR1. In another illustrative embodiment, the targeting moiety is CCL1, which is a chemokine that recognizes and binds to CCR8. In another illustrative embodiment, the targeting moiety is CCL2, which is a chemokine that recognizes and binds to CCR2 or CCR9. In another illustrative embodiment, the targeting moiety is CCL3, which is a chemokine that recognizes and binds to CCR1, CCR5, or CCR9. In another illustrative embodiment, the targeting moiety is CCL4, which is a chemokine that recognizes and binds to CCR1 or CCR5 or CCR9. In another illustrative embodiment, the targeting moiety is CCL5, which is a chemokine that recognizes and binds to CCR1 or CCR3 or CCR4 or CCR5. In another illustrative embodiment, the targeting moiety is CCL6, which is a chemokine that recognizes and binds to CCR1. In another illustrative embodiment, the targeting moiety is CCL7, which is a chemokine that recognizes and binds to CCR2 or CCR9. In another illustrative embodiment, the targeting moiety is CCL8, which is a chemokine that recognizes and binds to CCR1 or CCR2 or CCR2B or CCR5 or CCR9. In another illustrative embodiment, the targeting moiety is CCL9, which is a chemokine that recognizes and binds to CCR1. In another illustrative embodiment, the targeting moiety is CCL10, which is a chemokine that recognizes and binds to CCR1. In another illustrative embodiment, the targeting moiety is CCL11, which is a chemokine that recognizes and binds to CCR2 or CCR3 or CCR5 or CCR9. In another illustrative embodiment, the targeting moiety is CCL13, which is a chemokine that recognizes and binds to CCR2 or CCR3 or CCR5 or CCR9. In another illustrative embodiment, the targeting moiety is CCL14, which is a chemokine that recognizes and binds to CCR1 or CCR9. In another illustrative embodiment, the targeting moiety is CCL15, which is a chemokine that recognizes and binds to CCR1 or CCR3. In another illustrative embodiment, the targeting moiety is CCL16, which is a chemokine that recognizes and binds to CCR1, CCR2, CCR5, or CCR8. In another illustrative embodiment, the targeting moiety is CCL17, which is a chemokine that recognizes and binds to CCR4. In another illustrative embodiment, the targeting moiety is CCL19, which is a chemokine that recognizes and binds to CCR7. In another illustrative embodiment, the targeting moiety is CCL20, which is a chemokine that recognizes and binds to CCR6. In another illustrative embodiment, the targeting moiety is CCL21, which is a chemokine that recognizes and binds to CCR7. In another illustrative embodiment, the targeting moiety is CCL22, which is a chemokine that recognizes and binds to CCR4. In another illustrative embodiment, the targeting moiety is CCL23, 25 which is a chemokine that recognizes and binds to CCR1. In another illustrative embodiment, the targeting moiety is CCL24, which is a chemokine that recognizes and binds to CCR3. In another illustrative embodiment, the targeting moiety is CCL25, which is a chemokine that recognizes and binds to CCR9. In another illustrative embodiment, the targeting moiety is CCL26, which is a chemokine that recognizes and binds to CCR3. In another illustrative embodiment, the targeting moiety is CCL27, which is a chemokine that recognizes and binds to CCR10. In another illustrative embodiment, the targeting moiety is CCL28, which is a chemokine that recognizes and binds to CCR3 or CCR10. In another illustrative embodiment, the targeting moiety is CXCL1, which is a chemokine that recognizes and binds to CXCR1 or CXCR2. In another illustrative embodiment, the targeting moiety is CXCL2, which is a chemokine that recognizes and binds to CXCR2. In another illustrative embodiment, the targeting moiety is CXCL3, which is a chemokine that recognizes and binds to CXCR2. In another illustrative embodiment, the targeting moiety is CXCL4, which is a chemokine that recognizes and binds to CXCR3B. In another illustrative embodiment, the targeting moiety is CXCL5, which is a chemokine that recognizes and binds to CXCR2. In another illustrative embodiment, the targeting moiety is CXCL6, which is a chemokine that recognizes and binds to CXCR1 or CXCR2. In another illustrative embodiment, the targeting moiety is CXCL8, which is a chemokine that recognizes and binds to CXCR1 or CXCR2. In another illustrative embodiment, the targeting moiety is CXCL9, which is a chemokine that recognizes and binds to CXCR3. In another illustrative embodiment, the targeting moiety is CXCL10, which is a chemokine that recognizes and binds to CXCR3. In another illustrative embodiment, the targeting moiety is CXCL11, which is a chemokine that recognizes and binds to CXCR3 or CXCR7. In another illustrative embodiment, the targeting moiety is CXCL 12, which is a chemokine that recognizes and binds to CXCR4 or CXCR7. In another illustrative embodiment, the targeting moiety is CXCL13, which is a chemokine that recognizes and binds to CXCR5. In another illustrative embodiment, the targeting moiety is CXCL16, which is a chemokine that recognizes and binds to CXCR6. In another illustrative embodiment, the targeting moiety is LDGF-PBP, which is a chemokine that recognizes and binds to CXCR2. In another illustrative embodiment, the targeting moiety is XCL2, which is a chemokine that recognizes and binds to XCR1. In another illustrative embodiment, the targeting moiety is CX3CL1, which is a chemokine that recognizes and binds to CX3CR1.

In some embodiments, the targeting moiety is a natural ligand such as FMS-like tyrosine kinase 3 ligand (FIt3L) or a truncated region thereof (e.g., which is able to bind Flt3). In some embodiments, the targeting moiety is an extracellular domain of Flt3L. In some embodiments, the targeting moiety comprising a FIt3L domain, wherein the Flt3L domain is a single chain dimer, optionally where one FIt3L domain is connected to the other FIt3L domain via one or more linkers, wherein the linker is a flexible linker. In some embodiments, the targeting moiety of the present invention comprises Flt3L domain, wherein the FIt3L domain is a single chain dimer and a Fc domain, the Fc domain optionally having one or more mutations that reduces or eliminates one or more effector functions of the Fc domain, promotes Fc chain pairing in the Fc domain, and/or stabilizes a hinge region in the Fc domain. In some embodiments, the targeting moiety recognizes CD20. In some embodiments, the targeting moiety recognizes PD-L1. In some embodiments, the targeting moiety recognizes Clec9A.

In various embodiments, the present chimeric proteins or chimeric protein complexes such as Fc-based chimeric protein complex comprises targeting moieties in various combinations. In an illustrative embodiment, the present chimeric proteins or chimeric protein complexes such as Fc-based chimeric protein complex may comprise two targeting moieties, wherein both targeting moieties are antibodies or derivatives thereof. In another illustrative embodiment, the present chimeric proteins or chimeric protein complexes such as Fc-based chimeric protein complex may comprise two targeting moieties, wherein both targeting moieties are natural ligands for cell receptors. In a further illustrative embodiment, the present chimeric proteins or chimeric protein complexes such as Fc-based chimeric protein complex may comprise two targeting moieties, wherein one of the targeting moieties is an antibody or derivative thereof, and the other targeting moiety is a natural ligand for a cell receptor. In various embodiments, the recognition domain of the present chimeric proteins or chimeric protein complexes such as Fc-based chimeric protein complex functionally modulates (by way of non-limitation, partially or completely neutralizes) the target (e.g. antigen, receptor) of interest, e.g. substantially inhibiting, reducing, or neutralizing a biological effect that the antigen has. For example, various recognition domains may be directed against one or more tumor antigens that are actively suppressing, or have the capacity to suppress, the immune system of, for example, a patient bearing a tumor. For example, in some embodiments, the present chimeric proteins or chimeric protein complexes such as Fc-based chimeric protein complex functionally modulates immune inhibitory signals (e.g. checkpoint inhibitors), for example, one or more of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. For example, in some embodiments, the present chimeric proteins or chimeric protein complexes such as Fc-based chimeric protein complex is engineered to disrupt, block, reduce, and/or inhibit the transmission of an immune inhibitory signal, by way of non-limiting example, the binding of PD-1 with PD-L1 or PD-L2 and/or the binding of CTLA-4 with one or more of AP2M1, CD80, CD86, SHP-2, and PPP2R5A.

In various embodiments, the recognition domain of the present chimeric proteins or chimeric protein complexes such as Fc-based chimeric protein complex binds but does not functionally modulate the target (e.g. antigen, receptor) of interest, e.g. the recognition domain is, or is akin to, a binding antibody. For instance, in various embodiments, the recognition domain simply targets the antigen or receptor but does not substantially inhibit, reduce or functionally modulate a biological effect that the antigen or receptor has. For example, some of the smaller antibody formats described above (e.g. as compared to, for example, full antibodies) have the ability to target hard to access epitopes and provide a larger spectrum of specific binding locales. In various embodiments, the recognition domain binds an epitope that is physically separate from an antigen or receptor site that is important for its biological activity (e.g. the antigen's active site).

Such non-neutralizing binding finds use in various embodiments of the present invention, including methods in which the present chimeric proteins or chimeric protein complexes such as Fc-based chimeric protein complex is used to directly or indirectly recruit active immune cells to a site of need via an effector antigen, such as any of those described herein. For example, in various embodiments, the present chimeric proteins or chimeric protein complexes such as Fc-based chimeric protein complex may be used to directly or indirectly recruit cytotoxic T cells via CD8 to a tumor cell in a method of reducing or eliminating a tumor (e.g. the chimeric proteins or chimeric protein complexes such as Fc-based chimeric protein complex may comprise an anti-CD8 recognition domain and a recognition domain directed against a tumor antigen). In such embodiments, it is desirable to directly or indirectly recruit CD8-expressing cytotoxic T cells but not to functionally modulate the CD8 activity. On the contrary, in these embodiments, CD8 signaling is an important piece of the tumor reducing or eliminating effect. By way of further example, in various methods of reducing or eliminating tumors, the present chimeric proteins or chimeric protein complexes such as Fc-based chimeric protein complex is used to directly or indirectly recruit dendritic cells (DCs) via CLEC9A (e.g. the chimeric proteins or chimeric protein complexes such as Fc-based chimeric protein complex may comprise an anti-CLEC9A recognition domain and a recognition domain directed against a tumor antigen). In such embodiments, it is desirable to directly or indirectly recruit CLEC9A-expressing DCs but not to functionally modulate the CLEC9A activity. On the contrary, in these embodiments, CLEC9A signaling is an important piece of the tumor reducing or eliminating effect.

In various embodiments, the recognition domain of the present chimeric proteins or chimeric protein complexes such as Fc-based chimeric protein complex binds to XCR1 e.g. on dendritic cells. For instance, the recognition domain, in some embodiments comprises all or part of XCL1 or a non-neutralizing anti-XCR1 agent.

In various embodiments, the recognition domain of the present chimeric proteins or chimeric protein complexes such as Fc-based chimeric protein complex binds to an immune modulatory antigen (e.g. immune stimulatory or immune inhibitory). In various embodiments, the immune modulatory antigen is one or more of 4-1BB, OX-40, HVEM, GITR, CD27, CD28, CD30, CD40, ICOS ligand; OX-40 ligand, LIGHT (CD258), GITR ligand, CD70, B7-1, B7-2, CD30 ligand, CD40 ligand, ICOS, ICOS ligand, CD137 ligand and TL1A. In various embodiments, such immune stimulatory antigens are expressed on a tumor cell. In various embodiments, the recognition domain of the present chimeric proteins or chimeric protein complexes such as Fc-based chimeric protein complex binds but does not functionally modulate such immune stimulatory antigens and therefore allows recruitment of cells expressing these antigens without the reduction or loss of their potential tumor reducing or eliminating capacity.

In various embodiments, the recognition domain of the present chimeric proteins or chimeric protein complexes such as Fc-based chimeric protein complex may be in the context of chimeric proteins or chimeric protein complexes such as Fc-based chimeric protein complex that comprises two recognition domains that have neutralizing activity, or comprises two recognition domains that have non-neutralizing (e.g. binding) activity, or comprises one recognition domain that has neutralizing activity and one recognition domain that has non-neutralizing (e.g. binding) activity.

### Fc Domains

The fragment crystallizable domain (Fc domain) is the tail region of an antibody that interacts with Fc receptors located on the cell surface of cells that are involved in the immune system, e.g., B lymphocytes, dendritic cells, natural killer cells, macrophages, neutrophils, eosinophils, basophils, and mast cells. In IgG, IgA and IgD antibody isotypes, the Fc domain is composed of two identical protein fragments, derived from the second and third constant domains of the antibody's two heavy chains. In IgM and IgE antibody isotypes, the Fc domain contains three heavy chain constant domains (CH domains 2-4) in each polypeptide chain.

In some embodiments, the Fc-based chimeric protein of complex the present technology includes a Fc domain. In some embodiments, the Fc domains are from selected from IgG, IgA, IgD, IgM or IgE. In some embodiments, the Fc domains are from selected from IgG1, IgG2, IgG3, or IgG4.

In some embodiments, the Fc domains are from selected from human IgG, IgA, IgD, IgM or IgE. In some embodiments, the Fc domains are from selected from human IgG1, IgG2, IgG3, or IgG4.

In some embodiments, the Fc domains of the Fc-based chimeric protein complex comprise the CH₂ and CH₃ regions of IgG. In some embodiments, the IgG is human IgG. In some embodiments, the human IgG is selected from IgG1, IgG2, IgG3, or IgG4.

In some embodiments, the Fc domains comprise one or more mutations. In some embodiments, the mutation(s) to the Fc domains reduces or eliminates the effector function the Fc domains. In some embodiments, the mutated Fc domain has reduced affinity or binding to a target receptor. By way of example, in some embodiments, the mutation to the Fc domains reduces or eliminates the binding of the Fc domains to FcγR. In some embodiments, the FcγR is selected from FcγRI; FcγRIIa, 131 R/R; FcγRIIa, 131 H/H, FcγRIIb; and FcγRIII. In some embodiments, the mutation to the Fc domains reduces or eliminated binding to complement proteins, such as, e.g., Clq. In some embodiments, the mutation to the Fc domains reduces or eliminated binding to both FcγR and complement proteins, such as, e.g., Clq.

In some embodiments, the Fc domains comprise the LALA mutation to reduce or eliminate the effector function of the Fc domains. By way of example, in some embodiments, the LALA mutation comprises L234A and L235A substitutions in human IgG (e.g., IgG1) (wherein the numbering is based on the commonly used numbering of the CH₂ residues for human IgG1 according to EU convention (PNAS, Edelman et al., 1969; 63 (1) 78-85).

In some embodiments, the Fc domains of human IgG comprise a mutation at 46. to reduce or eliminate the effector function of the Fc domains. By way of example, in some embodiments, the mutations are selected from L234A, L234F, L235A, L235E, L235Q, K322A, K322Q, D265A, P329G, P329A, P331G, and P331S.

In some embodiments, the Fc domains comprise the FALA mutation to reduce or eliminate the effector function of the Fc domains. By way of example, in some embodiments, the FALA mutation comprises F234A and L235A substitutions in human IgG4.

In some embodiments, the Fc domains of human IgG4 comprise a mutation at one or more of F234, L235, K322, D265, and P329 to reduce or eliminate the effector function of the Fc domains. By way of example, in some embodiments, the mutations are selected from F234A, L235A, L235E, L235Q, K322A, K322Q, D265A, P329G, and P329A.

In some embodiments, the mutation(s) to the Fc domain stabilize a hinge region in the Fc domain. By way of example, in some embodiments, the Fc domain comprises a mutation at S228 of human IgG to stabilize a hinge region. In some embodiments, the mutation is S228P.

In some embodiments, the mutation(s) to the Fc domain promote chain pairing in the Fc domain. In some embodiments, chain pairing is promoted by ionic pairing (a/k/a charged pairs, ionic bond, or charged residue pair).

In some embodiments, the Fc domain comprises a mutation at one more of the following amino acid residues of IgG to promote of ionic pairing: D356, E357, L368, K370, K392, D399, and K409.

By way of example, in some embodiments, the human IgG Fc domain comprise one of the mutation combinations in Table 1 to promote of ionic pairing.

TABLE 1

| Substitution(s) on | Substitution(s) on |
|---|---|
| one Fc Chain | other Fc Chain |
| D356K D399K | K392D K409D |
| E357R L368R | K370D K409D |
| E357R L368K | K370D K409D |
| E357R D399K | K370D K409D |
| E357R | K370D |
| L368R D399K | K392D K409D |
| L368K D399K | K392D K409D |
| L368R D399K | K409D |
| L368K D399K | K409D |
| L368R | K409D |
| L368K | K409D |
| K370D K409D | E357R D399K |
| K370D K409D | E357R L368R |
| K370D K409D | E357R L368K |
| K370D K409D | E357R D399K |
| K370D K409D | E357R L368R |
| K370D K409D | E357R L368K |
| K370D | E357R |
| K370D | E357R |
| K392D K409D | D356K D399K |
| K392D K409D | L368R D399K |
| K392D K409D | L368K D399K |
| K392D K409D | D399K |
| D399K | K392D K409D |
| D399K | K409D |
| K409D | L368R |
| K409D | L368K |
| K409D | L368R D399K |
| K409D | L368K D399K |
| K409D | L368R |
| K409D | L368K |
| K409D | L368R D399K |
| K409D | L368K D399K |
| K409D | D399K |

In some embodiments, chain pairing is promoted by knob-in-hole mutations. In some embodiments, the Fc domain comprises one or more mutations to allow for a knob-in-hole interaction in the Fc domain. In some embodiments, a first Fc chain is engineered to express the “knob” and a second Fc chain is engineered to express the complementary “hole.” By way of example, in some embodiments, human IgG Fc domain comprises the mutations of Table 2 to allow for a knob-in-hole interaction.

TABLE 2

| Substitution(s) on | Substitution(s) on |
|---|---|
| one Fc Chain | other Fc Chain |
| T366Y | Y407T |
| T366Y/F405A | T394W/Y407T |
| T366W | Y407A |
| T366W | Y407V |
| T366Y | Y407A |
| T366Y | Y407V |
| T366Y | Y407T |

In some embodiments, the Fc domains in the Fc-based chimeric protein complexes of the present technology comprise any combination of the above disclosed mutations. By way of example, in some embodiments, the Fc domain comprises mutations that promote ionic pairing and/or a knob-in-hole interaction. By way of example, in some embodiments, the Fc domain comprises mutations that have one or more of the following properties: promote ionic pairing, induce a knob-in-hole interaction, reduce or eliminate the effector function of the Fc domain, and cause Fc stabilization (e.g. at hinge).

By way of example, in some embodiments, a human IgG Fc domains comprise mutations disclosed in Table 3, which promote ionic pairing and/or promote a knob-in-hole interaction in the Fc domain.

TABLE 3

| Substitution(s) on | Substitution(s) on |
|---|---|
| one Fc Chain | other Fc Chain |
| T366W K370D | E357R Y407A |
| T366W K370D | E357R Y407V |
| T366W K409D | L368R Y407A |
| T366W K409D | L368R Y407V |
| T366W K409D | L368K Y407A |
| T366W K409D | L368K Y407V |
| T366W K409D | L368R D399K Y407A |
| T366W K409D | L368R D399K Y407V |
| T366W K409D | L368K D399K Y407A |
| T366W K409D | L368K D399K Y407V |
| T366W K409D | D399K Y407A |
| T366W K409D | D399K Y407V |
| T366W K392D K409D | D399K Y407A |
| T366W K392D K409D | D399K Y407V |
| T366W K392D K409D | D356K D399K Y407A |
| T366W K392D K409D | D356K D399K Y407V |
| T366W K370D K409D | E357R D399K Y407A |
| T366W K370D K409D | E357R D399K Y407V |
| T366W K370D K409D | E357R L368R Y407A |
| T366W K370D K409D | E357R L368R Y407V |
| T366W K370D K409D | E357R L368K Y407A |
| T366W K370D K409D | E357R L368K Y407V |
| T366W K392D K409D | L368R D399K Y407A |
| T366W K392D K409D | L368R D399K Y407V |
| T366W K392D K409D | L368K D399K Y407A |
| T366W K392D K409D | L368K D399K Y407V |
| E357R T366W | K370D Y407A |
| E357R T366W | K370D Y407V |
| T366W L368R | Y407A K409D |
| T366W L368R | Y407V K409D |
| T366W L368K | Y407A K409D |
| T366W L368K | Y407V K409D |
| T366W L368R D399K | Y407A K409D |
| T366W L368R D399K | Y407V K409D |
| T366W L368K D399K | Y407A K409D |
| T366W L368K D399K | Y407V K409D |
| T366W D399K | Y407A K409D |
| T366W D399K | Y407V K409D |
| 1366W D399K | K392D Y407A K409D |
| T366W D399K | K392D Y407V K409D |
| T366W D356K D399K | K392D Y407A K409D |
| T366W D356K D399K | K392D Y407V K409D |
| E357R T366W D399K | K370D Y407A K409D |
| E357R T366W D399K | K370D Y407V K409D |
| E357R T366W L368R | K370D Y407A K409D |
| E357R T366W L368R | K370D Y407V K409D |
| E357R T366W L368K | K370D Y407A K409D |
| E357R T366W L368K | K370D Y407V K409D |
| T366W L368R D399K | K392D Y407A K409D |
| T366W L368R D399K | K392D Y407V K409D |
| T366W L368K D399K | K392D Y407A K409D |

By way of example, in some embodiments, a human IgG Fc domains comprise mutations disclosed in Table 4, which promote ionic pairing, promote a knob-in-hole interaction, or a combination thereof in the Fc domain. In embodiments, the “Chain 1” and “Chain 2” of Table 4 can be interchanged (e.g. Chain 1 can have Y407T and Chain 2 can have T366Y).

TABLE 4

| Chain 1 mutation | Chain 2 mutation | Reference | IgG |
|---|---|---|---|
| T366Y | Y407T | Ridgway et al., 1996 Protein | IgG1 |
| | | Engineering, Design and Selection, | |
| | | Volume 9, Issue 7, 1 Jul. 1996, Pages | |
| | | 617-62 | |
| T366Y/F405A | T394W/Y407T | Ridgway et al., 1996 Protein | IgG1 |
| | | Engineering, Design and Selection, | |
| | | Volume 9, Issue 7, 1 Jul. 1996, Pages | |
| | | 617-62 | |
| T366W | Y407A | Atwell et al., 1997 JMB | IgG1 |
| | | Volume 270, Issue 1, 4 Jul. 1997, | |
| | | Pages 26-35 | |
| T366W | T366S/L368V/Y407A | Atwell et al., 1997 JMB | IgG1 |
| | | Volume 270, Issue 1, 4 Jul. 1997, | |
| | | Pages 26-35 | |
| T366W | L368A/Y407A | Atwell et al., 1997 JMB | IgG1 |
| | | Volume 270, Issue 1, 4 Jul. 1997, | |
| | | Pages 26-35 | |
| T366W | T366S/L368A/Y407A | Atwell et al., 1997 JMB | IgG1 |
| | | Volume 270, Issue 1, 4 Jul. 1997, | |
| | | Pages 26-35 | |
| T366W | T366S/L368G/Y407V | Atwell et al., 1997 JMB | IgG1 |
| | | Volume 270, Issue 1, 4 Jul. 1997, | |
| | | Pages 26-35 | |
| T366W/D399C | T366S/L368A/K392C/Y407V | Merchant et al., 1998 Nature | IgG1 |
| | | Biotechnology volume 16, pages 677- | |
| | | 681 (1998) | |
| T366W/K392C | T366S/L368A/D399C/Y407V | Merchant et al., 1998 Nature | IgG1 |
| | | Biotechnology volume 16, pages 677- | |
| | | 681 (1998) | |
| S354C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | S354C/T366S/L368A/Y407V | Merchant et al., 1998 Nature | IgG1 |
| | | Biotechnology volume 16, pages 677- | |
| | | 681 (1998) | |
| E356C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | E356C/T366S/L368A/Y407V | Merchant et al., 1998 Nature | IgG1 |
| | | Biotechnology volume 16, pages 677- | |
| | | 681 (1998) | |
| E357C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | E357C/T366S/L368A/Y407V | Merchant et al., 1998 Nature | IgG1 |
| | | Biotechnology volume 16, pages 677- | |
| | | 681 (1998) | |
| D339R | K409E | Gunasekaran et al., 2010 The Journal of | IgG1 |
| | | Biological Chemistry 285, 19637-19646. | |
| D339K | K409E | Gunasekaran et al., 2010 The Journal of | IgG1 |
| | | Biological Chemistry 285, 19637-19646. | |
| D339R | K409D | Gunasekaran et al., 2010 The Journal of | IgG1 |
| | | Biological Chemistry 285, 19637-19646. | |
| D339K | K409D | Gunasekaran et al., 2010 The Journal of | IgG1 |
| | | Biological Chemistry 285, 19637-19646. | |
| D339K | K360D/K409E | Gunasekaran et al., 2010 The Journal of | IgG1 |
| | | Biological Chemistry 285, 19637-19646. | |
| D339K | K392D/K409E | Gunasekaran et al., 2010 The Journal of | IgG1 |
| | | Biological Chemistry 285, 19637-19646. | |
| D339K/E356K | K392D/K409E | Gunasekaran et al., 2010 The Journal of | IgG1 |
| | | Biological Chemistry 285, 19637-19646. | |
| D339K/E357K | K392D/K409E | Gunasekaran et al., 2010 The Journal of | IgG1 |
| | | Biological Chemistry 285, 19637-19646. | |
| D339K/E356K | K409E/K439D | Gunasekaran et al., 2010 The Journal of | IgG1 |
| | | Biological Chemistry 285, 19637-19646. | |
| D339K/E357K | K370D/K409E | Gunasekaran et al., 2010 The Journal of | IgG1 |
| | | Biological Chemistry 285, 19637-19646. | |
| D339K/E356K/E357K | K370D/K392D/K409E | Gunasekaran et al., 2010 The Journal of | IgG1 |
| | | Biological Chemistry 285, 19637-19646. | |
| S364H/F405A | Y349T/T394F | Moore et al., 2011 mAbs, 3:6, 546-557 | IgG1 |
| S364H/T394F | Y349T/F405A | Moore et al., 2011 mAbs, 3:6, 546-557 | IgG1 |
| D221R/P228R/K409R | D221E/P228E/L368E | Strop et al., 2012 JMB Volume 420, | IgG1 |
| | | Issue 3, 13 Jul. 2012, Pages 204-219 | |
| C223R/E225R/P228R/K409R | C223E/P228E/L368E | Strop et al., 2012 JMB Volume 420, | IgG2 |
| | | Issue 3, 13 Jul. 2012, Pages 204-219 | |
| F405L | K409R | Labrijn et al., 2013 PNAS Mar. 26, | IgG1 |
| | | 2013. 110 (13) 5145-5150 | |
| F405A/Y407V | T394W | Von Kreudenstein et al., 2013 mAbs | IgG1 |
| | | Volume 5, 2013 - Issue 5, pp.644-654 | |
| F405A/Y407V | T366l/T394W | Von Kreudenstein et al., 2013 mAbs | IgG1 |
| | | Volume 5, 2013 - Issue 5, pp.644-654 | |
| F405A/Y407V | T366L/T394W | Von Kreudenstein et al., 2013 mAbs | IgG1 |
| | | Volume 5, 2013 - Issue 5, pp.644-654 | |
| F405A/Y407V | T366L/K392M/T394W | Von Kreudenstein et al., 2013 mAbs | IgG1 |
| | | Volume 5, 2013 - Issue 5, pp.644-654 | |
| L351Y/F405A/Y407V | T366L/K392M/T394W | Von Kreudenstein et al., 2013 mAbs | IgG1 |
| | | Volume 5, 2013 - Issue 5, pp.644-654 | |
| T350V/L351Y/F405A/Y407V | T350V/T366L/K392M/T394W | Von Kreudenstein et al., 2013 mAbs | IgG1 |
| | | Volume 5, 2013 - Issue 5, pp.644-654 | |
| T350V/L351Y/F405A/Y407V | T350V/T366L/K392L/T394W | Von Kreudenstein et al., 2013 mAbs | IgG1 |
| | | Volume 5, 2013 - Issue 5, pp.644-654 | |
| K409W | D339V/F405T | Choi et al., 2013 PNAS Jan. 2, | IgG1 |
| | | 2013. 110 (1) 270-275 | |
| K360E | Q347R | Choi et al., 2013 PNAS Jan. 2, | IgG1 |
| | | 2013. 110 (1) 270-275 | |
| K360E/K409W | D339V/Q347R/F405T | Choi et al., 2013 PNAS Jan. 2, | IgG1 |
| | | 2013. 110 (1) 270-275 | |
| Y349C/K360E/K409W | D339V/Q347R/S354C/F405T | Choi et al., 2013 PNAS Jan. 2, | IgG1 |
| | | 2013. 110 (1) 270-275 | |
| K392A/K409D | E356K/D399K | Leaver-Fey et al., 2016 Structure | IgG1 |
| | | Volume 24, Issue 4, 5 Apr. 2016, Pages | |
| | | 641-651 | |
| T366W | T366S/L358A/Y407A | Leaver-Fey et al., 2016 Structure | IgG1 |
| | | Volume 24, Issue 4, 5 Apr. 2016, Pages | |
| | | 641-651 | |
| D339M/Y407A | T336V/K409V | Leaver-Fey et al., 2016 Structure | IgG1 |
| | | Volume 24, Issue 4, 5 Apr. 2016, Pages | |
| | | 641-651 | |
| D339M/K360D/Y407A | T336V/E345R/Q347R/K409V | Leaver-Fey et al., 2016 Structure | IgG1 |
| | | Volume 24, Issue 4, 5 Apr. 2016, Pages | |
| | | 641-651 | |
| Y349S/T366V/K370Y/K409V | E357D/S364Q/Y407A | Leaver-Fey et al., 2016 Structure | IgG1 |
| | | Volume 24, Issue 4, 5 Apr. 2016, Pages | |
| | | 641-651 | |
| Y349S/T366M/K370Y/K409V | E356G/E357D/S364Q/Y407A | Leaver-Fey et al., 2016 Structure | IgG1 |
| | | Volume 24, Issue 4, 5 Apr. 2016, Pages | |
| | | 641-651 | |
| Y349S/T366M/K370Y/K409V | E357D/S364R/Y407A | Leaver-Fey et al., 2016 Structure | IgG1 |
| | | Volume 24, Issue 4, 5 Apr. 2016, Pages | |
| | | 641-651 | |

And any combination as described in Tables 1-3 of US20150284475A1

By way of example, in some embodiments, a human IgG Fc domains comprise mutations disclosed in Table 5, which reduce or eliminate FcγR and/or complement binding in the Fc domain. In embodiments, the Table 5 mutations are in both chains.

TABLE 5

| Chain 1 mutation | Reference | IgG |
|---|---|---|
| L234A/L235A | Alegre et al., 1994 | IgG1 |
| | Transplantation 57: 1537- | |
| | 1543 | |
| F234A/L235A | Alegre et al., 1994 | IgG4 |
| | Transplantation 57: 1537- | |
| | 1543 | |
| L235E | Morgan et al., 1995 | IgG1 |
| | Immunology. 1995 October; | |
| | 86(2): 319-324. | |
| L235E | Morgan et al., 1995 | IgG4 |
| | Immunology. 1995 October; | |
| | 86(2): 319-324. | |
| L235A | Morgan et al., 1995 | IgG1 |
| | Immunology. 1995 October; | |
| | 86(2): 319-324. | |
| G237A | Morgan et al., 1995 | IgG1 |
| | Immunology. 1995 October; | |
| | 86(2): 319-324. | |
| N297H | Tao and Morrison, | IgG1 |
| | J. Immunol. 1989; 143: 2595- | |
| | 2601 | |
| N297Q | Tao and Morrison, | IgG1 |
| | J. Immunol. 1989; 143: 2595- | |
| | 2601 | |
| N297K | Tao and Morrison, | IgG3 |
| | J. Immunol. 1989; 143: 2595- | |
| | 2601 | |
| N297Q | Tao and Morrison, | IgG3 |
| | J. Immunol. 1989; 143: 2595- | |
| | 2601 | |
| D265A | Idusogie et al., 2000 J | IgG1 |
| | Immunol Apr. 15, 2000, 164 | |
| | (8) 4178-4184 | |
| D270A, V, K | Idusogie et al., 2000 J | IgG1 |
| | Immunol Apr. 15, 2000, 164 | |
| | (8) 4178-4184 | |
| K322A, L, M, D, E | Idusogie et al., 2000 J | IgG1 |
| | Immunol Apr. 15, 2000, 164 | |
| | (8) 4178-4184 | |
| P329A, X | Idusogie et al., 2000 J | IgG1 |
| | Immunol Apr. 15, 2000, 164 | |
| | (8) 4178-4184 | |
| P331A, S, G, X | Idusogie et al., 2000 J | IgG1 |
| | Immunol Apr. 15, 2000, 164 | |
| | (8) 4178-4184 | |
| D265A | Idusogie et al., 2000 J | IgG1 |
| | Immunol Apr. 15, 2000, 164 | |
| | (8) 4178-4184 | |
| L234A | Hezareh et al., 2001 J. Virol. | IgG1 |
| | December 2001 vol. 75 no. | |
| | 24 12161-12168 | |
| L234A/L235A | Hezareh et al., 2001 J. Virol. | IgG1 |
| | December 2001 vol. 75 no. | |
| | 24 12161-12168 | |
| L234F/L235E/P331S | Oganesyan et al., 2008 Acta | IgG1 |
| | Cryst. (2008). D64, 700-704 | |
| H268Q/V309L/A330S/P331S | An et al., 2009 mAbs | IgG1 |
| | Volume 1, 2009 - Issue 6, | |
| | pp. 572-579 | |
| G236R/L328R | Moore et al., 2011 mAbs | IgG1 |
| | Volume 3, 2011 - Issue 6, | |
| | pp. 546-557 | |
| N297G | Couch et al., 2013 Sci. | IgG1 |
| | Transl. Med., 5 (2013) | |
| | 183ra57, 1-12 | |
| N297G/D265A | Couch et al., 2013 Sci. | IgG1 |
| | Transl. Med., 5 (2013) | |
| | 183ra57, 1-12 | |
| V234A/G237A/P328S/H268A/V309L/A330S/P331S | Vafa et al., 2014 Methods | IgG2 |
| | Volume 65, Issue 1, 1 | |
| | Jan. 2014, Pages 114- | |
| | 126 | |
| L234A/L235A/P329G | Lo et al., 2016 The Journal | IgG1 |
| | of Biological Chemistry | |
| | 292, 3900-3908 | |
| N297D | Schlothauer et al., 2016 | IgG1 |
| | Protein Engineering, Design | |
| | and Selection, Volume 29, | |
| | Issue 10, 1 Oct. 2016, | |
| | Pages 457-466 | |
| S228P/L235E | Schlothauer et al., 2016 | IgG4 |
| | Protein Engineering, Design | |
| | and Selection, Volume 29, | |
| | Issue 10, 1 Oct. 2016, | |
| | Pages 457-466 | |
| S228P/L235E/P329G | Schlothauer et al., 2016 | IgG4 |
| | Protein Engineering, Design | |
| | and Selection, Volume 29, | |
| | Issue 10, 1 Oct. 2016, | |
| | Pages 457-466 | |
| L234F/L235A/K322Q | Borrok et al., 2017 J Pharm | IgG1 |
| | Sci April 2017 Volume 106, | |
| | Issue 4, Pages 1008-1017 | |
| L234F/L235Q/P331G | Borrok et al., 2017 J Pharm | IgG1 |
| | Sci April 2017 Volume 106, | |
| | Issue 4, Pages 1008-1017 | |
| L234F/L235Q/K322Q | Borrok et al., 2017 J Pharm | IgG1 |
| | Sci April 2017 Volume 106, | |
| | Issue 4, Pages 1008-1017 | |
| L234A/L235A/G237A/P328S/H268A/A330S/P331S | Tam et al., 2017 Open | IgG1 |
| | Access | |
| | Antibodies 2017, 6(3), 12; | |
| | doi: 10.3390/antib6030012 | |
| S228P/F234A/L235A | Tam et al., 2017 Open | IgG4 |
| | Access | |
| | Antibodies 2017, 6(3), 12; | |
| | doi: 10.3390/antib6030012 | |
| S228P/F234A/L235A/G237A/P238S | Tam et al., 2017 Open | IgG4 |
| | Access | |
| | Antibodies 2017, 6(3), 12; | |
| | doi: 10.3390/antib6030012 | |
| S228P/F234A/L235A/G236□/G237A/P238S | Tam et al., 2017 Open | IgG4 |
| | Antibodies 2017, 6(3), 12; | |
| | Access | |
| | doi: 10.3390/antib6030012 | |

In some embodiments, the Fc domains in the Fc-based chimeric protein complexes of the present technology are homodimeric, i.e., the Fc region in the chimeric protein complex comprises two identical protein fragments.

In some embodiments, the Fc domains in the Fc-based chimeric protein complexes of the present technology are heterodimeric, i.e., the Fc domain comprises two non-identical protein fragments.

In some embodiments, heterodimeric Fc domains are engineered using ionic pairing and/or knob-in-hole mutations described herein. In some embodiments, the heterodimeric Fc-based chimeric protein complexes have a trans orientation/configuration. In a trans orientation/configuration, the targeting moiety and signaling agent, e.g. IL-2, are, in embodiments, not found on the same polypeptide chain in the present Fc-based chimeric protein complexes.

In some embodiments, the Fc domains includes or starts with the core hinge region of wild-type human IgG1, which contains the sequence Cys-Pro-Pro-Cys (SEQ ID NO: 276). In some embodiments, the Fc domains also include the upper hinge, or parts thereof (e.g., DKTHTCPPC (SEQ ID NO: 451); see WO 2009053368), EPKSCDKTHTCPPC (SEQ ID NO: 452), or EPKSSDKTHTCPPC (SEQ ID NO: 453); see Lo et al., Protein Engineering vol. 11 no. 6 pp. 495-500, 1998).

### Fc-Based Chimeric Protein Complexes

The Fc-based chimeric protein complexes of the present technology comprise at least one Fc domain disclosed herein, at least one signaling agent (SA), e.g. IL-2, disclosed herein, and at least one targeting moiety TM disclosed herein.

It is understood that the present Fc-based chimeric protein complexes may encompass a complex of two fusion proteins, each comprising an Fc domain.

In some embodiments, the Fc-based chimeric protein complex is heterodimeric. In some embodiments, the heterodimeric Fc-based chimeric protein complex has a trans orientation/configuration. In some embodiments, the heterodimeric Fc-based chimeric protein complex has a cis orientation/configuration.

In some embodiments, heterodimeric Fc domains are engineered using ionic pairing and/or knob-in-hole mutations described herein. In some embodiments, the heterodimeric Fc-based chimeric protein complexes have a trans orientation.

In a trans orientation, the targeting moiety and signaling agent are, in embodiments, not found on the same polypeptide chain in the present Fc-based chimeric protein complexes. In a trans orientation, the targeting moiety and signaling agent are, in embodiments, found on separate polypeptide chains in the Fc-based chimeric protein complexes. In a cis orientation, the targeting moiety and signaling agent are, in embodiments, found on the same polypeptide chain in the Fc-based chimeric protein complexes.

In some embodiments, where more than one targeting moiety is present in the heterodimeric protein complexes described herein, one targeting moiety may be in trans orientation (relative to the signaling agent), whereas another targeting moiety may be in cis orientation (relative to the signaling agent). In some embodiments, the signaling agent and target moiety are on the same ends/sides (N-terminal or C-terminal ends) of an Fc domain. In some embodiments, the signaling agent and targeting moiety are on different sides/ends of a Fc domain (N-terminal and C-terminal ends).

In some embodiments, where more than one targeting moiety is present in the heterodimeric protein complexes described herein, the targeting moieties may be found on the same Fc chain or on two different Fc chains in the heterodimeric protein complex (in the latter case the targeting moieties would be in trans relative to each other, as they are on different Fc chains). In some embodiments, where more than one targeting moiety is present on the same Fc chain, the targeting moieties may be on the same or different sides/ends of a Fc chain (N-terminal or/and C-terminal ends).

In some embodiments, where more than one signaling agent is present in the heterodimeric protein complexes described herein, the signaling agents may be found on the same Fc chain or on two different Fc chains in the heterodimeric protein complex (in the latter case the signaling agents would be in trans relative to each other, as they are on different Fc chains). In some embodiments, where more than one signaling agent is present on the same Fc chain, the signaling agents may be on the same or different sides/ends of a Fc chain (N-terminal or/and C-terminal ends).

In some embodiments, where more than one signaling agent is present in the heterodimeric protein complexes described herein, one signaling agent may be in trans orientation (as relates to the targeting moiety), whereas another signaling agent may be in cis orientation (as relates to the targeting moiety).

In some embodiments, the heterodimeric Fc-based chimeric protein complex does not comprise the signaling agent, e.g. IL-2, and targeting moiety on a single polypeptide.

In some embodiments, the Fc-based chimeric protein has an improved in vivo half-life relative to a chimeric protein lacking an Fc or a chimeric protein which is not a heterodimeric complex. In some embodiments, the Fc-based chimeric protein has an improved solubility, stability and other pharmacological properties relative to a chimeric protein lacking an Fc or a chimeric protein which is not a heterodimeric complex.

Heterodimeric Fc-based chimeric protein complexes are composed of two different polypeptides. In embodiments described herein, the targeting domain is on a different polypeptide than the signaling agent, e.g. IL-2, and accordingly, proteins that contain only one targeting domain copy, and also only one signaling agent, e.g. IL-2, copy can be made (this provides a configuration in which potential interference with desired properties can be controlled). Further, in embodiments, one targeting domain (e.g. VHH) only can avoid cross-linking of the antigen on the cell surface (which could elicit undesired effects in some cases). Further, in embodiments, one signaling agent, e.g. IL-2, may alleviate molecular “crowding” and potential interference with avidity mediated induction or restoration of effector function in dependence of the targeting domain. Further, in embodiments, heterodimeric Fc-based chimeric protein complexes can have two targeting moieties and these can be placed on the two different polypeptides. For instance, in embodiments, the C-terminus of both targeting moieties (e.g. VHHs) can be masked to avoid potential autoantibodies or pre-existing antibodies (e.g. VHH autoantibodies or pre-existing antibodies). Further, in embodiments, heterodimeric Fc-based chimeric protein complexes, e.g. with the targeting domain on a different polypeptide than the signaling agent, e.g. IL-2 (e.g. wild type signaling agent, e.g. wild type IL-2), may favor “cross-linking” of two cell types (e.g. a tumor cell and an immune cell). Further, in embodiments, heterodimeric Fc-based chimeric protein complexes can have two signaling agent, each on different polypeptides to allow more complex effector responses.

Further, in embodiments, heterodimeric Fc-based chimeric protein complexes, e.g. with the targeting domain on a different polypeptide than the signaling agent, e.g. IL-2, combinatorial diversity of targeting moiety and signaling agent, e.g. IL-2, is provided in a practical manner. For instance, in embodiments, polypeptides with any of the targeting moieties described herein can be combined “off the shelf” with polypeptides with any of the signaling agents described herein to allow rapid generation of various combinations of targeting moieties and signaling agents in single Fc-based chimeric protein complexes.

In some embodiments, the Fc-based chimeric protein complex comprises one or more linkers. In some embodiments, the Fc-based chimeric protein complex includes a linker that connects the Fc domain, signaling agent, e.g. IL-2(s), and targeting moiety(ies). In some embodiments, the Fc-based chimeric protein complex includes a linker that connects each signaling agent, e.g. IL-2, and targeting moiety (or, if more than one targeting moiety, a signaling agent, e.g. IL-2, to one of the targeting moieties). In some embodiments, the Fc-based chimeric protein complex includes a linker that connects each signaling agent, e.g. IL-2, to the Fc domain. In some embodiments, the Fc-based chimeric protein complex includes a linker that connects each targeting moiety to the Fc domain. In some embodiments, the Fc-based chimeric protein complex includes a linker that connects a targeting moiety to another targeting moiety. In some embodiments, the Fc-based chimeric protein complex includes a linker that connects a signaling agent, e.g. IL-2, to another signaling agent.

In some embodiments, a Fc-based chimeric protein complex comprises two or more targeting moieties. In such embodiments, the targeting moieties can be the same targeting moiety or they can be different targeting moieties.

In some embodiments, a Fc-based chimeric protein complex comprises two or more signaling agents. In such embodiments, the signaling agents can be the same targeting moiety or they can be different targeting moieties.

By way of example, in some embodiments, the Fc-based chimeric protein complex comprises a Fc domain, at least two signaling agents (SA), and at least two targeting moieties TM, wherein the Fc domain, signaling agents, and targeting moieties are selected from any of the Fc domains, signaling agents, and targeting moieties disclosed herein. In some embodiments, the Fc domain is homodimeric.

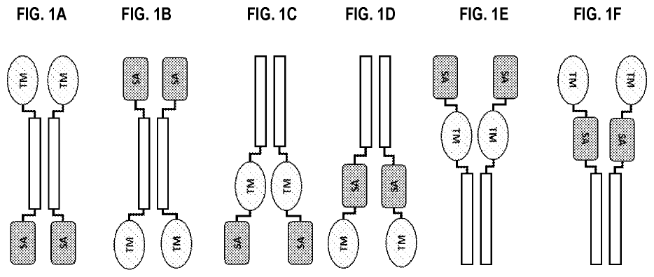

In various embodiments, the Fc-based chimeric protein complex takes the form of any of the schematics of FIGS. 1A-F.

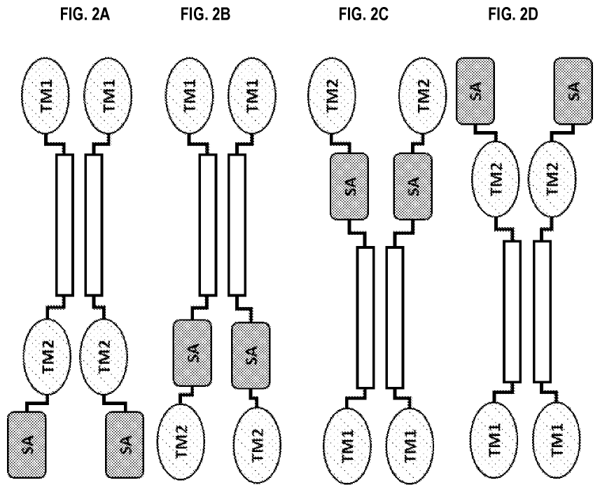

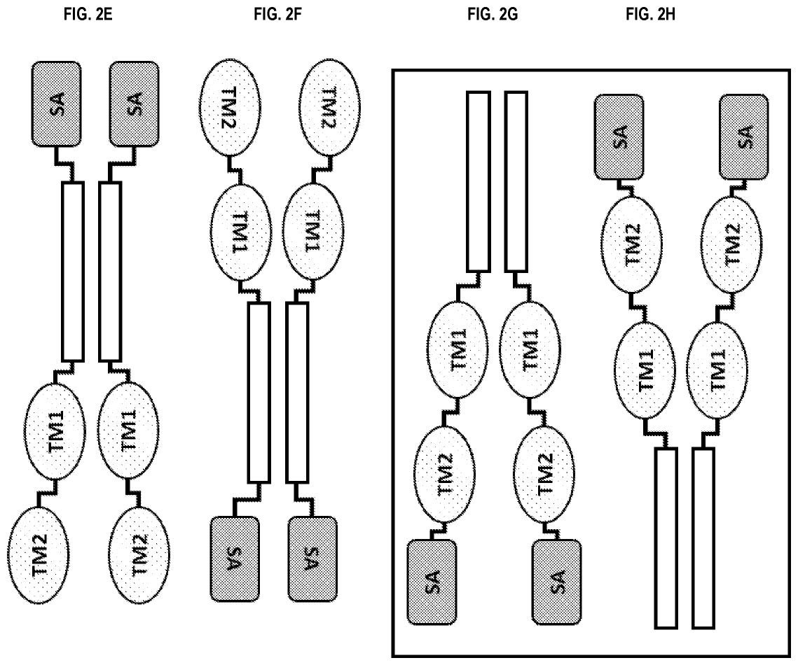

In various embodiments, the Fc-based chimeric protein complex takes the form of any of the schematics of FIGS. 2A-H.

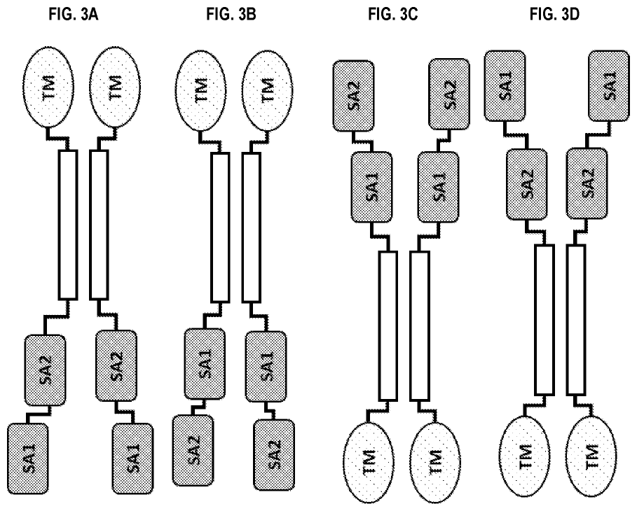

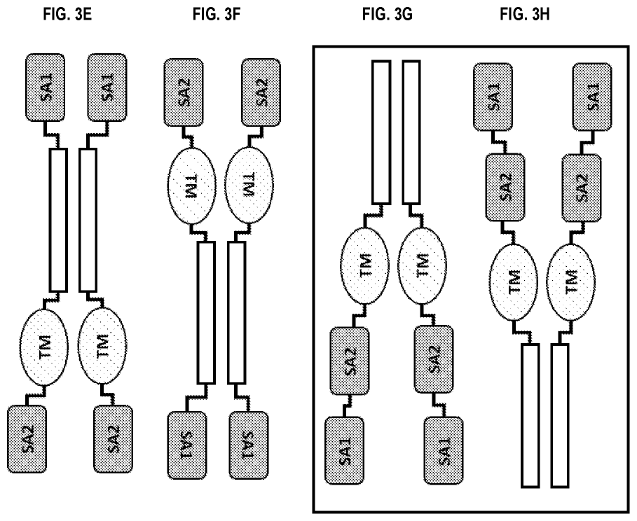

In various embodiments, the Fc-based chimeric protein complex takes the form of any of the schematics of FIGS. 3A-H.

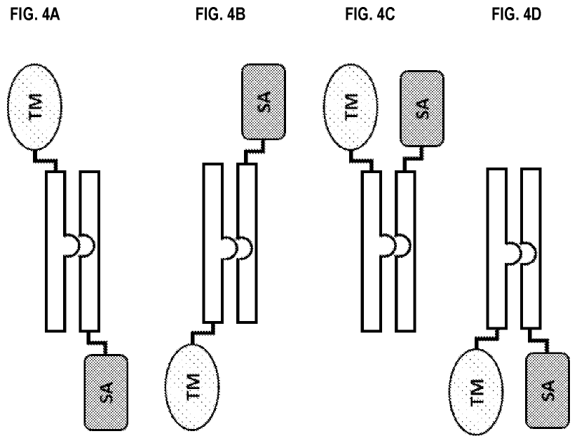

In various embodiments, the Fc-based chimeric protein complex takes the form of any of the schematics of FIGS. 4A-D.

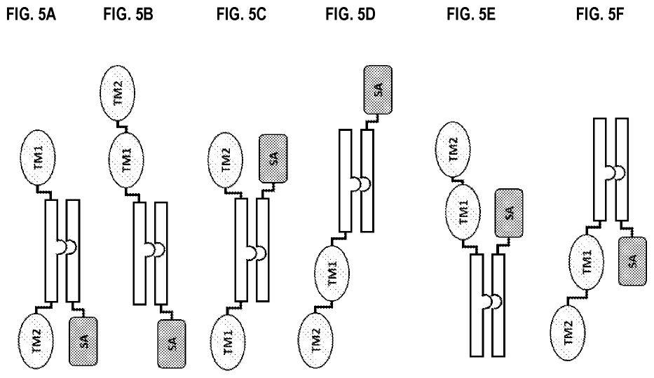

In various embodiments, the Fc-based chimeric protein complex takes the form of any of the schematics of FIGS. 5A-F.

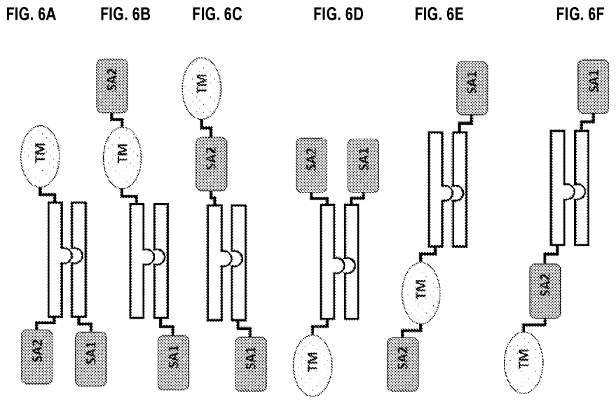

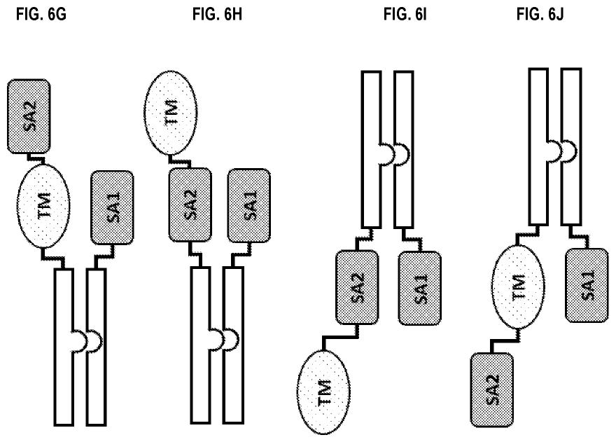

In various embodiments, the Fc-based chimeric protein complex takes the form of any of the schematics of FIGS. 6A-J.

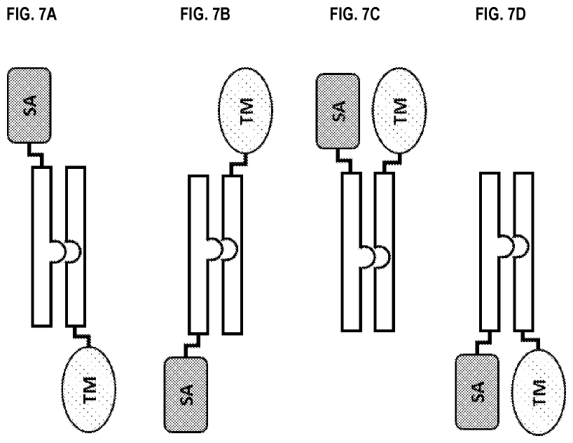

In various embodiments, the Fc-based chimeric protein complex takes the form of any of the schematics of FIGS. 7A-D.

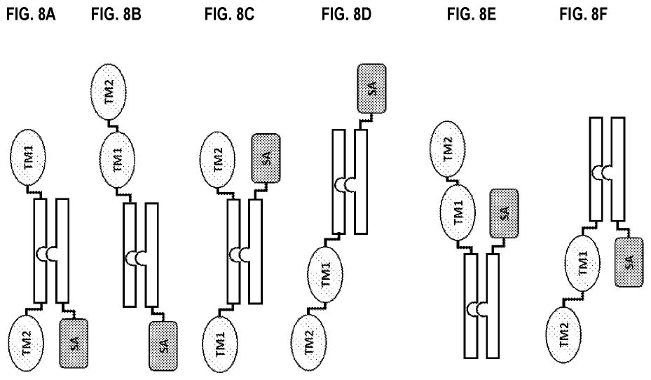

In various embodiments, the Fc-based chimeric protein complex takes the form of any of the schematics of FIGS. 8A-F.

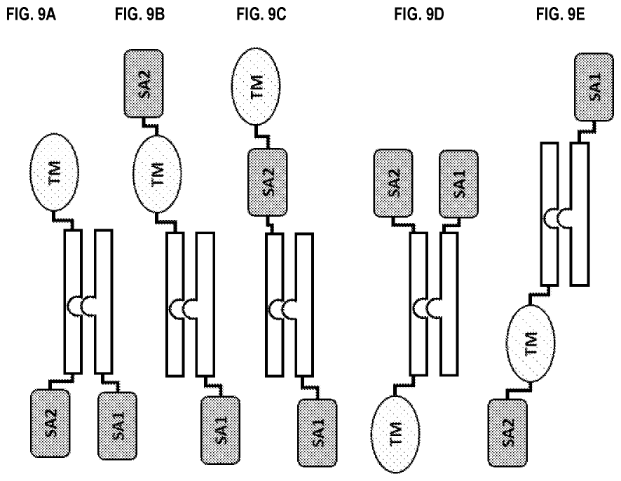

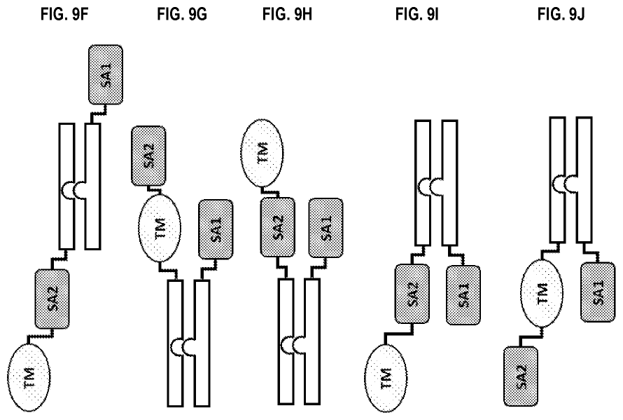

In various embodiments, the Fc-based chimeric protein complex takes the form of any of the schematics of FIGS. 9A-J.

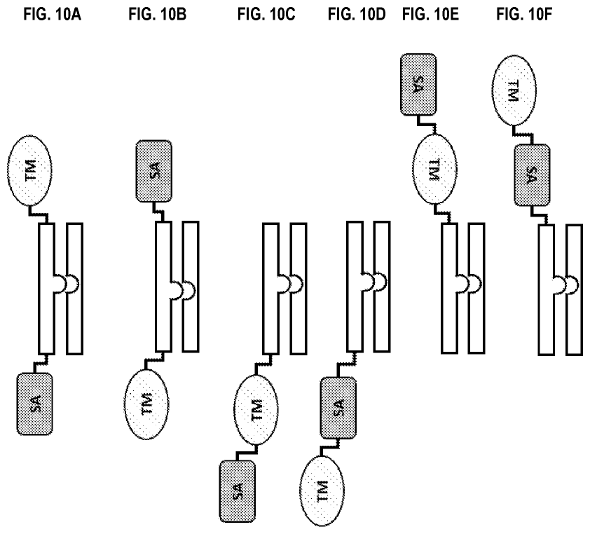

In various embodiments, the Fc-based chimeric protein complex takes the form of any of the schematics of FIGS. 10A-F.

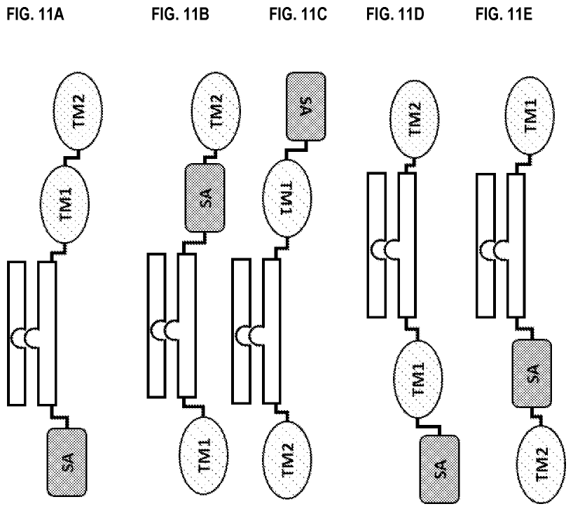

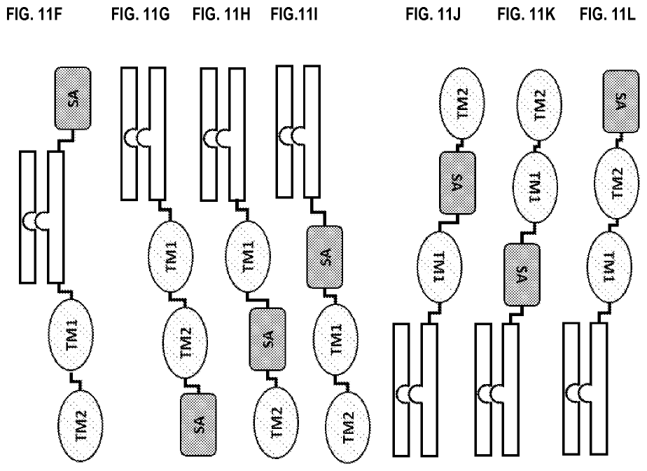

In various embodiments, the Fc-based chimeric protein complex takes the form of any of the schematics of FIGS. 11A-L.

In various embodiments, the Fc-based chimeric protein complex takes the form of any of the schematics of FIGS. 12A-L.

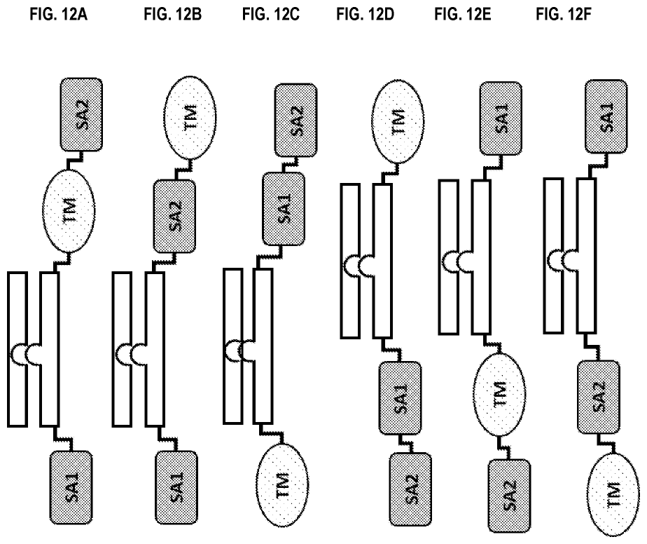

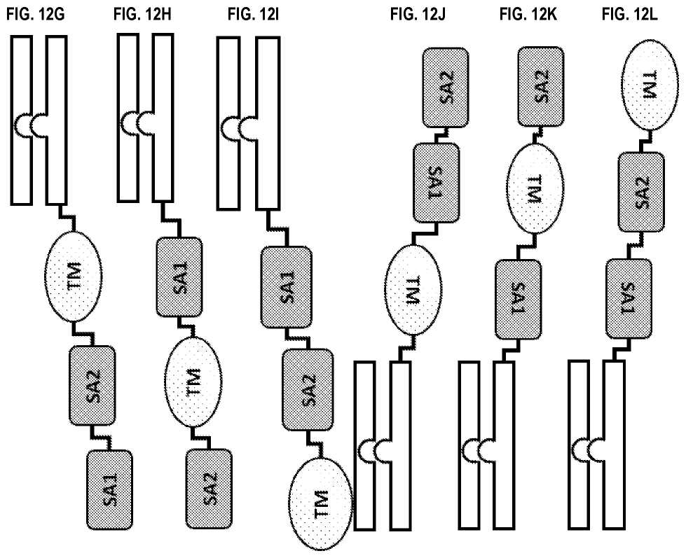

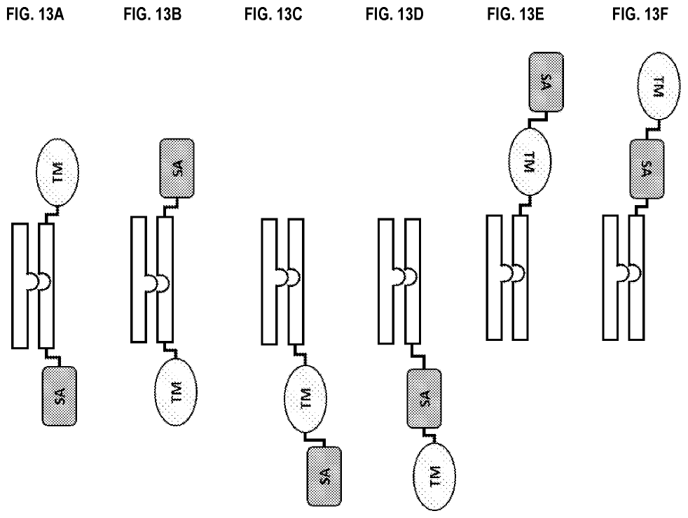

In various embodiments, the Fc-based chimeric protein complex takes the form of any of the schematics of FIGS. 13A-F.

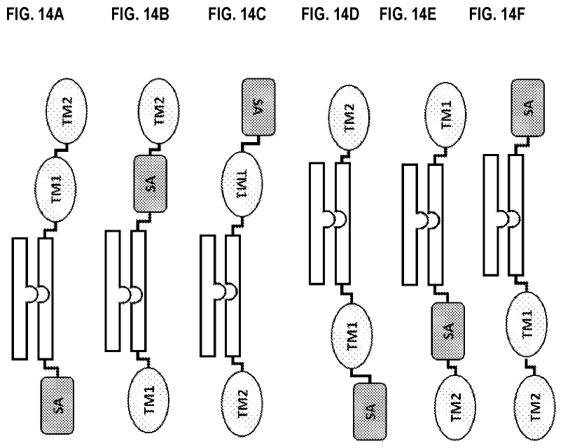

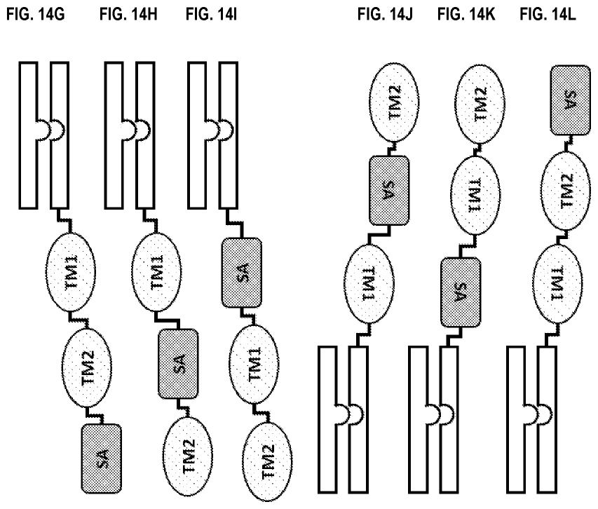

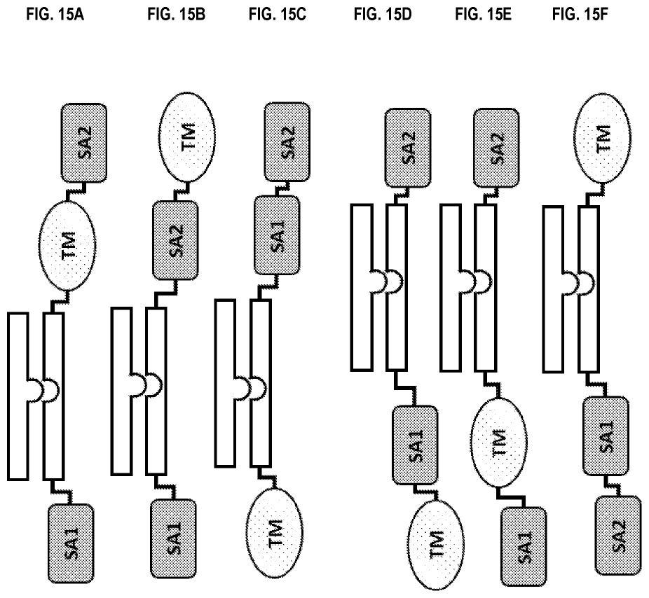

In various embodiments, the Fc-based chimeric protein complex takes the form of any of the schematics of FIGS. 14A-L.

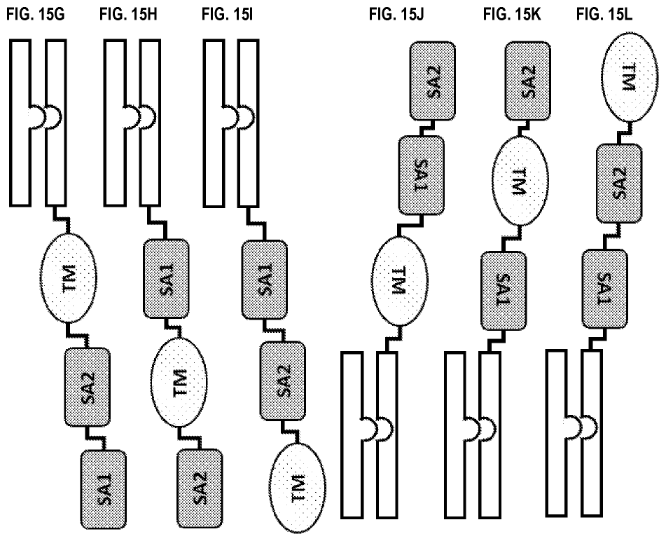

In various embodiments, the Fc-based chimeric protein complex takes the form of any of the schematics of FIGS. 15A-L.

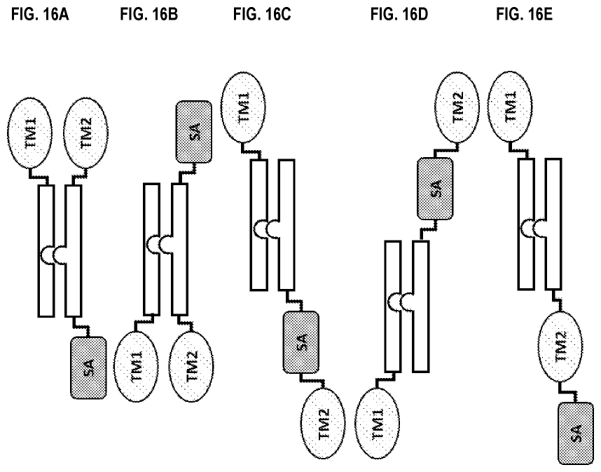

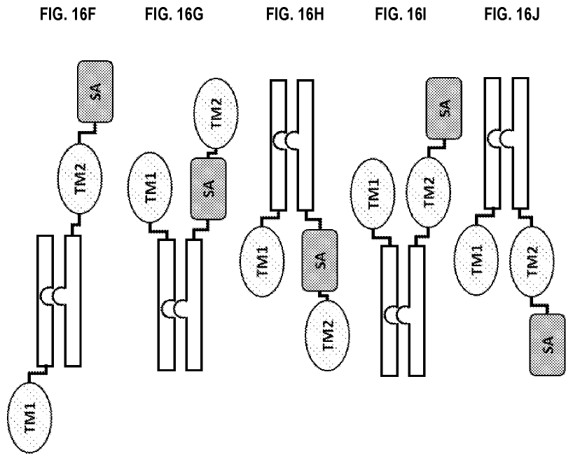

In various embodiments, the Fc-based chimeric protein complex takes the form of any of the schematics of FIGS. 16A-J.

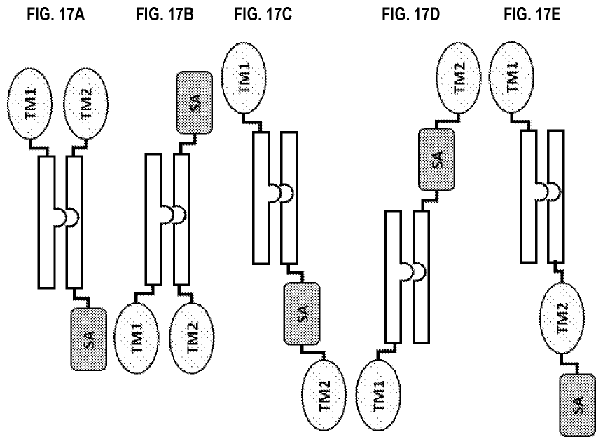

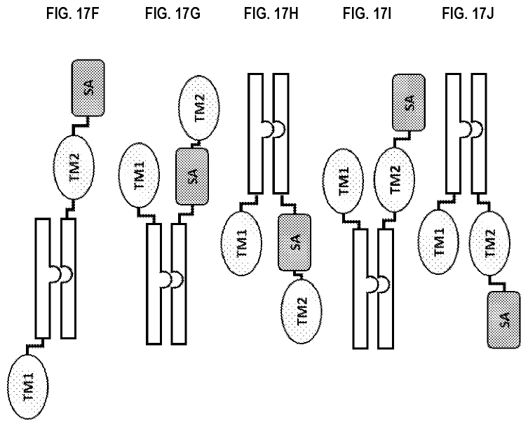

In various embodiments, the Fc-based chimeric protein complex takes the form of any of the schematics of FIGS. 17A-J.

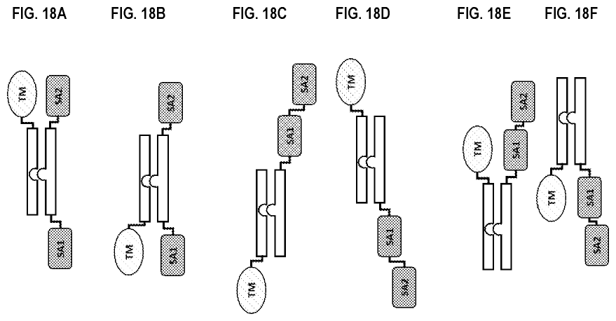

In various embodiments, the Fc-based chimeric protein complex takes the form of any of the schematics of FIGS. 18A-F.

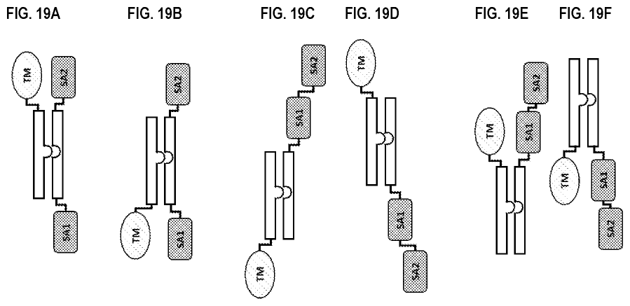

In various embodiments, the Fc-based chimeric protein complex takes the form of any of the schematics of FIGS. 19A-F.

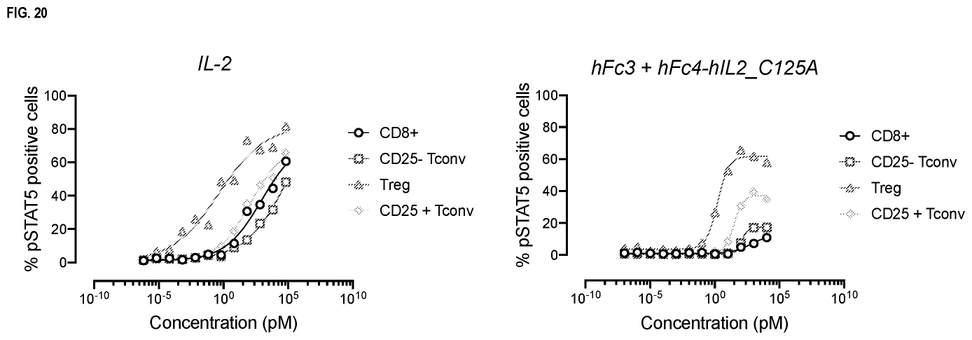

In various embodiments, the Fc-based chimeric protein complex takes the form of any of the schematics of FIGS. 20A-E.

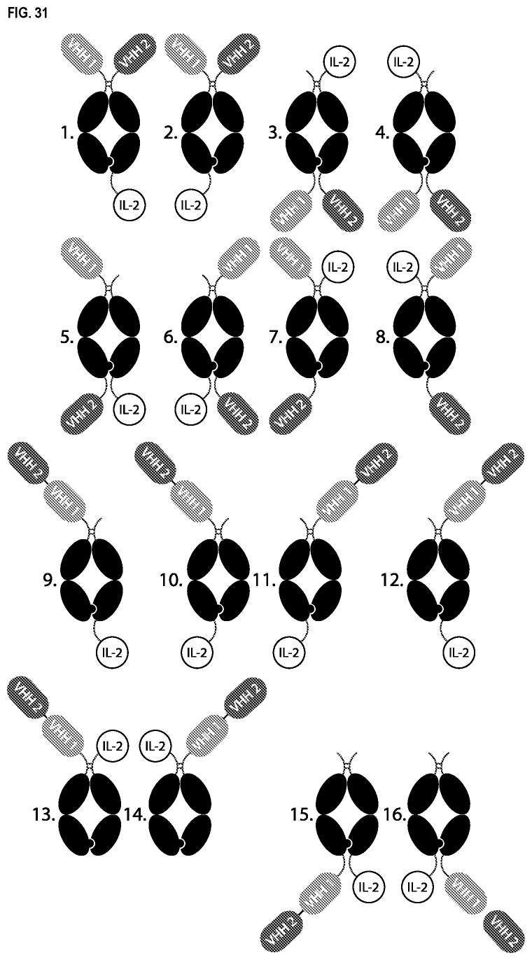

In various embodiments, the Fc-based chimeric protein complex takes the form of the schematic of FIG. 31.

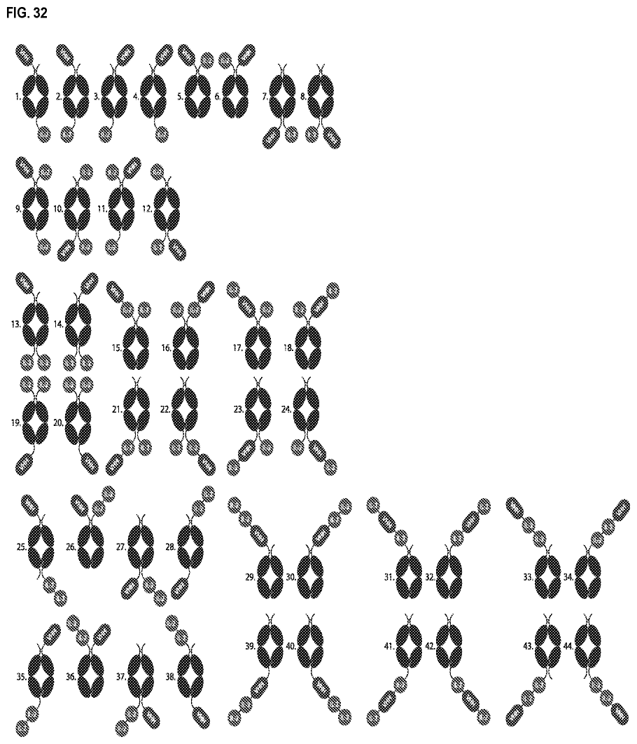

In various embodiments, the Fc-based chimeric protein complex takes the form of the schematic of FIG. 32.

In some embodiments, the present invention contemplates an Fc-based chimeric protein complex comprising one or more of the variants of Table 7 and/or one or more constructs listed in Table 7 within the column “ALN2 variant”.

In some embodiments, the signaling agents are linked to the targeting moieties and the targeting moieties are linked to the Fc domain on the same terminus (see FIGS. 1A-F). In some embodiments, the Fc domain is homodimeric.

In some embodiments, the signaling agents and targeting moieties are linked to the Fc domain, wherein the targeting moieties and signaling agents are linked on the same terminus (see FIGS. 1A-F). In some embodiments, the Fc domain is homodimeric.

In some embodiments, the targeting moieties are linked to signaling agents and the signaling agents are linked to the Fc domain on the same terminus (see FIGS. 1A-F). In some embodiments, the Fc domain is homodimeric.

In some embodiments, the homodimeric Fc-based chimeric protein complex has two or more targeting moieties.

In some embodiments, there are four targeting moieties and two signaling agents, the targeting moieties are linked to the Fc domain and the signaling agents are linked to targeting moieties on the same terminus (see FIGS. 2A-H). In some embodiments, the Fc domain is homodimeric. In some embodiments, where there are four targeting moieties and two signaling agents, two targeting moieties are linked to the Fc domain and two targeting moieties are linked to the signaling agents, which are linked to the Fc domain on the same terminus (see FIGS. 2A-H). In some embodiments, the Fc domain is homodimeric. In some embodiments, where there are four targeting moieties and two signaling agents, two targeting moieties are linked to each other and one of the targeting moieties of from each pair is linked to the Fc domain on the same terminus and the signaling agents are linked to the Fc domain on the same terminus (see FIGS. 2A-H). In some embodiments, the Fc domain is homodimeric. In some embodiments, where there are four targeting moieties and two signaling agents, two targeting moieties are linked to each other, wherein one of the targeting moieties of from each pair is linked to a signaling agent, e.g. IL-2, and the other targeting moiety of the pair is linked the Fc domain, wherein the targeting moieties linked to the Fc domain are linked on the same terminus (see FIGS. 2A-H). In some embodiments, the Fc domain is homodimeric.

In some embodiments, the homodimeric Fc-based chimeric protein complex has two or more signaling agents. In some embodiments, where there are four signaling agents and two targeting moieties, two signaling agents are linked to each other and one of the signaling agents of from pair is linked to the Fc domain on the same terminus and the targeting moieties are linked to the Fc domain on the same terminus (see FIGS. 3A-H). In some embodiments, the Fc domain is homodimeric. In some embodiments, where there are four signaling agents and two targeting moieties, two signaling agents are linked to the Fc domain one the same terminus and two of the signaling agents are each linked to a targeting moiety, wherein the targeting moieties are linked to the Fc domain at the same terminus (see FIGS. 3A-H). In some embodiments, the Fc domain is homodimeric. In some embodiments, where there are four signaling agents and two targeting moieties, two signaling agents are linked to each other and one of the signaling agents of from pair is linked to a targeting moiety and the targeting moieties are linked to the Fc domain on the same terminus (see FIGS. 3A-H). In some embodiments, the Fc domain is homodimeric.

By way of example, in some embodiments, the Fc-based chimeric protein complex comprise a Fc domain, wherein the Fc domain comprises ionic pairing mutation(s) and/or knob-in-hole mutation(s), at least one signaling agent, e.g. IL-2, and at least one targeting moiety, wherein the ionic pairing motif and/or a knob-in-hole motif, signaling agent, e.g. IL-2, and targeting moiety are selected from any of the ionic pairing motif and/or a knob-in-hole motif, signaling agents, and targeting moieties disclosed herein. In some embodiments, the Fc domain is heterodimeric. In some embodiments, the Fc domain comprises a mutation that reduces or eliminates its effector function.

In some embodiments, the signaling agent, e.g. IL-2, is linked to the targeting moiety, which is linked to the Fc domain (see FIGS. 10A-F and 13A-F). In some embodiments, the targeting moiety is linked to the signaling agent, e.g. IL-2, which is linked to the Fc domain (see FIGS. 10A-F and 13A-F). In some embodiments, the Fc domain is heterodimeric. In some embodiments, the Fc domain comprises a mutation that reduces or eliminates its effector function.

In some embodiments, the signaling agent, e.g. IL-2, and targeting moiety are linked to the Fc domain (see FIGS. 4A-D, 7A-D, 10A-F, and 13A-F). In some embodiments, the targeting moiety and the signaling agent, e.g. IL-2, are linked to different Fc chains on the same terminus (see FIGS. 4A-D and 7A-D). In some embodiments, the targeting moiety and the signaling agent, e.g. IL-2, are linked to different Fc chains on different termini (see FIGS. 4A-D and 7A-D). In some embodiments, the targeting moiety and the signaling agent, e.g. IL-2, are linked to the same Fc chain (see FIGS. 10A-F and 13A-F). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, the signaling agent, e.g. IL-2, is linked to the Fc domain and two targeting moieties can be: 1) linked to each other with one of the targeting moieties linked to the Fc domain; or 2) each linked to the Fc domain (see FIGS. 5A-F, 8A-F, 11A-L, 14A-L, 16A-J, and 17A-J). In some embodiments, the targeting moieties are linked on one Fc chain and the signaling agent, e.g. IL-2, is on the other Fc chain (see FIGS. 5A-F and 8A-F). In some embodiments, the paired targeting moieties and the signaling agent, e.g. IL-2 are linked to the same Fc chain (see FIGS. 11A-L and 14A-L). In some embodiments, a targeting moiety is linked to the Fc domain and the other targeting moiety is linked to the signaling agent, e.g. IL-2, and the paired targeting moiety is linked to the Fc domain (see FIGS. 11A-L, 14A-L, 16A-J, and 17A-J). In some embodiments, the unpaired targeting moiety and paired targeting moiety are linked to the same Fc chain (see FIGS. 11A-L and 14A-L). In some embodiments, the unpaired targeting moiety and paired targeting moiety are linked to different Fc chains (see FIGS. 16A-J and 17A-J). In some embodiments, the unpaired targeting moiety and paired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, a targeting moiety is linked to the signaling agent, e.g. IL-2, which is linked to the Fc domain, and the unpaired targeting moiety is linked the Fc domain (see FIGS. 11A-L, 14A-L, 16A-J, and 17A-J). In some embodiments, the paired signaling agent, e.g. IL-2, and unpaired targeting moiety are linked to the same Fc chain (see FIGS. 11A-L and 14A-L). In some embodiments, the paired signaling agent, e.g. IL-2, and unpaired targeting moiety are linked to different Fc chains (see FIGS. 16A-J and 17A-J). In some embodiments, the paired signaling agent, e.g. IL-2, and unpaired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, the targeting moieties are linked together and the signaling agent, e.g. IL-2, is linked to one of the paired targeting moieties, wherein the targeting moiety not linked to the signaling agent, e.g. IL-2, is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, the targeting moieties are linked together and the signaling agent, e.g. IL-2, is linked to one of the paired targeting moieties, wherein the signaling agent, e.g. IL-2, is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, the targeting moieties are both linked to the signaling agent, e.g. IL-2, wherein one of the targeting moieties is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-2, and two targeting moieties, the targeting moieties and the signaling agent, e.g. IL-2, are linked to the Fc domain (see FIGS. 16A-J and 17A-J). In some embodiments, the targeting moieties are linked on the terminus (see FIGS. 16A-J and 17A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked to the Fc domain on the same terminus and the targeting moiety is linked to the Fc domain (see FIGS. 6A-J and 9A-J). In some embodiments, the signaling agents are linked to the Fc domain on the same Fc chain and the targeting moiety is linked on the other Fc chain (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, a signaling agent, e.g. IL-2, is linked to the targeting moiety, which is linked to the Fc domain and the other signaling agent, e.g. IL-2, is linked to the Fc domain (see FIGS. 6A-J, 9A-J, 12A-L, and 15A-L). In some embodiments, the targeting moiety and the unpaired signaling agent, e.g. IL-2, are linked to different Fc chains (see FIGS. 6A-J and 9A-J). In some embodiments, the targeting moiety and the unpaired signaling agent, e.g. IL-2, are linked to different Fc chains on the same terminus (see FIGS. 6A-J and 9A-J). In some embodiments, the targeting moiety and the unpaired signaling agent, e.g. IL-2, are linked to different Fc chains on different termini (see FIGS. 6A-J and 9A-J). In some embodiments, the targeting moiety and the unpaired signaling agent, e.g. IL-2, are linked to the same Fc chains (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the targeting moiety is linked to a signaling agent, e.g. IL-2, which is linked to the Fc domain and the other signaling agent, e.g. IL-2, is linked to the Fc domain (see FIGS. 6A-J and 9A-J). In some embodiments, the paired signaling agent, e.g. IL-2, and the unpaired signaling agent, e.g. IL-2, are linked to different Fc chains (see FIGS. 6A-J and 9A-J). In some embodiments, the paired signaling agent, e.g. IL-2, and the unpaired signaling agent, e.g. IL-2, are linked to different Fc chains on the same terminus (see FIGS. 6A-J and 9A-J). In some embodiments, the paired signaling agent, e.g. IL-2, and the unpaired signaling agent, e.g. IL-2, are linked to different Fc chains on different termini (see FIGS. 6A-J and 9A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and the targeting moiety is linked to one of the paired signaling agents, wherein the targeting moiety is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the Fc domain and the targeting moiety is linked to the Fc domain (see FIGS. 12A-L, 15A-L, 18A-F, and 19A-F). In some embodiments, the paired signaling agents and targeting moiety are linked to the same Fc chain (see FIGS. 12A-L and 15A-L). In some embodiments, the paired signaling agents and targeting moiety are linked to different Fc chains (see FIGS. 18A-F and 19A-F). In some embodiments, the paired signaling agents and targeting moiety are linked to different Fc chains on the same terminus (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, the signaling agents are both linked to the targeting moiety, wherein one of the signaling agents is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the targeting moiety and the other signaling agent, e.g. IL-2, is linked to the Fc domain (see FIGS. 12A-L and 15A-L).

In some embodiments, where there are two signaling agents and one targeting moiety, each signaling agent, e.g. IL-2, is linked to the Fc domain and the targeting moiety is linked to one of the signaling agents (see FIGS. 12A-L and 15A-L). In some embodiments, the signaling agents are linked to the same Fc chain (see FIGS. 12A-L and 15A-L).

In some embodiments, a targeting moiety or signaling agent, e.g. IL-2, is linked to the Fc domain, comprising one or both of C_{H}2 and C_{H}3 domains, and optionally a hinge region. For example, vectors encoding the targeting moiety, signaling agent, e.g. IL-2, or combination thereof, linked as a single nucleotide sequence to an Fc domain can be used to prepare such polypeptides.

In some embodiments, the Fc-based chimeric protein complex comprises a polypeptide having an amino acid sequence having at least 90%, or at least 95%, or at least 96%, or at least 97%, or at least 98%, or at least 99% identity with any one of SEQ ID NOs: 291-296, 298-335, 340-345, 347-359, 361-368, 371-375, 377-449.

In some embodiments, the Fc-based chimeric protein complex comprises a polypeptide having an amino acid sequence having at least 95%, or at least 98%, or at least 99% identity with any one of SEQ ID NOs: 291-296, 298-335. In embodiments, the Fc-based chimeric protein complex comprises a polypeptide having an amino acid sequence selected from SEQ ID NOs: 291-296, 298-335 and less than 10 mutations to the amino acid sequence. In embodiments, the Fc-based chimeric protein complex comprises a polypeptide having an amino acid sequence selected from SEQ ID NOs: 291-296, 298-335, and less than 5 mutations to the amino acid sequence. In some embodiments, the Fc-based chimeric protein complex comprises a polypeptide having an amino acid sequence selected from SEQ ID NOs: 291-296, 298-335. In particular embodiments, the Fc-based chimeric protein complex comprises a polypeptide having an amino acid sequence having at least 95%, or at least 98%, or at least 99% identity with any one SEQ ID NOs: 292, 293, 294, 299, 301, 310, 311, 312, 315, 316, 319, 320, 325, 328, 332, 333, and 335. In particular embodiments, the Fc-based chimeric protein complex comprises a polypeptide having an amino acid sequence selected from SEQ ID NOs: 292, 293, 294, 299, 301, 310, 311, 312, 315, 316, 319, 320, 325, 328, 332, 333, and 335.

### Additional Signaling Agents

In one aspect, the present invention provides a chimeric proteins or chimeric protein complexes such as Fc-based chimeric protein complex comprising one or more signaling agents (for instance, an immune-modulating agent) in addition to the modified IL-2 described herein. In illustrative embodiments, the chimeric proteins or chimeric protein complexes such as Fc-based chimeric protein complex may comprise two, three, four, five, six, seven, eight, nine, ten or more signaling agents in addition to the modified IL-2 described herein. In various embodiments, the additional signaling agent is modified to have reduced affinity or activity for one or more of its receptors, which allows for attenuation of activity (inclusive of agonism or antagonism) and/or prevents non-specific signaling or undesirable sequestration of the chimeric proteins or chimeric protein complexes such as Fc-based chimeric protein complex.

In various embodiments, the additional signaling agent is antagonistic in its wild type form and bears one or more mutations that attenuate its antagonistic activity. In various embodiments, the additional signaling agent is antagonistic due to one or more mutations, e.g. an agonistic signaling agent is converted to an antagonistic signaling agent and, such a converted signaling agent, optionally, also bears one or more mutations that attenuate its antagonistic activity (e.g. as described in WO 2015/007520, the entire contents of which are hereby incorporated by reference).

In various embodiments, the additional signaling agent is selected from modified versions of cytokines, growth factors, and hormones. Illustrative examples of such cytokines, growth factors, and hormones include, but are not limited to, lymphokines, monokines, traditional polypeptide hormones, such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and tumor necrosis factor-β; mullerian-inhibiting substance; mouse gonadotropin-associated peptide; inhibin; activin; vascular endothelial growth factor; integrin; thrombopoietin (TPO); nerve growth factors such as NGF-α; platelet-growth factor; transforming growth factors (TGFs) such as TGF-α and TGF-β; insulin-like growth factor- and -II; osteo inductive factors; interferons such as, for example, interferon-α, interferon-β and interferon-γ (and interferon type I, II, and III), colony stimulating factors (CSFs) such as macrophage-CSF (M-CSF); granulocyte-macrophage-CSF (GM-CSF); and granulocyte-CSF (G-CSF); interleukins (ILs) such as, for example, IL-1B, IL-1a, IL-2, IL-3, IL-4, IL-5, IL-6, IL-7, IL-8, IL-9, IL-10, IL-11, IL-12, IL-13, IL-15, and IL-18; a tumor necrosis factor such as, for example, TNF-α or TNF-β; and other polypeptide factors including, for example, LIF and kit ligand (KL). As used herein, cytokines, growth factors, and hormones include proteins obtained from natural sources or produced from recombinant bacterial, eukaryotic or mammalian cell culture systems and biologically active equivalents of the native sequence cytokines.

In some embodiments, the additional signaling agent is a modified version of a growth factor selected from, but not limited to, transforming growth factors (TGFs) such as TGF-α and TGF-β, epidermal growth factor (EGF), insulin-like growth factor such as insulin-like growth factor-I and -II, fibroblast growth factor (FGF), heregulin, platelet-derived growth factor (PDGF), vascular endothelial growth factor (VEGF).

In an embodiment, the growth factor is a modified version of a fibroblast growth factor (FGF). Illustrative FGFs include, but are not limited to, FGF1, FGF2, FGF3, FGF4, FGF5, FGF6, FGF7, FGF8, FGF9, FGF10, FGF11, FGF12, FGF13, FGF14, murine FGF15, FGF16, FGF17, FGF18, FGF19, FGF20, FGF21, FGF22, and FGF23.

In an embodiment, the growth factor is a modified version of a vascular endothelial growth factor (VEGF). Illustrative VEGFs include, but are not limited to, VEGF-A, VEGF-B, VEGF-C, VEGF-D, and PGF and isoforms thereof including the various isoforms of VEGF-A such as VEGF₁₂₁, VEGF₁₂₁b, VEGF₁₄₅, VEGF₁₆₅, VEGF₁₆₅b, VEGF₁₈₉, and VEGF₂₀₆.

In an embodiment, the growth factor is a modified version of a transforming growth factor (TGF). Illustrative TGFs include, but are not limited to, TGF-α and TGF-β and subtypes thereof including the various subtypes of TGF-β including TGFβ1, TGFβ2, and TGFβ3.

In some embodiments, the additional signaling agent is a modified version of a hormone selected from, but not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, calcitriol, calciferol, atrial-natriuretic peptide, gastrin, secretin, cholecystokinin, neuropeptide Y, ghrelin, PYY3-36, insulin-like growth factor (IGF), leptin, thrombopoietin, erythropoietin (EPO), and angiotensinogen.

In some embodiments, the additional signaling agent is an immune-modulating agent, e.g. one or more of an interleukin, interferon, and tumor necrosis factor.

In some embodiments, the additional signaling agent is an interleukin, including for example IL-1B; IL-2; IL-3; IL-4; IL-5; IL-6; IL-7; IL-8; IL-9; IL-10; IL-11; IL-12; IL-13; IL-14; IL-15; IL-16; IL-17; IL-18; IL-19; IL-20; IL-21; IL-22; IL-23; IL-24; IL-25; IL-26; IL-27; IL-28; IL-29; IL-30; IL-31; IL-32; IL-33; IL-35; IL-36 or a fragment, variant, analogue, or family-member thereof. Interleukins are a group of multi-functional cytokines synthesized by lymphocytes, monocytes, and macrophages. Known functions include stimulating proliferation of immune cells (e.g., T helper cells, B cells, eosinophils, and lymphocytes), chemotaxis of neutrophils and T lymphocytes, and/or inhibition of interferons. Interleukin activity can be determined using assays known in the art: Matthews et al., in Lymphokines and Interferens: A Practical Approach, Clemens et al., eds, IRL Press, Washington, D.C. 1987, pp. 221-225; and Orencole & Dinarello (1989) Cytokine 1, 14-20.

In some embodiments, the signaling agent is a modified version of an interferon such as interferon types I, II, and III. Illustrative interferons, including for example, interferon-α-1, 2, 4, 5, 6, 7, 8, 10, 13, 14, 16, 17, and 21, interferon-β and interferon-γ, interferon κ, interferon ε, interferon τ, and interferon ω̅.

In embodiments, the additional signaling agent is a type I interferon. In embodiments, the type I interferon is selected from IFNα2, IFNα1, IFN-β, IFN-γ, Consensus IFN, IFN-ε, IFN-κ, IFN-τ, IFN-δ, and IFN-v.

In some embodiments, the additional signaling agent is a modified version of a tumor necrosis factor (TNF) or a protein in the TNF family, including but not limited to, TNF-α, TNF-β, LT-β, CD40L, CD27L, CD30L, FASL, 4-1BBL, OX40L, and TRAIL.

In various embodiments, the additional signaling agent is a modified (e.g. mutant) form of the signaling agent having one or more mutations. In various embodiments, the mutations allow for the modified signaling agent to have one or more of attenuated activity such as one or more of reduced binding affinity, reduced endogenous activity, and reduced specific bioactivity relative to unmodified or unmutated, i.e. the wild type form of the signaling agent (e.g. comparing the same signaling agent in a wild type form versus a modified (e.g. mutant) form). In various embodiments, the mutations allow for the modified signaling agent to have one or more of attenuated activity such as one or more of reduced binding affinity, reduced endogenous activity, and reduced specific bioactivity relative to unmodified or unmutated signaling agent, i.e. the unmutated IL-2. In some embodiments, the mutations which attenuate or reduce binding or affinity include those mutations which substantially reduce or ablate binding or activity. In some embodiments, the mutations which attenuate or reduce binding or affinity are different than those mutations which substantially reduce or ablate binding or activity. Consequentially, in various embodiments, the mutations allow for the signaling agent to be more safe, e.g. have reduced systemic toxicity, reduced side effects, and reduced off-target effects relative to unmutated, i.e. wild type, signaling agent (e.g. comparing the same signaling agent in a wild type form versus a modified (e.g. mutant) form). In various embodiments, the mutations allow for the signaling agent to be safer, e.g. have reduced systemic toxicity, reduced side effects, and reduced off-target effects relative to unmutated signaling agent, e.g. the unmutated sequence of IL-2.

In various embodiments, the additional signaling agent is modified to have one or more mutations that reduce its binding affinity or activity for one or more of its receptors. In some embodiments, the signaling agent is modified to have one or more mutations that substantially reduce or ablate binding affinity or activity for the receptors. In some embodiments, the activity provided by the wild type signaling agent is agonism at the receptor (e.g. activation of a cellular effect at a site of therapy). For example, the wild type signaling agent may activate its receptor. In such embodiments, the mutations result in the modified signaling agent to have reduced or ablated activating activity at the receptor. For example, the mutations may result in the modified signaling agent to deliver a reduced activating signal to a target cell or the activating signal could be ablated. In some embodiments, the activity provided by the wild type signaling agent is antagonism at the receptor (e.g. blocking or dampening of a cellular effect at a site of therapy). For example, the wild type signaling agent may antagonize or inhibit the receptor. In these embodiments, the mutations result in the modified signaling agent to have a reduced or ablated antagonizing activity at the receptor. For example, the mutations may result in the modified signaling agent to deliver a reduced inhibitory signal to a target cell or the inhibitory signal could be ablated. In various embodiments, the signaling agent is antagonistic due to one or more mutations, e.g. an agonistic signaling agent is converted to an antagonistic signaling agent (e.g. as described in WO 2015/007520, the entire contents of which are hereby incorporated by reference) and, such a converted signaling agent, optionally, also bears one or more mutations that reduce its binding affinity or activity for one or more of its receptors or that substantially reduce or ablate binding affinity or activity for one or more of its receptors.

In some embodiments, the reduced affinity or activity at the receptor is inducible or restorable by attachment with one or more of the targeting moieties or upon inclusion in the Fc-based chimeric protein complex disclosed herein. In other embodiments, the reduced affinity or activity at the receptor is not substantially inducible or restorable by the activity of one or more of the targeting moieties or upon inclusion in the Fc-based chimeric protein complex disclosed herein.

In various embodiments, the additional signaling agent is active on target cells because the targeting moiety(ies) compensates for the missing/insufficient binding (e.g., without limitation and/or avidity) required for substantial activation. In various embodiments, the modified signaling agent is substantially inactive en route to the site of therapeutic activity and has its effect substantially on specifically targeted cell types which greatly reduces undesired side effects.

In some embodiments, the additional signaling agent may include one or more mutations that attenuate or reduce binding or affinity for one receptor (i.e., a therapeutic receptor) and one or more mutations that substantially reduce or ablate binding or activity at a second receptor. In such embodiments, these mutations may be at the same or at different positions (i.e., the same mutation or multiple mutations). In some embodiments, the mutation(s) that reduce binding and/or activity at one receptor is different than the mutation(s) that substantially reduce or ablate at another receptor. In some embodiments, the mutation(s) that reduce binding and/or activity at one receptor is the same as the mutation(s) that substantially reduce or ablate at another receptor. In some embodiments, the present chimeric proteins or chimeric protein complexes such as Fc-based chimeric protein complexes have a modified signaling agent that has both mutations that attenuate binding and/or activity at a therapeutic receptor and therefore allow for a more controlled, on-target therapeutic effect (e.g. relative wild type signaling agent) and mutations that substantially reduce or ablate binding and/or activity at another receptor and therefore reduce side effects (e.g. relative to wild type signaling agent).

In some embodiments, the substantial reduction or ablation of binding or activity is not substantially inducible or restorable with a targeting moiety or upon inclusion in the Fc-based chimeric protein complex disclosed herein. In some embodiments, the substantial reduction or ablation of binding or activity is inducible or restorable with a targeting moiety or upon inclusion in the Fc-based chimeric protein complex disclosed herein. In various embodiments, substantially reducing or ablating binding or activity at a second receptor also may prevent deleterious effects that are mediated by the other receptor. Alternatively, or in addition, substantially reducing or ablating binding or activity at the other receptor causes the therapeutic effect to improve as there is a reduced or eliminated sequestration of the therapeutic chimeric proteins or chimeric protein complexes such as Fc-based chimeric protein complexes away from the site of therapeutic action. For instance, in some embodiments, this obviates the need of high doses of the chimeric proteins or chimeric protein complexes such as Fc-based chimeric protein complexes that compensate for loss at the other receptor. Such ability to reduce dose further provides a lower likelihood of side effects.

In various embodiments, the additional modified signaling agent comprises one or more mutations that cause the signaling agent to have reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity (inclusive of agonism or antagonism). In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type signaling agent. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type signaling agent (including, by way of non-limitation, relative to the unmutated IL-2).

In embodiments wherein the chimeric proteins or chimeric protein complexes such as Fc-based chimeric protein complex has mutations that reduce binding at one receptor and substantially reduce or ablate binding at a second receptor, the attenuation or reduction in binding affinity of a modified signaling agent for one receptor is less than the substantial reduction or ablation in affinity for the other receptor. In some embodiments, the attenuation or reduction in binding affinity of a modified signaling agent for one receptor is less than the substantial reduction or ablation in affinity for the other receptor by about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95%. In various embodiments, substantial reduction or ablation refers to a greater reduction in binding affinity and/or activity than attenuation or reduction.

In various embodiments, the additional modified signaling agent comprises one or more mutations that reduce the endogenous activity of the signaling agent to about 75%, or about 70%, or about 60%, or about 50%, or about 40%, or about 30%, or about 25%, or about 20%, or about 10%, or about 5%, or about 3%, or about 1%, e.g., relative to the wild type signaling agent (including, by way of non-limitation, relative to the unmutated IL-2).

In various embodiments, the additional modified signaling agent comprises one or more mutations that cause the signaling agent to have reduced affinity and/or activity for a receptor of any one of the cytokines, growth factors, and hormones as described herein.

In some embodiments, the additional modified signaling agent comprises one or more mutations that cause the signaling agent to have reduced affinity for its receptor that is lower than the binding affinity of the targeting moiety(ies) for its(their) receptor(s). In some embodiments, this binding affinity differential is between signaling agent/receptor and targeting moiety/receptor on the same cell. In some embodiments, this binding affinity differential allows for the signaling agent, e.g. mutated signaling agent, to have localized, on-target effects and to minimize off-target effects that underlie side effects that are observed with wild type signaling agent. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold.

Receptor binding activity may be measured using methods known in the art. For example, affinity and/or binding activity may be assessed by Scatchard plot analysis and computer-fitting of binding data (e.g. Scatchard, 1949) or by reflectometric interference spectroscopy under flow through conditions, as described by Brecht et al. (1993), the entire contents of all of which are hereby incorporated by reference. In some embodiments, receptor binding activity is measured using bio-layer interferometry (BLI).

The amino acid sequences of the wild type signaling agents described herein are well known in the art. Accordingly, in various embodiments the additional modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known wild type amino acid sequences of the signaling agents described herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments the additional modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments, the additional modified signaling agent comprises an amino acid sequence having one or more amino acid mutations. In some embodiments, the one or more amino acid mutations may be independently selected from substitutions, insertions, deletions, and truncations.

In some embodiments, the amino acid mutations are amino acid substitutions, and may include conservative and/or non-conservative substitutions as described herein.

As described herein, the additional modified signaling agents bear mutations that affect affinity and/or activity at one or more receptors. In various embodiments, there is reduced affinity and/or activity at a therapeutic receptor, e.g. a receptor through which a desired therapeutic effect is mediated (e.g. agonism or antagonism). In various embodiments, the modified signaling agents bear mutations that substantially reduce or ablate affinity and/or activity at a receptor, e.g. a receptor through which a desired therapeutic effect is not mediated (e.g. as the result of promiscuity of binding). The receptors of any modified signaling agents, e.g. one of the cytokines, growth factors, and hormones as described herein, are known in the art.

Illustrative mutations which provide reduced affinity and/or activity (e.g. agonistic) at a receptor are found in WO 2013/107791 (e.g. with regard to interferons), WO 2015/007542 (e.g. with regard to interleukins), and WO 2015/007903 (e.g. with regard to TNF), the entire contents of each of which are hereby incorporated by reference. Illustrative mutations which provide reduced affinity and/or activity (e.g. antagonistic) at a therapeutic receptor are found in WO 2015/007520, the entire contents of which are hereby incorporated by reference.

In some embodiments, the additional modified signaling agent comprises one or more mutations that cause the signaling agent to have reduced affinity and/or activity for a type I cytokine receptor, a type II cytokine receptor, a chemokine receptor, a receptor in the Tumor Necrosis Factor Receptor (TNFR) superfamily, TGF-beta Receptors, a receptor in the immunoglobulin (Ig) superfamily, and/or a receptor in the tyrosine kinase superfamily.

In various embodiments, the receptor for the additional signaling agent is a Type I cytokine receptor. Type I cytokine receptors are known in the art and include, but are not limited to receptors for IL2 (beta-subunit), IL3, IL4, IL5, IL6, IL7, IL9, IL11, IL12, GM-CSF, G-CSF, LIF, CNTF, and also the receptors for Thrombopoietin (TPO), Prolactin, and Growth hormone. Illustrative type I cytokine receptors include, but are not limited to, GM-CSF receptor, G-CSF receptor, LIF receptor, CNTF receptor, TPO receptor, and type I IL receptors.

In various embodiments, the receptor for the additional signaling agent is a Type II cytokine receptor. Type II cytokine receptors are multimeric receptors composed of heterologous subunits and are receptors mainly for interferons. This family of receptors includes, but is not limited to, receptors for interferon-α, interferon-β and interferon-γ, IL10, IL22, and tissue factor. Illustrative type II cytokine receptors include, but are not limited to, IFNα receptor (e.g. IFNAR1 and IFNAR2), IFNβ receptor, IFNγ receptor (e.g. IFNGR1 and IFNGR2), and type II IL receptors.

In various embodiments, the receptor for the additional signaling agent is a G protein-coupled receptor. Chemokine receptors are G protein-coupled receptors with seven transmembrane structure and coupled to G-protein for signal transduction. Chemokine receptors include, but are not limited to, CC chemokine receptors, CXC chemokine receptors, CX3C chemokine receptors, and XC chemokine receptor (XCR1). Illustrative chemokine receptors include, but are not limited to, CCR1, CCR2, CCR3, CCR4, CCR5, CCR6, CCR7, CCR8, CCR9, CCR10, CXCR1, CXCR2, CXCR3, CXCR3B, CXCR4, CXCR5, CSCR6, CXCR7, XCR1, and CX3CR1.

In various embodiments, the receptor for the additional signaling agent is a TNFR family member. Tumor necrosis factor receptor (TNFR) family members share a cysteine-rich domain (CRD) formed of three disulfide bonds surrounding a core motif of CXXCXXC creating an elongated molecule. Illustrative tumor necrosis factor receptor family members include: CDI 20a (TNFRSFIA), CD 120b (TNFRSFIB), Lymphotoxin beta receptor (LTBR, TNFRSF3), CD 134 (TNFRSF4), CD40 (CD40, TNFRSF5), FAS (FAS, TNFRSF6), TNFRSF6B (TNFRSF6B), CD27 (CD27, TNFRSF7), CD30 (TNFRSF8), CD137 (TNFRSF9), TNFRSFIOA (TNFRSFIOA), TNFRSFIOB, (TNFRSFIOB), TNFRSFIOC (TNFRSFIOC), TNFRSFIOD (TNFRSFIOD), RANK (TNFRSFI IA), Osteoprotegerin (TNFRSFI IB), TNFRSF12A (TNFRSF12A), TNFRSF13B (TNFRSF13B), TNFRSF13C (TNFRSF13C), TNFRSF14 (TNFRSF14), Nerve growth factor receptor (NGFR, TNFRSF16), TNFRSF17 (TNFRSF17), TNFRSF18 (TNFRSF18), TNFRSF19 (TNFRSF19), TNFRSF21 (TNFRSF21), and TNFRSF25 (TNFRSF25).

In various embodiments, the receptor for the additional signaling agent is a TGF-beta receptor. TGF-beta receptors are single pass serine/threonine kinase receptors. TGF-beta receptors include, but are not limited to, TGFBR1, TGFBR2, and TGFBR3.

In various embodiments, the receptor for the additional signaling agent is an Ig superfamily receptor. Receptors in the immunoglobulin (Ig) superfamily share structural homology with immunoglobulins. Receptors in the Ig superfamily include, but are not limited to, interleukin-1 receptors, CSF-1R, PDGFR (e.g. PDGFRA and PDGFRB), and SCFR.

In various embodiments, the receptor for the additional signaling agent is a tyrosine kinase superfamily receptor. Receptors in the tyrosine kinase superfamily are well known in the art. There are about 58 known receptor tyrosine kinases (RTKs), grouped into 20 subfamilies. Receptors in the tyrosine kinase superfamily include, but are not limited to, FGF receptors and their various isoforms such as FGFR1, FGFR2, FGFR3, FGFR4, and FGFR5.

In an embodiment, the additional modified signaling agent is interferon α. In such embodiments, the modified IFNα agent has reduced affinity and/or activity for the IFN-α/β receptor (IFNAR), i.e., IFNAR1 and/or IFNAR2 chains. In some embodiments, the modified IFN-α agent has substantially reduced or ablated affinity and/or activity for the IFN-α/β receptor (IFNAR), i.e., IFNAR1 and/or IFNAR2 chains.

Mutant forms of interferon α are known to the person skilled in the art. In an illustrative embodiment, the modified signaling agent is the allelic form IFNα2a, having the amino acid sequence of SEQ ID NO:233.

In an illustrative embodiment, the modified signaling agent is the allelic form IFNα2b having the amino acid sequence of SEQ ID NO: 234 (which differs from IFNα2a at amino acid position 23).

In some embodiments, said IFNα2 mutant (IFNα2a or IFNα2b) is mutated at one or more amino acids at positions 144-154, such as amino acid positions 145, 148, 149 and/or 153. In some embodiments, the IFNα2 mutant comprises one or more mutations selected from A145G, L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFNα2 mutants have reduced affinity and/or activity for IFNAR1. In some embodiments, the IFNα2 mutant comprises one or more mutations selected from F64A, N65A, T69A, L80A, Y85A, and Y89A, as described in WO2010/030671, the entire contents of which is hereby incorporated by reference.

In some embodiments, the IFNα2 mutant comprises one or more mutations selected from K133A, R144A, R149A, and L153A as described in WO2008/124086, the entire contents of which is hereby incorporated by reference.

In some embodiments, the IFNα2 mutant comprises one or more mutations selected from R120E and R120E/K121E, as described in WO2015/007520 and WO2010/030671, the entire contents of which are hereby incorporated by reference. In such embodiments, said IFNα2 mutant antagonizes wildtype IFNα2 activity. In such embodiments, said mutant IFNα2 has reduced affinity and/or activity for IFNAR1 while affinity and/or activity of IFNR2 is retained.

In some embodiments, the human IFNα2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFNα2 mutant comprises R120E and L153A.

In some embodiments, the human IFNα2 mutant comprises one or more mutations selected from, L15A, A19W, R22A, R23A, L26A, F27A, L30A, L30V, K31A, D32A, R33K, R33A, R33Q, H34A, D35A, Q40A, D114R, L117A, R120A, R125A, K134A, R144A, A145G, A145M, M148A, R149A, S152A, L153A, and N156A as disclosed in WO 2013/059885, the entire disclosures of which are hereby incorporated by reference. In some embodiments, the human IFNα2 mutant comprises the mutations H57Y, E58N, Q61S, and/or L30A as disclosed in WO 2013/059885. In some embodiments, the human IFN-α2 mutant comprises the mutations H57Y, E58N, Q61S, and/or R33A as disclosed in WO 2013/059885. In some embodiments, the human IFNα2 mutant comprises the mutations H57Y, E58N, Q61S, and/or M148A as disclosed in WO 2013/059885. In some embodiments, the human IFNα2 mutant comprises the mutations H57Y, E58N, Q61S, and/or L153A as disclosed in WO 2013/059885. In some embodiments, the human IFNα2 mutant comprises the mutations N65A, L80A, Y85A, and/or Y89A as disclosed in WO 2013/059885. In some embodiments, the human IFNα2 mutant comprises the mutations N65A, L80A, Y85A, Y89A, and/or D114A as disclosed in WO 2013/059885. In some embodiments, the human IFNα2 mutant comprises one or more mutations selected from R144X₁, A145X₂, and R33A, wherein X₁ is selected from A, S, T, Y, L, and I, and wherein X₂ is selected from G, H, Y, K, and D. In some embodiments, the human IFNα2 mutant comprises one or more mutations selected from R33A, T106X₃, R120E, R144X₁ A145X₂, M148A, R149A, and L153A with respect to amino acid sequence of SEQ ID NO: 233 or 234, wherein X₁ is selected from A, S, T, Y, L, and I, wherein X₂ is selected from G, H, Y, K, and D, and wherein X₃ is selected from A and E.

In some embodiments, the additional modified signaling agent is IFNα1. In such embodiments, the modified IFNα 1 signaling agent also has reduced affinity and/or activity for the IFN-α/β receptor (IFNAR), i.e., IFNAR1 and/or IFNAR2 chains. In some embodiments, the modified IFNα1 agent has substantially reduced or ablated affinity and/or activity for the IFN-α/β receptor (IFNAR), i.e., IFNAR1 and/or IFNAR2 chains.

In some embodiments, the additional modified IFNα1 signaling agent is modified to have a mutation at one or more amino acids at positions L15, A19, R23, S25, L30, D32, R33, H34, Q40, D115, L118, K121, R126, E133, K134, K135, R145, A146, M149, R150, S153, L154, and N157 with reference to SEQ ID NO: 450. The mutations can optionally be a hydrophobic mutation and can be, e.g., selected from alanine, valine, leucine, and isoleucine. In some embodiments, the IFNα1 interferon is modified to have a one or more mutations selected from L15A, A19W, R23A, S25A, L30A, L30V, D32A, R33K, R33A, R33Q, H34A, Q40A, D115R, L118A, K121A, K121E, R126A, R126E, E133A, K134A, K135A, R145A, R145D, R145E, R145G, R145H, R1451, R145K, R145L, R145N, R145Q, R145S, R145T, R145V, R145Y, A146D, A146E, A146G, A146H, A1461, A146K, A146L, A146M, A146N, A146Q, A146R, A146S, A146T, A146V, A146Y, M149A, M149V, R150A, S153A, L154A, and N157A with reference to SEQ ID NO: 450. In some embodiments, the IFNα1 mutant comprises one or more multiple mutations selected from L30A/H58Y/E59N/Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121A/K122A, R121A/K122A, and R121E/K 122E with reference to SEQ ID NO: 450.

In an embodiment, the additional modified IFNα1 signaling agent, or variant thereof, is modified to have one or more mutations at amino acid positions C1, C29, C86, C99, or C139 with reference to SEQ ID NO: 450. In this regard, Beilharz et al., Journal of interferon research 6.6 (1986): 677-685 (which is hereby incorporated by reference in its entirety) describes various mutations of IFNα1 that may be used introduced in the modified IFNα1 of the present invention. The mutation at position C86 can be, e.g., C86S or C86A or C86Y. These C86 mutants of IFNα1 are called reduced cysteine-based aggregation mutants. In some embodiment, the IFNα1 variant includes mutations at positions C1, C86 and C99 with reference to SEQ ID NO: 450.

In an embodiment, the additional modified signaling agent is interferon β. In such embodiments, the modified interferon β agent also has reduced affinity and/or activity for the IFN-α/β receptor (IFNAR), i.e., IFNAR1 and/or IFNAR2 chains. In some embodiments, the modified interferon β agent has substantially reduced or ablated affinity and/or activity for the IFN-α/β receptor (IFNAR), i.e., IFNAR1 and/or IFNAR2 chains.

In an illustrative embodiment, the modified additional signaling agent is IFNβ. In various embodiments, the IFNβ encompasses functional derivatives, analogs, precursors, isoforms, splice variants, or fragments of IFNβ. In various embodiments, the IFNβ encompasses IFNβ derived from any species. In an embodiment, the chimeric proteins or chimeric protein complexes such as Fc-based chimeric protein complex comprises a modified version of mouse IFNβ. In another embodiment, the chimeric proteins or chimeric protein complexes such as Fc-based chimeric protein complex comprises a modified version of human IFNβ. Human IFNβ is a polypeptide with a molecular weight of about 22 kDa comprising 166 amino acid residues. The amino acid sequence of human IFNβ is SEQ ID NO: 277.

In some embodiments, the human IFNβ is IFNβ-1a which is a glycosylated form of human IFNβ. In some embodiments, the human IFNβ is IFNβ-1b which is a non-glycosylated form of human IFNβ that has a Met-1 deletion and a Cys-17 to Ser mutation.

In various embodiments, the modified IFNβ has one or more mutations that reduce its binding to or its affinity for the IFNAR1 subunit of IFNAR. In one embodiment, the modified IFNβ has reduced affinity and/or activity at IFNAR1. In various embodiments, the modified IFNβ is human IFNβ and has one or more mutations at positions F67, R71, L88, Y92, 195, N96, K123, and R124. In some embodiments, the one or more mutations are substitutions selected from F67G, F67S, R71A, L88G, L88S, Y92G, Y92S, 195A, N96G, K123G, and R124G. In an embodiment, the modified IFNβ comprises the F67G mutation. In an embodiment, the modified IFNβ comprises the K123G mutation. In an embodiment, the modified IFNβ comprises the F67G and R71A mutations. In an embodiment, the modified IFNβ comprises the L88G and Y92G mutations. In an embodiment, the modified IFNβ comprises the Y92G, 195A, and N96G mutations. In an embodiment, the modified IFNβ comprises the K123G and R124G mutations. In an embodiment, the modified IFNβ comprises the F67G, L88G, and Y92G mutations. In an embodiment, the modified IFNβ comprises the F67S, L88S, and Y92S mutations.

In some embodiments, the modified IFNβ has one or more mutations that reduce its binding to or its affinity for the IFNAR2 subunit of IFNAR. In one embodiment, the modified IFNβ has reduced affinity and/or activity at IFNAR2. In various embodiments, the modified IFNβ is human IFNβ and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G. In an embodiment, the modified IFNβ comprises the W22G mutation. In an embodiment, the modified IFNβ comprises the L32A mutation. In an embodiment, the modified IFNβ comprises the L32G mutation. In an embodiment, the modified IFNβ comprises the R35A mutation. In an embodiment, the modified IFNβ comprises the R35G mutation.

In an embodiment, the modified IFNβ comprises the V148G mutation. In an embodiment, the modified IFNβ comprises the R152A mutation. In an embodiment, the modified IFNβ comprises the R152G mutation. In an embodiment, the modified IFNβ comprises the Y155G mutation. In an embodiment, the modified IFNβ comprises the W22G and R27G mutations. In an embodiment, the modified IFNβ comprises the L32A and R35A mutation. In an embodiment, the modified IFNβ comprises the L151G and R152A mutations. In an embodiment, the modified

IFNβ comprises the V148G and R152A mutations.

In some embodiments, the modified IFNβ has one or more of the following mutations: R35A, R35T, E42K, M62I, G78S, A141Y. A142T. E149K, and R152H. In some embodiments, the modified IFNβ has one or more of the following mutations: R35A, R35T, E42K, M62I, G78S, A141Y, A142T, E149K, and R152H in combination with C173 or C17A.

In some embodiments, the modified IFNβ has one or more of the following mutations: R35A, R35T, E42K, M62I, G78S, A141Y, A142T, E149K, and R152H in combination with any of the other IFNβ mutations described herein.

The crystal structure of human IFNβ is known and is described in Karpusas et al., (1998) PNAS, 94(22): 11813-11818. Specifically, the structure of human IFNβ has been shown to include five α-helices (i.e., A, B, C, D, and E) and four loop regions that connect these helices (i.e., AB, BC, CD, and DE loops). In various embodiments, the modified IFNβ has one or more mutations in the A, B, C, D, E helices and/or the AB, BC, CD, and DE loops which reduce its binding affinity or activity at a therapeutic receptor such as IFNAR. Illustrative mutations are described in WO2000/023114 and US20150011732, the entire contents of which are hereby incorporated by reference. In an illustrative embodiment, the modified IFNβ is human IFNβ comprising alanine substitutions at amino acid positions 15, 16, 18, 19, 22, and/or 23. In an illustrative embodiment, the modified IFNβ is human IFNβ comprising alanine substitutions at amino acid positions 28-30, 32, and 33. In an illustrative embodiment, the modified IFNβ is human IFNβ comprising alanine substitutions at amino acid positions 36, 37, 39, and 42. In an illustrative embodiment, the modified IFNβ is human IFNβ comprising alanine substitutions at amino acid positions 64 and 67 and a serine substitution at position 68. In an illustrative embodiment, the modified IFNβ is human IFNβ comprising alanine substitutions at amino acid positions 71-73. In an illustrative embodiment, the modified IFNβ is human IFNβ comprising alanine substitutions at amino acid positions 92, 96, 99, and 100. In an illustrative embodiment, the modified IFNβ is human IFNβ comprising alanine substitutions at amino acid positions 128, 130, 131, and 134. In an illustrative embodiment, the modified IFNβ is human IFNβ comprising alanine substitutions at amino acid positions 149, 153, 156, and 159. In some embodiments, the mutant IFNβ comprises SEQ ID NO:277 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO:277 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L88, the mutation being an aliphatic hydrophobic residue selected from glycine (G), alanine (A), isoleucine (I), methionine (M), and valine (V).

In some embodiments, the mutant IFNβ comprises SEQ ID NO:277 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at 195, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at N96, the mutation being an aliphatic hydrophobic residue selected from glycine (G), alanine (A), leucine (L), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at Y92, the mutation being an aliphatic hydrophobic residue selected from glycine (G), alanine (A), leucine (L), isoleucine (I), methionine (M), and valine (V) and a mutation at 195, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at K123, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at K123, the mutation being an aliphatic hydrophobic residue selected from glycine (G), alanine (A), leucine (L), isoleucine (I), methionine (M), and valine (V) and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at L151, the mutation being an aliphatic hydrophobic residue selected from glycine (G), alanine (A), isoleucine (I), methionine (M), and valine (V).

In some embodiments, the mutant IFNβ comprises SEQ ID NO: 277 and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at L151, the mutation being an aliphatic hydrophobic residue selected from glycine (G), alanine (A), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at V148, the mutation being an aliphatic hydrophobic residue selected from glycine (G), alanine (A), leucine (L), isoleucine (I), and methionine (M).

In some embodiments, the mutant IFNβ comprises SEQ ID NO: 277 and a mutation at V148, the mutation being an aliphatic hydrophobic residue selected from glycine (G), alanine (A), leucine (L), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at Y155, the mutation being an aliphatic hydrophobic residue selected from glycine (G), alanine (A), leucine (L), isoleucine (I), methionine (M), and valine (V).

In some embodiments, the present invention relates to a chimeric proteins or chimeric protein complexes such as Fc-based chimeric protein complex comprising: (a) a modified IFNβ, having the amino acid sequence of SEQ ID NO: 277 and a mutation at position W22, wherein the mutation is an aliphatic hydrophobic residue and a modified IL-2 or modified IL-2 variant disclosed here; and (b) one or more targeting moieties, said targeting moieties comprising recognition domains which specifically bind to antigens or receptors of interest, the modified IFNβ and the one or more targeting moieties are optionally connected with one or more linkers. In various embodiments the mutation at position W22 is aliphatic hydrophobic residue is selected from G, A, L, I, M, and V. In various embodiments the mutation at position W22 is G.

Additional illustrative IFNβ mutants are provided in PCT/EP2017/061544, the entire disclosure of which is incorporated by reference herein.

In some embodiments, the modified additional signaling agent is interferon γ. In such embodiments, the modified interferon γ agent has reduced affinity and/or activity for the interferon-gamma receptor (IFNGR), i.e., IFNGR1 and IFNGR2 chains. In some embodiments, the modified interferon γ agent has substantially reduced or ablated affinity and/or activity for the interferon-gamma receptor (IFNGR), i.e., IFNGR1 and/or IFNGR2 chains.

In some embodiments, the modified additional signaling agent is a consensus interferon. The consensus interferon is generated by scanning the sequences of several human non-allelic IFNα subtypes and assigning the most frequently observed amino acid in each corresponding position. The consensus interferon differs from IFNα2b at out of 166 amino acids (88% homology), and comparison with IFNβ shows identity at over 30% of the amino acid positions. In various embodiments, the consensus interferon comprises the following amino acid sequence of SEQ ID NO: 278.

In some embodiments, the consensus interferon comprises the amino acid sequence of SEQ ID NO: 279, which differs from the amino acid sequence of SEQ ID NO: 278 by one amino acid, i.e., SEQ ID NO: 279 lacks the initial methionine residue of SEQ ID NO: 278:

In various embodiments, the consensus interferon comprises a modified version of the consensus interferon, i.e., a consensus interferon variant, as a signaling agent. In various embodiments, the consensus interferon variant encompasses functional derivatives, analogs, precursors, isoforms, splice variants, or fragments of the consensus interferon.

In an embodiment, the consensus interferon variants are selected form the consensus interferon variants disclosed in U.S. Pat. Nos. 4,695,623, 4,897,471, 5,541,293, and 8,496,921, the entire contents of all of which are hereby incorporated by reference. For example, the consensus interferon variant may comprise the amino acid sequence of IFN-CON₂ or IFN-CON₃ as disclosed in U.S. Pat. Nos. 4,695,623, 4,897,471, and 5,541,293. In an embodiment, the consensus interferon variant comprises the amino acid sequence of IFN-CON₂: SEQ ID NO: 280.

In an embodiment, the consensus interferon variant comprises the amino acid sequence of IFN-CON₃: SEQ ID NO: 281.

In an embodiment, the consensus interferon variant comprises the amino acid sequence of any one of the variants disclosed in U.S. Pat. No. 8,496,921. For example, the consensus variant may comprise the amino acid sequence of: SEQ ID NO: 282.

In another embodiment, the consensus interferon variant may comprise the amino acid sequence of: SEQ ID NO: 283.

In some embodiments, the consensus interferon variant may be PEGylated, i.e., comprises a PEG moiety. In an embodiment, the consensus interferon variant may comprise a PEG moiety attached at the S156C position of SEQ ID NO: 283.

In some embodiments, the engineered interferon is a variant of human IFNα2a, with an insertion of Asp at approximately position 41 in the sequence Glu-Glu-Phe-Gly-Asn-Gln (SEQ ID NO: 284) to yield Glu-Glu-Phe-Asp-Gly-Asn-Gln (SEQ ID NO: 285) (which resulted in a renumbering of the sequence relative to IFNα2a sequence) and the following mutations of Arg23Lys, Leu26Pro, Glu53GIn, Thr54Ala, Pro56Ser, Asp86Glu, Ile104Thr, Glyl06Glu, Thr110Glu, Lys117Asn, Arg125Lys, and Lys136Thr. All embodiments herein that describe consensus interferons apply equally to this engineered interferon

In some embodiments, the additional modified signaling agent is vascular endothelial growth factor (VEGF). VEGF is a potent growth factor that plays major roles in physiological but also pathological angiogenesis, regulates vascular permeability and can act as a growth factor on cells expressing VEGF receptors. Additional functions include, among others, stimulation of cell migration in macrophage lineage and endothelial cells. Several members of the VEGF family of growth factors exist, as well as at least three receptors (VEGFR-1, VEGFR-2, and VEGFR-3). Members of the VEGF family can bind and activate more than one VEGFR type. For example, VEGF-A binds VEGFR-1 and -2, while VEGF-C can bind VEGFR-2 and -3. VEGFR-1 and VEGFR-2 activation regulate angiogenesis while VEGFR-3 activation is associated with lymphangiogenesis. The major pro-angiogenic signal is generated from activation of VEGFR-2. VEGFR-1 activation has been reported to be possibly associated with negative role in angiogenesis. It has also been reported that VEGFR-1 signaling is important for progression of tumors in vivo via bone marrow-derived VEGFR-1 positive cells (contributing to formation of premetastatic niche in the bone). Several therapies based on VEGF-A directed/neutralizing therapeutic antibodies have been developed, primarily for use in treatment of various human tumors relying on angiogenesis. These are not without side effects though. This may not be surprising considering that these operate as general, non-cell/tissue specific VEGF/VEGFR interaction inhibitors. Hence, it would be desirable to restrict VEGF (e.g. VEGF-A)/EGFR-2 inhibition to specific target cells (e.g. tumor vasculature endothelial cells).

In some embodiments, the VEGF is VEGF-A, VEGF-B, VEFG-C, VEGF-D, or VEGF-E and isoforms thereof including the various isoforms of VEGF-A such as VEGF 121, VEGF 121b, VEGF 145, VEGF 165, VEGF 165b, VEGF 189, and VEGF206. In some embodiments, the modified signaling agent has reduced affinity and/or activity for VEGFR-1 (Flt-1) and/or VEGFR-2 (KDR/Flk-1). In some embodiments, the modified signaling agent has substantially reduced or ablated affinity and/or activity for VEGFR-1 (Flt-1) and/or VEGFR-2 (KDR/Flk-1). In an embodiment, the modified signaling agent has reduced affinity and/or activity for VEGFR-2 (KDR/Flk-1) and/or substantially reduced or ablated affinity and/or activity for VEGFR-1 (Flt-1). Such an embodiment finds use, for example, in wound healing methods or treatment of ischmia-related diseases (without wishing to be bound by theory, mediated by VEGFR-2's effects on endothelial cell function and angiogenesis). In various embodiments, binding to VEGFR-1 (Flt-1), which is linked to cancers and pro-inflammatory activities, is avoided. In various embodiments, VEGFR-1 (Flt-1) acts a decoy receptor and therefore substantially reduces or ablates affinity at this receptor avoids sequestration of the therapeutic agent. In an embodiment, the modified signaling agent has substantially reduced or ablated affinity and/or activity for VEGFR-1 (Flt-1) and/or substantially reduced or ablated affinity and/or activity for VEGFR-2 (KDR/Flk-1). In some embodiments, the VEGF is VEGF-C or VEGF-D. In such embodiments, the modified signaling agent has reduced affinity and/or activity for VEGFR-3. Alternatively, the modified signaling agent has substantially reduced or ablated affinity and/or activity for VEGFR-3.

Proangiogenic therapies are also important in various diseases (e.g. ischemic heart disease, bleeding etc.), and include VEGF-based therapeutics. Activation of VEGFR-2 is proangiogenic (acting on endothelial cells). Activation of VEFGR-1 can cause stimulation of migration of inflammatory cells (including, for example, macrophages) and lead to inflammation associated hypervascular permeability. Activation of VEFGR-1 can also promote bone marrow associated tumor niche formation. Thus, VEGF based therapeutic selective for VEGFR-2 activation would be desirable in this case. In addition, cell specific targeting, e.g. to endothelial cells, would be desirable.

In some embodiments, the additional modified signaling agent has reduced affinity and/or activity (e.g. antagonistic) for VEGFR-2 and/or has substantially reduced or ablated affinity and/or activity for VEGFR-1. When targeted to tumor vasculature endothelial cells via a targeting moiety that binds to a tumor endothelial cell marker (e.g. PSMA and others), such construct inhibits VEGFR-2 activation specifically on such marker-positive cells, while not activating VEGFR-1 en route and on target cells (if activity ablated), thus eliminating induction of inflammatory responses, for example. This would provide a more selective and safe anti-angiogenic therapy for many tumor types as compared to VEGF-A neutralizing therapies.

In some embodiments, the additional modified signaling agent has reduced affinity and/or activity (e.g. agonistic) for VEGFR-2 and/or has substantially reduced or ablated affinity and/or activity for VEGFR-1. Through targeting to vascular endothelial cells, such construct, in some embodiments, promotes angiogenesis without causing VEGFR-1 associated induction of inflammatory responses. Hence, such a construct would have targeted proangiogenic effects with substantially reduced risk of side effects caused by systemic activation of VEGFR-2 as well as VEGR-1.

In an illustrative embodiment, the modified signaling agent is VEGF 165, which has the amino acid sequence of SEQ ID NO:235.

In another illustrative embodiment, the additional modified signaling agent is VEGF 165b, which has the amino acid sequence of SEQ ID NO:236.

In these embodiments, the modified signaling agent has a mutation at amino acid I83 (e.g., a substitution mutation at I83, e.g., I83K, I83R, or I83H). Without wishing to be bound by theory, it is believed that such mutations may result in reduced receptor binding affinity. See, for example, U.S. Pat. No. 9,078,860, the entire contents of which are hereby incorporated by reference.

In an embodiment, the additional modified signaling agent is TNFα. TNF is a pleiotropic cytokine with many diverse functions, including regulation of cell growth, differentiation, apoptosis, tumorigenesis, viral replication, autoimmunity, immune cell functions and trafficking, inflammation, and septic shock. It binds to two distinct membrane receptors on target cells: TNFR1 (p55) and TNFR2 (p75). TNFR1 exhibits a very broad expression pattern whereas TNFR2 is expressed preferentially on certain populations of lymphocytes, Tregs, endothelial cells, certain neurons, microglia, cardiac myocytes and mesenchymal stem cells. Very distinct biological pathways are activated in response to receptor activation, although there is also some overlap. As a general rule, without wishing to be bound by theory, TNFR1 signaling is associated with induction of apoptosis (cell death) and TNFR2 signaling is associated with activation of cell survival signals (e.g. activation of NFκB pathway). Administration of TNF is systemically toxic, and this is largely due to TNFR1 engagement. However, it should be noted that activation of TNFR2 is also associated with a broad range of activities and, as with TNFR1, in the context of developing TNF based therapeutics, control over TNF targeting and activity is important.

In some embodiments, the additional modified signaling agent has reduced affinity and/or activity for TNFR1 and/or TNFR2. In some embodiments, the modified signaling agent has substantially reduced or ablated affinity and/or activity for TNFR1 and/or TNFR2. TNFR1 is expressed in most tissues, and is involved in cell death signaling while, by contrast, TNFR2 is involved in cell survival signaling. Accordingly, in embodiments directed to methods of treating cancer, the modified signaling agent has reduced affinity and/or activity for TNFR1 and/or substantially reduced or ablated affinity and/or activity for TNFR2. In these embodiments, the chimeric proteins or chimeric protein complexes such as Fc-based chimeric protein complex may be targeted to a cell for which apoptosis is desired, e.g. a tumor cell or a tumor vasculature endothelial cell. In embodiments directed to methods of promoting cell survival, for example, in neurogenesis for the treatment of neurodegenerative disorders, the modified signaling agent has reduced affinity and/or activity for TNFR2 and/or substantially reduced or ablated affinity and/or activity for TNFR1. Stated another way, the present chimeric proteins or chimeric protein complexes such as Fc-based chimeric protein complex, in some embodiments, comprise modified TNF-α agent that allows of favoring either death or survival signals.

In some embodiments, the chimeric proteins or chimeric protein complexes such as Fc-based chimeric protein complex has a modified TNF having reduced affinity and/or activity for TNFR1 and/or substantially reduced or ablated affinity and/or activity for TNFR2. Such a chimera, in some embodiments, is a more potent inducer of apoptosis as compared to a wild type TNF and/or a chimera bearing only mutation(s) causing reduced affinity and/or activity for TNFR1. Such a chimera, in some embodiments, finds use in inducing tumor cell death or a tumor vasculature endothelial cell death (e.g. in the treatment of cancers). Also, in some embodiments, these chimeras avoid or reduce activation of Treg cells via TNFR2, for example, thus further supporting TNFR1-mediated antitumor activity in vivo.

In some embodiments, the chimeric proteins or chimeric protein complexes such as Fc-based chimeric protein complex has a modified TNF having reduced affinity and/or activity for TNFR2 and/or substantially reduced or ablated affinity and/or activity for TNFR1. Such a chimera, in some embodiments, is a more potent activator of cell survival in some cell types, which may be a specific therapeutic objective in various disease settings, including without limitation, stimulation of neurogenesis. In addition, a TNFR2-favoring chimera is also useful in the treatment of autoimmune diseases (e.g. Crohn's, diabetes, MS, colitis etc. and many others described herein). In some embodiments, the chimera is targeted to auto-reactive T cells. In some embodiments, the chimera promotes Treg cell activation and indirect suppression of cytotoxic T cells.

In some embodiments, the chimera causes the death of auto-reactive T cells, e.g. by activation of TNFR2 and/or avoidance of TNFR1 (e.g. a modified TNF having reduced affinity and/or activity for TNFR2 and/or substantially reduced or ablated affinity and/or activity for TNFR1). Without wishing to be bound by theory these auto-reactive T cells, have their apoptosis/survival signals altered e.g. by NFκB pathway activity/signaling alterations. In some embodiments, a TNFR2 based chimera has additional therapeutic applications in diseases, including various autoimmune diseases, heart disease, de-myelinating and neurodegenerative disorders, and infectious disease, among others.

In an embodiment, the wild type TNFα has the amino acid sequence of SEQ ID NO:237.

In such embodiments, the modified TNFα agent has mutations at one or more amino acid positions 29, 31, 32, 84, 85, 86, 87, 88, 89, 145, 146 and 147 which produces a modified TNFα with reduced receptor binding affinity. See, for example, U.S. Pat. No. 7,993,636, the entire contents of which are hereby incorporated by reference.

In some embodiments, the modified human TNFα moiety has mutations at one or more amino acid positions R32, N34, Q67, H73, L75, T77, S86, Y87, V91, 197, T105, P106, A109, P113, Y115, E127, N137, D143, and A145, as described, for example, in WO/2015/007903, the entire contents of which is hereby incorporated by reference (numbering according to the human TNF sequence, Genbank accession number BAG70306, version BAG70306.1 GI: 197692685). In some embodiments, the modified human TNFα moiety has substitution mutations selected from R32G, N34G, Q67G, H73G, L75G, L75A, L75S, T77A, S86G, Y87Q, Y87L, Y87A, Y87F, V91G, V91A, 197A, 197Q, 197S, T105G, P106G, A109Y, P113G, Y115G, Y115A, E127G, N137G, D143N, A145G and A145T. In an embodiment, the human TNFα moiety has a mutation selected from Y87Q, Y87L, Y87A, and Y87F. In another embodiment, the human TNFα moiety has a mutation selected from 197A, 197Q, and 197S. In a further embodiment, the human TNFα moiety has a mutation selected from Y115A and Y115G.

In some embodiments, the modified TNFα agent has one or more mutations selected from N39Y, S147Y, and Y87H, as described in WO2008/124086, the entire contents of which is hereby incorporated by reference.

In an embodiment, the additional modified signaling agent is TNFβ. TNFβ can form a homotrimer or a heterotrimer with LT-β (LT-α1β2). In some embodiments, the modified signaling agent has substantially reduced or ablated affinity and/or activity for TNFR1 and/or TNFR2 and/or herpes virus entry mediator (HEVM) and/or LT-BR.

In an embodiment, the wild type TNFβ has the amino acid sequence of SEQ ID NO:238.

In such embodiments, the modified TNFβ agent may comprise mutations at one or more amino acids at positions 106-113, which produce a modified TNFβ with reduced receptor binding affinity to TNFR2. In an embodiment, the modified signaling agent has one or more substitution mutations at amino acid positions 106-113. In illustrative embodiments, the substitution mutations are selected from Q107E, Q107D, S106E, S106D, Q107R, Q107N, Q107E/S106E, Q107E/S106D, Q107D/S106E, and Q107D/S 106D. In another embodiment, the modified signaling agent has an insertion of about 1 to about 3 amino acids at positions 106-113.

In some embodiments, the additional modified agent is a TNF family member (e.g. TNF-alpha, TNF-beta) which can be a single chain trimeric version as described in WO 2015/007903, the entire contents of which are incorporated by reference.

In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR1. In these embodiments, the modified agent is a TNF family member (e.g. TNF-alpha, TNF-beta) which also, optionally, has substantially reduced or ablated affinity and/or activity for TNFR2. In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR2. In these embodiments, the modified agent is a TNF family member (e.g. TNF-alpha, TNF-beta) which also, optionally, has substantially reduced or ablated affinity and/or activity for TNFR1. The constructs of such embodiments find use in, for example, methods of dampening TNF response in a cell specific manner. In some embodiments, the antagonistic TNF family member (e.g. TNF-alpha, TNF-beta) is a single chain trimeric version as described in WO 2015/007903.

In an embodiment, the additional modified signaling agent is TRAIL. In some embodiments, the modified TRAIL agent has reduced affinity and/or activity for DR4 (TRAIL-RI) and/or DR5 (TRAIL-RII) and/or DcR1 and/or DcR2. In some embodiments, the modified TRAIL agent has substantially reduced or ablated affinity and/or activity for DR4 (TRAIL-RI) and/or DR5 (TRAIL-RII) and/or DcR1 and/or DcR2.

In an embodiment, the wild type TRAIL has the amino acid sequence of SEQ ID NO:239.

In such embodiments, the modified TRAIL agent may comprise a mutation at amino acid positions T127-R132, E144-R149, E155-H161, Y189-Y209, T214-1220, K224-A226, W231, E236-L239, E249-K251, T261-H264 and

H270-E271 (Numbering based on the human sequence, Genbank accession number NP_003801, version 10 NP_003801.1, GI: 4507593; see above).

In an embodiment, the additional modified signaling agent is TGFα. In such embodiments, the modified TGFα agent has reduced affinity and/or activity for the epidermal growth factor receptor (EGFR). In some embodiments, the modified TGFα agent has substantially reduced or ablated affinity and/or activity for the epidermal growth factor receptor (EGFR).

In an embodiment, the additional modified signaling agent is TGFβ. In such embodiments, the modified signaling agent has reduced affinity and/or activity for TGFBR1 and/or TGFBR2. In some embodiments, the modified signaling agent has substantially reduced or ablated affinity and/or activity for TGFBR1 and/or TGFBR2. In some embodiments, the modified signaling agent optionally has reduced or substantially reduced or ablated affinity and/or activity for TGFBR3 which, without wishing to be bound by theory, may act as a reservoir of ligand for TGF-beta receptors. In some embodiments, the TGFB may favor TGFBR1 over TGFBR2 or TGFBR2 over TGFBR1. Similarly, LAP, without wishing to be bound by theory, may act as a reservoir of ligand for TGF-beta receptors. In some embodiments, the modified signaling agent has reduced affinity and/or activity for TGFBR1 and/or TGFBR2 and/or substantially reduced or ablated affinity and/or activity for Latency Associated Peptide (LAP). In some embodiments, such chimeras find use in Camurati-Engelmann disease, or other diseases associated with inappropriate TGFβ signaling.

In some embodiments, the additional modified agent is a TGF family member (e.g. TGFα, TGFβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at one or more of TGFBR1, TGFBR2, TGFBR3. In these embodiments, the modified agent is a TGF family member (e.g. TGFα, TGFβ) which also, optionally, has substantially reduced or ablated affinity and/or activity at one or more of TGFBR1, TGFBR2, TGFBR3.

In some embodiments, the additional modified agent is a TGF family member (e.g. TGFα, TGFβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TGFBR1 and/or TGFBR2. In these embodiments, the modified agent is a TGF family member (e.g. TGFα, TGFβ) which also, optionally, has substantially reduced or ablated affinity and/or activity at TGFBR3.

In an embodiment, the additional modified signaling agent is IL-1. In an embodiment, the modified signaling agent is IL-1α or IL-1β. In some embodiments, the modified signaling agent has reduced affinity and/or activity for IL-1R1 and/or IL-1RAcP. In some embodiments, the modified signaling agent has substantially reduced or ablated affinity and/or activity for IL-1R1 and/or IL-1RAcP. In some embodiments, the modified signaling agent has reduced affinity and/or activity for IL-1R2. In some embodiments, the modified signaling agent has substantially reduced or ablated affinity and/or activity for IL-1R2. For instance, in some embodiments, the present modified IL-1 agents avoid interaction at IL-1R2 and therefore substantially reduce its function as a decoy and/or sink for therapeutic agents.

In an embodiment, the wild type IL-1β has the amino acid sequence of SEQ ID NO:240.

IL-1β is a proinflammatory cytokine and an important immune system regulator. It is a potent activator of CD4 T cell responses, increases proportion of Th17 cells and expansion of IFNγ and IL-4 producing cells. IL-1β is also a potent regulator of CD8⁺ T cells, enhancing antigen-specific CD8⁺ T cell expansion, differentiation, migration to periphery and memory. IL-13 receptors comprise IL-1R1 and IL-1R2. Binding to and signaling through the IL-1R1 constitutes the mechanism whereby IL-1β mediates many of its biological (and pathological) activities. IL1-R2 can function as a decoy receptor, thereby reducing IL-1β availability for interaction and signaling through the IL-1R1.

In some embodiments, the modified IL-1β has reduced affinity and/or activity (e.g. agonistic activity) for IL-1R1. In some embodiments, the modified IL-1β has substantially reduced or ablated affinity and/or activity for IL-1R2. In such embodiments, there is inducible or restorable IL-1β/IL-1R1 signaling and prevention of loss of therapeutic chimeras at IL-R2 and therefore a reduction in dose of IL-1 that is required (e.g. relative to wild type or a chimera bearing only an attenuation mutation for IL-R1). Such constructs find use in, for example, methods of treating cancer, including, for example, stimulating the immune system to mount an anti-cancer response.

In some embodiments, the modified IL-1β has reduced affinity and/or activity (e.g. antagonistic activity, e.g. natural antagonistic activity or antagonistic activity that is the result of one or more mutations, see, e.g., WO 2015/007520, the entire contents of which are hereby incorporated by reference) for IL-1R1. In some embodiments, the modified IL-1β has substantially reduced or ablated affinity and/or activity for IL-1R2. In such embodiments, there is the IL-1B/IL-1R1 signaling is not inducible or restorable and prevention of loss of therapeutic chimeras at IL-R2 and therefore a reduction in dose of IL-1β that is required (e.g. relative to wild type or a chimera bearing only an attenuation mutation for IL-R1). Such constructs find use in, for example, methods of treating autoimmune diseases, including, for example, suppressing the immune system.

In such embodiments, the modified signaling agent has a deletion of amino acids 52-54 which produces a modified human IL-1β with reduced binding affinity for type I IL-1R and reduced biological activity. See, for example, WO 1994/000491, the entire contents of which are hereby incorporated by reference. In some embodiments, the modified human IL-1β has one or more substitution mutations selected from A117G/P118G, R120X, L122A, T125G/L126G, R127G, Q130X, Q131G, K132A, S137G/Q138Y, L145G, H146X, L145A/L147A, Q148X, Q148G/Q150G, Q150G/D151A, M152G, F162A, F162A/Q164E, F166A, Q164E/E167K, N169G/D170G, I172A, V174A, K208E, K209X, K209A/K210A, K219X, E221X, E221 S/N224A, N224S/K225S, E244K, N245Q (where X can be any change in amino acid, e.g., a non-conservative change), which exhibit reduced binding to IL-1R, as described, for example, in WO2015/007542 and WO/2015/007536, the entire contents of which is hereby incorporated by reference (numbering base on the human IL-1β sequence, Genbank accession number NP_000567, version NP-000567.1, GI: 10835145). In some embodiments, the modified human IL-1β may have one or more mutations selected from R120A, R120G, Q130A, Q130W, H146A, H146G, H146E, H146N, H146R, Q148E, Q148G, Q148L, K209A, K209D, K219S, K219Q, E221S and E221K. In an embodiment, the modified human IL-1β comprises the mutations Q131G and Q148G. In an embodiment, the modified human IL-1β comprises the mutations Q148G and K208E. In an embodiment, the modified human IL-1β comprises the mutations R120G and Q131G. In an embodiment, the modified human IL-1β comprises the mutations R120G and H146G. In an embodiment, the modified human IL-1β comprises the mutations R120G and K208E. In an embodiment, the modified human IL-1β comprises the mutations R120G, F162A, and Q164E.

In an embodiment, the additional modified signaling agent is IL-3. In some embodiments, the modified signaling agent has reduced affinity and/or activity for the IL-3 receptor, which is a heterodimer with a unique alpha chain paired with the common beta (beta c or CD131) subunit. In some embodiments, the modified signaling agent has substantially reduced or ablated affinity and/or activity for the IL-3 receptor, which is a heterodimer with a unique alpha chain paired with the common beta (beta c or CD131) subunit.

In an embodiment, the additional modified signaling agent is IL-4. In such an embodiment, the modified signaling agent has reduced affinity and/or activity for type 1 and/or type 2 IL-4 receptors. In such an embodiment, the modified signaling agent has substantially reduced or ablated affinity and/or activity for type 1 and/or type 2 IL-4 receptors. Type 1 IL-4 receptors are composed of the IL-4Rα subunit with a common γ chain and specifically bind IL-4. Type 2 IL-4 receptors include an IL-4Rα subunit bound to a different subunit known as IL-13Rα1. In some embodiments, the modified signaling agent has substantially reduced or ablated affinity and/or activity the type 2 IL-4 receptors.

In an embodiment, the wild type IL-4 has the amino acid sequence of SEQ ID NO:242.

In such embodiments, the modified IL-4 agent has one or more mutations at amino acids R121 (R121A, R121D, R121E, R121F, R121H, R1211, R121K, R121N, R121P, R121T, R121W), E122 (E122F), Y124 (Y124A, Y124Q, Y124R, Y124S, Y124T) and S125 (S125A). Without wishing to be bound by theory, it is believed that these modified IL-4 agents maintain the activity mediated by the type I receptor, but significantly reduces the biological activity mediated by the other receptors. See, for example, U.S. Pat. No. 6,433,157, the entire contents of which are hereby incorporated by reference.

In an embodiment, the additional modified signaling agent is IL-6. IL-6 signals through a cell-surface type I cytokine receptor complex including the ligand-binding IL-6R chain (CD126), and the signal-transducing component gp 130. IL-6 may also bind to a soluble form of IL-6R (sIL-6R), which is the extracellular portion of IL-6R. The sIL-6R/IL-6 complex may be involved in neurites outgrowth and survival of neurons and, hence, may be important in nerve regeneration through remyelination. Accordingly, in some embodiments, the modified signaling agent has reduced affinity and/or activity for IL-6R/gp130 and/or sIL-6R. In some embodiments, the modified signaling agent has substantially reduced or ablated affinity and/or activity for IL-6R/gp130 and/or sIL-6R.

In an embodiment, the wild type IL-6 has the amino acid sequence of SEQ ID NO:243.

In such embodiments, the modified signaling agent has one or more mutations at amino acids 58, 160, 163, 171 or 177. Without wishing to be bound by theory, it is believed that these modified IL-6 agents exhibit reduced binding affinity to IL-6Ralpha and reduced biological activity. See, for example, WO 97/10338, the entire contents of which are hereby incorporated by reference.

In an embodiment, the additional modified signaling agent is IL-10. In such an embodiment, the modified signaling agent has reduced affinity and/or activity for IL-10 receptor-1 and IL-10 receptor-2. In some embodiments, the modified signaling agent has substantially reduced or ablated affinity and/or activity for IL-10 receptor-1 and IL-10 receptor-2 In an embodiment, the additional modified signaling agent is IL-11. In such an embodiment, the modified signaling agent has reduced affinity and/or activity for IL-11Rα and/or IL-11RB and/or gp 130. In such an embodiment, the modified signaling agent has substantially reduced or ablated affinity and/or activity for IL-11Rα and/or IL-11RB and/or gp 130.

In an embodiment, the additional modified signaling agent is IL-12. In such an embodiment, the modified signaling agent has reduced affinity and/or activity for IL-12Rβ1 and/or IL-12Rβ2. In such an embodiment, the modified signaling agent has substantially reduced or ablated affinity and/or activity for IL-12Rβ1 and/or IL-12Rβ2.

In an embodiment, the additional modified signaling agent is IL-13. In such an embodiment, the modified signaling agent has reduced affinity and/or activity for the IL-4 receptor (IL-4Rα) and IL-13Rα1. In some embodiments, the modified signaling agent has substantially reduced or ablated affinity and/or activity for IL-4 receptor (IL-4Rα) or IL-13Rα1.

In an embodiment, the wild type IL-13 has the amino acid sequence of SEQ ID NO:244.

In such embodiments, the modified IL-13 agent has one or more mutations at amino acids 13, 16, 17, 66, 69, 99, 102, 104, 105, 106, 107, 108, 109, 112, 113 and 114. Without wishing to be bound by theory, it is believed that these modified IL-13 agents exhibit reduced biological activity. See, for example, WO 2002/018422, the entire contents of which are hereby incorporated by reference.

In an embodiment, the signaling agent is a wild type or modified IL-15. In embodiments, the modified IL-15 has reduced affinity and/or activity for interleukin 15 receptor.

In an embodiment, the wild type IL-15 has the amino acid sequence of:
NWVNVISDLKKIEDLIQSMHIDATLYTESDVHPSCKVTAMKCFLLELQVISHESGDTDIHDTVEN LIILANNILSSNGNITESGCKECEELEEKNIKEFLQSFVHIVQMFINTS (SEQ ID NO: 292).

In such embodiments, the modified IL-15 agent has one or more mutations at amino acids S7, D8, K10, K11, E46, L47, V49, 150, D61, N65, L66, 167, 168, L69, N72, Q108 with respect to SEQ ID NO: 292.

In an embodiment, the additional modified signaling agent is IL-18. In some embodiments, the modified signaling agent has reduced affinity and/or activity for IL-18Rα and/or IL-18Rβ. In some embodiments, the modified signaling agent has substantially reduced or ablated affinity and/or activity for IL-18Rα and/or IL-18Rβ. In some embodiments, the modified signaling agent has substantially reduced or ablated affinity and/or activity for IL-18Rα type II, which is an isoform of IL-18Rα that lacks the TIR domain required for signaling.

In an embodiment, the wild type IL-18 has the amino acid sequence of SEQ ID NO:245.

In such embodiments, the modified IL-18 agent may comprise one or more mutations in amino acids or amino acid regions selected from Y37-K44, R49-Q54, D59-R63, E67-C74, R80, M87-A97, N127-K129, Q139-M149, K 165-K171, R183 and Q190-N191, as described in WO/2015/007542, the entire contents of which are hereby incorporated by reference (numbering based on the human IL-18 sequence, Genbank accession number AAV38697, version AAV38697.1, GI: 54696650).

In an embodiment, the additional modified signaling agent is IL-33. In such an embodiment, the modified signaling agent has reduced affinity and/or activity for the ST-2 receptor and IL-1RAcP. In some embodiments, the modified signaling agent has substantially reduced or ablated affinity and/or activity for the ST-2 receptor and IL-1RAcP.

In an embodiment, the wild type IL-33 has the amino acid sequence of SEQ ID NO:246.

In such embodiments, the modified IL-33 agent may comprise one or more mutations in amino acids or amino acid regions selected from 1113-Y122, S127-E139, E144-D157, Y163-M183, E200, Q215, L220-C227 and T260-E269, as described in WO/2015/007542, the entire contents of which are hereby incorporated by reference (numbering based on the human sequence, Genbank accession number NP_254274, version NP_254274.1, GI: 15559209).

In an embodiment, the modified signaling agent is epidermal growth factor (EGF). EGF is a member of a family of potent growth factors. Members include EGF, HB-EGF, and others such as TGFalpha, amphiregulin, neuregulins, epiregulin, betacellulin. EGF family receptors include EGFR (ErbB1), ErbB2, ErbB3 and ErbB4. These may function as homodimeric and/or heterodimeric receptor subtypes. The different EGF family members exhibit differential selectivity for the various receptor subtypes. For example, EGF associates with ErbB1/ErbB1, ErbB1/ErbB2, ErbB4/ErbB2 and some other heterodimeric subtypes. HB-EGF has a similar pattern, although it also associates with ErbB4/4. Modulation of EGF (EGF-like) growth factor signaling, positively or negatively, is of considerable therapeutic interest. For example, inhibition of EGFRs signaling is of interest in the treatment of various cancers where EGFR signaling constitutes a major growth promoting signal. Alternatively, stimulation of EGFRs signaling is of therapeutic interest in, for example, promoting wound healing (acute and chronic), oral mucositis (a major side-effect of various cancer therapies, including, without limitation radiation therapy).

In some embodiments, the additional modified signaling agent has reduced affinity and/or activity for ErbB1, ErbB2, ErbB3, and/or ErbB4. Such embodiments find use, for example, in methods of treating wounds. In some embodiments, the modified signaling agent binds to one or more ErbB1, ErbB2, ErbB3, and ErbB4 and antagonizes the activity of the receptor. In such embodiments, the modified signaling agent has reduced affinity and/or activity for ErbB1, ErbB2, ErbB3, and/or ErbB4 which allows for the activity of the receptor to be antagonized in an attenuated fashion. Such embodiments find use in, for example, treatments of cancer. In an embodiment, the modified signaling agent has reduced affinity and/or activity for ErbB1. ErbB1 is the therapeutic target of kinase inhibitors-most have side effects because they are not very selective (e.g., gefitinib, erlotinib, afatinib, brigatinib and icotinib). In some embodiments, attenuated antagonistic ErbB1 signaling is more on-target and has less side effects than other agents targeting receptors for EGF.

In some embodiments, the additional modified signaling agent has reduced affinity and/or activity (e.g. antagonistic e.g. natural antagonistic activity or antagonistic activity that is the result of one or more mutations, see, e.g., WO 2015/007520, the entire contents of which are hereby incorporated by reference) for ErbB1 and/or substantially reduced or ablated affinity and/or activity for ErbB4 or other subtypes it may interact with. Through specific targeting via the targeting moiety, cell-selective suppression (antagonism e.g. natural antagonistic activity or antagonistic activity that is the result of one or more mutations, see, e.g., WO 2015/007520, the entire contents of which are hereby incorporated by reference) of ErbB1/ErbB1 receptor activation would be achieved—while not engaging other receptor subtypes potentially associated with inhibition-associated side effects. Hence, in contrast to EGFR kinase inhibitors, which inhibit EGFR activity in all cell types in the body, such a construct would provide a cell-selective (e.g., tumor cell with activated EGFR signaling due to amplification of receptor, overexpression etc.) anti-EGFR (ErbB1) drug effect with reduced side effects.

In some embodiments, the additional modified signaling agent has reduced affinity and/or activity (e.g. agonistic) for ErbB4 and/or other subtypes it may interact with. Through targeting to specific target cells through the targeting moiety, a selective activation of ErbB1 signaling is achieved (e.g. epithelial cells). Such a construct finds use, in some embodiments, in the treatment of wounds (promoting would healing) with reduced side effects, especially for treatment of chronic conditions and application other than topical application of a therapeutic (e.g. systemic wound healing)

In an embodiment, the modified signaling agent is insulin or insulin analogs. In some embodiments, the modified insulin or insulin analog has reduced affinity and/or activity for the insulin receptor and/or IGF1 or IGF2 receptor.

In some embodiments, the modified insulin or insulin analog has substantially reduced or ablated affinity and/or activity for the insulin receptor and/or IGF1 or IGF2 receptor. Attenuated response at the insulin receptor allows for the control of diabetes, obesity, metabolic disorders and the like while directing away from IGF 1 or IGF2 receptor avoids pro-cancer effects.

In an embodiment, the modified signaling agent is insulin-like growth factor-I or insulin-like growth factor-II (IGF-1 or IGF-2). In an embodiment, the modified signaling agent is IGF-1. In such an embodiment, the modified signaling agent has reduced affinity and/or activity for the insulin receptor and/or IGF1 receptor. In an embodiment, the modified signaling agent may bind to the IGF1 receptor and antagonize the activity of the receptor. In such an embodiment, the modified signaling agent has reduced affinity and/or activity for IGF1 receptor which allows for the activity of the receptor to be antagonized in an attenuated fashion. In some embodiments, the modified signaling agent has substantially reduced or ablated affinity and/or activity for the insulin receptor and/or IGF1 receptor. In some embodiments, the modified signaling agent has reduced affinity and/or activity for IGF2 receptor which allows for the activity of the receptor to be antagonized in an attenuated fashion. In an embodiment, the modified signaling agent has substantially reduced or ablated affinity and/or activity for the insulin receptor and accordingly does not interfere with insulin signaling. In various embodiments, this applies to cancer treatment. In various embodiments, the present agents may prevent IR isoform A from causing resistance to cancer treatments.

In an embodiment, the modified signaling agent is EPO. In various embodiments, the modified EPO agent has reduced affinity and/or activity for the EPO receptor (EPOR) receptor and/or the ephrin receptor (EphR) relative to wild type EPO or other EPO based agents described herein. In some embodiments, the modified EPO agent has substantially reduced or ablated affinity and/or activity for the EPO receptor (EPOR) receptor and/or the Eph receptor (EphR). Illustrative EPO receptors include, but are not limited to, an EPOR homodimer or an EPOR/CD131 heterodimer. Also included as an EPO receptor is beta-common receptor (BcR). Illustrative Eph receptors include, but are not limited to, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, and EPHB6. In some embodiments, the modified EPO protein comprises one or more mutations that cause the EPO protein to have reduced affinity for receptors that comprise one or more different EPO receptors or Eph receptors (e.g. heterodimer, heterotrimers, etc., including by way of non-limitation: EPOR-EPHB4, EPOR-BcR-EPOR). Also provided are the receptors of EP Patent Publication No. 2492355 the entire contents of which are hereby incorporated by reference, including by way of non-limitation, NEPORs.

In an embodiment, the human EPO has the amino acid sequence of SEQ ID NO:247 (first 27 amino acids are the signal peptide).

In an embodiment, the human EPO protein is the mature form of EPO (with the signal peptide being cleaved off) which is a glycoprotein of 166 amino acid residues having the sequence of SEQ ID NO:248.

The structure of the human EPO protein is predicted to comprise four-helix bundles including helices A, B, C, and D. In various embodiments, the modified EPO protein comprises one or more mutations located in four regions of the EPO protein which are important for bioactivity, i.e., amino acid residues 10-20, 44-51, 96-108, and 142-156. In some embodiments, the one or more mutations are located at residues 11-15, 44-51, 100-108, and 147-151. These residues are localized to helix A (Val11, Arg14, and Tyr15), helix C (Ser100, Arg103, Ser104, and Leu108), helix D (Asn147, Arg150, Glyl51, and Leu155), and the A/B connecting loop (residues 42-51). In some embodiments, the modified EPO protein comprises mutations in residues between amino acids 41-52 and amino acids 147, 150, 151, and 155. Without wishing to be bound by theory, it is believed that mutations of these residues have substantial effects on both receptor binding and in vitro biological activity. In some embodiments, the modified EPO protein comprises mutations at residues 11, 14, 15, 100, 103, 104, and 108. Without wishing to be bound by theory, it is believed that mutations of these residues have modest effects on receptor binding activity and much greater effects on in vitro biological activity. Illustrative substitutions include, but are not limited to, one or more of Val11Ser, Arg14Ala, Arg14Gln, Tyr15lle, Pro42Asn, Thr44lle, Lys45Asp, Val46Ala, Tyr51Phe, Ser100Glu, Ser100Thr, Arg103Ala, Ser104lle, Ser104Ala, Leu108Lys, Asn147Lys, Arg150Ala, Glyl51Ala, and Leu155Ala.

In some embodiments, the modified EPO protein comprises mutations that effect bioactivity and not binding, e.g. those listed in Eliot, et al. Mapping of the Active Site of Recombinant Human Erythropoietin Jan. 15, 1997; Blood: 89 (2), the entire contents of which are hereby incorporated by reference.

In some embodiments, the modified EPO protein comprises one or more mutations involving surface residues of the EPO protein which are involved in receptor contact. Without wishing to be bound by theory, it is believed that mutations of these surface residues are less likely to affect protein folding thereby retaining some biological activity. Illustrative surface residues that may be mutated include, but are not limited to, residues 147 and 150. In illustrative embodiments, the mutations are substitutions including, one or more of N147A, N147K, R150A and R150E.

In some embodiments, the modified EPO protein comprises one or more mutations at residues N59, E62, L67, and L70, and one or more mutations that affect disulfide bond formation. Without wishing to be bound by theory, it is believed that these mutations affect folding and/or are predicted be in buried positions and thus affects biological activity indirectly.

In an embodiment, the modified EPO protein comprises a K20E substitution which significantly reduces receptor binding. See Elliott, et al., (1997) Blood, 89:493-502, the entire contents of which are hereby incorporated by reference.

Additional EPO mutations that may be incorporated into the chimeric EPO protein of the invention are disclosed in, for example, Elliott, et al., (1997) Blood, 89:493-502, the entire contents of which are hereby incorporated by reference and Taylor et al., (2010) PEDS, 23(4): 251-260, the entire contents of which are hereby incorporated by reference.

In various embodiments, the signaling agent is a toxin or toxic enzyme. In some embodiments, the toxin or toxic enzyme is derived from plants and bacteria. Illustrative toxins or toxic enzymes include, but are not limited to, the diphtheria toxin, Pseudomonas toxin, anthrax toxin, ribosome-inactivating proteins (RIPs) such as ricin and saporin, modeccin, abrin, gelonin, and poke weed antiviral protein. Additional toxins include those disclosed in Mathew et al., (2009) Cancer Sci 100(8): 1359-65, the entire disclosures are hereby incorporated by reference. In such embodiments, the chimeric proteins or chimeric protein complexes such as Fc-based chimeric protein complex of the invention may be utilized to induce cell death in cell-type specific manner. In such embodiments, the toxin may be modified, e.g. mutated, to reduce affinity and/or activity of the toxin for an attenuated effect, as described with other signaling agents herein.

### Linkers and Functional Groups

In some embodiments, the present chimeric proteins or chimeric protein complexes, such as Fc-based chimeric protein complexes, optionally comprise one or more linkers. In some embodiments, the present chimeric proteins or chimeric protein complexes, such as Fc-based chimeric protein complexes, comprise a linker connecting the targeting moiety and the signaling agent (e.g., a modified IL-2). In some embodiments, the present chimeric proteins or chimeric protein complexes, such as Fc-based chimeric protein complexes, comprise a signaling agent (e.g., a modified IL-2) that comprises a linker. In some embodiments, the linker may be utilized to link various functional groups, residues, or moieties as described herein to the chimeric proteins or chimeric protein complexes, such as Fc-based chimeric protein complexes. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, vectors encoding the present chimeric proteins or chimeric protein complexes, such as Fc-based chimeric protein complexes, linked as a single nucleotide sequence to any of the linkers described herein are provided and may be used to prepare such chimeric proteins or chimeric protein complexes, such as Fc-based chimeric protein complexes. In embodiments, the substituents of the Fc-based chimeric protein complex are expressed as nucleotide sequences in a vector.

In some embodiments, the linker length allows for efficient binding of a targeting moiety and the signaling agent (e.g., a modified IL-2) to their receptors. For instance, in some embodiments, the linker length allows for efficient binding of one of the targeting moieties and the signaling agent to receptors on the same cell.

In some embodiments the linker length is at least equal to the minimum distance between the binding sites of one of the targeting moieties and the signaling agent to receptors on the same cell. In some embodiments the linker length is at least twice, or three times, or four times, or five times, or ten times, or twenty times, or 25 times, or 50 times, or one hundred times, or more the minimum distance between the binding sites of one of the targeting moieties and the signaling agent to receptors on the same cell.

As described herein, the linker length allows for efficient binding of one of the targeting moieties and the signaling agent to receptors on the same cell, the binding being sequential, e.g. targeting moiety/receptor binding preceding signaling agent/receptor binding.

In some embodiments, there are two linkers in a single chimera, each connecting the signaling agent to a targeting moiety. In various embodiments, the linkers have lengths that allow for the formation of a site that has a disease cell and an effector cell without steric hindrance that would prevent modulation of the either cell.

The invention contemplates the use of a variety of linker sequences. In various embodiments, the linker may be derived from naturally occurring multi-domain proteins or are empirical linkers as described, for example, in Chichili et al., (2013), Protein Sci. 22(2):153-167, Chen et al., (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present chimeric proteins or chimeric protein complexes such as Fc-based chimeric protein complex.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In some embodiments directed to chimeric proteins or chimeric protein complexes, such as Fc-based chimeric protein complexes, having two or more targeting moieties, a linker connects the two targeting moieties to each other, and this linker has a short length and a linker connects a targeting moiety and a signaling agent this linker is longer than the linker connecting the two targeting moieties. For example, the difference in amino acid length between the linker connecting the two targeting moieties and the linker connecting a targeting moiety and a signaling agent may be about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NO:249-SEQ ID NO:256, respectively). For example, in some embodiments, the linker is (Gly₃Ser)ₙ, where n is from about 1 to about 8, e.g. 1, 2, 3, 4, 5, 6, 7, or 8 (SEQ ID NO: 457-SEQ ID NO: 464, respectively). In an embodiment, the linker sequence is GGSGGSGGGGSGGGGS (SEQ ID NO:257). Additional illustrative linkers include, but are not limited to, linkers having the sequence LE, GGGGS (SEQ ID NO:249), (GGGGS)ₙ (n=1-4) (SEQ ID NO:249-SEQ ID NO:252), (Gly): (SEQ ID NO:258), (Gly)₆ (SEQ ID NO:259), (EAAAK)ₙ (n=1-3) (SEQ ID NO:260-SEQ ID NO:262), A(EAAAK)ₙA (n=2-5) (SEQ ID NO:263-SEQ ID NO:266), AEAAAKEAAAKA (SEQ ID NO:263), A(EAAAK)₄ALEA(EAAAK)₄A (SEQ ID NO:267), PAPAP (SEQ ID NO:268), KESGSVSSEQLAQFRSLD (SEQ ID NO:269), EGKSSGSGSESKST (SEQ ID NO:270), GSAGSAAGSGEF (SEQ ID NO:271), and (XP)n, with X designating any amino acid, e.g., Ala, Lys, or Glu. In various embodiments, the linker is GGS.

In some embodiments, the linker is one or more of GGGSE (SEQ ID NO: 272), GSESG (SEQ ID NO: 273), GSEGS (SEQ ID NO: 274), GEGGSGEGSSGEGSSSEGGGSEGGGSEGGGSEGGS (SEQ ID NO: 275), and a linker of randomly placed G, S, and E every 4 amino acid intervals.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CHI to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys (SEQ ID NO: 276), which when dimerized by disulfide bond formation, results in a cyclic octapeptide believed to act as a pivot, thus conferring flexibility. In various embodiments, the present linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker of the present invention comprises one or more glycosylation sites. In various embodiments, the linker is a hinge-CH2-CH3 domain of a human IgG4 antibody.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the present chimeric proteins or chimeric protein complexes such as Fc-based chimeric protein complex. In another example, the linker may function to target the chimeric proteins or chimeric protein complexes such as Fc-based chimeric protein complex to a particular cell type or location.

In various embodiments, the present chimeric proteins or chimeric protein complexes such as Fc-based chimeric protein complex may include one or more functional groups, residues, or moieties. In various embodiments, the one or more functional groups, residues, or moieties are attached or genetically fused to any of the signaling agents or targeting moieties described herein. In some embodiments, such functional groups, residues or moieties confer one or more desired properties or functionalities to the chimeric proteins or chimeric protein complexes such as Fc-based chimeric protein complex of the invention. Examples of such functional groups and of techniques for introducing them into the chimeric proteins or chimeric protein complexes such as Fc-based chimeric protein complex are known in the art, for example, see Remington's Pharmaceutical Sciences, 16th ed., Mack Publishing Co., Easton, Pa. (1980).

In various embodiments, each of the chimeric proteins or chimeric protein complexes such as Fc-based chimeric protein complex may by conjugated and/or fused with another agent to extend half-life or otherwise improve pharmacodynamic and pharmacokinetic properties. In some embodiments, the chimeric proteins or chimeric protein complexes such as Fc-based chimeric protein complex may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In various embodiments, each of the individual chimeric proteins or chimeric protein complexes such as Fc-based chimeric protein complex is fused to one or more of the agents described in BioDrugs (2015) 29:215-239, the entire contents of which are hereby incorporated by reference.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenecity of the chimeric proteins or chimeric protein complexes such as Fc-based chimeric protein complex. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the chimeric proteins or chimeric protein complexes such as Fc-based chimeric protein complex of the invention is modified so as to suitably introduce one or more cysteine residues for attachment of PEG, or an amino acid sequence comprising one or more cysteine residues for attachment of PEG may be fused to the amino- and/or carboxy-terminus of the chimeric proteins or chimeric protein complexes such as Fc-based chimeric protein complex, using techniques known in the art.

In some embodiments, the functional groups, residues, or moieties comprise N-linked or O-linked glycosylation. In some embodiments, the N-linked or O-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled VHHs and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label.

In some embodiments, the functional groups, residues, or moieties comprise a tag that is attached or genetically fused to the chimeric proteins or chimeric protein complexes such as Fc-based chimeric protein complex. In some embodiments, the chimeric proteins or chimeric protein complexes such as Fc-based chimeric protein complex may include a single tag or multiple tags. The tag for example is a peptide, sugar, or DNA molecule that does not inhibit or prevent binding of the chimeric proteins or chimeric protein complexes such as Fc-based chimeric protein complex to its target or any other antigen of interest such as tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the chimeric proteins or chimeric protein complexes such as Fc-based chimeric protein complex comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept)avidin binding pair. Such a functional group may be used to link the chimeric proteins or chimeric protein complexes such as Fc-based chimeric protein complex of the invention to another protein, polypeptide or chemical compound that is bound to the other half of the binding pair, i.e., through formation of the binding pair. For example, a chimeric proteins or chimeric protein complexes such as Fc-based chimeric protein complex of the invention may be conjugated to biotin, and linked to another protein, polypeptide, compound or carrier conjugated to avidin or streptavidin. For example, such a conjugated chimeric proteins or chimeric protein complexes such as Fc-based chimeric protein complex may be used as a reporter, for example, in a diagnostic system where a detectable signal-producing agent is conjugated to avidin or streptavidin. Such binding pairs may, for example, also be used to bind the chimeric proteins or chimeric protein complexes such as Fc-based chimeric protein complex to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the chimeric proteins or chimeric protein complexes such as Fc-based chimeric protein complex of the invention.

Production of Chimeric Proteins or Chimeric Protein Complexes, such as Fc-Based Chimeric Protein Complexes

Methods for producing the chimeric proteins or chimeric protein complexes, such as Fc-based chimeric protein complexes, of the invention are described herein. For example, DNA sequences encoding the chimeric proteins or chimeric protein complexes such as Fc-based chimeric protein complex of the invention (e.g., DNA sequences encoding the signaling agent (e.g., a modified IL-2) and the targeting moiety and the linker) can be chemically synthesized using methods known in the art. Synthetic DNA sequences can be ligated to other appropriate nucleotide sequences, including, e.g., expression control sequences, to produce gene expression constructs encoding the desired chimeric proteins or chimeric protein complexes, such as Fc-based chimeric protein complexes. Accordingly, in various embodiments, the present invention provides for isolated nucleic acids comprising a nucleotide sequence encoding the chimeric proteins or chimeric protein complexes. such as Fc-based chimeric protein complexes, of the invention.

Nucleic acids encoding the chimeric proteins or chimeric protein complexes, such as Fc-based chimeric protein complexes, of the invention can be incorporated (ligated) into expression vectors, which can be introduced into host cells through transfection, transformation, or transduction techniques. For example, nucleic acids encoding the chimeric proteins or chimeric protein complexes, such as Fc-based chimeric protein complexes, of the invention can be introduced into host cells by retroviral transduction. Illustrative host cells are E. coli cells, Chinese hamster ovary (CHO) cells, human embryonic kidney 293 (HEK 293) cells, Hela cells, baby hamster kidney (BHK) cells, monkey kidney cells (COS), human hepatocellular carcinoma cells (e.g., Hep G2), insect Sf9 cells, and myeloma cells. Transformed host cells can be grown under conditions that permit the host cells to express the genes that encode the chimeric proteins or chimeric protein complexes, such as Fc-based chimeric protein complexes, of the invention. Accordingly, in various embodiments, the present invention provides expression vectors comprising nucleic acids that encode the chimeric proteins or chimeric protein complexes, such as Fc-based chimeric protein complexes, of the invention. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The chimeric proteins or chimeric protein complexes, such as Fc-based chimeric protein complexes, of the invention can be produced by growing a host cell transfected with an expression vector encoding the chimeric proteins or chimeric protein complexes, such as Fc-based chimeric protein complexes, under conditions that permit expression of the protein. Following expression, the protein can be harvested and purified using techniques well known in the art, e.g., affinity tags such as glutathione-S-transferase (GST) and histidine tags or by chromatography.

Accordingly, in various embodiments, the present invention provides for a nucleic acid encoding a chimeric proteins or chimeric protein complexes, such as Fc-based chimeric protein complexes, of the present invention. In various embodiments, the present invention provides for a host cell comprising a nucleic acid encoding a chimeric proteins or chimeric protein complexes, such as Fc-based chimeric protein complexes, of the present invention.

In various embodiments, IL-2, its variant, or a chimeric proteins or chimeric protein complexes, such as Fc-based chimeric protein complexes, comprising the IL-2 or its variant may be expressed in vivo, for instance, in a patient. For example, in various embodiments, the IL-2, its variant, or a chimeric proteins or chimeric protein complexes such as Fc-based chimeric protein complexes comprising the IL-2 or its variant may administered in the form of nucleic acid which encodes for the IL-2 or its variant or chimeric proteins or chimeric protein complexes such as Fc-based chimeric protein complex comprising IL2 or its variant. In various embodiments, the nucleic acid is DNA or RNA. In some embodiments, the IL-2, its variant, or a chimeric proteins or chimeric protein complexes such as Fc-based chimeric protein complex comprising the IL-2 or its variant is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, Ψ, and 2′-O-methyl-U. In some embodiments, the present invention relates to administering a modified mRNA encoding one or more of the present chimeric proteins or chimeric protein complexes, such as Fc-based chimeric protein complexes. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

In various embodiments, the chimeric proteins or chimeric protein complexes, such as Fc-based chimeric protein complexes, comprises a targeting moiety that is a VHH. In various embodiments, the VHH is not limited to a specific biological source or to a specific method of preparation. For example, the VHH can generally be obtained: (1) by isolating the VHH domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized VHH domain; (4) by “camelization” of a naturally occurring VH domain from any animal species, such as from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known in the art; (7) by preparing a nucleic acid encoding a VHH using techniques for nucleic acid synthesis known in the art, followed by expression of the nucleic acid thus obtained; and/or (8) by any combination of one or more of the foregoing.

In an embodiment, the chimeric proteins or chimeric protein complexes such as Fc-based chimeric protein complex comprises a VHH that corresponds to the VHH domains of naturally occurring heavy chain antibodies directed against a target of interest. In some embodiments, such VHH sequences can generally be generated or obtained by suitably immunizing a species of Camelid with a molecule of based on the target of interest (e.g., XCR1, Clec9a, CD8, SIRP1α, FAP, etc.) (i.e., so as to raise an immune response and/or heavy chain antibodies directed against the target of interest), by obtaining a suitable biological sample from the Camelid (such as a blood sample, or any sample of B-cells), and by generating V_{H}H sequences directed against the target of interest, starting from the sample, using any suitable known techniques. In some embodiments, naturally occurring V_{H}H domains against the target of interest can be obtained from naive libraries of Camelid V_{H}H sequences, for example, by screening such a library using the target of interest or at least one part, fragment, antigenic determinant or epitope thereof using one or more screening techniques known in the art. Such libraries and techniques are, for example, described in WO 9937681, WO 0190190, WO 03025020 and WO 03035694, the entire contents of which are hereby incorporated by reference. In some embodiments, improved synthetic or semi-synthetic libraries derived from naive V_{H}H libraries may be used, such as V_{H}H libraries obtained from naive V_{H}H libraries by techniques such as random mutagenesis and/or CDR shuffling, as for example, described in WO 0043507, the entire contents of which are hereby incorporated by reference. In some embodiments, another technique for obtaining VHH sequences directed against a target of interest involves suitably immunizing a transgenic mammal that is capable of expressing heavy chain antibodies (i.e., so as to raise an immune response and/or heavy chain antibodies directed against the target of interest), obtaining a suitable biological sample from the transgenic mammal (such as a blood sample, or any sample of B-cells), and then generating VHH sequences directed against XCR1 starting from the sample, using any suitable known techniques. For example, for this purpose, the heavy chain antibody-expressing mice and the further methods and techniques described in WO 02085945 and in WO 04049794 (the entire contents of which are hereby incorporated by reference) can be used.

In an embodiment, the chimeric proteins or chimeric protein complexes such as Fc-based chimeric protein complex comprises a VHH that has been “humanized” i.e., by replacing one or more amino acid residues in the amino acid sequence of the naturally occurring V_{H}H sequence (and in particular in the framework sequences) by one or more of the amino acid residues that occur at the corresponding position(s) in a VH domain from a conventional 4-chain antibody from a human being. This can be performed using humanization techniques known in the art. In some embodiments, possible humanizing substitutions or combinations of humanizing substitutions may be determined by methods known in the art, for example, by a comparison between the sequence of a VHH and the sequence of a naturally occurring human VH domain. In some embodiments, the humanizing substitutions are chosen such that the resulting humanized VHHs still retain advantageous functional properties. Generally, as a result of humanization, the VHHs of the invention may become more “human-like,” while still retaining favorable properties such as a reduced immunogenicity, compared to the corresponding naturally occurring V_{H}H domains. In various embodiments, the humanized VHHs of the invention can be obtained in any suitable manner known in the art and thus are not strictly limited to polypeptides that have been obtained using a polypeptide that comprises a naturally occurring VHH domain as a starting material.

In an embodiment, the chimeric proteins or chimeric protein complexes such as Fc-based chimeric protein complex comprises a VHH that has been “camelized,” i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody of a camelid. In some embodiments, such “camelizing” substitutions are inserted at amino acid positions that form and/or are present at the VH-VL interface, and/or at the so-called Camelidae hallmark residues (see, for example, WO9404678, the entire contents of which are hereby incorporated by reference). In some embodiments, the VH sequence that is used as a starting material or starting point for generating or designing the camelized VHH is a VH sequence from a mammal, for example, the VH sequence of a human being, such as a VH3 sequence. In various embodiments, the camelized VHHs can be obtained in any suitable manner known in the art (i.e., as indicated under points (1)-(8) above) and thus are not strictly limited to polypeptides that have been obtained using a polypeptide that comprises a naturally occurring VH domain as a starting material.

In various embodiments, both “humanization” and “camelization” can be performed by providing a nucleotide sequence that encodes a naturally occurring V_{H}H domain or VH domain, respectively, and then changing, in a manner known in the art, one or more codons in the nucleotide sequence in such a way that the new nucleotide sequence encodes a “humanized” or “camelized” VHH, respectively. This nucleic acid can then be expressed in a manner known in the art, so as to provide the desired VHH of the invention. Alternatively, based on the amino acid sequence of a naturally occurring V_{H}H domain or VH domain, respectively, the amino acid sequence of the desired humanized or camelized VHH of the invention, respectively, can be designed and then synthesized de novo using techniques for peptide synthesis known in the art. Also, based on the amino acid sequence or nucleotide sequence of a naturally occurring V_{H}H domain or VH domain, respectively, a nucleotide sequence encoding the desired humanized or camelized VHH, respectively, can be designed and then synthesized de novo using techniques for nucleic acid synthesis known in the art, after which the nucleic acid thus obtained can be expressed in a manner 20) known in the art, so as to provide the desired VHH of the invention. Other suitable methods and techniques for obtaining the VHHs of the invention and/or nucleic acids encoding the same, starting from naturally occurring VH sequences or V_{H}H sequences, are known in the art, and may, for example, comprise combining one or more parts of one or more naturally occurring VH sequences (such as one or more FR sequences and/or CDR sequences), one or more parts of one or more naturally occurring V_{H}H sequences (such as one or more FR sequences or CDR sequences), and/or one or more synthetic or semi-synthetic sequences, in a suitable manner, so as to provide a VHH of the invention or a nucleotide sequence or nucleic acid encoding the same.

### Pharmaceutically Acceptable Salts and Excipients

The chimeric proteins or chimeric protein complexes, such as Fc-based chimeric protein complexes, described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl) amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Pharmaceutical Compositions and Formulations

In various embodiments, the present invention pertains to pharmaceutical compositions comprising the chimeric proteins or chimeric protein complexes, such as Fc-based chimeric protein complexes, described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, dessicated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the chimeric proteins or chimeric protein complexes such as Fc-based chimeric protein complex described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any chimeric proteins or chimeric protein complexes such as Fc-based chimeric protein complex described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598, 123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the chimeric proteins or chimeric protein complexes, such as Fc-based chimeric protein complexes, to be administered according to the present invention will vary according to the particular dosage form, and the mode of administration. Many factors that may modify the action of the chimeric proteins or chimeric protein complexes, such as Fc-based chimeric protein complexes. For example, body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of the chimeric proteins or chimeric protein complexes, such as Fc-based chimeric protein complexes, is in a range of about 0.01 μg/kg to about 100 mg/kg of body weight of the subject, about 0.01 μg/kg to about 10 mg/kg of body weight of the subject, or about 0.01 μg/kg to about 1 mg/kg of body weight of the subject for example, about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, or about 100 mg/kg body weight, inclusive of all values and ranges therebetween.

Individual doses of the chimeric proteins or chimeric protein complexes, such as Fc-based chimeric protein complexes, can be administered in unit dosage forms (e.g., tablets, capsules, or liquid formulations) containing, for example, from about 1 μg to about 100 mg, from about 1 μg to about 90 mg, from about 1 μg to about 80 mg, from about 1 μg to about 70 mg, from about 1 μg to about 60 mg, from about 1 μg to about 50 mg, from about 1 μg to about 40 mg, from about 1 μg to about 30 mg, from about 1 μg to about 20 mg, from about 1 μg to about 10 mg, from about 1 μg to about 5 mg, from about 1 μg to about 3 mg, from about 1 μg to about 1 mg per unit dosage form, or from about 1 μg to about 50 μg per unit dosage form. For example, a unit dosage form can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In one embodiment, the chimeric proteins or chimeric protein complexes, such as Fc-based chimeric protein complexes, is administered at an amount of from about 1 μg to about 100 mg daily, from about 1 μg to about 90 mg daily, from about 1 μg to about 80 mg daily, from about 1 μg to about 70 mg daily, from about 1 μg to about 60 mg daily, from about 1 μg to about 50 mg daily, from about 1 μg to about 40 mg daily, from about 1 μg to about 30 mg daily, from about 1 μg to about 20 mg daily, from about 01 μg to about 10 mg daily, from about 1 μg to about 5 mg daily, from about 1 μg to about 3 mg daily, or from about 1 μg to about 1 mg daily. In various embodiments, the chimeric proteins or chimeric protein complexes such as Fc-based chimeric protein complex is administered at a daily dose of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg,, about 21 μg, about 22 μg, about 23 μg, about 24 jug, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In accordance with certain embodiments of the invention, the pharmaceutical composition comprising the chimeric proteins or chimeric protein complexes, such as Fc-based chimeric protein complexes, may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year. In an embodiment, the pharmaceutical composition comprising the chimeric proteins or chimeric protein complexes, such as Fc-based chimeric protein complexes, is administered about three times a week.

In various embodiments, the present chimeric proteins or chimeric protein complexes, such as Fc-based chimeric protein complexes, may be administered for a prolonged period. For example, the chimeric proteins or chimeric protein complexes, such as Fc-based chimeric protein complexes, may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks. For example, the chimeric proteins or chimeric protein complexes, such as Fc-based chimeric protein complexes, may be administered for 12 weeks, 24 weeks, 36 weeks or 48 weeks. In some embodiments, the chimeric proteins or chimeric protein complexes, such as Fc-based chimeric protein complexes, are administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. n some embodiments, the chimeric proteins or chimeric protein complexes, such as Fc-based chimeric protein complexes, may be administered for at least about 1 year, at least about 2 years, at least about 3 years, at least about 4 years, or at least about 5 years.

### Combination Therapy and Additional Therapeutic Agents

In various embodiments, the pharmaceutical composition of the present invention is co-administered in conjunction with additional therapeutic agent(s). Co-administration can be simultaneous or sequential.

In one embodiment, the additional therapeutic agent and the chimeric proteins or chimeric protein complexes, such as Fc-based chimeric protein complexes, of the present invention are administered to a subject simultaneously. The term “simultaneously” as used herein, means that the additional therapeutic agent and the chimeric proteins or chimeric protein complexes, such as Fc-based chimeric protein complexes, are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the chimeric proteins or chimeric protein complexes, such as Fc-based chimeric protein complexes, can be by simultaneous administration of a single formulation (e.g., a formulation comprising the additional therapeutic agent and the chimeric proteins or chimeric protein complexes such as Fc-based chimeric protein complex) or of separate formulations (e.g., a first formulation including the additional therapeutic agent and a second formulation including the chimeric proteins or chimeric protein complexes such as Fc-based chimeric protein complex).

Co-administration does not require the therapeutic agents to be administered simultaneously, if the timing of their administration is such that the pharmacological activities of the additional therapeutic agent and the chimeric proteins or chimeric protein complexes such as Fc-based chimeric protein complex overlap in time, thereby exerting a combined therapeutic effect. For example, the additional therapeutic agent and the chimeric proteins or chimeric protein complexes such as Fc-based chimeric protein complex can be administered sequentially. The term “sequentially” as used herein means that the additional therapeutic agent and the chimeric proteins or chimeric protein complexes such as Fc-based chimeric protein complex are administered with a time separation of more than about 60 minutes. For example, the time between the sequential administration of the additional therapeutic agent and the chimeric proteins or chimeric protein complexes such as Fc-based chimeric protein complex can be more than about 60 minutes, more than about 2 hours, more than about 5 hours, more than about hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the chimeric proteins or chimeric protein complexes such as Fc-based chimeric protein complex being administered. Either the additional therapeutic agent or the chimeric proteins or chimeric protein complexes such as Fc-based chimeric protein complex cell may be administered first.

Co-administration also does not require the therapeutic agents to be administered to the subject by the same route of administration. Rather, each therapeutic agent can be administered by any appropriate route, for example, parenterally or non-parenterally.

In some embodiments, the chimeric proteins or chimeric protein complexes such as Fc-based chimeric protein complex described herein acts synergistically when co-administered with another therapeutic agent. In such embodiments, the chimeric proteins or chimeric protein complexes such as Fc-based chimeric protein complex and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of the present chimeric proteins or chimeric protein complexes such as Fc-based chimeric protein complex and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, inclusive of, without limitation, infectious disease applications, the present invention pertains to anti-infectives as additional therapeutic agents. In some embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicilin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In illustrative embodiments, the present invention pertains to the use of hepatitis therapeutics as additional therapeutic agents. In various embodiments, the hepatitis therapeutics include, but are not limited to, IFN-α such as INTRON A or pegylated IFN-α such as Pegasys or PEG-INTRON, ribavirin, boceprevir, simeprevir, sofosbuvir, simeprevir, daclatasvir, ledipasvir/sofosbuvir (Harvoni), ombitasvir/paritaprevir/ritonavir (Technivie), ombitasvir/paritaprevir/ritonavir/dasabuvir (Viekira Pak), lamivudine, adefovir, entecavir, telbivudine, entecavir, tenofovir, velpatasvir, elbasvir, grazoprevir, dasabuvir, and any combinations thereof. In an embodiment, the additional therapeutic agent is IFN-α (e.g., INTRON A) or pegylated IFN-α (e.g., Pegasys or PEG-INTRON). In another embodiment, the additional therapeutic agent is ribavirin.

In some embodiments, the present invention relates to combination therapies using the chimeric proteins or chimeric protein complexes such as Fc-based chimeric protein complex and an immunosuppressive agent. In some embodiments, the present invention relates to administration of the Clec9A binding agent to a patient undergoing treatment with an immunosuppressive agent.

In an embodiment, the immunosuppressive agent is TNF. In illustrative embodiments, the chimeric proteins or chimeric protein complexes such as Fc-based chimeric protein complex act synergistically when co-administered with TNF. In an illustrative embodiment, the chimeric proteins or chimeric protein complexes such as Fc-based chimeric protein complex acts synergistically when co-administered with TNF for use in treating tumor or cancer. For example, co-administration of the chimeric proteins or chimeric protein complexes such as Fc-based chimeric protein complex of the present invention and TNF may act synergistically to reduce or eliminate the tumor or cancer, or slow the growth and/or progression and/or metastasis of the tumor or cancer. In some embodiments, the combination of the chimeric proteins or chimeric protein complexes such as Fc-based chimeric protein complex and TNF may exhibit improved safety profiles when compared to the agents used alone in the context of monotherapy. In some embodiments, the chimeric proteins or chimeric protein complexes such as Fc-based chimeric protein complex and TNF may be administered at doses that are lower than the doses employed when the agents are used in the context of monotherapy.

In some embodiments, inclusive, without limitation, of autoimmune applications, the additional therapeutic agent is an immunosuppressive agent that is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In some embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin). Additional anti-inflammatory agents are described, for example, in U.S. Pat. No. 4,537,776, the entire contents of which is incorporated by reference herein.

In some embodiments, the present invention pertains to various agents used for treating obesity as additional therapeutic agents. Illustrative agents used for treating obesity include, but are not limited to, orlistat (e.g. ALL1, XENICAL), loracaserin (e.g. BELVIQ), phentermine-topiramate (e.g. QSYMIA), sibutramme (e.g. REDUCTIL or MERJDIA), rimonabant (ACOMPLLA), exenatide (e.g. BYETTA), pramlintide (e.g. SYMLIN) phentermine, benzphetamine, diethylpropion, phendimetrazme, bupropion, and metformin. Agents that interfere with the body's ability to absorb specific nutrients in food are among the additional agents, e.g. orlistat (e.g. ALU, XENICAL), glucomannan, and guar gum. Agents that suppress apetite are also among the additional agents, e.g. catecholamines and their derivatives (such as phenteimine and other amphetamine-based drugs), various antidepressants and mood stabilizers (e.g. bupropion and topiramate), anorectics (e.g. dexedrine, digoxin). Agents that increase the body's metabolism are also among the additional agents.

In some embodiments, additional therapeutic agents may be selected from among appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, peroxisome proliferator-activated receptor (PPAR) modulators; dipeptidyl peptidase 4 (DPP-4) antagonists, agents for treating cardiovascular disease, agents for treating elevated triglyceride levels, agents for treating low HDL, agents for treating hypercholesterolemia, and agents for treating hypertension. Some agents for cardiovascular disease include statins (e.g. lovastatin, atorvastatin, fluvastatin, rosuvastatin, simvastatin and pravastatin) and omega-3 agents (e.g. LOVAZA, EPANQVA, VASCEPA, esterified omega-3's in general, fish oils, krill oils, algal oils). In some embodiments, additional agents may be selected from among amphetamines, benzodiazepines, suifonyl ureas, meglitinides, thiazolidinediones, biguanides, beta-blockers, XCE inhibitors, diuretics, nitrates, calcium channel blockers, phenlermine, sibutramine, iorcaserin, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, rcpaglini.de, nateglinide, glimepiride, metformin, pioglitazone, rosiglilazone, and sitagliptin.

In some embodiments, the present invention pertains to an agent used for treating diabetes as additional therapeutic agents. Illustrative anti-diabetic agents include, but are not limited to, sulfonylurea (e.g., DYMELOR (acetohexamide), DIABINESE (chlorpropamide), ORINASE (tolbutamide), and TOLINASE (tolazamide), GLUCOTROL (glipizide), GLUCOTROL XL (extended release), DIABETA (glyburide), MICRONASE (glyburide), GLYNASE PRESTAB (glyburide), and AMARYL (glimepiride)); a Biguanide (e.g. metformin (GLUCOPHAGE, GLUCOPHAGE XR, RIOMET, FORTAMET, and GLUMETZA)); a thiazolidinedione (e.g. ACTOS (pioglitazone) and AVANDIA (rosiglitazone); an alpha-glucosidase inhibitor (e.g., PRECOSE (acarbose) and GLYSET (miglitol); a Meglitinide (e.g., PRANDIN (repaglinide) and STARLIX (nateglinide)); a Dipeptidyl peptidase IV (DPP-IV) inhibitor (e.g., JANUVIA (sitagliptin), NESINA (alogliptin), ONGLYZA (saxagliptin), and TRADJENTA (linagliptin); Sodium-glucose co-transporter 2 (SGLT2) inhibitor (e.g. INVOKANA (canaglifozin)); and a combination pill (e.g. GLUCOVANCE, which combines glyburide (a sulfonylurea) and metformin, METAGLIP, which combines glipizide (a sulfonylurea) and metformin, and AVANDAMET, which uses both metformin and rosiglitazone (AVANDIA) in one pill, KAZANO (alogliptin and metformin), OSENI (alogliptin plus pioglitazone), METFORMIN oral, ACTOS oral, BYETTA subcutaneous, JANUVIA oral, WELCHOL oral, JANUMET oral, glipizide oral, glimepiride oral, GLUCOPHAGE oral, LANTUS subcutaneous, glyburide oral, ONGLYZA oral, AMARYI oral, LANTUS SOLOSTAR subcutaneous, BYDUREON subcutaneous, LEVEMIR FLEXPEN subcutaneous, ACTOPLUS MET oral, GLUMETZA oral, TRADJENTA oral, bromocriptine oral, KOMBIGLYZE XR oral, INVOKANA oral, PRANDIN oral, LEVEMIR subcutaneous, PARLODEL oral, pioglitazone oral, NOVOLOG subcutaneous, NOVOLOG FLEXPEN subcutaneous, VICTOZA 2-PAK subcutaneous, HUMALOG subcutaneous, STARLIX oral, FORTAMET oral, GLUCOVANCE oral, GLUCOPHAGE XR oral, NOVOLOG Mix 70-30 FLEXPEN subcutaneous, GLYBURIDE-METFORMIN oral, acarbose oral, SYMLINPEN 60 subcutaneous, GLUCOTROI XL oral, NOVOLIN R inj, GLUCOTROL oral, DUETACT oral, sitagliptin oral, SYMLINPEN 120 subcutaneous, HUMALOG KWIKPEN subcutaneous, JANUMET XR oral, GLIPIZIDE-METFORMIN oral, CYCLOSET oral, HUMALOG MIX 75-25 subcutaneous, nateglinide oral, HUMALOG Mix 75-25 KWIKPEN subcutaneous, HUMULIN 70/30 subcutaneous, PRECOSE oral, APIDRA subcutaneous, Humulin R inj, Jentadueto oral, Victoza 3-Pak subcutaneous, Novolin 70/30 subcutaneous, NOVOLIN N subcutaneous, insulin detemir subcutaneous, glyburide micronized oral, GLYNASE oral, HUMULIN N subcutaneous, insulin glargine subcutaneous, RIOMET oral, pioglitazone-metformin oral, APIDRA SOLOSTAR subcutaneous, insulin lispro subcutaneous, GLYSET oral, HUMULIN 70/30 Pen subcutaneous, colesevelam oral, sitagliptin-metformin oral, DIABETA oral, insulin regular human inj, HUMULIN N Pen subcutaneous, exenatide subcutaneous, HUMALOG Mix 50-50 KWIKPEN subcutaneous, liraglutide subcutaneous, KAZANO oral, repaglinide oral, chlorpropamide oral, insulin aspart subcutaneous, NOVOLOG Mix 70-30 subcutaneous, HUMALOG Mix 50-50 subcutaneous, saxagliptin oral, ACTOPLUS Met XR oral, miglitol oral, NPH insulin human recomb subcutaneous, insulin NPH and regular human subcutaneous, tolazamide oral, mifepristone oral, insulin aspart protam-insulin aspart subcutaneous, repaglinide-metformin oral, saxagliptin-metformin oral, linagliptin-metformin oral, NESINA oral, OSENI oral, tolbutamide oral, insulin lispro protamine and lispro subcutaneous, pramlintide subcutaneous, insulin glulisine subcutaneous, pioglitazone-glimepiride oral, PRANDIMET oral, NOVOLOG PenFill subcutaneous, linagliptin oral, exenatide microspheres subcutaneous, KORLYM oral, alogliptin oral, alogliptin-pioglitazone oral, alogliptin-metformin oral, canagliflozin oral, Lispro (HUMALOG); Aspart (NOVOLOG); Glulisine (APIDRA); Regular (NOVOLIN R or HUMULIN R); NPH (NOVOLIN N or HUMULIN N); Glargine (LANTUS); Detemir (LEVEMIR); HUMULIN or NOVOLIN 70/30; and NOVOLOG Mix 70/30 HUMALOG Mix 75/25 or 50/50.

In some embodiments, the chimeric proteins or chimeric protein complexes such as Fc-based chimeric protein complex of the present invention act synergistically when used in combination with Chimeric Antigen Receptor (CAR) T-cell therapy. In an illustrative embodiment, the chimeric proteins or chimeric protein complexes such as Fc-based chimeric protein complex acts synergistically when used in combination with CAR T-cell therapy in treating tumor or cancer. In an embodiment, the chimeric proteins or chimeric protein complexes such as Fc-based chimeric protein complex agent acts synergistically when used in combination with CAR T-cell therapy in treating blood-based tumors. In an embodiment, the chimeric proteins or chimeric protein complexes such as Fc-based chimeric protein complex acts synergistically when used in combination with CAR T-cell therapy in treating solid tumors. For example, use of the chimeric proteins or chimeric protein complexes such as Fc-based chimeric protein complex and CAR T-cells may act synergistically to reduce or eliminate the tumor or cancer, or slow the growth and/or progression and/or metastasis of the tumor or cancer. In various embodiments, the chimeric proteins or chimeric protein complexes such as Fc-based chimeric protein complex of the invention induces CAR T-cell division. In various embodiments, the chimeric proteins or chimeric protein complexes such as Fc-based chimeric protein complex of the invention induces CAR T-cell proliferation. In various embodiments, the chimeric proteins or chimeric protein complexes such as Fc-based chimeric protein complex of the invention prevents anergy of the CAR T cells.

In various embodiments, the CAR T-cell therapy comprises CAR T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, 20) Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp 100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Illustrative CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In some embodiments, the chimeric proteins or chimeric protein complexes such as Fc-based chimeric protein complex of the present invention is used in a method of treating multiple sclerosis (MS) in combination with one or more MS therapeutics including, but not limited to, 3-interferons, glatiramer acetate, T-interferon, IFN-β-2 (U.S. Patent Publication No. 2002/0025304), spirogermaniums (e.g., N-(3-dimethylaminopropyl)-2-aza-8,8-dimethyl-8-germanspiro [4:5] decane, N-(3-dimethylaminopropyl)-2-aza-8,8-diethyl-8-germaspiro [4:5] decane, N-(3-dimethylaminopropyl)-2-aza-8,8-dipropyl-8-germaspiro [4:5] decane, and N-(3-dimethylaminopropyl)-2-aza-8, 8-dibutyl-8-germaspiro [4:5] decane), vitamin D analogs (e.g., 1,25 (OH) 2D3, (see, e.g., U.S. Pat. No. 5,716,946)), prostaglandins (e.g., latanoprost, brimonidine, PGE1, PGE2 and PGE3, see, e.g., U.S. Patent Publication No. 2002/0004525), tetracycline and derivatives (e.g., minocycline and doxycycline, see, e.g., U.S. Patent Publication No. 20020022608), a VLA-4 binding antibody (see, e.g., U.S. Patent Publication No. 2009/0202527), adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, fumarate, anti-CD20 antibody (e.g., rituximab), and tizanidine hydrochloride.

In some embodiments, the chimeric proteins or chimeric protein complexes such as Fc-based chimeric protein complex is used in combination with one or more therapeutic agents that treat one or more symptoms or side effects of MS. Such agents include, but are not limited to, amantadine, baclofen, papaverine, meclizine, hydroxyzine, sulfamethoxazole, ciprofloxacin, docusate, pemoline, dantrolene, desmopressin, dexamethasone, tolterodine, phenyloin, oxybutynin, bisacodyl, venlafaxine, amitriptyline, methenamine, clonazepam, isoniazid, vardenafil, nitrofurantoin, psyllium hydrophilic mucilloid, alprostadil, gabapentin, nortriptyline, paroxetine, propantheline bromide, modafinil, fluoxetine, phenazopyridine, methylprednisolone, carbamazepine, imipramine, diazepam, sildenafil, bupropion, and sertraline.

In some embodiments, the chimeric proteins or chimeric protein complexes such as Fc-based chimeric protein complex is used in a method of treating multiple sclerosis in combination with one or more of the disease modifying therapies (DMTs) described herein (e.g. the agents of Table 6). In some embodiments, the present invention provides an improved therapeutic effect as compared to use of one or more of the DMTs described herein (e.g. the agents listed in Table 6 below) without the one or more disclosed binding agent. In an embodiment, the combination of the chimeric proteins or chimeric protein complexes such as Fc-based chimeric protein complex and the one or more DMTs produces synergistic therapeutic effects.

Illustrative disease modifying therapies include, but are not limited to:

TABLE 6

| Generic Name | Branded Name/Company | Frequency/Route of Delivery/Usual Dose |
|---|---|---|
| teriflunomide | AUBAGIO (GENZYME) | Every day; pill taken orally; 7 mg or 14 mg. |
| interferon beta-1a | AVONEX (BIOGEN IDEC) | Once a week; intramuscular (into the muscle) |
| | | injection; 30 mcg |
| interferon beta-1b | BETASERON (BAYER | Every other day; subcutaneous (under the skin) |
| | HEALTHCARE | injection; 250 mcg. |
| | PHARMACEUTICALS, INC.) | |
| glatiramer acetate | COPAXONE (TEVA | Every day; subcutaneous (under the skin) |
| | NEUROSCIENCE) | injection; 20 mg (20,000 mcg) OR Three times a |
| | | week; subcutaneous (under the skin) injection; 40 |
| | | mg (40,000 mcg) |
| interferon beta-1b | EXTAVIA (NOVARTIS | Every other day; subcutaneous (under the skin) |
| | PHARMACEUTICALS CORP.) | injection; 250 mcg. |
| fingolimod | GILENYA (NOVARTIS | Every day; capsule taken orally; 0.5 mg. |
| | PHARMACEUTICALS CORP.) | |
| Alemtuzumab (anti-CD52 | LEMTRADA (GENZYME) | Intravenous infusion on five consecutive days, |
| monoclonal antibody) | | followed by intravenous infusion on three |
| | | consecutive days one year later (12 mg) |
| mitoxantrone | NOVANTRONE (EMD | Four times a year by IV infusion in a medical |
| | SERONO) | facility. Lifetime cumulative dose limit of |
| | | approximately 8-12 doses over 2-3 years (140 |
| | | mg/m2). |
| pegylated interferon beta-1a | PLEGRIDY (BIOGEN IDEC) | Every 14 days; subcutaneous (under the skin) |
| | | injection; 125 mcg |
| interferon beta-1a | REBIF (EMD SERONO, INC.) | Three times a week; subcutaneous (under the |
| | | skin) injection; 44 mcg |
| dimethyl fumarate (BG-12) | TECFIDERA (BIOGEN IDEC) | Twice a day; capsule taken orally; 120 mg for one |
| | | week and 240 mg therafter |
| Natalizumab (humanized | TYSABRI (BIOGEN IDEC) | Every four weeks by IV infusion in a registered |
| monoclonal antibody VLA-4 | | infusion facility; 300 mg |
| antagonist) | | |

DMTs in Development

| | | |
|---|---|---|
| Amiloride (targets Acid- | PAR PHARMACEUTICAL, | Oral |
| sensing ion channel-1 | PERRIGO COMPANY, | |
| Epithelial sodium channel | SIGMAPHARM | |
| Na+/H+ exchanger) | LABORATORIES | |
| ATX-MS-1467 (targets Major | APITOPE/MERCK SERONO | Intradermal Subcutaneous |
| histocompatibility complex | | |
| class II T cell responses to | | |
| myelin basic protein) | | |
| BAF312 (targets | NOVARTIS PHARMA | Oral |
| Sphingosine 1-phosphate | | |
| (S1P) receptor subtypes | | |
| S1P1 and S1P5 B cell | | |
| distrubution T cell | | |
| distribution) | | |
| BGC20-0134 (targets | BTG PLC | Oral |
| Proinflammatory and anti- | | |
| inflammatory cytokines) | | |
| BIIB033 (targets LINGO-1 | BIOGEN | Intravenous infusion used in Phase I and Phase II |
| (“leucine-rich repeat and | | trials Subcutaneous injection used in Phase I trial |
| immunoglobulin-like domain- | | |
| containing, Nogo receptor- | | |
| interacting protein”)) | | |
| Cladribine (targets CD4+ T | MERCK SERONO | Oral |
| cells DNA synthesis and | | |
| repair E-selectin Intracellular | | |
| adhesion molecule-1 Pro- | | |
| inflammatory cytokines | | |
| interleukin 2 and interleukin | | |
| 2R Pro-inflammatory | | |
| cytokines interleukin 8 and | | |
| RANTES Cytokine secretion | | |
| Monocyte and lymphocyte | | |
| migration) | | |
| Cyclophosphamide (targets | BAXTER HEALTHCARE | Oral, monthly intravenous pulses |
| T cells, particularly CD4+ | CORPORATION | |
| helper T cells B cells) | | |
| Daclizumab (humanized | BIOGEN IDEC/ABBVIE | Projected to be IM injection once monthly |
| monoclonal antibody | BIOTHERAPEUTICS | |
| targeting CD25 Immune | | |
| modulator of T cells) | | |
| Dalfampridine (targets | ACORDA THERAPEUTICS/ | One tablet every 12 hours (extended release), 10 |
| Voltage-gated potassium | BIOGEN IDEC | mg twice a day |
| channels | | |
| Degenerin/epithelial sodium | | |
| channels L-type calcium | | |
| channels that contain | | |
| subunit Cavbeta3) | | |
| Dronabinol (targets | ABBVIE INC. | Oral |
| Cannabinoid receptor CB1 | | |
| Cannabinoid receptor CB2) | | |
| Firategrast (targets | GLAXOSMITHKLINE | Oral |
| Alpha4beta1 integrin) | | |
| GNbAC1MSRV-Env (targets | GENEURO SA/SERVIER | Intravenous infusion |
| envelope protein of the MS- | | |
| associated retrovirus) | | |
| Idebenone (targets Reactive | SANTHERA | Oral Dose in clinical trial for PPMS is 2250 mg per |
| oxygen species) | PHARMACEUTICALS | day (750 mg dose, 3 times per day) |
| Imilecleucel-T (targets | OPEXA THERAPEUTICS/ | Subcutaneous Given 5 times per year, according |
| Myelin-specific, autoreactive | MERCK SERONO | to information from the manufacturer |
| T cells) | | |
| Laquinimod | TEVA | Projected to be 0.6 mg or 1.2 mg oral tablet taken |
| | | daily |
| Masitinib (targets KIT (a | AB SCIENCE | Oral |
| stem cell factor, also called | | |
| c-KIT) receptor as well as | | |
| select other tyrosine kinases | | |
| Mast cells) | | |
| MEDI-551 (targets CD19, a | MEDIMMUNE | Intravenous Subcutaneous |
| B cell-specific antigen that is | | |
| part of the B cell receptor | | |
| complex and that functions | | |
| in determining the threshold | | |
| for B cell activation B cells | | |
| Plasmablasts, B cells that | | |
| express CD19 (but not | | |
| CD20) and that secrete large | | |
| quantities of antibodies; | | |
| depletion of plasmablasts | | |
| may be useful in | | |
| autoimmune diseases | | |
| involving pathogenic | | |
| autoantibodies) | | |
| Minocycline (targets T cells | VARIOUS | Oral Available as pellet-filled capsules and an oral |
| Microglia Leukocyte | | suspension |
| migration Matrix | | |
| metalloproteinases) | | |
| MIS416 (targets Innate | INNATE | Intravenous |
| immune system Pathogen- | IMMUNOTHERAPEUTICS | |
| associated molecular pattern | | |
| recognition receptors of the | | |
| innate immune system | | |
| Myeloid cells of the innate | | |
| immune system, which might | | |
| be able to remodel the | | |
| deregulated immune system | | |
| activity that occurs in SPMS) | | |
| Mycophenolate mofetil | MANUFACTURED BY | Oral |
| (targets Purine synthesis) | GENENTECH | |
| Naltrexone (targets Opioid | VARIOUS | Given at low doses (3 to 4.5 mg per day) in oral |
| receptors Toll-like receptor | | form as“Low-dose naltrexone” (or “LDN”) |
| 4) | | |
| Ocrelizumab and | ROCHE/GSK | Projected to be IV infusion |
| Ofatumumab (humanized | | |
| monoclonal antibodies | | |
| targeting CD20 B cell | | |
| suppression | | |
| ONO-4641 (targets | ONO PHARMACEUTICAL CO. | Oral |
| Sphingosine 1-phosphate | | |
| receptor) | | |
| Phenytoin (targets Sodium | PFIZER | Intravenous Intramuscular (less favored option) |
| channels) | | Oral |
| Ponesimod | ACTELION | To be determined |
| Raltegravir (targets | MERCK | Oral 400 mg tablet twice daily, according to |
| Retroviral integrase | | information from the manufacturer |
| Herpesvirus DNA packaging | | |
| terminase) | | |
| RHB-104 | REDHILL BIOPHARMA | 95 mg clarithromycin, 45 mg rifabutin, and 10 mg |
| | LIMITED | clofazimine |
| Riluzole (targets | COVIS PHARMA/SANOFI | Oral |
| Glutamatergic | | |
| neurotransmission | | |
| Glutamate uptake and | | |
| release Voltage-gated | | |
| sodium channels Protein | | |
| kinase C) | | |

In some embodiments, the present invention relates to combination therapy with a blood transfusion. For instance, the present compositions may supplement a blood transfusion. In some embodiments, the present invention relates to combination therapy with iron supplements.

In some embodiments, the present invention relates to combination therapy with one or more EPO-based agents. For example, the present compositions may be used as an adjuvant to other EPO-based agents. In some embodiments, the present compositions are used as a maintenance therapy to other EPO-based agents. Other EPO-based agents include the following: epoetin alfa, including without limitation, DARBEPOETIN (ARANESP), EPOCEPT (LUPIN PHARMA), NANOKINE (NANOGEN PHARMACEUTICAL), EPOFIT (INTAS PHARMA), EPOGEN (AMGEN), EPOGIN, EPREX, (JANSSEN-CILAG), BINOCRIT7 (SANDOZ), PROCRIT; epoetin beta, including without limitation, NEORECORMON (HOFFMANN-LA ROCHE), RECORMON, Methoxy polyethylene glycol-epoetin beta (MIRCERA, ROCHE); epoetin delta, including without limitation, DYNEPO (erythropoiesis stimulating protein, SHIRE PLC); epoetin omega, including without limitation, EPOMAX; epoetin zeta, including without limitation, SILAPO (STADA) and RETACRIT (HOSPIRA) and other EPOs, including without limitation, EPOCEPT (LUPIN PHARMACEUTICALS), EPOTRUST (PANACEA BIOTEC LTD), ERYPRO SAFE (BIOCON LTD.), REPOITIN (SERUM INSTITUTE OF INDIA LIMITED), VINTOR (EMCURE PHARMACEUTICALS), EPOFIT (INTAS PHARMA), ERYKINE (INTAS BIOPHARMACEUTICA), WEPOX (WOCKHARDT BIOTECH), ESPOGEN (LG LIFE SCIENCES), RELIPOIETIN (RELIANCE LIFE SCIENCES), SHANPOIETIN (SHANTHA BIOTECHNICS LTD), ZYROP (CADILA HEALTHCARE LTD.), EPIAO (RHUEPO) (SHENYANG SUNSHINE PHARMACEUTICAL CO. LTD), CINNAPOIETIN (CINNAGEN).

In some embodiments, the present invention relates to combination therapy with one or more immune-modulating agents, for example, without limitation, agents that modulate immune checkpoint. In various embodiments, the immune-modulating agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL3280A (ROCHE). In some embodiments, the immune-modulating agent targets one or more of CD137 or CD137L. In various embodiments, the immune-modulating agent is an antibody specific for one or more of CD137 or CD137L. For instance, in some embodiments, the immune-modulating agent is an antibody such as, by way of non-limitation, urelumab (also known as BMS-663513 and anti-4-1BB antibody). In some embodiments, the present chimeric proteins or chimeric protein complexes such as Fc-based chimeric protein complex is combined with urelumab (optionally with one or more of nivolumab, lirilumab, and urelumab) for the treatment of solid tumors and/or B-cell non-Hodgkins lymphoma and/or head and neck cancer and/or multiple myeloma. In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the present chimeric proteins or chimeric protein complexes such as Fc-based chimeric protein complex is combined with ipilimumab (optionally with bavituximab) for the treatment of one or more of melanoma, prostate cancer, and lung cancer. In various embodiments, the immune-modulating agent targets CD20. In various embodiments, the immune-modulating agent is an antibody specific CD20. For instance, in some embodiments, the immune-modulating agent is an antibody such as, by way of non-limitation, Ofatumumab (GENMAB), obinutuzumab (GAZYVA), AME-133v (APPLIED MOLECULAR EVOLUTION), Ocrelizumab (GENENTECH), TRU-015 (TRUBION/EMERGENT), veltuzumab (IMMU-106).

In some embodiments, the present invention relates to combination therapy with one or more chimeric agents described in WO 2013/10779, WO 2015/007536, WO 2015/007520, WO 2015/007542, and WO 2015/007903, the entire contents of which are hereby incorporated by reference in their entireties.

In some embodiments, the chimeric proteins or chimeric protein complexes such as Fc-based chimeric protein complex described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

In still other embodiments, the chimeric proteins or chimeric protein complexes such as Fc-based chimeric protein complex described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The chimeric proteins or chimeric protein complexes such as Fc-based chimeric protein complex described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Illustrative cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the chimeric proteins or chimeric protein complexes such as Fc-based chimeric protein complex, with or without the use of a complex-forming agent. Such radionuclides include beta-emitters such as Phosphorus-32, Scandium-47, Copper-67, Gallium-67, Yttrium-88, Yttrium-90, Iodine-125, Iodine-131, Samarium-153, Lutetium-177, Rhenium-186 or Rhenium-188, and alpha-emitters such as Astatine-211, Lead-212, Bismuth-212, Bismuth-213 or Actinium-225.

Illustrative detectable moieties further include, but are not limited to, horseradish peroxidase, acetylcholinesterase, alkaline phosphatase, beta-galactosidase and luciferase. Further illustrative fluorescent materials include, but are not limited to, rhodamine, fluorescein, fluorescein isothiocyanate, umbelliferone, dichlorotriazinylamine, phycoerythrin and dansyl chloride. Further illustrative chemiluminescent moieties include, but are not limited to, luminol. Further illustrative bioluminescent materials include, but are not limited to, luciferin and aequorin. Further illustrative radioactive materials include, but are not limited to, Iodine-125, Carbon-14, Sulfur-35, Tritium and Phosphorus-32.

### Methods of Treatment

Methods and compositions described herein have application to treating various diseases and disorders, including, but not limited to cancer, infections, immune disorders, anemia, autoimmune diseases, cardiovascular diseases, wound healing, ischemia-related diseases, neurodegenerative diseases, metabolic diseases and many other diseases and disorders.

Further, any of the present agents may be for use in the treating, or the manufacture of a medicament for treating, various diseases and disorders, including, but not limited to cancer, infections, immune disorders, inflammatory diseases or conditions, and autoimmune diseases.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, cancer, heart failure, autoimmune disease, sickle cell disease, thalassemia, blood loss, transfusion reaction, diabetes, vitamin B12 deficiency, collagen vascular disease, Shwachman syndrome, thrombocytopenia purpura, Celiac disease, endocrine deficiency state such as hypothyroidism or Addison's disease, autoimmune disease such as Crohn's Disease, systemic lupus erythematosis, rheumatoid arthritis or juvenile rheumatoid arthritis, ulcerative colitis immune disorders such as eosinophilic fasciitis, hypoimmunoglobulinemia, or thymoma/thymic carcinoma, graft versus host disease, preleukemia, Nonhematologic syndrome (e.g. Down's, Dubowwitz, Seckel), Felty syndrome, hemolytic uremic syndrome, myelodysplasic syndrome, nocturnal paroxysmal hemoglobinuria, osteomyelofibrosis, pancytopenia, pure red-cell aplasia, Schoenlein-Henoch purpura, malaria, protein starvation, menorrhagia, systemic sclerosis, liver cirrhosis, hypometabolic states, and congestive heart failure.

In some embodiments, the present invention is related to a method for treating cancer, comprising administering an effective amount of i) the chimeric protein, the chimeric protein complex and/or the Fc-based chimeric protein complex to a patient in need thereof; ii) a recombinant nucleic acid encoding the chimeric protein, the chimeric protein complex and/or the Fc-based chimeric protein complex to a patient in need thereof; or iii) a host cell comprising the recombinant nucleic acid encoding the chimeric protein, the chimeric protein complex and/or the Fc-based chimeric protein complex to a patient in need thereof.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, mycobacterial infections, cancer, scleroderma, hepatitis, hepatitis C, septic shock, and rheumatoid arthritis.

In some embodiments, the present invention relates to the treatment of, or a patient having cancer. As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In an embodiment, the present invention relates to the treatment of leukemia including hairy cell leukemia. In another embodiment, the present invention relates to the treatment of melanoma including malignant melanoma. In a further embodiment, the present invention relates to the treatment of Kaposi's sarcoma including AIDS-related Kaposi's sarcoma.

In some embodiments, the present invention relates to the treatment of, or a patient having a microbial infection and/or chronic infection. Illustrative infections include, but are not limited to, Chagas disease, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In some embodiments, the present invention relates to the treatment of hepatitis. Illustrative hepatitis that may be treated include, but is not limited to, hepatitis A, hepatitis B, hepatitis C, hepatitis D, hepatitis E, autoimmune hepatitis, alcoholic hepatitis, acute hepatitis, and chronic hepatitis.

In an illustrative embodiment, the present invention relates to the treatment of chronic hepatitis C. In an embodiment, the chimeric proteins or chimeric protein complexes such as Fc-based chimeric protein complex of the invention may be utilized to treat a patient infected with any one of the hepatitis C genotypes, including genotype 1 (e.g., 1a, 1b), genotype 2 (e.g. 2a, 2b, 2c and 2d), genotype 3 (e.g., 3a, 3b, 3c, 3d, 3e, and 3f), genotype 4 (e.g., 4a, 4b, 4c, 4d, 4e, 4f, 4g, 4h, 4i and 4j), genotype 5a, and genotype 6a.

In various embodiments, the chimeric proteins or chimeric protein complexes such as Fc-based chimeric protein complex of the invention may be utilized to treat patients who are poorly or non-responsive to standard of care antiviral therapy or who are otherwise difficult to treat with standard of care hepatitis C treatment. In an embodiment, the chimeric proteins or chimeric protein complexes such as Fc-based chimeric protein complex may be utilized to treat a patient who shows low or no response to IL-2 therapy with or without ribavirin. In an embodiment, the chimeric proteins or chimeric protein complexes such as Fc-based chimeric protein complex may be utilized to treat a patient who shows low or no response to combination therapy of pegylated interferon and ribavirin. In an embodiment, the present invention is directed to the treatment of patients infected with hepatitis C genotype 1 or any other genotype who did not respond to previous IL-2 therapy. In an embodiment, the chimeric proteins or chimeric protein complexes such as Fc-based chimeric protein complex of the invention may be used to treat a patient with high baseline viral load (e.g., greater than 800,000 IU/mL). In an embodiment, the chimeric proteins or chimeric protein complexes such as Fc-based chimeric protein complex of the invention may be utilized to treat patients with severe liver damage including those patients with advanced liver fibrosis and/or liver cirrhosis.

In some embodiments, the present invention relates to the treatment of patients who are naive to antiviral therapy. In other embodiments, the present invention relates to the treatment of patients who did not respond to previous antiviral therapy. In some embodiments, the present chimeric proteins or chimeric protein complexes such as Fc-based chimeric protein complex may be used to treat relapsed patients.

In some embodiments, the present chimeric proteins or chimeric protein complexes such as Fc-based chimeric protein complex may be effective in treating hepatitis infection in all ethnic groups including white, African-American, Hispanic, and Asian. In an embodiment, the present chimeric proteins or chimeric protein complexes such as Fc-based chimeric protein complex may be particularly effective in treating African-Americans who are otherwise poorly responsive to IL-2 therapy with or without ribavirin.

In various embodiments, the targeted chimeric proteins or chimeric protein complexes such as Fc-based chimeric protein complex of the invention provides improved safety compared to, e.g., untargeted IFNα1 or an unmodified, wildtype IL-2 or a modified IL-2. In illustrative embodiments, administration of the present chimeric proteins or chimeric protein complexes such as Fc-based chimeric protein complex is associated with minimal side effects (e.g., influenza-like symptoms, myalgia, leucopenia, thrombocytopenia, neutropenia, depression, and weight loss) such as those side effects associated with the use of the untargeted IL-2 or an unmodified, wildtype IL-2 or a modified IL-2.

In some embodiments, the targeted chimeric proteins or chimeric protein complexes such as Fc-based chimeric protein complex of the invention shows improved therapeutic activity compared to untargeted IL-2 or an unmodified, wildtype IL-2, or a modified IL-2. In some embodiments, the targeted chimeric proteins or chimeric protein complexes such as Fc-based chimeric protein complex of the invention shows improved pharmacokinetic profile (e.g., longer serum half-life and stability) compared to untargeted IL-2 or an unmodified, wildtype IL-2 or a modified IL-2.

Without wishing to be bound by theory, it is believed that due to such advantageous safety and pharmacokinetic and therapeutic profiles, the present chimeric proteins or chimeric protein complexes such as Fc-based chimeric protein complex may be used to treat patients at high dosages and/or for prolonged periods of time. For example, the present chimeric proteins or chimeric protein complexes such as Fc-based chimeric protein complex may be used at high dosages for initial induction therapy against chronic hepatitis C infection. In another example, the present chimeric proteins or chimeric protein complexes such as Fc-based chimeric protein complex may be used for long-term maintenance therapy to prevent disease relapse.

In various embodiments, the present compositions are used to treat or prevent one or more inflammatory diseases or conditions, such as inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses.

In various embodiments, the present compositions are used to treat or prevent one or more autoimmune diseases or conditions, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In various embodiments, the present compositions are used to treat, control or prevent cardiovascular disease, such as a disease or condition affecting the heart and vasculature, including but not limited to, coronary heart disease (CHD), cerebrovascular disease (CVD), aortic stenosis, peripheral vascular disease, atherosclerosis, arteriosclerosis, myocardial infarction (heart attack), cerebrovascular diseases (stroke), transient ischemic attacks (TIA), angina (stable and unstable), atrial fibrillation, arrhythmia, vavular disease, and/or congestive heart failure.

In various embodiments, the present compositions are used to treat or prevent one or more metabolic-related disorders. In various embodiments, the present invention is useful for the treatment, controlling or prevention of diabetes, including Type 1 and Type 2 diabetes and diabetes associated with obesity. The compositions and methods of the present invention are useful for the treatment or prevention of diabetes-related disorders, including without limitation diabetic nephropathy, hyperglycemia, impaired glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, irritable bowel syndrome, inflamatory bowel disease, including Crohn's disease and ulcerative colitis, other inflammatory conditions, pancreatitis, abdominal obesity, neurodegenerative disease, retinopathy, neoplastic conditions, adipose cell tumors, adipose cell carcinomas, such as liposarcoma, prostate cancer and other cancers, including gastric, breast, bladder and colon cancers, angiogenesis, Alzheimer's disease, psoriasis, high blood pressure, Metabolic Syndrome (e.g. a person has three or more of the following disorders: abdominal obesity, hypertriglyceridemia, low HDL cholesterol, high blood pressure, and high fasting plasma glucose), ovarian hyperandrogenism (polycystic ovary syndrome), and other disorders where insulin resistance is a component, such as sleep apnea. The compositions and methods of the present invention are useful for the treatment, control, or prevention of obesity, including genetic or environmental, and obesity-related disorders. The obesity-related disorders herein are associated with, caused by, or result from obesity. Examples of obesity-related disorders include obesity, diabetes, overeating, binge eating, and bulimia, hypertension, elevated plasma insulin concentrations and insulin resistance, dyslipidemia, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovary disease, craniopharyngioma, Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are Metabolic Syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. The compositions and methods of the present invention are also useful to treat Alzheimer's disease.

In various embodiments, the present compositions are used to treat or prevent one or more respiratory diseases, such as idiopathic pulmonary fibrosis (IPF), asthma, chronic obstructive pulmonary disease (COPD), bronchiectasis, allergic rhinitis, sinusitis, pulmonary vasoconstriction, inflammation, allergies, impeded respiration, respiratory distress syndrome, cystic fibrosis, pulmonary hypertension, pulmonary vasoconstriction, emphysema, Hantavirus pulmonary syndrome (HPS), Loeffler's syndrome, Goodpasture's syndrome, Pleurisy, pneumonitis, pulmonary edema, pulmonary fibrosis, Sarcoidosis, complications associated with respiratory syncitial virus infection, and other respiratory diseases.

In some embodiments, the present invention is used to treat or prevent one or more neurodegenerative disease. Illustrative neurodegenerative diseases include, but are not limited to, Friedreich's Ataxia, multiple sclerosis (including without limitation, benign multiple sclerosis; relapsing-remitting multiple sclerosis (RRMS); secondary progressive multiple sclerosis (SPMS); progressive relapsing multiple sclerosis (PRMS); and primary progressive multiple sclerosis (PPMS), Alzheimer's. disease (including, without limitation, Early-onset Alzheimer's, Late-onset Alzheimer's, and Familial Alzheimer's disease (FAD), Parkinson's disease and parkinsonism (including, without limitation, Idiopathic Parkinson's disease, Vascular parkinsonism, Drug-induced parkinsonism, Dementia with Lewy bodies, Inherited Parkinson's, Juvenile Parkinson's), Huntington's disease, Amyotrophic lateral sclerosis (ALS, including, without limitation, Sporadic ALS, Familial ALS, Western Pacific ALS, Juvenile ALS, Hiramaya Disease).

In various embodiments, the present chimeric proteins or chimeric protein complexes such as Fc-based chimeric protein complex finds use in treating wounds, e.g., a non-healing wound, an ulcer, a burn, or frostbite, a chronic or acute wound, open or closed wound, internal or external wound (illustrative external wounds are penetrating and non-penetrating wound.

In various embodiments, the present chimeric proteins or chimeric protein complexes such as Fc-based chimeric protein complexes find use in treating ischemia, by way of non-limiting example, ischemia associated with acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, frostbite, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, or wounds to tissues or organs

In various embodiments, the present invention relates to the treatment of one or more of anemia, including anemia resulting from chronic kidney disease (e.g. from dialysis) and/or an anti-cancer agent (e.g. chemotherapy and/or HIV treatment (e.g. Zidovudine (INN) or azidothymidine (AZT)), inflammatory bowel disease (e.g. Crohn's disease and ulcer colitis), anemia linked to inflammatory conditions (e.g. arthritis, lupus, IBD), anemia linked to diabetes, schizophrenia, cerebral malaria, as aplastic anemia, and myelodysplasia from the treatment of cancer (e.g. chemotherapy and/or radiation), and various myelodysplastic syndrome diseases (e.g. sickle cell anemia, hemoglobin SC disease, hemoglobin C disease, alpha- and beta-thalassemias, neonatal anemia after premature birth, and comparable conditions).

In some embodiments, the present invention relates to the treatment of, or a patient having anemia, i.e. a condition in which the number of red blood cells and/or the amount of hemoglobin found in the red blood cells is below normal. In various embodiments, the anemia may be acute or chronic. For example, the present anemias include but are not limited to iron deficiency anemia, renal anemia, anemia of chronic diseases/inflammation, pernicious anemia such as macrocytic achylic anemia, juvenile pernicious anemia and congenital pernicious anemia, cancer-related anemia, anti-cancer-related anemia (e.g. chemotherapy-related anemia, radiotherapy-related anemia), pure red cell aplasia, refractory anemia with excess of blasts, aplastic anemia, X-lined siderobalstic anemia, hemolytic anemia, sickle cell anemia, anemia caused by impaired production of ESA, myelodysplasia syndromes, hypochromic anemia, microcytic anemia, sideroblastic anemia, autoimmune hemolytic anemia, Cooley's anemia, Mediterranean anemia, Diamond Blackfan anemia, Fanconi's anemia and drug-induced immune hemolytic anemia. Anemia may cause serious symptoms, including hypoxia, chronic fatigue, lack of concentration, pale skin, low blood pressure, dizziness and heart failure.

In some embodiments, the present invention relates to the treatment of anemia resulting from chronic renal failure. In some embodiments, the present invention relates to the treatment of anemia resulting from the use of one or more renal replacement therapies, inclusive of dialysis, hemodialysis, peritoneal dialysis, hemofiltration, hemodiafiltration, and renal transplantation.

In some embodiments, the present invention relates to the treatment of anemia in patients with chronic kidney disease who are not on dialysis. For instance, the present invention relates to patients in stage 1 CKD, or stage 2 CKD, or stage 3 CKD, or stage 4 CKD, or stage 5 CKD. In some embodiments, the present patient is stage 4 CKD or stage 5 CKD. In some embodiments, the present patient has undergone a kidney transplant. In some embodiments, the present invention relates to the treatment of anemia is a patient having an acute kidney injury (AKI).

In some embodiments, the anemia is induced by chemotherapy. For instance, the chemotherapy may be any myelosuppressive chemotherapy. In some embodiment, the chemotherapy is one or more of Revlimid, Thalomid, dexamethasone, Adriamycin and Doxil. In some embodiments, the chemotherapy is one or more platinum-based drugs including cisplatin (e.g. PLATINOL) and carboplatin (e.g. PARAPLATIN). In some embodiments, the chemotherapy is any one of the chemotherapeutic agents described herein. In some embodiments, the chemotherapy is any agent described in Groopman et al. J Natl Cancer Inst (1999) 91 (19): 1616-1634, the contents of which are hereby incorporated by reference in their entireties. In some embodiments, the present compositions and methods are used in the treatment of chemotherapy-related anemia in later stage cancer patients (e.g. a stage IV, or stage III, or stage II cancer). In some embodiments, the present compositions and methods are used in the treatment of chemotherapy-related anemia in cancer patients receiving dose-dense chemotherapy or other aggressive chemotherapy regimens.

In some embodiments, the present invention relates to the treatment of anemia in a patient having one or more blood-based cancers, such as leukemia, lymphoma, and multiple myeloma. Such cancers may affect the bone marrow directly. Further, the present invention relates to metastatic cancer that has spread to the bone or bone marrow. In some embodiments, the present invention relates to the treatment of anemia in a patient undergoing radiation therapy. Such radiation therapy may damage the bone marrow, lowering its ability to make red blood cells. In further embodiments, the present invention relates to the treatment of anemia in a patient having a reduction or deficiency of one or more of iron, vitamin B12, and folic acid. In further embodiments, the present invention relates to the treatment of anemia in a patient having excessive bleeding including without limitation, after surgery or from a tumor that is causing internal bleeding. In further embodiments, the present invention relates to the treatment of anemia in a patient having anemia of chronic disease.

In some embodiments, the present methods and compositions stimulate red blood cell production. In some embodiments, the present methods and compositions stimulate division and differentiation of committed erythroid progenitors in the bone marrow.

Certain embodiments of the present invention are particularly useful for treating chemotherapy-induced anemia in cancer patients. In some embodiments, the present methods and compositions allows for continued administration of the chimeric proteins or chimeric protein complexes such as Fc-based chimeric protein complex after a cancer patient's chemotherapy is finished. In some embodiments, the present methods and compositions allows for treatment of a cancer patient without dose reduction relative to a non-cancer patient. In some embodiments, the present methods and compositions allows for treatment of a cancer patient receiving chemotherapy and considered curable. In various embodiments, the cancer patient has one or more of a history of blood clots, recent surgery, prolonged periods of bed rest or limited activity, and treatment with a chemotherapeutic agent.

### Kits

The invention also provides kits for the administration of any agent described herein (e.g. the chimeric proteins or chimeric protein complexes, such as Fc-based chimeric protein complexes, with or without various additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Definitions

Throughout the application, a chimeric protein or protein complex of the present invention may be indicated by the term “AcTaleukin-2” and/or “ALN2”.

As used herein, “a,” “an,” or “the” can mean one or more than one.

Unless specifically stated or obvious from context, as used herein, the term “or” is understood to be inclusive and covers both “or” and “and”.

Further, the term “about” when used in connection with a referenced numeric indication means the referenced numeric indication plus or minus up to 10% of that referenced numeric indication, e.g., within (plus or minus) 10%, 9%, 8%, 7%, 6%, 5%, 4%, 3%, 2%, 1%, 0.5%, 0.1%, 0.05%, or 0.01% of the stated value. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein. This invention is further illustrated by the following non-limiting examples.

### EXAMPLES

For the purposes of the following Examples, and throughout the application, a chimeric protein or protein complex of the present invention may be indicated by the term “AcTaleukin-2” and/or “ALN2”.

### Example 1: IL-2 and Fc-IL-2 Drive STAT5 Phosphorylation in Peripheral Blood Mononuclear Cells (PBMCs)

This Example describes how Interleukin-2 (IL-2) can be modified in such a way that only targeted cells are activated, and cells with negative or adverse effects are no longer activated. In this example, anti-tumor activities of CD8 expressing T cells (e.g. cytotoxic T cells or CTLs) were boosted, while immune-suppressing functions of regulatory T cells (Tregs) were restricted. This uncoupling of cellular functions can, in part, be achieved based on the use of IL-2 receptors (IL-2R) in these cells: while signaling in CTLs is mediated by an intermediate-affinity IL-2R complex composed of the IL-2Rβ (CD122) and γ common (γc; CD132) chains, in T_{regs} the high-affinity IL-2R complex additionally includes an IL-2Rα (CD25) chain. The strategy for development of CTL-specific IL-2 activity includes: (i) CD8 targeting, (ii) elimination of the CD25 binding and thus involvement of this receptor in signaling, and (iii) the selection of a loss-of-function beta- and gamma-mutations that can be restored upon (CD8) targeting.

First, the effect of Fc-fusion on the activity of IL-2 was examined. To this end, the cytokine was cloned (in the pcDNA3.4 vector) via a flexible 20*GGS-linker C-terminally to a heterodimeric, ‘knob-in-hole’ human IgG1 Fc backbone. Fc sequences contained the L234A_L235A_K322Q effector mutations and the ‘hole’ modifications Y349C_T366S_L368A_Y407V (in sequence hFc3) or ‘knob’ mutations S354C_T366W (in sequence hFc4-hIL-2_C125A). For stability and manufacturability reasons, the free cysteine on position 125 in IL-2 was mutated to an alanine (C125A)

To produce this ‘knob-in-hole’ Fc-IL-2, a combination of both ‘hole’ and ‘knob’ plasmids was transfected in ExpiCHO cells (ThermoFisher) according to the manufacturer's instructions. Seven days post transfection, recombinant proteins were purified using protein A spin plates (ThermoFisher), quantified and purity tested using SDS-PAGE.

Resulting AcTaleukin-2 (ALN2) was tested for STAT5 phosphorylation in (i) CD8 positive T cells (CD8⁺); (ii) CD25 negative conventional T cells (CD25− Tconv); (iii) CD25 positive conventional T cells (CD25⁺ Tconv); or (iv) regulatory T cells (Treg) defined as CD4⁺CD25+FoxP3+. In brief, PBMCs from buffy coats of healthy donors were isolated using density gradient centrifugation using Lymphoprep (StemCell technologies). Cells were stimulated with a serial dilution wild type recombinant IL-2 or Fc-IL-2 for 30 minutes at 37° C. After centrifugation, cells were resuspended in Lyse/Fix buffer (BD Biosciences) and further incubated for 10 minutes at 37° C. Cells were washed and incubated with human FcR Blocking Reagent (Miltenyi Biotec) and stained with anti-CD25 and anti-CD8 for 30 minutes at room temperature. Cells were subsequently permeabilized using the Perm Buffer III (BD Biosciences) at 4ºC for 30 minutes. Cells were finally stained with anti-CD3, anti-CD4, anti-FoxP3, and anti-pSTAT5 for 1 hour. Samples were acquired on a MACSQuant X instrument (Miltenyi Biotec) and analyzed using the FlowLogic software (Miltenyi Biotec).

FIG. 20 illustrates that (i) the Treg cells are much more sensitive to IL-2 compared to other cell-types, and (ii) that Fc-fusion affects signaling in all tested populations. As an example, Fc-IL-2 is +20-fold less active than IL-2 on Tregs and the fusion is hardly active on CD8 positive cells.

### Example 2: CD25 Knock Out Diminishes Fc-IL-2 Signaling in all Cell Types

This Example examined the effect of five mutant combinations that are described to reduce CD25 binding on the behavior of the Fc-IL-2 fusion: (i) R38A_F42Y_Y45A_E62A; (ii) F42Y_Y45A_L72G; (iii) F42K; (iv) R38A_F42K; and (v) E61Q.

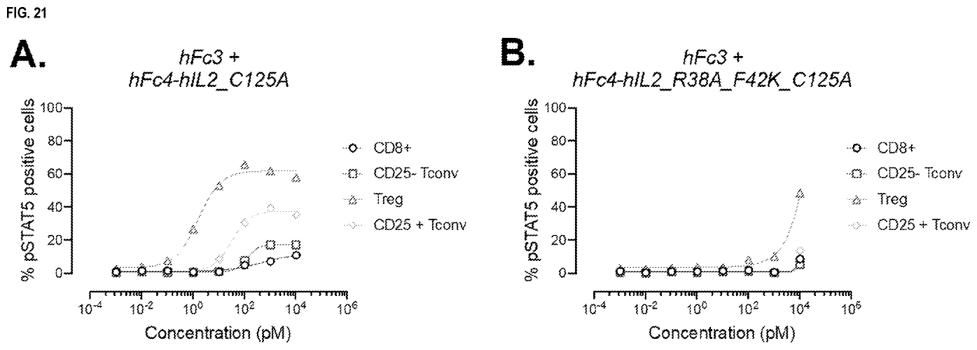

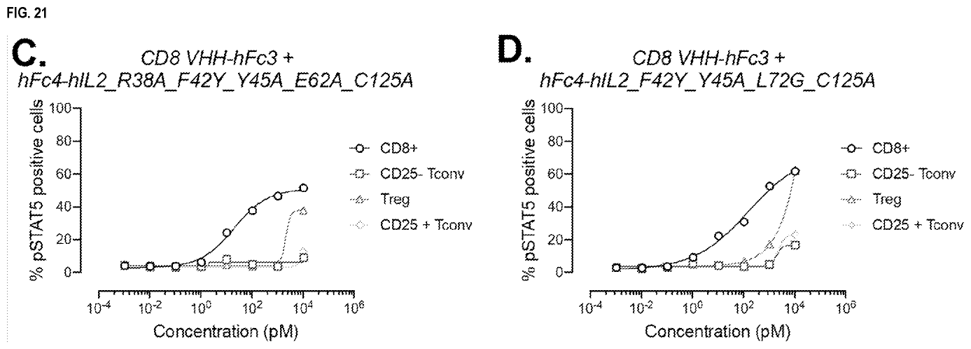

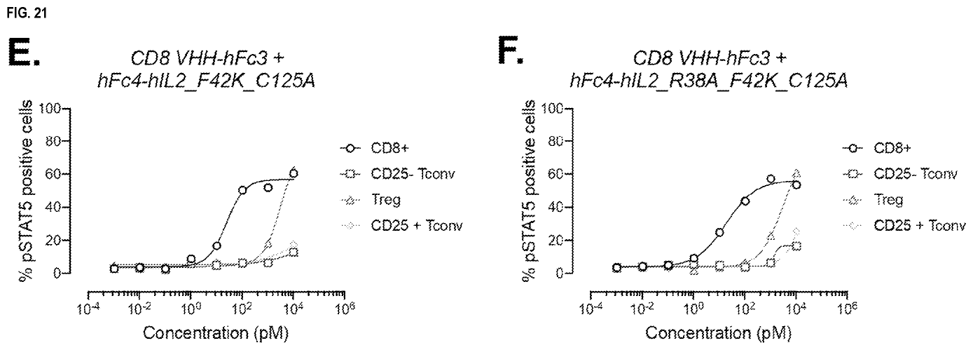

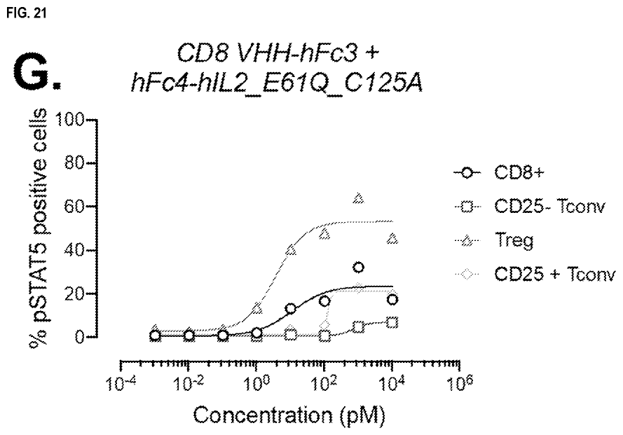

These residues were mutated in the hFc4-hIL-2_C125A construct (see resulting sequences below). These knob constructs were combined with the hole-fusion hCD8 VHH-hFc3, in which hCD8 VHH 1CDA65 sequence was fused via a 20*GGS-linker to the Fc with hole mutations (sequence below), which allows for the evaluation of the CD8 targeting efficiency. A combination of plasmids was transfected in ExpiCHO and purified as described above. Resulting proteins were tested for STAT5 phosphorylation in the different lymphocyte populations (FIGS. 21A-G). Data illustrate that reducing the CD25 interaction (e.g., via the R38A_F42K mutation) affects signaling in all sub-populations, and this effect is independent of the presence of CD25 (FIGS. 21A-B). This likely illustrates that the CD25 mutations not only directly affect CD25 binding, but also have some conformational effects. If the CD25 knock-out mutant is targeted to CD8 expressing cells using the CD8 VHH, there is a clear increase of signaling in these cells, while signaling in other cell-types remains mostly unaltered (FIGS. 21C-G). An exception is seen for the E61Q mutations (FIG. 21G) for which the attenuation is only modest compared to the other four variants.

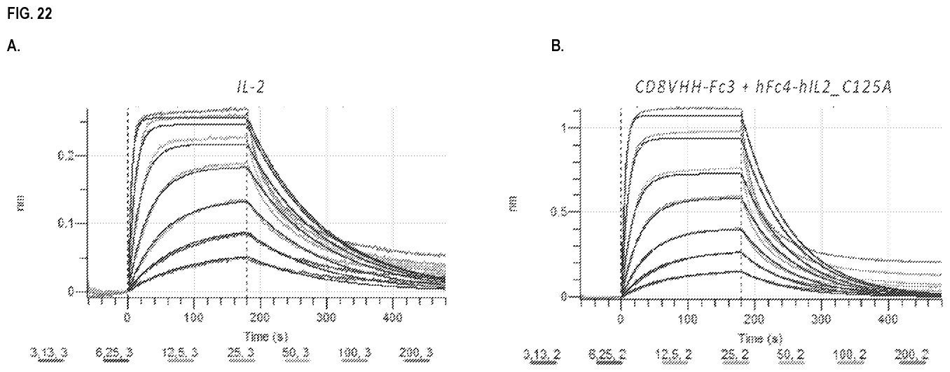

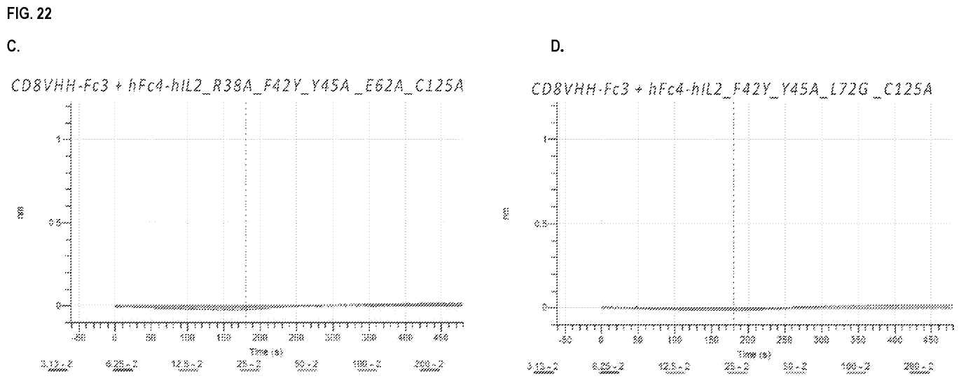

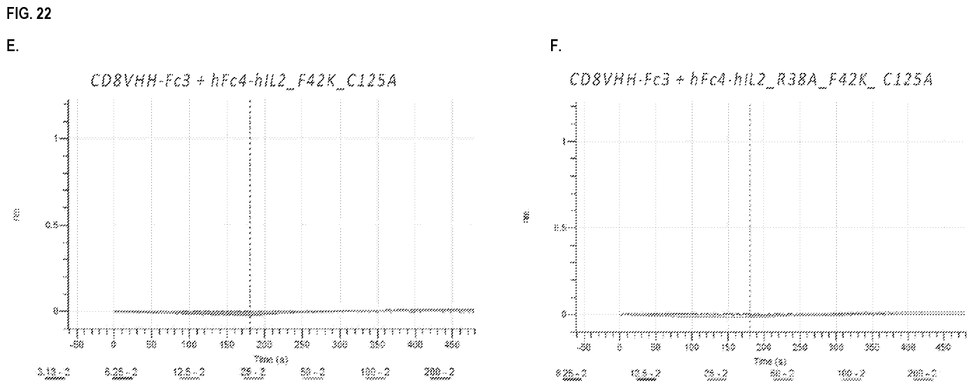

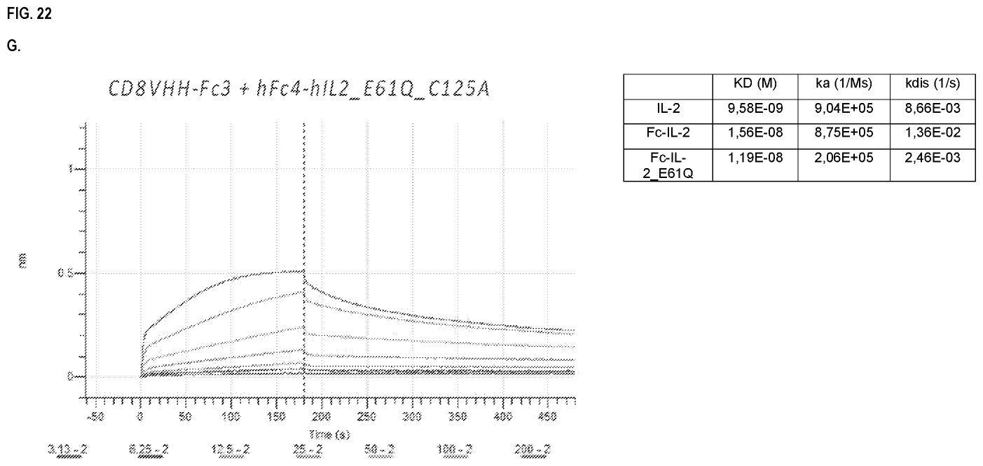

Bio-layer interferometry (BLI) on an Octet RED96 instrument (ForteBio) was also used to show that the CD25 mutations indeed reduce binding to CD25. In brief, recombinant CD25 (Acro Biosystems) was biotinylated using the Pierce Antibody Biotinylation Kit for IP and loaded on Streptavidin sensors. Association and dissociation of seven concentrations of wild type IL-2, Fc-IL-2 or Fc-ALN2 with mutated CD25 binding-site (all CD8 targeted) were monitored and used to calculate the association and dissociation constants and hence affinity (FIGS. 22A-G). Fusion to a Fc appears not to affect binding of IL-2 to CD25 and suggests that the loss in activity observed in Example 1 is due to a less efficient binding to IL-2Rβ and/or IL-2Rγ. chains. On the other hand, the combination mutations R38A_F42Y_Y45A_E62A; F42Y_Y45A_L72G; F42K; and R38A_F42K completely abolish detectable CD25 binding (i.e., no specific binding could be measured). Also, here the effect of mutation E61Q is only modest and explains the more potent STAT5 phosphorylation described earlier.

In conclusion, CD25 knock-out mutations diminish IL-2 signaling in all cell-types (independent of CD25 expression), and CD8 targeting partly restores signaling in CD8 expressing cells. However, such ALN2 molecules with mutated CD25 binding-site still clearly signal in Treg cells with an EC50 of 50 to 250 ng/ml.

### Example 3: Identification of Modified IL-2 Having Mutations of Residues Involved in IL-2Rβ Binding

Previous experiments illustrated that mutation of the CD25 binding-site results in an attenuation in signaling, and that CD8 targeting, at least in part, restores this loss in signaling. Resulting ALN2 variants were still able to induce STAT5 phosphorylation in the hyper-sensitive Treg cells. To further reduce this latter signaling (and thus the CD8-selectivity of ALN2 activity), an option of a restorable (upon targeting) loss-of-function mutation of residues involved in IL-2Rβ binding was examined.

Candidate residues D20 and N88 were mutated to any other amino acid (excluding C and M) in the hFc4-hIL-2_C125A construct (see sequences below). Resulting constructs were combined with CD8-hFc3 for expression in ExpiCHO. Recombinant proteins were purified from the supernatant using protein A spin plates (ThermoFisher), quantified and purity tested using SDS-PAGE.

Resulting ALN2 variants were tested for STAT5 phosphorylation in CD8⁺, CD4⁺CD25, and CD4⁺CD25⁺ PBMC populations as follows: PBMCs were blocked human FcR Blocking Reagent (Miltenyi Biotec) and stained fluorescently labelled Ab's specific for CD4, CD8, and CD25. After staining and washing, cells were stimulated with a serial dilution ALN2 variants as indicated for 30 min at 37° C. Cells were subsequently fixed with Lyse/Fix buffer (BD Biosciences) and permeabilized using the Perm Buffer III (BD Biosciences) according to the manufacturer's guidelines. After overnight staining with a pSTAT5 specific Ab, samples were analyzed on a MACSQuant X instrument (Miltenyi Biotec) and analyzed using the FlowLogic software (Miltenyi Biotec).

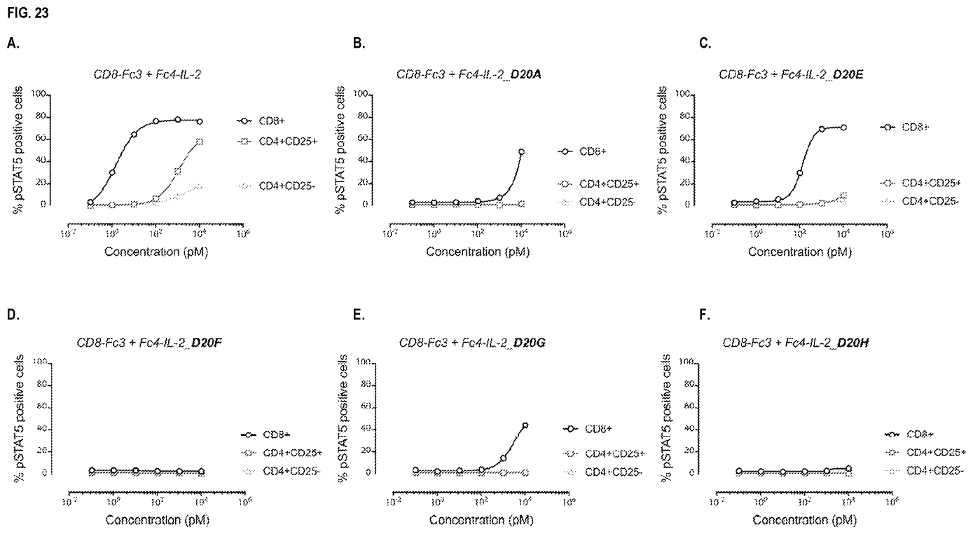

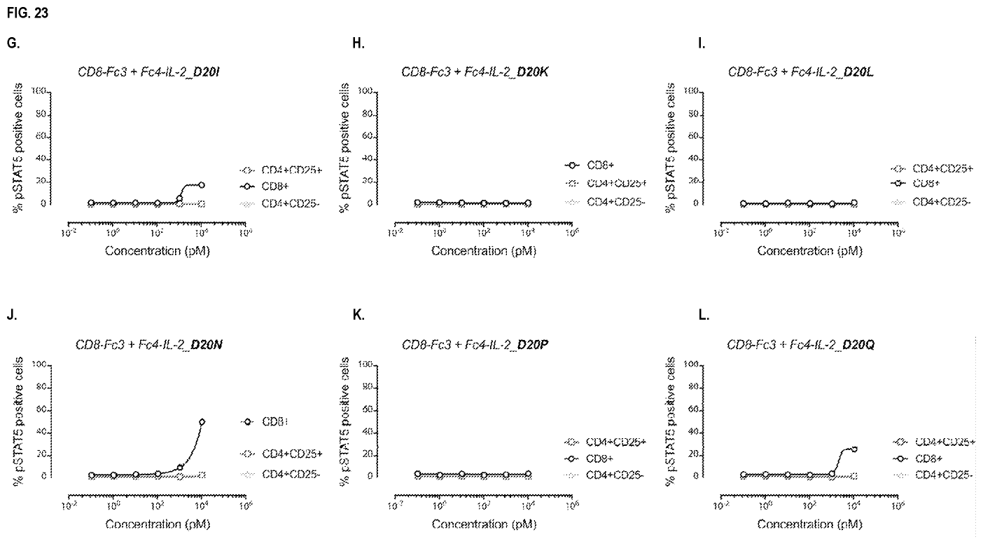

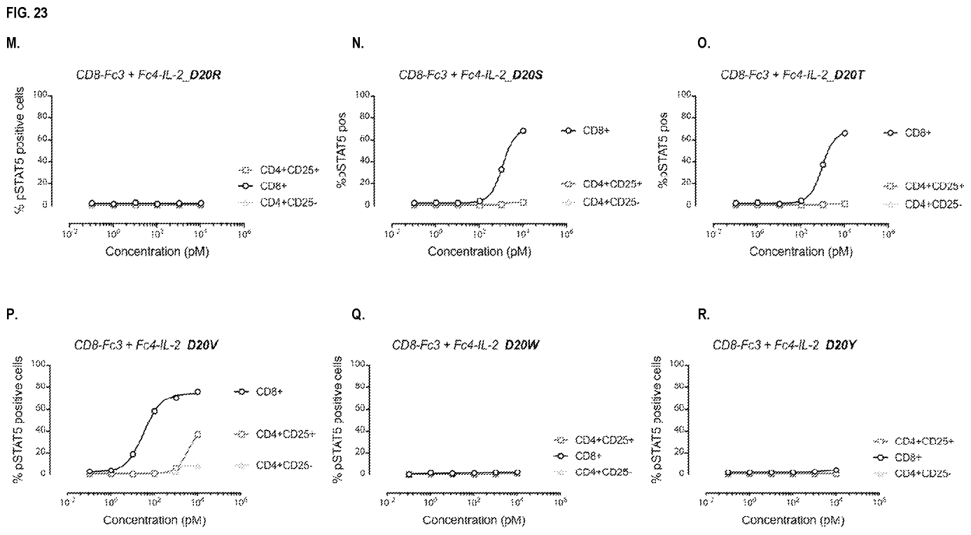

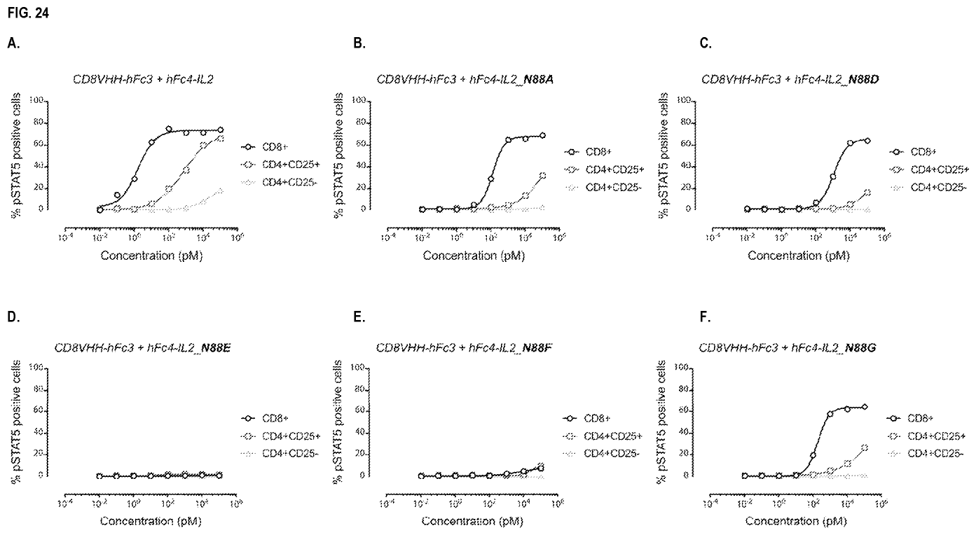

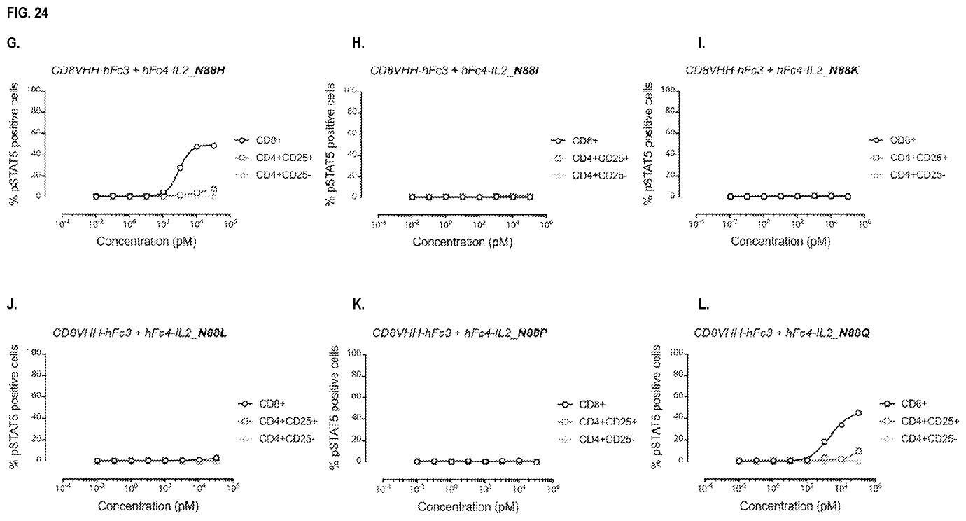

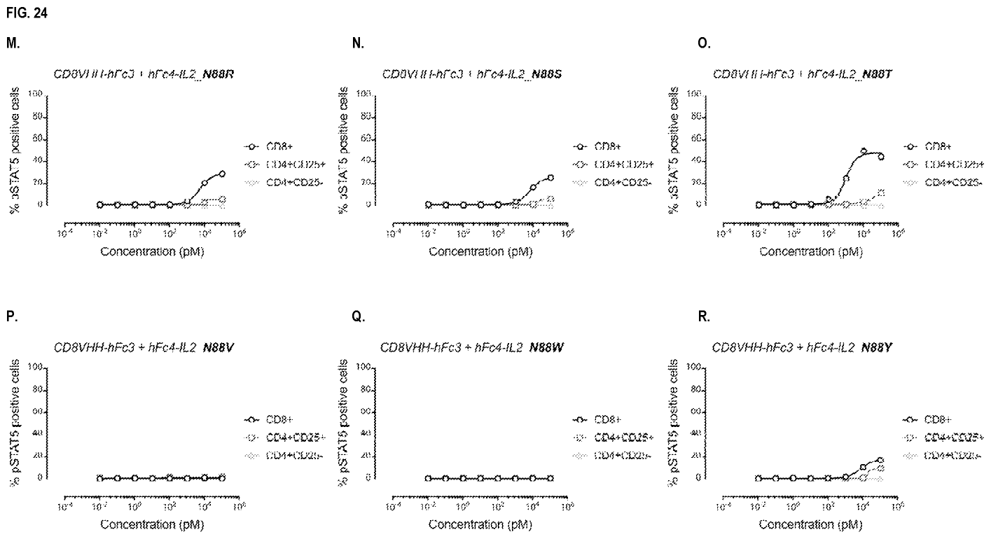

FIGS. 23A-R (screening of D20 mutants) and FIGS. 24A-R (screening of N88 mutants) illustrate that most mutations have a drastic effect on the pSTAT5 outcome. Variants that retained most activity (compared to wild type) were D20E, D20V, N88A, N88G (and to a lesser extent D20S, D20T, N88D, N88Q, N88H, and N88T) with EC50 values between 3 and 18 ng/ml. Data also clearly illustrate that even with a mutation of the IL-2Rβ site and CD8 targeting, clear pSTAT5 can be detected at concentrations of 1 μg/ml in the CD4⁺CD25⁺ population, of which the hyper-sensitive Tregs are very small portion.

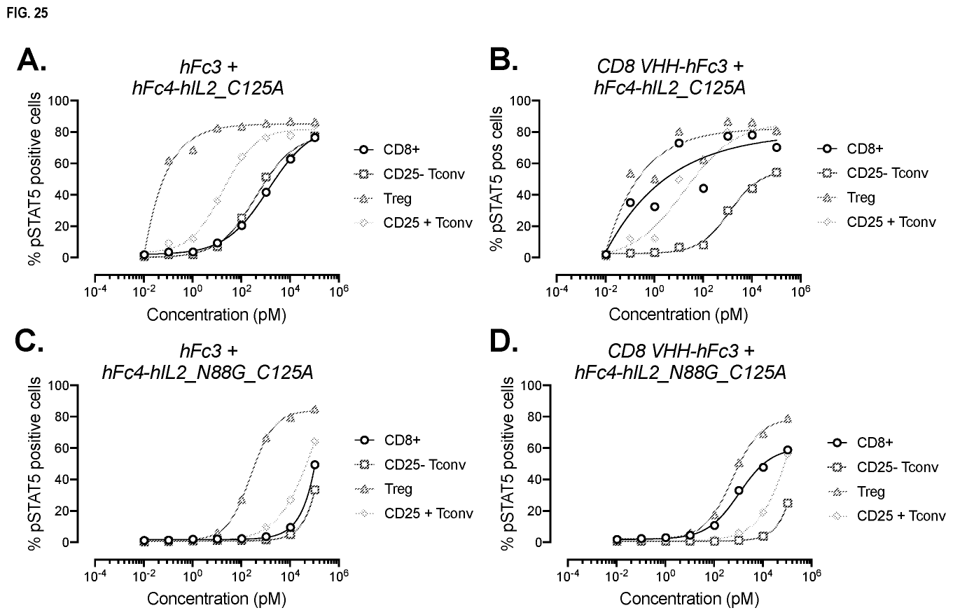

Then, the effect of the beta mutations (here N88G as an example) on STAT5 phosphorylation was evaluated in CD8⁺, CD25⁻ Tconv, CD25⁺ Tconv and Treg cells. Therefore, wild type IL-2 or IL-2_N88G (both with the C125A) were expressed as untargeted (combination with hFc3) or CD8 targeted (combination with CD8 VHH-hFc3) as heterodimeric Fc fusion-proteins. Production, purification and pSTAT5 in PBMCs were performed as described in Example 1. The results shown in FIGS. 25A-D clearly illustrate that the N88 mutation diminishes signaling in all PBMC populations (FIG. 25A vs FIG. 25C), and that CD8 targeting increases signaling in antigen expressing cells, while pSTAT5 in other sub-populations remains mostly unaffected (FIG. 25A vs FIG. 25B and FIG. 25C vs FIG. 25D).

### Example 4: Combination of CD25 Knock-Out and Beta Mutations

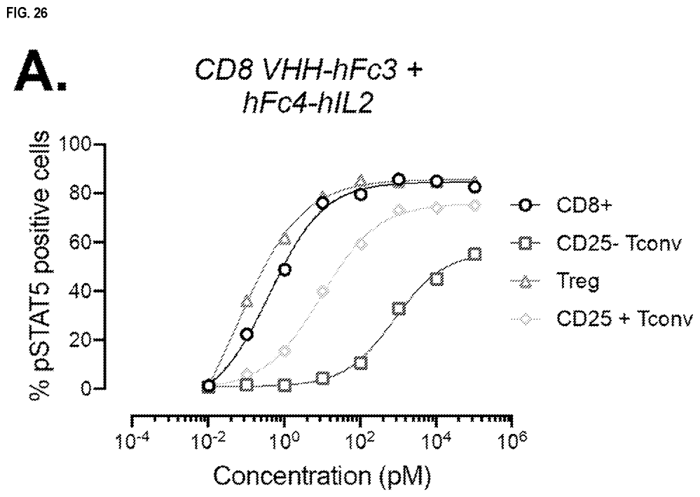

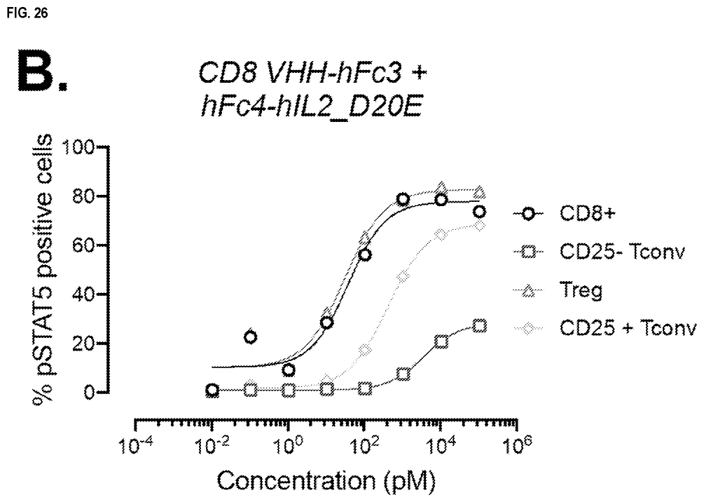

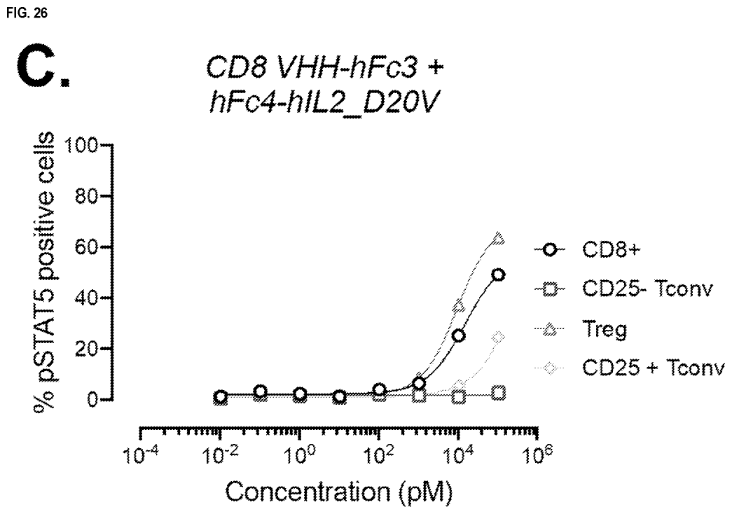

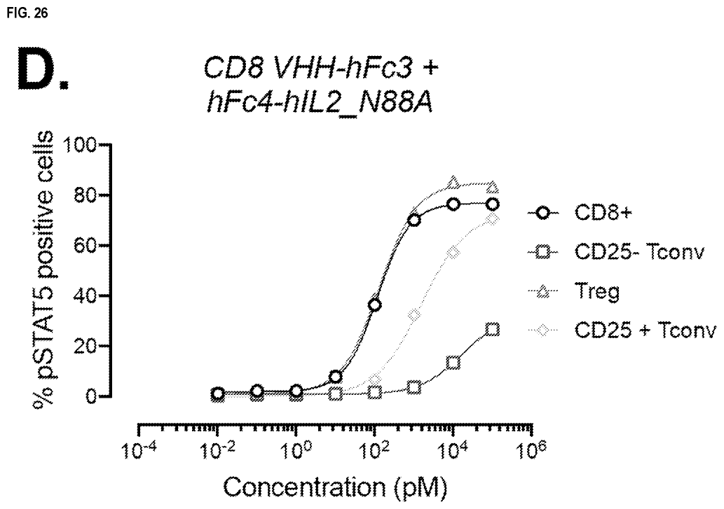

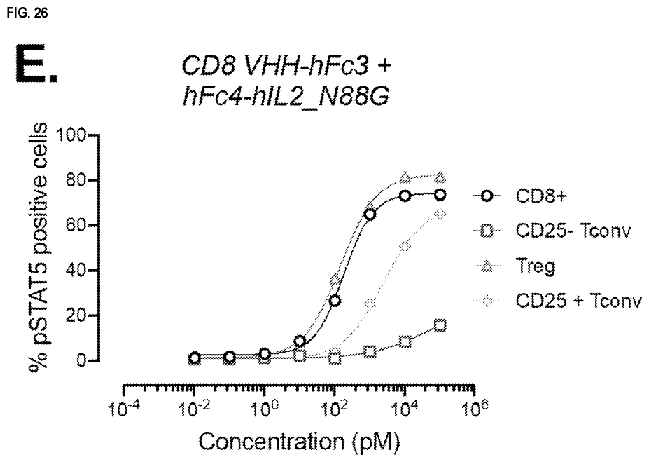

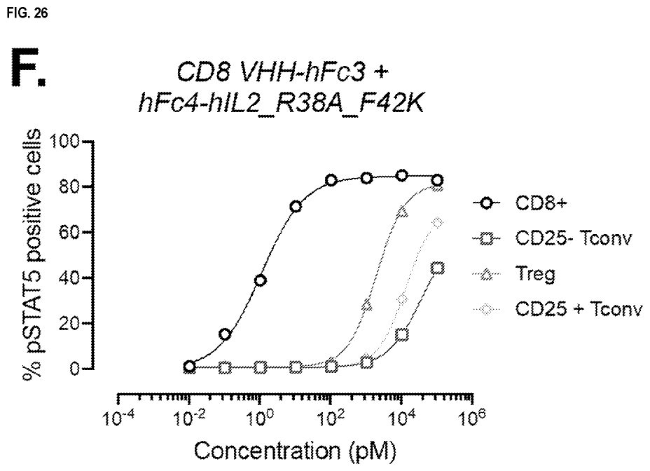

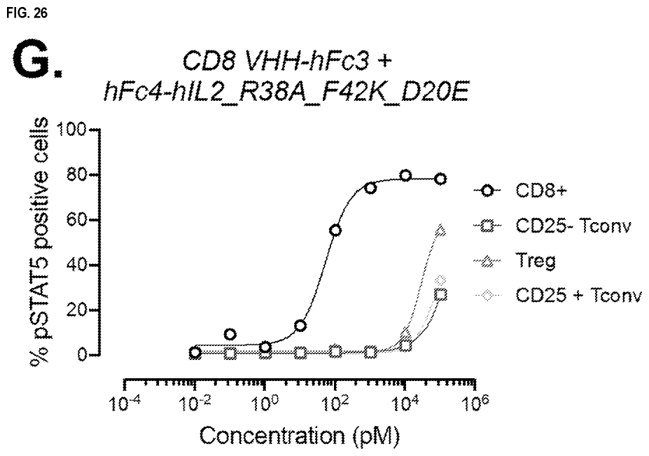

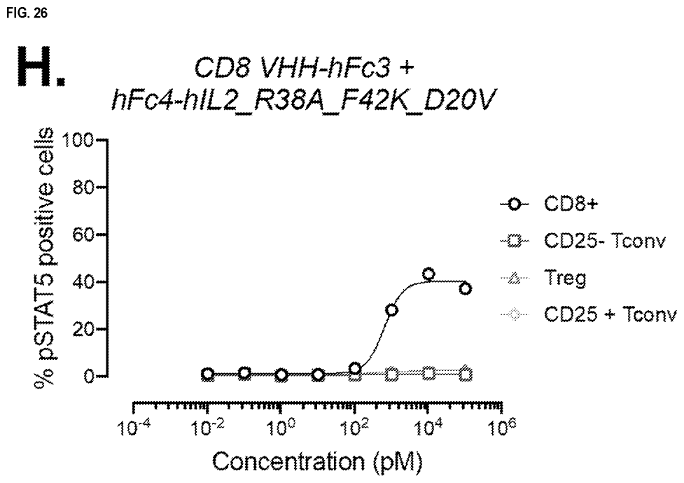

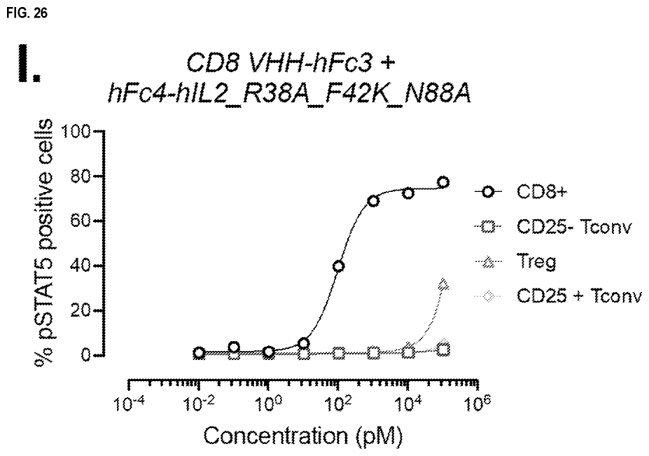

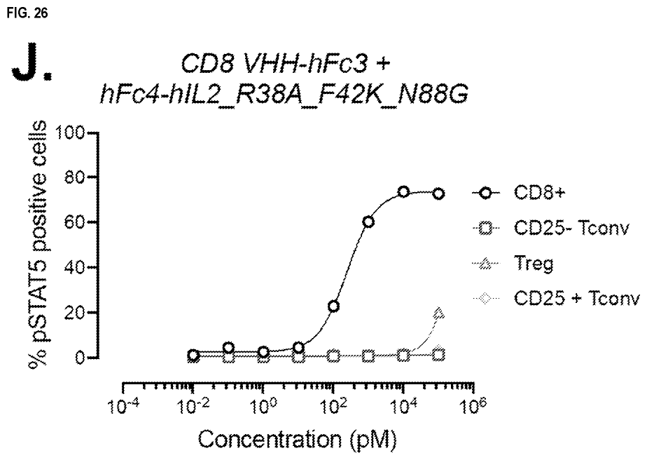

The purpose of this Example was to combine CD25 knock-out mutations (e.g., R38A_F42K) with the best performing IL-2Rβ-mutations (e.g., D20E, D20V, N88A and N88G). Resulting constructs (see below for sequences) were combined with the CD8 VHH-hFc3 partner and tested for STAT5 phosphorylation in CD8⁺, CD25⁻ Tconv, CD25⁺ Tconv and Treg cells as described in Example 1. Data in FIGS. 26A-J and summary Table 7 illustrate that:
i. CD25 knock-out (e.g., R38A_K42K) mutations affect signaling in all sub-populations independent of CD25 expression (FIG. 26A and FIG. 26F).
ii. IL-2Rβ mutations (e.g., D20E, D20V, N88A, N88G) also affect signaling in all sub-populations.
iii. On CD8 positive cells, EC50 values for variants with mutated CD25 or combination mutation of CD25 and beta-sites are comparable, suggesting that CD8 targeting compensates for the loss in signaling due to the beta mutation (FIG. 26B vs FIG. 26G, FIG. 26C vs FIG. 26H, FIG. 26D vs FIG. 26I, and FIG. 26E vs FIG. 26J).
iv. Although with reduced efficacy, ALN2 variants with mutated CD25 or IL-2Rβ binding-sites still clearly signal in Treg cells (FIG. 26B, FIG. 26C, FIG. 26D, FIG. 26E, and FIG. 26F).
v. A combination of CD25 and IL-2Rβ mutations is needed to almost abolish completely signaling in the hyper-sensitive Treg cells (FIGS. 26G-J).
vi. This combination of CD25 binding reduction and selected IL-2Rβ mutations and targeting to CD8, finally resulted in up to a 2000-fold specificity CD8⁺ over Tregs (FIG. 26F, FIG. 26G, FIG. 26I, and FIG. 26J).

TABLE 7
With reference to panels depicted in FIGS. 26A-J, EC₅₀ (in pM; extrapolated if necessary)
of combination of CD25 and IL-2Rβ mutations and their effect on pSTAT5 in PMBCs populations

| | | | CD25− | | CD25+ | Ratio CD8+ |
|---|---|---|---|---|---|---|
| Panel | ALN2 variant | CD8+ | Tconv | Treg | Tconv | over Treg |
| A | CD8 VHH-hFc3 + hFc4-hIL-2_C125A | 0.4057 | 5498 | 0.08581 | 10.92 | 0.21 |
| B | CD8 VHH-hFc3 + hFc4-hIL-2_D20E_C125A | 41.13 | ~ | 27.03 | 770.3 | 0.66 |
| C | CD8 VHH-hFc3 + hFc4-hIL-2_D20V_C125A | 49021 | ~ | 14980 | 293171 | 0.31 |
| D | CD8 VHH-hFc3 + hFc4-hIL-2_N88A_C125A | 134.5 | ~ | 114 | 2333 | 0.85 |
| E | CD8 VHH-hFc3 + hFc4-hIL-2_N88G_C125A | 237.5 | ~ | 135.2 | 4860 | 0.57 |
| F | CD8 VHH-hFc3 + hFc4-hIL-2_R38A_F42K_C125A | 1.041 | ~ | 1865 | 19248 | 1791.55 |
| G | CD8 VHH-hFc3 + hFc4-hIL-2_R38A_F42K_{——}D20E_C125A | 57.56 | ~ | 55529 | 140458 | 964.72 |
| H | CD8 VHH-hFc3 + hFc4-hIL-2_R38A_F42K_D20V_C125A | 662.7 | ~ | ~ | ~ | ND |
| I | CD8 VHH-hFc3 + hFc4-hIL-2_R38A_F42K_{——}N88A_C125A | 124.1 | ~ | 147584 | ~ | 1189.23 |
| J | CD8 VHH-hFc3 + hFc4-hIL-2_R38A_F42K_N88G_C125A | 333.8 | ~ | 241760 | ~ | 724.27 |

~: an accurate EC₅₀ could not be determined

In conclusion, Treg cells are on average 100,000-fold more sensitive to wild type IL-2 compared to CD8⁺ T cells. A combination of CD8-targeting, CD25-binding knock-out, and selected recoverable IL-2Rβ mutations resulted in an ALN2 molecule with a specificity of 1,000-fold in favor of the CD8 expressing cells and no detectable Treg activity up to 1,000 or even 10,000 pM which equals concentrations of about 0.1-1 μg/ml. Of note IL-2 molecules with only IL-2Rα mutations already display maximal activation of Tregs at 10,000 pM (FIGS. 26A-J).

### Example 5: CD25 Targeting of IL-2 Activity

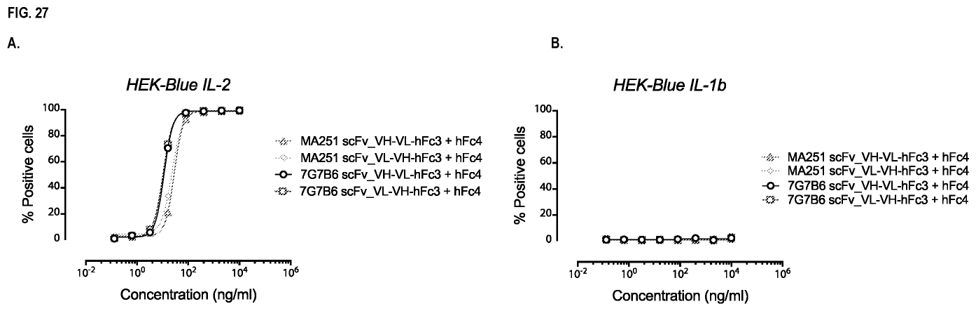

In the context of autoimmune diseases, it is believed that it may be desirable to specifically activate (e.g. through IL-2) Treg cells while leaving other (IL-2) responsive cells untouched. This example explored the possibility to activate Tregs with modified IL-2 molecules that are targeted to the CD25 receptor which is highly expressed on these cells. As targeting domains, scFv variants of two non-neutralizing anti-CD25 antibodies (MA251 and 7G7B6) were chosen. To this end, VH and VL sequences were fused via a 3*GGGGS-liner in two orientations (VH-VL and VL-VH) and linked to the hFc3 sequence with effector and hole mutations (see sequences below). Resulting constructs were combined with a hFc4 expressing plasmid (in order to generate unarmed knob-in-hole Fc constructs) for production in ExpiCHO cells. Resulting proteins were purified as described above and initially tested for (i) binding to CD25 expressing cells, and (ii) for neutralization of IL-2 signaling. For binding, two HEK derived cell-lines HEK-Blue-IL-2 (expressing a functional IL-2R; InvivoGen) and HEK-Blue-IL-1 (expressing a functional IL-1R; InvivoGen) were incubated with a serial dilution of MA251 and 7G7B6 scFv unarmed proteins for 2 hours at 4ºC. Binding was measured using a fluorescently labeled anti-human Ab in FACS. Samples were acquired on a MACSQuant X instrument (Miltenyi Biotec) and analyzed using the FlowLogic software (Miltenyi Biotec). Data in FIGS. 27A-B clearly illustrate that CD25 scFv targeted Fc-fusions only bind to CD25 expressing HEK-Blue-IL-2 cells, but not to the related HEK-Blue-IL-1 cells.

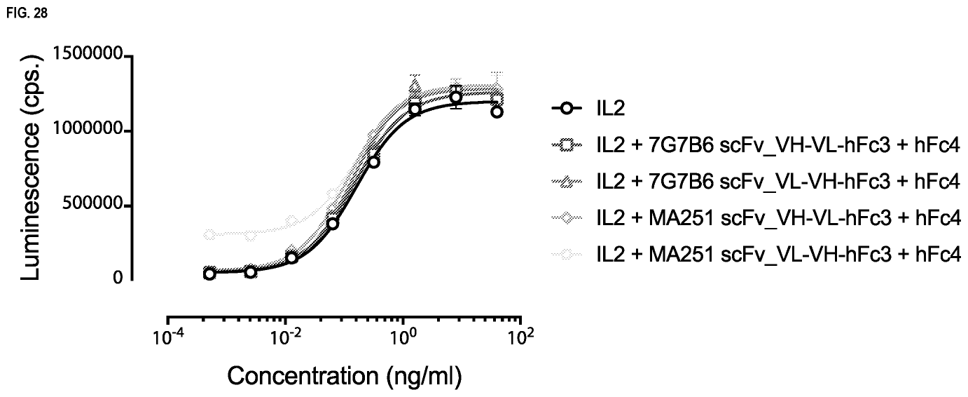

The effect on IL-2 signaling was measured in the HEK-Blue IL-2 cells. Cells were pre-incubated with and without an excess (10 μg/ml) of unarmed CD25 scFv Fc-fusion before adding a serial dilution of wild type IL-2 (FIG. 28). After overnight stimulation, secreted phosphatase activity was measured using the Phospha-Light™ SEAP Reporter Gene Assay System (ThermoFisher) and plotted in function of the concentration (FIG. 28). This experiment illustrated that none of the tested proteins interfere with IL-2 signaling.

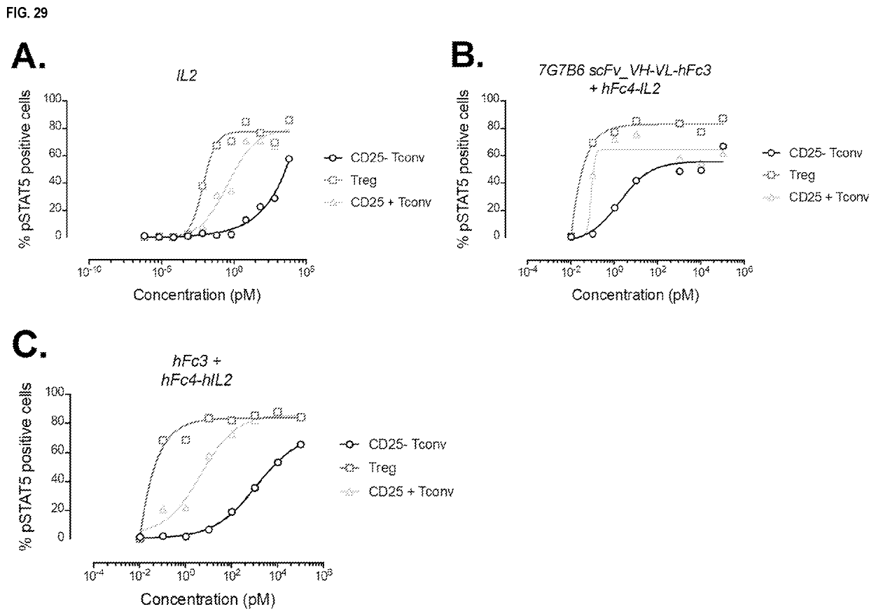

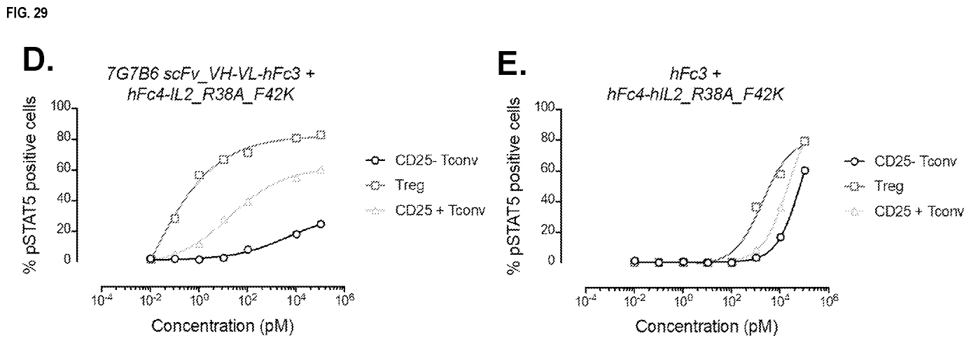

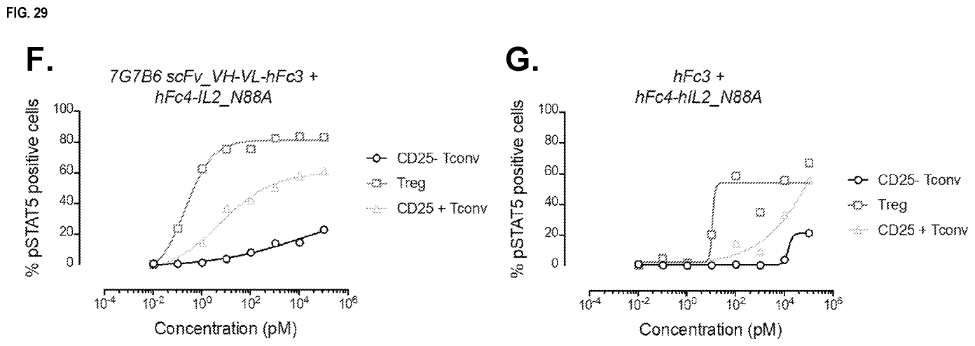

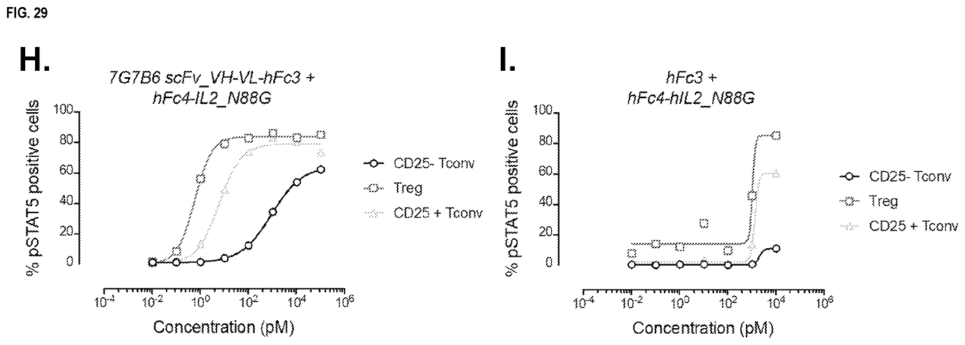

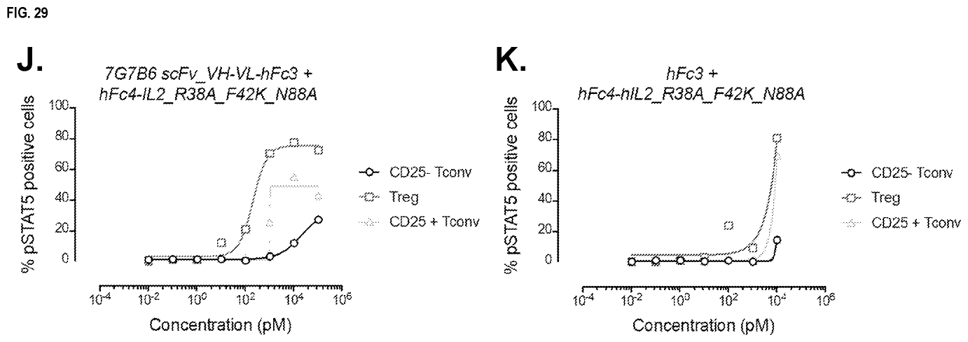

Then, the ability of CD25 targeting (here, using the 7G7B6 scFv_VH-VL targeting domain) to restore the loss in biological activity of CD25 (here, F38A_F42K) and IL2Rbeta mutations (here, N88A and N88G) or combinations thereof (R38A_F42K_N88A and R38A_F42K_N88G) was investigated. hFc4-hIL-2 constructs with these mutations were combined with CD8 VHH-hFc3 or with hFc3 constructs for production and purification in ExpiCHO cells. Resulting ALN2 variants were compared for STAT5 phosphorylation in CD25⁻ Tconv, CD25⁺ Tconv and Treg cells as described earlier. Data in FIGS. 29A-N illustrate that:
i. CD25 targeting has no clear effect on wild type Fc-IL-2 signaling (FIG. 29B vs FIG. 29C)
ii. CD25 or IL2Rbeta mutations clearly impact signaling in all tested sub-populations (FIG. 29C vs FIG. 29F, FIG. 29H, FIG. 29J, FIG. 29L and FIG. 29N). Combined CD25 and beta mutations (R38A_F42K_N88G) completely abolishes STAT5 phosphorylation up to concentrations of 100 nM (FIG. 29N)
iii. CD25 targeting clearly restores activity of both CD25 and IL2Rbeta ALN2 variants in CD25 expressing cells (FIG. 29E vs FIG. 29F and FIG. 29G vs FIG. 29H). This results in variants which only signal in CD25⁺ but no longer in CD25⁻ population
iv. Restoration of the combined mutations (R38A_F42K_N88A and R38A_F42K_N88G) is only partial when compared to the wild type Fc-IL-2 fusion (FIG. 29B vs FIG. 29K and FIG. 29B vs FIG. 29M)

In conclusion, CD25 targeting (as illustrated here by scFv formats of non-neutralizing anti-CD25 antibodies) allows specific restoration of loss-of-function caused by CD25 and/or β mutations in CD25 expressing cells vs. CD25 negative cells.

### Example 6: Targeting of IL-2 Mimic (Neoleukin) and Mutants Thereof

Based on the crystal structure of IL-2, an IL-2/IL-15 mimic that lacks the CD25 binding-site but still can efficiently signal through IL-2Rβ and γ_{c} receptors was designed de novo (see, Silva et al., “De novo design of potent and selective mimics of IL-2 and IL-15,” Nature, vol. 565, pp. 186-191, 2019). This 101 amino acid IL-2_mimic elicits downstream cell signaling (e.g. STAT5 phosphorylation) independently of IL-2Rα and IL-15Rα, and has superior therapeutic activity to IL-2 in mouse models of melanoma and colon cancer.

In this example, this IL-2_mimic sequence was fused via a flexible linker 20*GGS-linker C-terminally of an hIgG1 Fc with effector and knob mutations (hFc4-IL-2_mimic; see sequence below). In this construct, residue D15 (equivalent to D20 in human IL-2) was mutated to T or H, and residue N40 (equivalent to N88 in human IL-2) was mutated to I, G, and R in order to try to attenuate binding to the IL-2Rβ-chain. Resulting constructs (wild type or mutant) were combined with CD8 VHH-hFc3 for production in ExpiCHO cells. Resulting IL-2_mimic (neoleukin) variants were tested for STAT5 phosphorylation in CD8⁺, CD25 Tconv, CD25⁺ Tconv and Treg cells as described in Example 1.

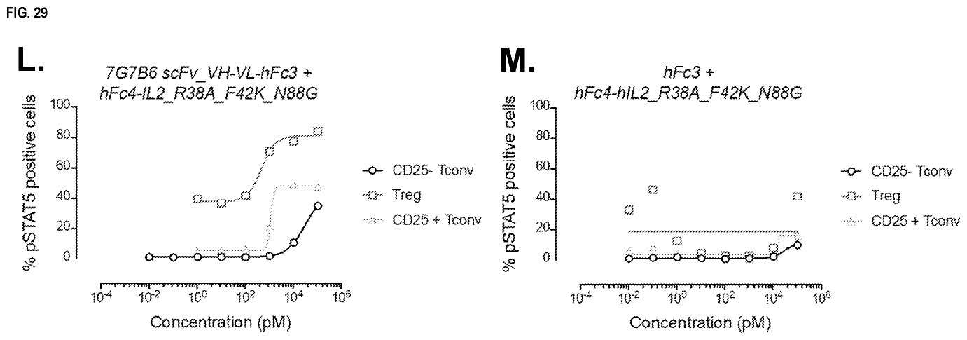

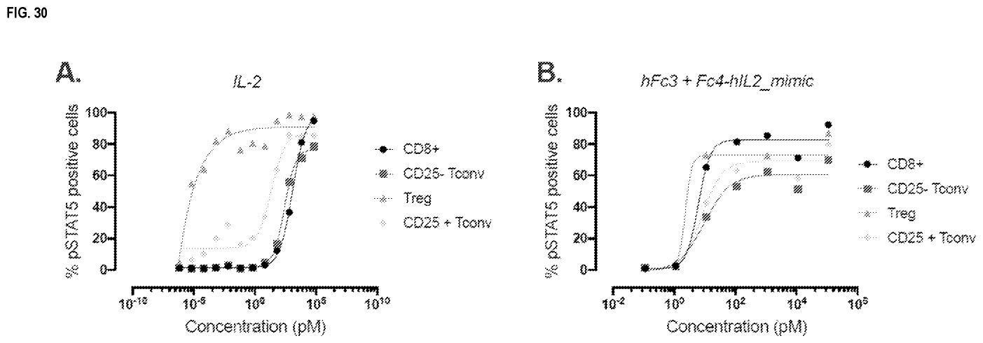

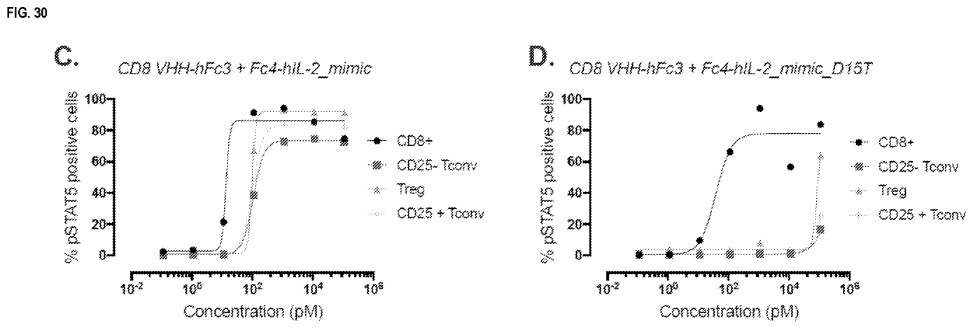

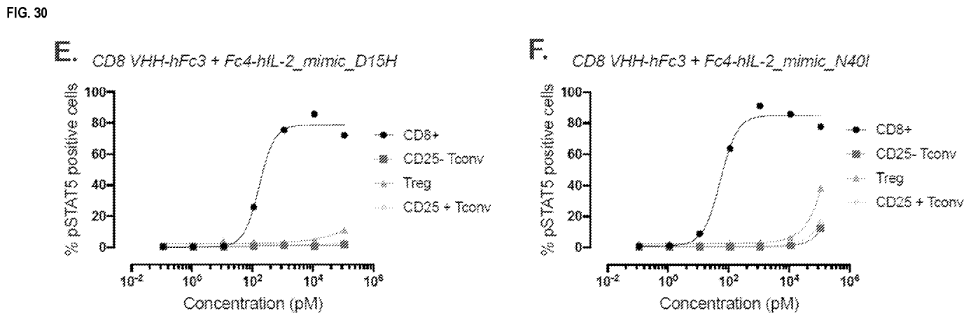

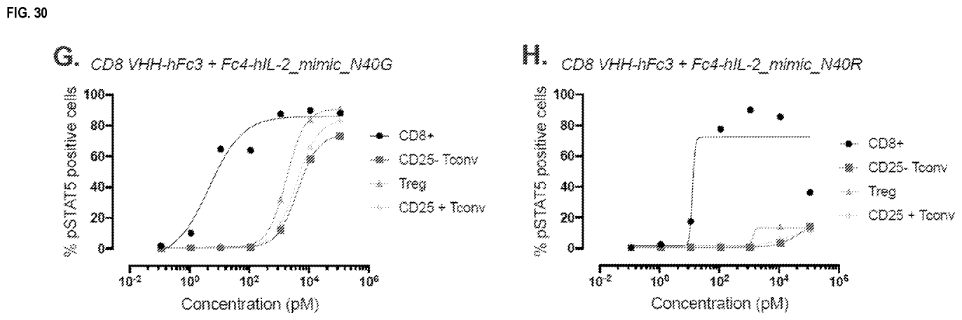

Data in FIGS. 30A-H illustrate that:
i. Different cell populations depicted the following sensitivity to wild type IL-2: CD8+<CD25− Tconv<CD25+ Tconv<<<Treg (FIG. 30A).
ii. An untargted IL-2_mimic Fc-variant is more or less equally active on all tested PBMC subpopulations (e.g., EC50 values of 6.1 pM; 9.6 pM; 2.3 pM; and 8.0 pM for respectively CD8+; CD25− Tconv; Treg; and CD25⁺ Tconv cells) (FIG. 30B).
iii. CD8 targeting of wild type IL-2_mimic increases signaling in CD8 positive cells, but not in other cell types (FIG. 30C) resulting in a selectivity index of about 10-fold.
iv. Mutations D15T, D15H, N401, and N40R almost completely abolished IL-2_mimic signaling in CD8 negative cells. Only at very high concentrations (100 nM) could a pSTAT5 signature be observed in these cells (FIGS. 30D, 30E, 30F and 30H).
V. CD8 targeting is able to restore, at least in part, loss of signaling resulting in EC50 values of 38 pM (D15T); 169 pM (D15H); 53 pM (N401); and 12 pM (N40R) (FIGS. 30D, 30E, 30F and 30H) and a selectivity index which is improved by 10-100 fold compared to targeted wild type IL-2 mimic in FIG. 30C.
vi. The loss-of-function is less severe for the N40G mutation, both on CD8 positive and CD8 negative cells (FIG. 30G) but the selectivity index is improved by 10-100 fold compared to targeted wild type IL-2 mimic in FIG. 30C.

In conclusion, CD8 targeting and mutation of residues D15 and N40 of IL-2_mimic clearly increases selectivity towards signaling in CD8 positive over CD8 negative cells. Targeting of wild type IL-2_mimic results only in a modest increase in selectivity.

Amino Acid Sequences of above Examples 1-6:
hFc3 (SEQ ID NO: 290)
hFc4-hIL-2_C125A (SEQ ID NO: 291)
hFc4-hIL-2_R38A_F42Y_Y45A_E62A_C125A (SEQ ID NO: 292)
hFc4-hIL-2_F42Y_Y45A_L72G_C125A (SEQ ID NO: 293)
hFc4-hIL-2_F42K_C125A (SEQ ID NO: 294)
hFc4-hIL-2_R38A_F42K_C125A (SEQ ID NO: 295)
hFc4-hIL-2_E61Q_C125A (SEQ ID NO: 296)
hCD8 VHH-hFc3 (SEQ ID NO: 297)
hFc4-hIL-2_D20A_C125A (SEQ ID NO: 298)
hFc4-hIL-2_D20E_C125A (SEQ ID NO: 299)
hFc4-hIL-2_D20F_C125A (SEQ ID NO: 300)
hFc4-hIL-2_D20G_C125A (SEQ ID NO: 301)
hFc4-hIL-2_D20H_C125A (SEQ ID NO: 302)
hFc4-hIL-2_D20I_C125A (SEQ ID NO: 303)
hFc4-hIL-2_D20K_C125A (SEQ ID NO: 304)
hFc4-hIL-2_D20L_C125A (SEQ ID NO: 305)
hFc4-hIL-2_D20N_C125A (SEQ ID NO: 306)
hFc4-hIL-2_D20P_C125A (SEQ ID NO: 307)
hFc4-hIL-2_D20Q_C125A (SEQ ID NO: 308)
hFc4-hIL-2_D20R_C125A (SEQ ID NO: 309)
hFc4-hIL-2_D20S_C125A (SEQ ID NO: 310)
hFc4-hIL-2_D20T_C125A (SEQ ID NO: 311)
hFc4-hIL-2_D20V_C125A (SEQ ID NO: 312)
hFc4-hIL-2_D20W_C125A (SEQ ID NO: 313)
hFc4-hIL-2_D20Y_C125A (SEQ ID NO: 314)
hFc4-hIL-2_N88A_C125A (SEQ ID NO: 315)
hFc4-hIL-2_N88D_C125A (SEQ ID NO: 316)
hFc4-hIL-2_N88E_C125A (SEQ ID NO: 317)
hFc4-hIL-2_N88F_C125A (SEQ ID NO: 318)
hFc4-hIL-2_N88G_C125A (SEQ ID NO: 319)
hFc4-hIL-2_N88H_C125A (SEQ ID NO: 320)
hFc4-hIL-2_N88I_C125A (SEQ ID NO: 321)
hFc4-hIL-2_N88K_C125A (SEQ ID NO: 322)
hFc4-hIL-2_N88L_C125A (SEQ ID NO: 323)
hFc4-hIL-2_N88P_C125A (SEQ ID NO: 324)
hFc4-hIL-2_N88Q_C125A (SEQ ID NO: 325)
hFc4-hIL-2_N88R_C125A (SEQ ID NO: 326)
hFc4-hIL-2_N88S_C125A (SEQ ID NO: 327)
hFc4-hIL-2_N88T_C125A (SEQ ID NO: 328)
hFc4-hIL-2_N88V_C125A (SEQ ID NO: 329)
hFc4-hIL-2_N88W_C125A (SEQ ID NO: 330)
hFc4-hIL-2_N88Y_C125A (SEQ ID NO: 331)
hFc4-hIL-2_D20E_R38A_F42K_C125A (SEQ ID NO: 332)
hFc4-hIL-2_D20V_R38A_F42K_C125A (SEQ ID NO: 333)
hFc4-hIL-2_ R38A_F42K_N88A_C125A (SEQ ID NO: 334)
hFc4-hIL-2_R38A_F42K_N88G_C125A (SEQ ID NO: 335)
MA251 scFv_VH-VL-hFc3 (SEQ ID NO: 336)
MA251 scFv_VL-VH-hFc3 (SEQ ID NO: 337)
7G7B6 scFv_VH-VL-hFc3 (SEQ ID NO: 338)
7G7B6 scFv_VL-VH-hFc3 (SEQ ID NO: 339)
hFc4-IL-2_mimic (SEQ ID NO: 340)
hFc4-IL-2_mimic_D15T (SEQ ID NO: 341)
hFc4-IL-2_mimic_D15H (SEQ ID NO: 342)
hFc4-IL-2_mimic_N40I (SEQ ID NO: 343)
hFc4-IL-2_mimic_N40G (SEQ ID NO: 344)
hFc4-IL-2_mimic_N40R (SEQ ID NO: 345)

### Example 7: Deletion of T3 O-Glycosylation in IL-2

IL-2 contains a threonine O-glycosylation site on position 3 (T3). In this example, the possibility to remove this site in the context of production, manufacturability and biological activity is evaluated. Two strategies are used: (i) site-specific mutations in which T3 is mutated to a A, F, H, L, V, or Y, or (ii) deletion-mutagenesis in which the first 3, 4, 5, 6, 7, or 8 residues of the IL-2 sequence are deleted. Mutations are made using standard mutagenesis techniques in the CD8-targeted ALN2 format. The IL-2 sequence is cloned (via a flexible 20*GGS linker)C-terminal of the hIgG1 Fc sequence with L234A_L235A_K322Q effector and S354C_T366W ‘knob’ mutations (referred to as hFc4). The IL-2 warhead contains following mutations: (i) R38A_F42K which block binding to CD25 or IL-2Ralpha, (ii) N88G that attenuates in a restorable fashion then interaction with IL-2Rbeta, and (iii) C125A which removes a free cysteine residue resulting in a superior manufacturability. The resulting construct is combined with a fusion of CD8 VHH 1CDA65 and hlG1 Fc with L234A_L235A_K322Q effector and Y349C_T366S_L368A_Y407V ‘hole’ mutations (referred to as hFc3). To produce this ‘knob-in-hole’ Fc-ALN2, a combination of both ‘hole’ and ‘knob’ plasmids is transfected in ExpiCHO cells (ThermoFisher) according to the manufacturer's instructions. One week after transfection, supernatant is collected, and cells removed by centrifugation. Recombinant proteins are purified based on protein A binding properties (Hitrap MabSelect SuRe column, GE Healthcare) and by subsequent size exclusion chromatography (Superdex 200 increase HiScale 16/40 column, GE Healthcare), both on an Äkta purifier (GE Healthcare). Concentrations are measured with a spectrophotometer (NanoDrop instrument, Thermo Scientific) and purity estimated on SDS-PAGE.

Resulting ALN2 O-glycosylation variants are tested for STAT5 phosphorylation in (i) CD8 positive T cells (CD8⁺); (ii) CD25 negative conventional T cells (CD25⁻ Tconv); (iii) CD25 positive conventional T cells (CD25⁺ Tconv); or (iv) regulatory T cells (Treg) defined as CD4⁺CD25+FoxP3+. In brief, PBMCs from buffy coats of healthy donors are isolated using density gradient centrifugation using Lymphoprep (StemCell technologies). Cells are stimulated with a serial dilution wild type recombinant IL-2 or Fc-ALN2 for 30 minutes at 37° C. After centrifugation, cells are resuspended in Lyse/Fix buffer (BD Biosciences) and further incubated for 10 minutes at 37° C. Cells are washed and incubated with human FcR Blocking Reagent (Miltenyi Biotec) and stained with anti-CD25 and anti-CD8 for 30 minutes at room temperature. Cells are subsequently permeabilized using the Perm Buffer III (BD Biosciences) at 4° C. for 30 minutes. Cells are finally stained with anti-CD3, anti-CD4, anti-FoxP3, and anti-pSTAT5 for 1 hour. Samples are acquired on a MACSQuant X instrument (Miltenyi Biotec) and analyzed using the FlowLogic software (Miltenyi Biotec).

To evaluate the stability of the O-glycosylation ALN2 variants, purified proteins are concentrated up to 10 mg/ml and subjected to five freeze (−20° C.)-thaw-cycles. After each cycle the sample are centrifuged, and protein concentrations measured on a Nanodrop spectrophotometer. Before and after the freeze-thaw cycles, the samples are analyzed by size exclusion chromatography (Superdex 200 increase HiScale 16/40 column, GE Healthcare) on an Äkta purifier.

The following amino acid sequences are used in this Example:
hCD8 VHH-hFc3 (SEQ ID NO: 346)
hFc4-hIL-2_R38A_F42K_N88G_C125A (SEQ ID NO: 347)
hFc4-hIL-2_T3A_R38A_F42K_N88G_C125A (SEQ ID NO: 348)
hFc4-hIL-2_T3F_R38A_F42K_N88G_C125A (SEQ ID NO: 349)
hFc4-hIL-2_T3H_R38A_F42K_N88G_C125A (SEQ ID NO: 350)
hFc4-hIL-2_T3L_R38A_F42K_N88G_C125A (SEQ ID NO: 351)
hFc4-hIL-2_T3V_R38A_F42K_N88G_C125A (SEQ ID NO: 352)
hFc4-hIL-2_T3Y_R38A_F42K_N88G_C125A (SEQ ID NO: 353)
hFc4-hIL-2_R38A_F42K_N88G_C125A_del3 (SEQ ID NO: 354)
hFc4-hIL-2_R38A_F42K_N88G_C125A_del4 (SEQ ID NO: 355)
hFc4-hIL-2_R38A_F42K_N88G_C125A_del5 (SEQ ID NO: 356)
hFc4-hIL-2_R38A_F42K_N88G_C125A_del6 (SEQ ID NO: 357)
hFc4-hIL-2_R38A_F42K_N88G_C125A_del7 (SEQ ID NO: 358)
hFc4-hIL-2_R38A_F42K_N88G_C125A_del8 (SEQ ID NO: 359)

### Example 8: Bi-Valent and Bi-Paratopic Targeting of IL-2 Activity

In this example, the efficiency of targeting of IL-2 activity to CD8 positive cells is compared using formats with one or two VHHs. These VHHs can be the same (resulting in a bi-valent configuration) or different (resulting in so-called bi-paratopic formats). On a knob-in-hole scaffold, VHHs and IL-2 warhead can be cloned in different configurations, as outlined in FIG. 31.

Specifically, seven different CD8 VHHs are cloned: 1CDA65, 2CDA5, 3CDA19, 2CDA47, 2CDA68, R2HCD26 and R3HCD129 in vectors encoding ALN2 variants with configuration 1 depicted in FIG. 31. As described in Example 7, the IL-2 warhead contains the R38A_F42K, N88G, and C125A mutations for respectively CD25 knock-out, restorable beta attenuation, and manufacturability. Resulting ALN2 formats are produced by transient transfection of ExpiCHO cells with following combinations of plasmids:
hCD8 VHH 1CDA65-hFc3+hFc4-IL-2_R38A_F42K_N88G_C125A
hCD8 VHH 1CDA65-hFc3+hCD8 VHH 1CDA65-hFc4-IL-2_R38A_F42K_N88G_C125A
hCD8 VHH 1CDA65-hFc3+hCD8 VHH 2CDA5-hFc4-IL-2_R38A_F42K_N88G_C125A
hCD8 VHH 1CDA65-hFc3+hCD8 VHH 3CDA19-hFc4-IL-2_R38A_F42K_N88G_C125A
hCD8 VHH 1CDA65-hFc3+hCD8 VHH 2CDA47-hFc4-IL-2_R38A_F42K_N88G_C125A
hCD8 VHH 1CDA65-hFc3+hCD8 VHH 2CDA68-hFc4-IL-2_R38A_F42K_N88G_C125A
hCD8 VHH 1CDA65-hFc3+hCD8 VHH R2HCD26-hFc4-IL-2_R38A_F42K_N88G_C125A
hCD8 VHH 1CDA65-hFc3+hCD8 VHH R3HCD129-hFc4-IL-2_R38A_F42K_N88G_C125A
hFc3+hCD8 VHH 1CDA65-hFc4-IL-2_R38A_F42K_N88G_C125A
hFc3+hCD8 VHH 2CDA5-hFc4-IL-2_R38A_F42K_N88G_C125A
hFc3+hCD8 VHH 3CDA19-hFc4-IL-2_R38A_F42K_N88G_C125A
hFc3+hCD8 VHH 2CDA47-hFc4-IL-2_R38A_F42K_N88G_C125A
hFc3+hCD8 VHH 2CDA68-hFc4-IL-2_R38A_F42K_N88G_C125A
hFc3+hCD8 VHH R2HCD26-hFc4-IL-2_R38A_F42K_N88G_C125A
hFc3+hCD8 VHH R3HCD129-hFc4-IL-2_R38A_F42K_N88G_C125A
hFc3+hFc4-IL-2_R38A_F42K_N88G_C125A

Resulting proteins are purified from the supernatans and tested for STAT5 phosphorylation in CD8⁺; CD25⁻ Tconv; CD25⁺ Tconv; and CD4⁺CD25⁺FoxP3⁺ Treg cells as described earlier.

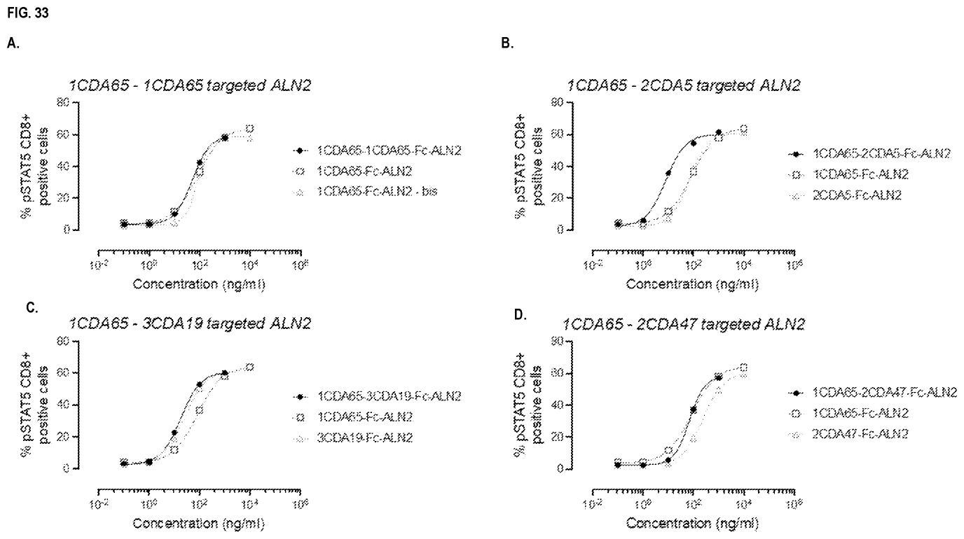

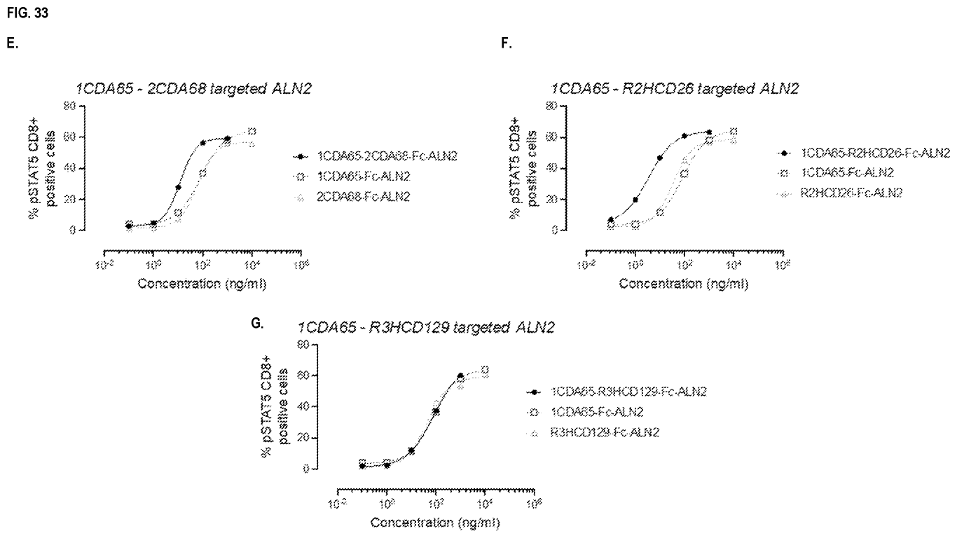

While the monospecific constructs have the configuration 4 of FIG. 32, the VHH 1CDA65 is a construct having the configuration 1 of FIG. 32 and was generated as a monospecific construct. It is referred to as 1CDA-65-Fc-ALN2 bis in FIGS. 33A-G. Data in FIGS. 33A-G illustrate that on CD8 cells:
i. The constructs 1CDA65-Fc-ALN2 and 1CDA65-Fc-ALN2 bis have similar potency;
ii. A bi-valent 1CDA65-1CDA65-Fc-ALN2 has similar activity as its mono-valent counterparts 1CDA65-Fc-ALN2 or 1CDA65-Fc-ALN2-bis; and
iii. A gain in biological activity is observed with the bi-paratopic configurations 1CDA65-2CDA5-Fc-ALN2, 1CDA65-2CDA68-Fc-ALN2 and 1CDA65-R2HCD26-Fc-ALN2. Of note, VHHs 1CDA65 and 2CDA4774 belong to the same epitope bin, while VHHs 2CDA5, 3CDA19, 2CDA68 and R2HCD26 belong to a second bin. Due to its lower affinity, no epitope bin was determined for VHH R3HCD129. Remarkably, ALN2 variants with two targeting domains from a different epitope bin are on average 10-fold more active than their mono-specific counterparts.

The following amino acid sequences were used in this Example:
hCD8 VHH 1CDA65-hFc3 (SEQ ID NO: 360)
hFc4-hIL-2_R38A_F42K_N88G_C125A (SEQ ID NO: 361)
hCD8 VHH 1CDA65-hFc4-IL-2_R38A_F42K_N88G_C125A (SEQ ID NO: 362)
hCD8 VHH 2CDA5-hFc4-IL-2_R38A_F42K_N88G_C125A (SEQ ID NO: 363)
hCD8 VHH 3CDA19-hFc4-IL-2_R38A_F42K_N88G_C125A (SEQ ID NO: 364)
hCD8 VHH 2CDA47-hFc4-IL-2_R38A_F42K_N88G_C125A (SEQ ID NO: 365)
hCD8 VHH 2CDA68-hFc4-IL-2_R38A_F42K_N88G_C125A (SEQ ID NO: 366)
hCD8 VHH R2HCD26-hFc4-IL-2_R38A_F42K_N88G_C125A (SEQ ID NO: 367)
hCD8 VHH R3HCD129-hFc4-IL-2_R38A_F42K_N88G_C125A (SEQ ID NO: 368)
hFc3 (SEQ ID NO: 369)

### Example 9: Evaluation of Alternative ALN2 Configurations

In this example, different CD8 targeted ALN2 configurations are compared. Specifically, VHH and/or IL-2 warhead are cloned N- or C-terminal of the knob-in-hole Fc scaffold, on the same or opposite sites of the molecule. Formats can harbor one or two warheads, cloned in tandem or on different Fc-chains. VHH and warheads can also be cloned in series, or VHH in between two IL-2 moieties. All possible configurations are schematically outlined in FIG. 32.

The following ALN2 plasmid combinations are transfected in ExpiCHO cells, purified from the supernatans, tested for pSTAT5 in CD8+; CD25⁻ Tconv; CD25⁺ Tconv; and CD4⁺CD25+FoxP3⁺ Treg cells and screened for stability as described earlier:
hCD8 VHH 1CDA65-hFc3+hFc4-IL-2_R38A_F42K_N88G_C125A (configuration 1 in FIG. 32)
hCD8 VHH 1CDA65-hFc3+IL-2_R38A_F42K_N88G_C125A-hFc4-IL-2_R38A_F42K_N88G_C125A (configuration 9 in FIG. 32)
hCD8 VHH 1CDA65-hFc3-hFc4-IL-2_R38A_F42K_N88G_C125A+hFc4-IL-2_R38A_F42K_N88G_C125A (configuration 13 in FIG. 32)
hCD8 VHH 1CDA65-hFc3+hFc4-IL-2_R38A_F42K_N88G_C125A-IL-2_R38A_F42K_N88G_C125A (configuration 25 in FIG. 32)
hCD8 VHH 1CDA65-hFc3+IL-2_R38A_F42K_N88G_C125A-IL-2_R38A_F42K_N88G_C125A-hFc4 (configuration 26 in FIG. 32)

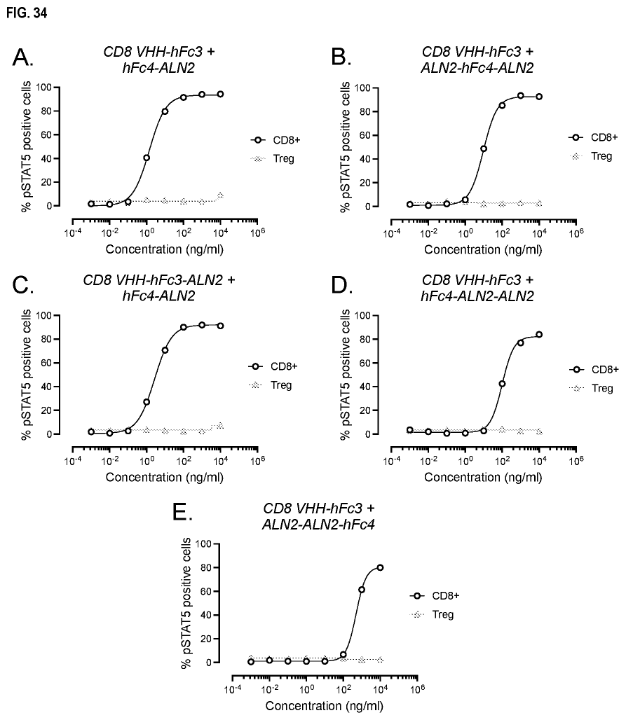

Further, data in FIGS. 34A-E illustrate that all tested variants gave clear pSTAT5 in CD8+ but not in Treg cells, thereby illustrating their selectivity.

The following amino acid sequences are used in this Example:
hCD8 VHH 1CDA65-hFc3 (SEQ ID NO: 370)
hFc4-hIL-2_R38A_F42K_N88G_C125A (SEQ ID NO: 371)
IL-2_R38A_F42K_N88G_C125A-hFc4-IL-2_R38A_F42K_N88G_C125A (SEQ ID NO: 372)
hCD8 VHH 1CDA65-hFc3-hFc4-IL-2_R38A_F42K_N88G_C125A (SEQ ID NO: 373)
hFc4-IL-2_R38A_F42K_N88G_C125A-IL-2_R38A_F42K_N88G_C125A (SEQ ID NO: 374)
IL-2_R38A_F42K_N88G_C125A-IL-2_R38A_F42K_N88G_C125A-hFc4 (SEQ ID NO: 375)

### Example 10: Combination of Beta and Gamma Mutations

The purpose of this example is to evaluate the effect of combining a loss-of-function mutation in the IL-2Rbeta binding-site (N88A, N88G, D20E or D20V) with a mutation that hampers interaction with the γ. chain in the IL-2R. For this latter mutation, residue Q126 has been identified as being crucial and is mutated.

The mutations are summarized in Table 8:

| Binding-site | Mutations |
|---|---|
| Beta | D20E, D20V, N88A, and N88G |
| Gamma | Q126A, Q126D, Q126E, Q126F, Q126G, Q126H, Q1261, Q126K, Q126L, Q126M, |
| | Q126N, Q126P, Q126R, Q126S, Q126T, Q126V, Q126W, and Q126Y |
| Beta-gamma | D20E_Q126A, D20E_Q126D, D20E_Q126E, D20E_Q126F, D20E_Q126G, |
| | D20E_Q126H, D20E_Q1261, D20E_Q126K, D20E_Q126L, D20E_Q126M, |
| | D20E_Q126N, D20E_Q126P, D20E_Q126R, D20E_Q126S, D20E_Q126T, |
| | D20E_Q126V, D20E_Q126W, D20E_Q126Y, D20V_Q126A, D20V_Q126D, |
| | D20V_Q126E, D20V_Q126F, D20V_Q126G, D20V_Q126H, D20V_Q1261, |
| | D20V_Q126K, D20V_Q126L, D20V_Q126M, D20V_Q126N, D20V_Q126P, |
| | D20V_Q126R, D20V_Q126S, D20V_Q126T, D20V_Q126V, D20V_Q126W, |
| | D20V_Q126Y, N88A_Q126A, N88A_Q126D, N88A_Q126E, N88A_Q126F, |
| | N88A_Q126G, N88A_Q126H, N88A_Q1261, N88A_Q126K, N88A_Q126L, |
| | N88A_Q126M, N88A_Q126N, N88A_Q126P, N88A_Q126R, N88A_Q126S, |
| | N88A_Q126T, N88A_Q126V, N88A_Q126W, N88A_Q126Y, N88G_Q126A, |
| | N88G_Q126D, N88G_Q126E, N88G_Q126F, N88G_Q126G, N88G_Q126H, |
| | N88G_Q1261, N88G_Q126K, N88G_Q126L, N88G_Q126M, N88G_Q126N, |
| | N88G_Q126P, N88G_Q126R, N88G_Q126S, N88G_Q126T, N88G_Q126V, |
| | N88G_Q126W, and N88G_Q126Y |

Mutations or combinations thereof are made using standard mutagenesis techniques in the CD8-targeted ALN2 format (as described in examples above). Here, the IL-2 warhead contains only the C125A mutation for manufacturability reasons. Resulting plasmids are combined with CD8 VHH-hFc3 plasmid for transient transfection in ExpiCHO cells. Proteins are purified using protein A spin plates (ThermoFisher), quantified and purity tested using SDS-PAGE.

Resulting ALN2 variants are tested for STAT5 phosphorylation in CD8⁺, CD4⁺CD25, and CD4⁺CD25⁺ PBMC populations as follows: PBMCs are blocked human FcR Blocking Reagent (Miltenyi Biotec) and stained fluorescently labelled Ab's specific for CD4, CD8, and CD25. After staining and washing, cells are stimulated with a serial dilution IL-2 or ALN2 variants for 30 min at 37° C. Cells are subsequently fixed with Lyse/Fix buffer (BD Biosciences) and permeabilized using the Perm Buffer III (BD Biosciences) according to the manufacturer's guidelines. After overnight staining with a pSTAT5 specific Ab, samples are analyzed on a MACSQuant X instrument (Miltenyi Biotec) and analyzed using the FlowLogic software (Miltenyi Biotec).

The following amino acid sequences are used in this Example:
hCD8 VHH-hFc3 (SEQ ID NO: 376)
hFc4-hIL-2_C125A (SEQ ID NO: 377)
hFc4-hIL-2_C125A_D20E_Q126A (SEQ ID NO: 378)
hFc4-hIL-2_C125A_D20E_Q126D (SEQ ID NO: 379)
hFc4-hIL-2_C125A_D20E_Q126E (SEQ ID NO: 380)
hFc4-hIL-2_C125A_D20E_Q126F (SEQ ID NO: 381)
hFc4-hIL-2_C125A_D20E_Q126G (SEQ ID NO: 382)
hFc4-hIL-2_C125A_D20E_Q126H (SEQ ID NO: 383)
hFc4-hIL-2_C125A_D20E_Q126I (SEQ ID NO: 384)
hFc4-hIL-2_C125A_D20E_Q126K (SEQ ID NO: 385)
hFc4-hIL-2_C125A_D20E_Q126L (SEQ ID NO: 386)
hFc4-hIL-2_C125A_D20E_Q126M (SEQ ID NO: 387)
hFc4-hIL-2_C125A_D20E_Q126N (SEQ ID NO: 388)
hFc4-hIL-2_C125A_D20E_Q126P (SEQ ID NO: 389)
hFc4-hIL-2_C125A_D20E_Q126R (SEQ ID NO: 390)
hFc4-hIL-2_C125A_D20E_Q126S (SEQ ID NO: 391)
hFc4-hIL-2_C125A_D20E_Q126T (SEQ ID NO: 392)
hFc4-hIL-2_C125A_D20E_Q126V (SEQ ID NO: 393)
hFc4-hIL-2_C125A_D20E_Q126W (SEQ ID NO: 394)
hFc4-hIL-2_C125A_D20E_Q126Y (SEQ ID NO: 395)
hFc4-hIL-2_C125A_D20V_Q126A (SEQ ID NO: 396)
hFc4-hIL-2_C125A_D20V_Q126D (SEQ ID NO: 397)
hFc4-hIL-2_C125A_D20V_Q126E (SEQ ID NO: 398)
hFc4-hIL-2_C125A_D20V_Q126F (SEQ ID NO: 399)
hFc4-hIL-2_C125A_D20V_Q126G (SEQ ID NO: 400)
hFc4-hIL-2_C125A_D20V_Q126H (SEQ ID NO: 401)
hFc4-hIL-2_C125A_D20V_Q126I (SEQ ID NO: 402)
hFc4-hIL-2_C125A_D20V_Q126K (SEQ ID NO: 403)
hFc4-hIL-2_C125A_D20V_Q126L (SEQ ID NO: 404)
hFc4-hIL-2_C125A_D20V_Q126M (SEQ ID NO: 405)
hFc4-hIL-2_C125A_D20V_Q126N (SEQ ID NO: 406)
hFc4-hIL-2_C125A_D20V_Q126P (SEQ ID NO: 407)
hFc4-hIL-2_C125A_D20V_Q126R (SEQ ID NO: 408)
hFc4-hIL-2_C125A_D20V_Q126S (SEQ ID NO: 409)
hFc4-hIL-2_C125A_D20V_Q126T (SEQ ID NO: 410)
hFc4-hIL-2_C125A_D20V_Q126V (SEQ ID NO: 411)
hFc4-hIL-2_C125A_D20V_Q126W (SEQ ID NO: 412)
hFc4-hIL-2_C125A_D20V_Q126Y (SEQ ID NO: 413)
hFc4-hIL-2_C125A_N88A_Q126A (SEQ ID NO: 414)
hFc4-hIL-2_C125A_N88A_Q126D (SEQ ID NO: 415)
hFc4-hIL-2_C125A_N88A_Q126E (SEQ ID NO: 416)
hFc4-hIL-2_C125A_N88A_Q126F (SEQ ID NO: 417)
hFc4-hIL-2_C125A_N88A_Q126G (SEQ ID NO: 418)
hFc4-hIL-2_C125A_N88A_Q126H (SEQ ID NO: 419)
hFc4-hIL-2_C125A_N88A_Q126I (SEQ ID NO: 420)
hFc4-hIL-2_C125A_N88A_Q126K (SEQ ID NO: 421)
hFc4-hIL-2_C125A_N88A_Q126L (SEQ ID NO: 422)
hFc4-hIL-2_C125A_N88A_Q126M (SEQ ID NO: 423)
hFc4-hIL-2_C125A_N88A_Q126N (SEQ ID NO: 424)
hFc4-hIL-2_C125A_N88A_Q126P (SEQ ID NO: 425)
hFc4-hIL-2_C125A_N88A_Q126R (SEQ ID NO: 426)
hFc4-hIL-2_C125A_N88A_Q126S (SEQ ID NO: 427)
hFc4-hIL-2_C125A_N88A_Q126T (SEQ ID NO: 428)
hFc4-hIL-2_C125A_N88A_Q126V (SEQ ID NO: 429)
hFc4-hIL-2_C125A_N88A_Q126W (SEQ ID NO: 430)
hFc4-hIL-2_C125A_N88A_Q126Y (SEQ ID NO: 431)
hFc4-hIL-2_C125A_N88G_Q126A (SEQ ID NO: 432)
hFc4-hIL-2_C125A_N88G_Q126D (SEQ ID NO: 433)
hFc4-hIL-2_C125A_N88G_Q126E (SEQ ID NO: 434)
hFc4-hIL-2_C125A_N88G_Q126F (SEQ ID NO: 435)
hFc4-hIL-2_C125A_N88G_Q126G (SEQ ID NO: 436)
hFc4-hIL-2_C125A_N88G_Q126H (SEQ ID NO: 437)
hFc4-hIL-2_C125A_N88G_Q126I (SEQ ID NO: 438)
hFc4-hIL-2_C125A_N88G_Q126K (SEQ ID NO: 439)
hFc4-hIL-2_C125A_N88G_Q126L (SEQ ID NO: 440)
hFc4-hIL-2_C125A_N88G_Q126M (SEQ ID NO: 441)
hFc4-hIL-2_C125A_N88G_Q126N (SEQ ID NO: 442)
hFc4-hIL-2_C125A_N88G_Q126P (SEQ ID NO: 443)
hFc4-hIL-2_C125A_N88G_Q126R (SEQ ID NO: 444)
hFc4-hIL-2_C125A_N88G_Q126S (SEQ ID NO: 445)
hFc4-hIL-2_C125A_N88G_Q126T (SEQ ID NO: 446)
hFc4-hIL-2_C125A_N88G_Q126V (SEQ ID NO: 447)
hFc4-hIL-2_C125A_N88G_Q126W (SEQ ID NO: 448)
hFc4-hIL-2_C125A_N88G_Q126Y (SEQ ID NO: 449)

### Example 11: Single-Peptide ALN2 Variants

This Example shows the effects of single-peptide ALN2 variants. The no-alpha IL-2 variant (mutations R38A_F42K) was cloned into a single-peptide A-Kine format. To this end, sequences encoding the CD8 VHH 1CDA65 and IL-2 were fused via a flexible 20*GGS-linker in the pcDNA3.4 expression vector for eukaryotic expression. A C-terminal 6*His tag was added for purification purposes. D20E, D20V, N88A, and N88G mutants were made using standard mutagenesis techniques and resulting plasmids were transfected in ExpiCHO cells (ThermoFisher) according to the manufacturer's guidelines. One week after transfection, supernatant was collected, and cells removed by centrifugation. Recombinant proteins were purified based on the His-tag (His Trap Excel column; Cytiva) and by subsequent size exclusion chromatography (Superdex 200 increase HiScale 16/40 column, Cytiva), both on an Äkta purifier (GE Healthcare). Concentrations were measured with a spectrophotometer (NanoDrop instrument, Thermo Scientific) and purity was estimated on SDS-PAGE.

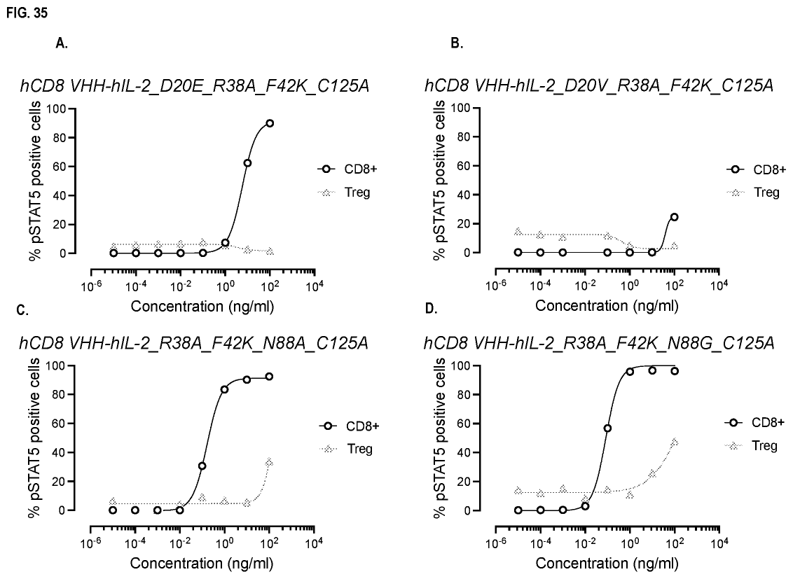

The resulting proteins were tested for STAT5 phosphorylation in CD8⁺ versus Treg cells, as described earlier. As shown in FIGS. 35A-D, all except for the mutation R38A_F42K_D20V allow, in the concentration range tested, selective signaling for CD8+ over Treg cells. The N88 variants R38A_F42K_N88A (EC₅₀: 0.17 ng/ml) or R38A_F42K_N88G (EC₅₀: 0.08 ng/ml) variants appear more potent than the R38A_F42K_D20E mutant (EC₅₀: 5.8 ng/ml) on CD8⁺ cells.

The following amino acid sequences were used in this Example:
hCD8 VHH-hIL-2_D20E_R38A_F42K_C125A (SEQ ID NO: 478)
hCD8 VHH-hIL-2_D20V_R38A_F42K_C125A (SEQ ID NO: 479)
hCD8 VHH-hIL-2_R38A_F42K_N88A_C125A (SEQ ID NO: 480)
hCD8 VHH-hIL-2_R38A_F42K_N88G_C125A (SEQ ID NO: 481)

### Example 12: Removal of T3 O-Glycosylation Site in IL-2 Warhead

IL-2 contains a threonine O-glycosylation site on position 3 (T3). In this example, the possibility to remove this site in the context of production, manufacturability and biological activity is evaluated. Two strategies are used: (i) site-specific mutations in which T3 is mutated to a A, F, H, L, V, or Y, or (ii) deletion-mutagenesis in which the first 3, 4, 5, 6, 7, or 8 residues of the IL-2 sequence are deleted. Mutations were made using standard mutagenesis techniques in the CD8-targeted ALN2 format. The IL-2 sequence was cloned (via a flexible 20*GGS linker)C-terminal of the hIgG1 Fc sequence with L234A_L235A_K322Q effector and S354C_T366W ‘knob’ mutations (referred to as hFc4). The IL-2 warhead contains following mutations: (i) R38A_F42K which block binding to CD25 or IL-2Ralpha, (ii) N88G that attenuates in a restorable fashion then interaction with IL-2Rbeta, and (iii) C125A which removes a free cysteine residue resulting in a superior manufacturability. The resulting construct was combined with a fusion of CD8 VHH 1CDA65 and hlG1 Fc with L234A_L235A_K322Q effector and Y349C_T366S_L368A_Y407V ‘hole’ mutations (referred to as hFc3). To produce this ‘knob-in-hole’ Fc-ALN2, a combination of both ‘hole’ and ‘knob’ plasmids was transfected in ExpiCHO cells (ThermoFisher) according to the manufacturer's instructions. One week after transfection, supernatant was collected, and cells removed by centrifugation. Recombinant proteins were purified using protein A spin plates (ThermoFisher), quantified and purity tested using SDS-PAGE. Resulting variants were tested for STAT5 phosphorylation in CD8 positive (CD8⁺), and CD8 negative (CD8−) PBMC populations as follows: PBMCs were blocked human FcR Blocking Reagent (Miltenyi Biotec) and stained fluorescently labelled Ab's specific for CD8. After staining and washing, cells were stimulated with a serial dilution CD8 VHH-Fc-IL-2 variants as indicated for 30 min at 37° C. Cells were subsequently fixed with Lyse/Fix buffer (BD Biosciences) and permeabilized using the Perm Buffer III (BD Biosciences) according to the manufacturer's guidelines. After overnight staining with a pSTAT5 specific Ab, samples were analyzed on a MACSQuant X instrument (Miltenyi Biotec) and analyzed using the FlowLogic software (Miltenyi Biotec).

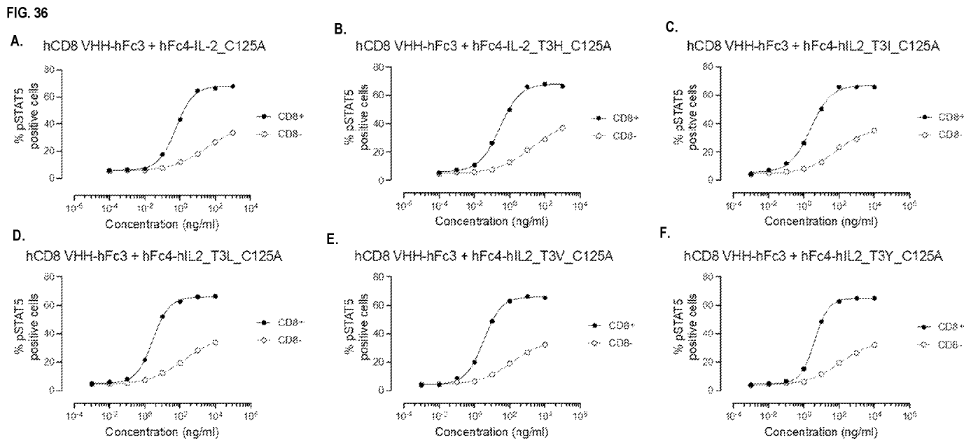

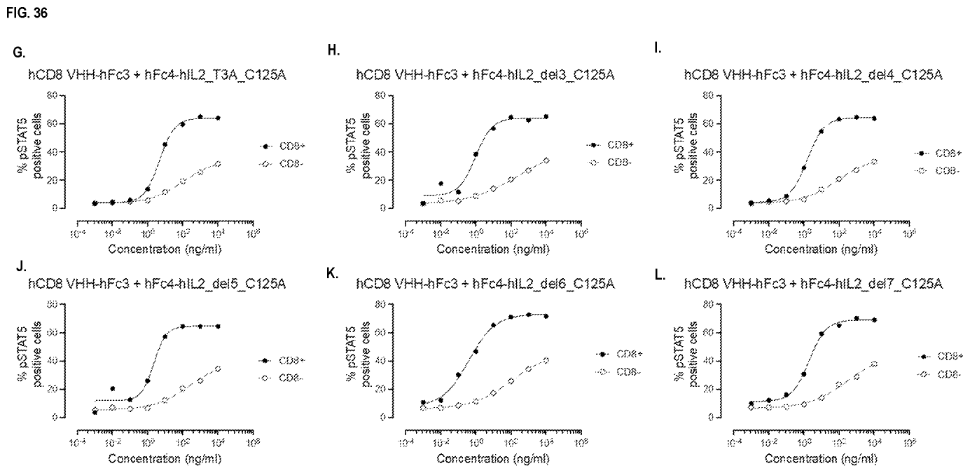

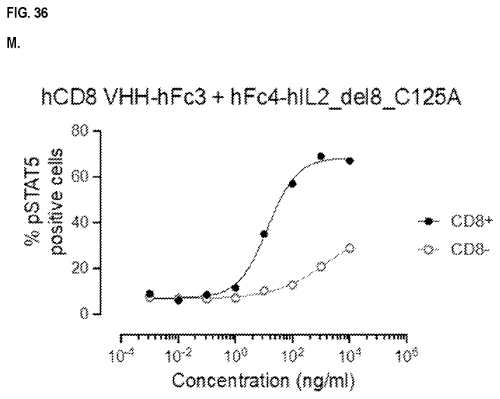

Data in FIGS. 36A-M illustrate that substitution of T3 to H, I, L V, Y or A, or deletion of up to 7 amino acids, have a comparable activity on CD8⁺ cells with EC50 values varying from 1 to 5 ng/ml. However, the IL-2_del8 variant was 10-fold less active compared to wild type IL-2. In contrast, the T3F (not shown) mutation no longer showed detectable activity. Effects on signaling in CD8− populations were shown to be very similar.

The following amino acid sequences were used in this Example:
hFc4-hIL-2_C125A (SEQ ID NO: 482)
hCD8 VHH-hFc3 (SEQ ID NO: 483)
hFc4-hIL-2_T3A_C125A (SEQ ID NO: 484)
hFc4-hIL-2_T3H_C125A (SEQ ID NO: 485)
hFc4-hIL-2_T3L_C125A (SEQ ID NO: 486)
hFc4-hIL-2_T3V_C125A (SEQ ID NO: 487)
hFc4-hIL-2_T3Y_C125A (SEQ ID NO: 488)
hFc4-hIL-2_C125A_del3 (SEQ ID NO: 489)
hFc4-hIL-2_C125A_del4 (SEQ ID NO: 490)
hFc4-hIL-2_C125A_del5 (SEQ ID NO: 491)
hFc4-hIL-2_C125A_del6 (SEQ ID NO: 492)
hFc4-hIL-2_C125A_del7 (SEQ ID NO: 493)
hFc4-hIL-2_C125A_del8 (SEQ ID NO: 494)

### Example 13: In Vivo Evaluation of the Alpha: Beta Combination

Since human IL-2 is cross-reactive on the mouse IL-2 receptor, the activity of the human alpha:beta ALN2 variants on mouse cells was tested. To this end, the VHH specific for human CD8 (1CDA65) was replaced by the mouse CD8 specific VHH R2CDE47 in the VHH-Fc fusion with effector and whole mutations. The resulting construct (mCD8 VHH-hFc3) was combined with the knob Fc fused to the ALN2 warheads with the R38A_F42K alpha knock-out mutation and one of the restorable beta mutations D20E, D20V, N88A or N88G. Mouse CD8 targeted ALN2 variants were expressed in ExpiCHO and purified as described above. Initially, these variants were tested for STAT5 phosphorylation in primary mouse splenocytes. In brief, splenocytes (RBC lysed with ACK buffer) from C57BI/6 mice were stained for cell surface markers (Viability, CD3, CD4, CD8 alpha, CD8 beta) and stimulated ex vivo for 30 minutes different mouse CD8 targeted ALN2 variants. Stimulation was stopped by adding 1×TFP Fix/Perm buffer (BD Pharmingen) and incubated for 50 minutes at 4° C., followed by 2 wash steps with 1×TFP Perm/Wash (BD Pharmingen). Cell pellets were resuspended in Perm/Wash III (Transcription Factor Phospho buffer set, BD Pharmingen) and incubated ON at −20° C. followed by intracellular FoxP3 and pSTAT5 staining. Samples were acquired on a MACSQuant16 flow cytometer and analyzed with FlowLogic 8.4 Software (Miltenyi Biotec).

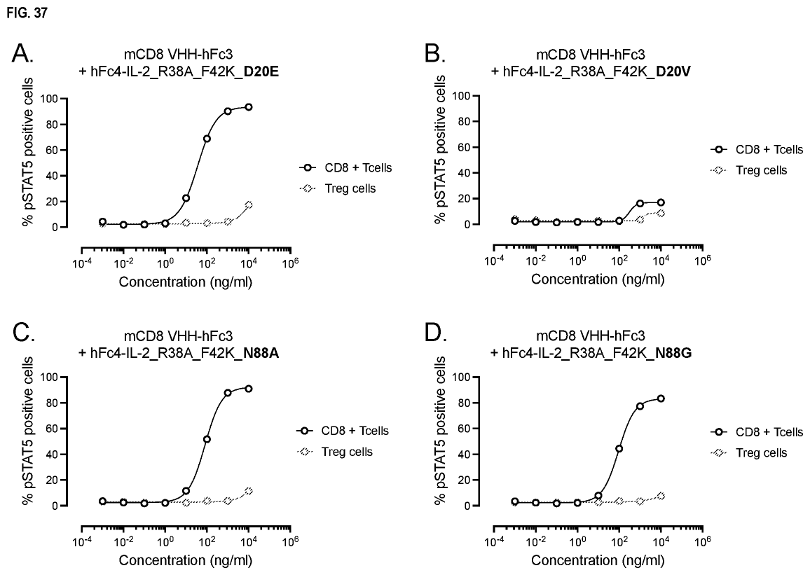

Activity on CD8⁺ cells for the D20E, N88A or N88G combinations were comparable (EC50 values between 36 and 94 ng/ml), while only signaling at very high concentrations could be observed in Treg cells for these variants (FIGS. 37A-D). The D20V combo mutant appears inactive on both tested splenocyte subpopulations. Based on these data and on preliminary production and stability observations, the mouse CD8 targeted human R38A_F42K_N88G IL-2 mutant was used (in the rest of this example referred to as CD8-Fc-ALN2) for further in vivo evaluation.

The anti-tumor efficacy of the mouse CD8 targeted ALN2 variant (CD8-Fc-ALN2) with combined mutations R38A_F42K_N88G was evaluated in two syngeneic murine models for colon cancer, MC38 and CT26, and compared to an untargeted ALN2 variant (Fc-ALN2). In brief, C57BI/6J mice (female, 9 weeks old) were injected subcutaneously (s.c.) with 6E+5 MC38 cells or Balb/c mice (female, 9 weeks old) with 6E+5 CT26 cells. Seven days later, tumors volumes reached 20-90 mm³ (MC38) or 40-120 mm³ (CT26), and treatments were started: mice received intravenously (i.v.) either buffer, or equimolar amounts untargeted Fc-ALN2 (21 μg/mouse) or targeted CD8-Fc-ALN2 (25 μg/mouse) on days 7 and 14 after tumor inoculation.

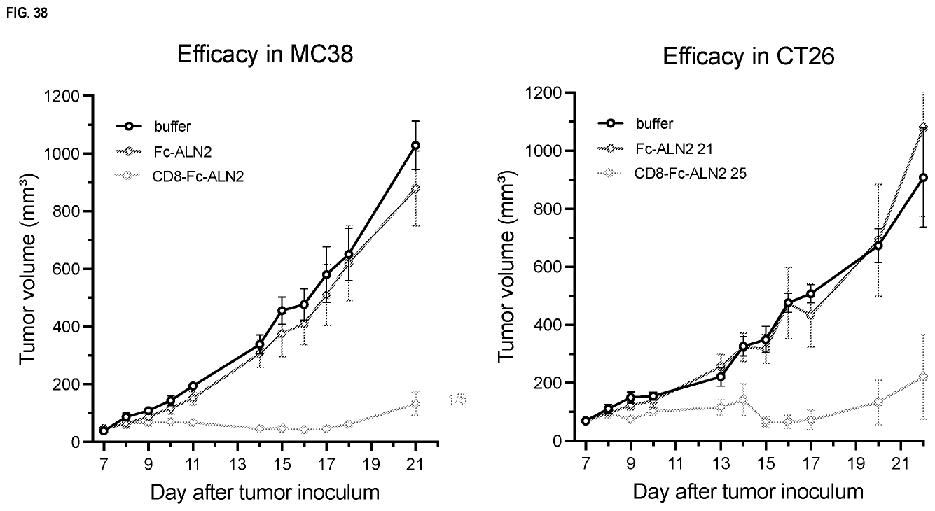

Tumor sizes (means+/−SEM; n=5 per group) were measured daily and plotted in FIG. 38. The data clearly illustrate that while the untargeted ALN2 had, compared to the buffer treated animals, no significant effect, the CD8-Fc-ALN2 clearly and substantially inhibited tumor growth in both models. At day 21, even one CD8-Fc-ALN2 treated animal was completely tumor-free in the MC38 model.

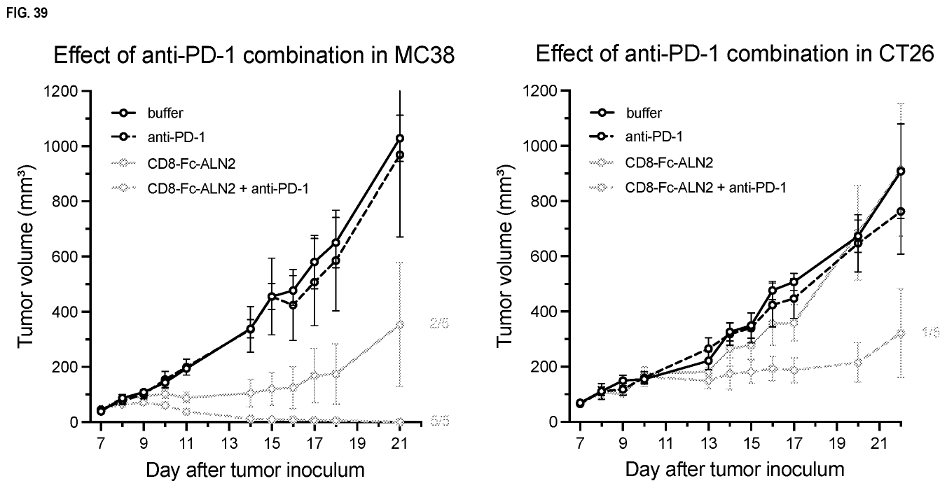

In a parallel experiment, the combined action of the CD8-Fc-ALN2 and an anti-PD-1 Ab as check-point inhibitor in both MC38 and CT26 models were investigated. To this end, tumor-bearing C57BI/6J (MC38) or Balb/c mice (CT26) received i.v. buffer or CD8-Fc-ALN2 (12.5 μg/mouse) on days 7, 10, 14 and 17 after tumor inoculation. This buffer or ALN2 treatment was supplemented by intraperitoneal (i.p.) anti-PD-1 Ab treatments (200 μg/mouse) on the same days as the i.v. treatments. Shown in FIG. 39 are tumor sizes as means+/−SEM (n=5 per group). The number at the end of each curve indicates the amount of completely tumor-free mice at the end of the experiment (day 22). In the MC38 model, anti-PD-1 did not have an effect on its own, the CD8-Fc-ALN2 alone had an intermediate effect (with 2 out of 5 mice completely tumor-free), but the combination resulted in a complete tumor regression in all animals. A similar combined/additional effect was observed in the CT26 model: while both monotherapies (CD8-Fc-ALN2 and anti-PD-1) had no significant effect, the combined treatment slowed tumor growth and resulted even in complete eradication in one animal.

Of note, tumor-free MC38 mice were subsequently re-challenged on the other flank 56 days after the first MC38 tumor inoculation. All tumor-free and re-challenged mice were still completely tumor-free 2 months later, while control animals developed a tumor that necessitated euthanasia (reaching 1500 mm³) 17 to 21 days after tumor inoculation.

The following amino acid sequences were used in the Example:
mCD8 VHH-hFc3 (SEQ ID NO: 495)
hFc4-hIL-2_D20E_R38A_F42K_C125A (SEQ ID NO: 332)
hFc4-hIL-2_D20V_R38A_F42K_C125A (SEQ ID NO: 333)
hFc4-hIL-2_ R38A_F42K_N88A_C125A (SEQ ID NO: 334)
hFc4-hIL-2_R38A_F42K_N88G_C125A (SEQ ID NO: 335)
hFc3 (SEQ ID NO: 369)

### Example 14: Screening of Gamma Common Mutations

In a cancer context it may be desirable to selectively activate cytotoxic T lymphocytes (CTLs) with IL-2 while minimizing the effects on the Treg cells that dampen these immune responses. One therapeutic strategy is to use these so-called no-alpha IL-2 variants that no longer bind the IL-2R alpha chain that is mainly causing the IL-2 hyper-sensitivity of Treg cells. As described above, a CD8-targeted no-alpha Fc variant is still able to activate Treg cells with an EC50 of 1 to 2 ng/ml. The combination with a restorable (upon CD8 targeting) IL-2R beta mutation (D20E, D20V, N88A, or N88G) further decreases Treg activation and improves the selectivity towards CD8 expressing cells (see Example 4; FIGS. 26A-J).

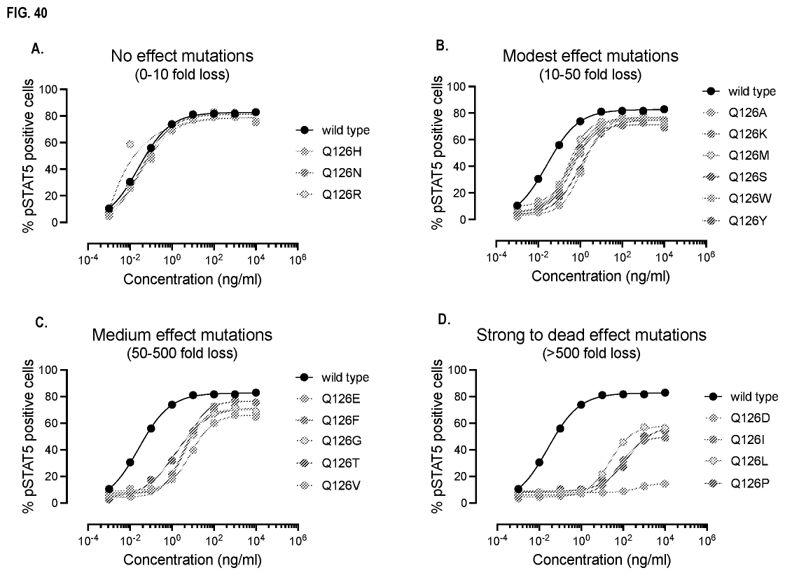

In this example, mutation of the interaction-site with gamma common receptor was evaluated to evaluate to what extent such a mutation has the same beneficial effect as the beta mutations. Likely candidates for A-Kine mutation are substitutions of residue Q126, central in the interaction between IL-2 and the gamma common chain. Therefore, this residue was mutated to any other (except for cysteine) amino acid, in the Fc4-IL-2_C125A chain and combined with CD8 VHH-hFc3 for production in ExpiCHO. One week after transfection, proteins were purified from the supernatans using protein A spin plates (ThermoFisher), quantified, and purity tested using SDS-PAGE. The effect of these mutations was compared to wild type IL-2 (with C125A mutation) on STAT5 phosphorylation in CD8 positive PBMCs, as described earlier. Based on these responses, mutations were classified in four categories (FIGS. 40A-D):
i. No effect mutations (0-10-fold loss in activity)
ii. Modest effect mutations (10-50-fold loss in activity)
iii. Medium effect mutations (50-500-fold loss in activity)
iv. Strong mutations (>500-fold loss in activity) or in case of Q126D almost complete loss in activity

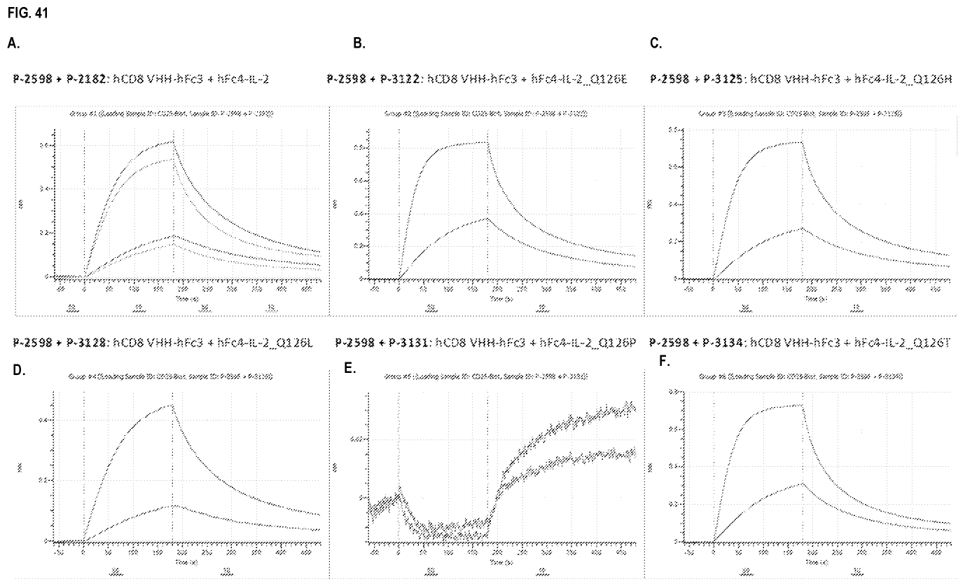

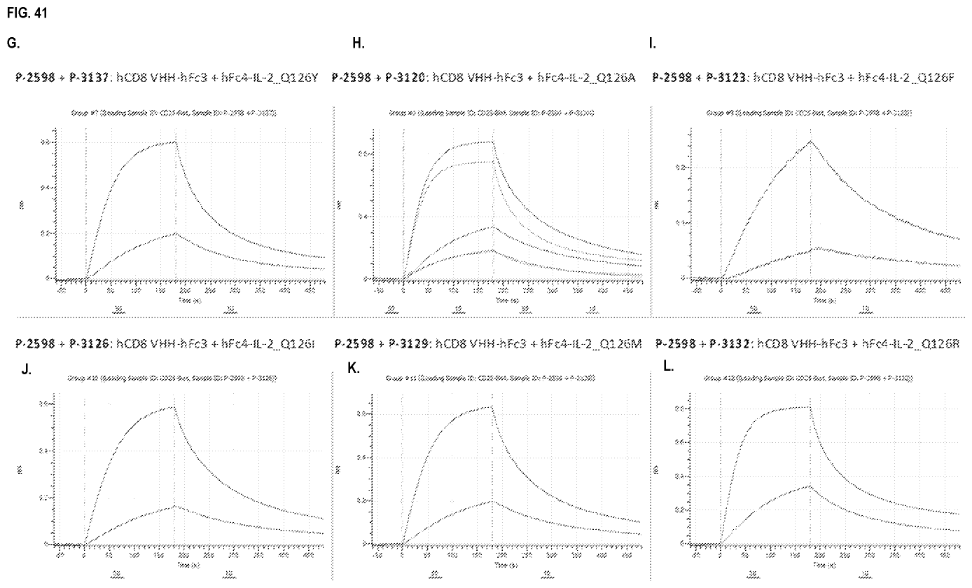

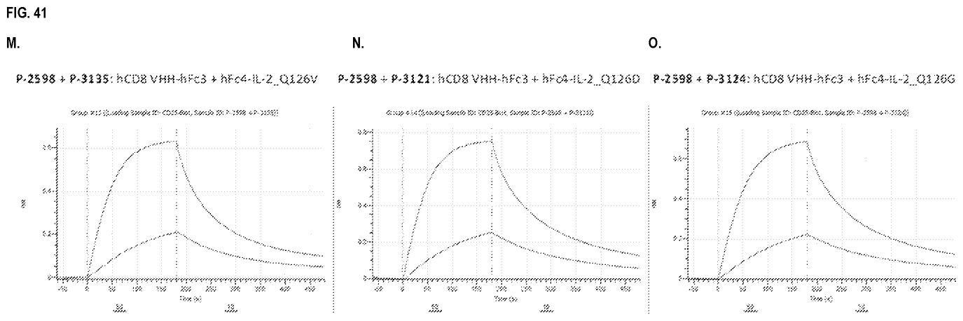

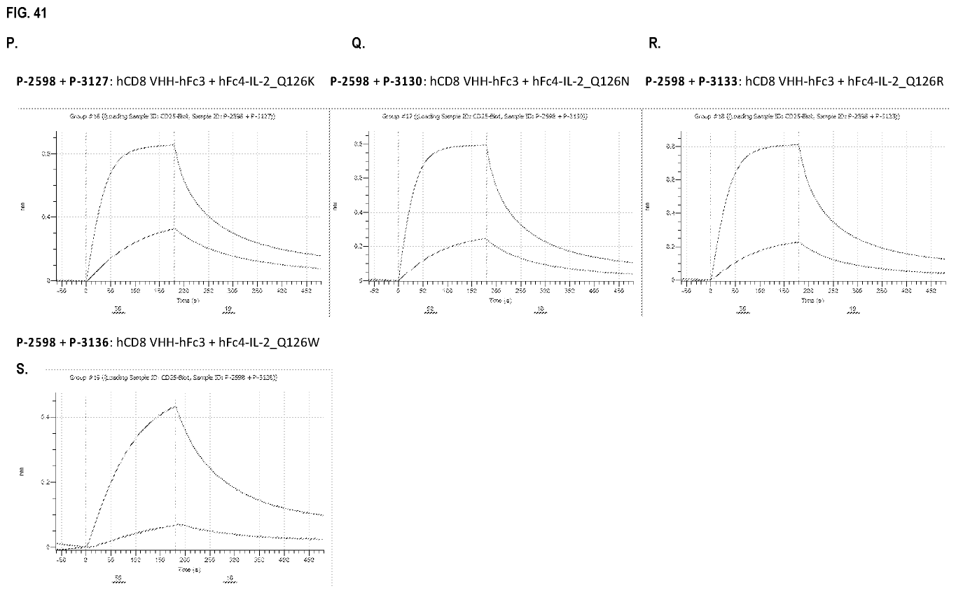

Subsequently, an Octet set-up was used to investigate to what extent the Q126 mutations affect the interaction/binding of IL-2 to the IL-2R alpha chain. In brief: biotinylated CD25 (IL-2R alpha) was loaded onto streptavidin sensors, association and dissociation of two concentrations (50 and 10 nM) CD8 VHH-hFc3+hFc4-IL-2, or Q126 mutants thereof were monitored and used to calculate the association and dissociation constants and hence affinity. Data in FIGS. 41A-S illustrate that most mutations do not affect this interaction, with two exceptions: mutations Q126F (10-fold decrease in affinity, due to lower on-rate) and Q126P, which almost completely abolishes binding. The latter suggesting that the proline substitution results in a conformational shift that is influencing other parts of the IL-2 molecule.

The table below provides association and dissociation constants and also affinity:

| | KD (M) | ka (1/Ms) | kdis (1/s) |
|---|---|---|---|
| CD8 VHH-Fc-hIL-2_C125A | 3.23E−08 | 2.36E+05 | 7.64E−03 |
| CD8 VHH-Fc-hIL-2_C125A | 2.61E−08 | 2.51E+05 | 6.56E−03 |
| CD8 VHH-Fc-hIL-2_C125A_Q126A | 1.91E−08 | 4.74E+05 | 9.06E−03 |
| CD8 VHH-Fc-hIL-2_C125A_Q126A | 1.50E−08 | 4.64E+05 | 6.95E−03 |
| CD8 VHH-Fc-hIL-2_C125A_Q126D | 2.08E−08 | 3.45E+05 | 7.16E−03 |
| CD8 VHH-Fc-hIL-2_C125A_Q126E | 1.35E−08 | 5.65E+05 | 7.61E−03 |
| CD8 VHH-Fc-hIL-2_C125A_Q126F | 1.02E−07 | 4.41E+04 | 4.49E−03 |
| CD8 VHH-Fc-hIL-2_C125A_Q126G | 2.20E−08 | 3.10E+05 | 6.82E−03 |
| CD8 VHH-Fc-hIL-2_C125A_Q126H | 1.78E−08 | 4.10E+05 | 7.31E−03 |
| CD8 VHH-Fc-hIL-2_C125A_Q126I | 2.68E−08 | 2.45E+05 | 6.55E−03 |
| CD8 VHH-Fc-hIL-2_C125A_Q126K | 1.36E−08 | 5.28E+05 | 7.21E−03 |
| CD8 VHH-Fc-hIL-2_C125A_Q126L | 3.19E−08 | 2.04E+05 | 6.49E−03 |
| CD8 VHH-Fc-hIL-2_C125A_Q126M | 2.54E−08 | 2.93E+05 | 7.46E−03 |
| CD8 VHH-Fc-hIL-2_C125A_Q126N | 1.60E−08 | 5.95E+05 | 9.52E−03 |
| CD8 VHH-Fc-hIL-2_C125A_Q126P | NA | NA | NA |
| CD8 VHH-Fc-hIL-2_C125A_Q126R | 1.26E−08 | 5.42E+05 | 6.82E−03 |
| CD8 VHH-Fc-hIL-2_C125A_Q126S | 1.83E−08 | 4.82E+05 | 8.82E−03 |
| CD8 VHH-Fc-hIL-2_C125A_Q126T | 1.90E−08 | 4.76E+05 | 9.03E−03 |
| CD8 VHH-Fc-hIL-2_C125A_Q126V | 2.44E−08 | 3.29E+05 | 8.03E−03 |
| CD8 VHH-Fc-hIL-2_C125A_Q126W | 5.38E−08 | 1.14E+05 | 6.10E−03 |
| CD8 VHH-Fc-hIL-2_C125A_Q126Y | 2.83E−08 | 2.88E+05 | 8.14E−03 |

Based on both datasets, one mutation of each of the above categories (modest: Q126Y, medium: Q126G and strong: Q1261) were retained for further analysis in the following examples.

The following amino acid sequences were used in the Example:
hCD8 VHH-hFc3 (SEQ ID NO: 501)
hFc4-hIL-2_C125A (SEQ ID NO: 502)
hFc4_C125A_Q126A (SEQ ID NO: 503)
hFc4_C125A_Q126D (SEQ ID NO: 504)
hFc4_C125A_Q126E (SEQ ID NO: 505)
hFc4_C125A_Q126F (SEQ ID NO: 506)
hFc4_C125A_Q126G (SEQ ID NO: 507)
hFc4_C125A_Q126H (SEQ ID NO: 508)
hFc4_C125A_Q1261 (SEQ ID NO: 509)
hFc4_C125A_Q126K (SEQ ID NO: 510)
hFc4_C125A_Q126L (SEQ ID NO: 511)
hFc4_C125A_Q126M (SEQ ID NO: 512)
hFc4_C125A_Q126N (SEQ ID NO: 513)
hFc4_C125A_Q126P (SEQ ID NO: 514)
hFc4_C125A_Q126R (SEQ ID NO: 515)
hFc4_C125A_Q126S (SEQ ID NO: 516)
hFc4_C125A_Q126T (SEQ ID NO: 517)
hFc4_C125A_Q126V (SEQ ID NO: 518)
hFc4_C125A_Q126W (SEQ ID NO: 519)
hFc4_C125A_Q126Y (SEQ ID NO: 520)

### Example 15: Combination of Alpha and Gamma Mutations

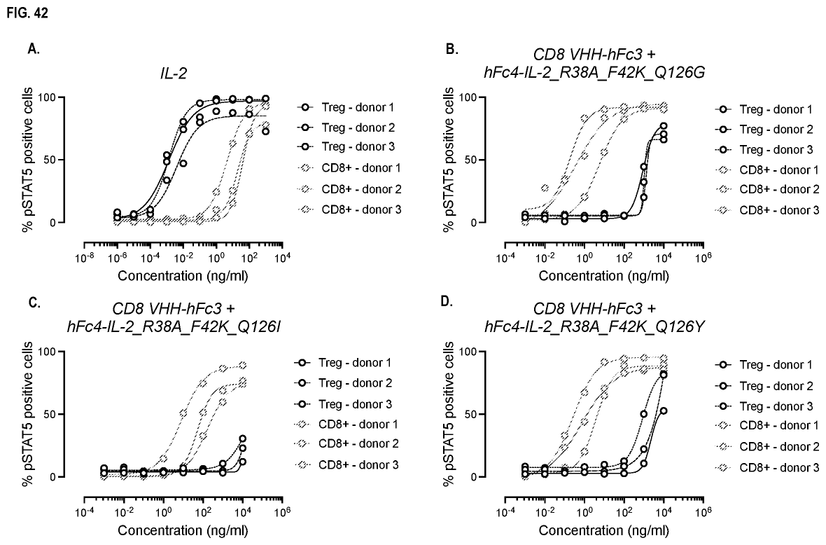

In analogy with the combinations alpha:beta, the alpha mutation (R38A_F42K) was combined with three selected gamma mutations (Q126G, Q1261 and Q126Y) in de ALN2 warhead. The three resulting ALN2's were produced, purified and tested for STAT5 phosphorylation in CD8+ and Treg PBMC subsets of three different healthy donors as described earlier. Results in FIGS. 42A-D illustrate that:
As observed previously: Tregs are 4 logs more sensitive than CD8+ cells to IL-2
In contrast, CD8+ cells are about 3 logs more sensitive than Tregs to ALN2's with any of the combined alpha:gamma mutations.
The combination with the strong Q1261 mutation allows clear signaling in the CD8+ cells while STAT5 phosphorylation in Treg cells is absent almost up to 10 μg/mL.

The following amino acid sequences were used in the Example:
hCD8 VHH-hFc3 (SEQ ID NO: 521)
hFc4-IL-2_R38A_F42K_Q126G (SEQ ID NO: 522)
hFc4-IL-2_R38A_F42K_Q126I (SEQ ID NO: 523)
hFc4-IL-2_R38A_F42K_Q126Y (SEQ ID NO: 524)

### Example 16: Combination of Beta and Gamma Mutations for Selectivity Towards Activated CD8 Cells

A first major shortcoming of the use of no-alpha IL-2 variants (see above) in the treatment of cancer is that these molecules are not able to compete with endogenously expressed IL-2 for binding the IL-2R alpha chain. Indeed, activation of mainly CD4⁺, but also CD8⁺, cells results in the secretion of IL-2 in a tumor setting. This ‘newly secreted’ IL-2 will preferentially activate IL-2R alpha expressing cells, like Tregs, resulting in dampening of the anti-tumor response. A second disadvantage is that no-alpha molecules cannot take advantage of the fact that activated CTL's have upregulated IL-2R alpha levels, and IL-2R alpha remains the driving force of IL-2R activation. Based on these two concepts, the potential of combining a beta A-Kine mutation (here D20E) with a gamma common A-Kine mutations (here Q126G or Q126Y) in the IL-2 warhead was evaluated.

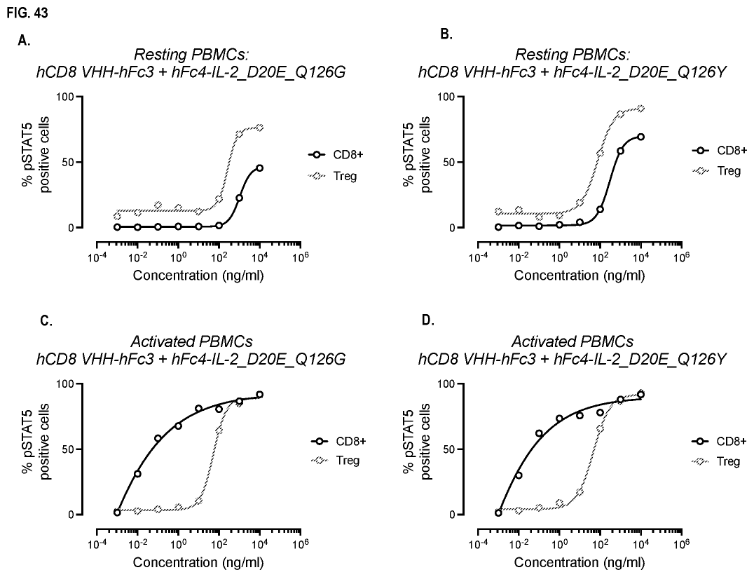

To mimic the activation (and thus up-regulation of CD25) of CD8+ cells, freshly isolated PBMC of healthy donors were treated with TransAct (nanomatrix conjugated with recombinant humanized CD3 and CD28 agonists; Miltenyi Biotec) for two days, prior to stimulation with the D20E_Q126G or D20E_Q126Y ALN2 variants, with STAT5 phosphorylation as functional read-out. What can be appreciated from FIGS. 43A-D is that while both beta:gamma mutants are more active (approximately a 10-fold) on Treg compared to CD8⁺ cells, this sensitivity is completely reversed on activated PBMCs. Here, likely due to the up-regulation of IL-2R alpha, the CD8⁺ cells become hyper-sensitive to the beta:gamma ALN2 variants. These data illustrate that these ALN2 variants are selective for activated CD8 positive cells.

The following amino acid sequences were used in the Example:
hCD8 VHH-hFc3 (SEQ ID NO: 297)
hFc4-IL-2_D20E_Q126G (SEQ ID NO: 382)
hFc4-IL-2_D20E_Q126Y (SEQ ID NO: 395)
hFc4-hIL-2_C125A (SEQ ID NO: 482)

### Example 17: CD8-Fc-ALN2 is Specific and Superior to Wild Type FC-IL-2 to Activate and Proliferate CD8-Positive T Cells

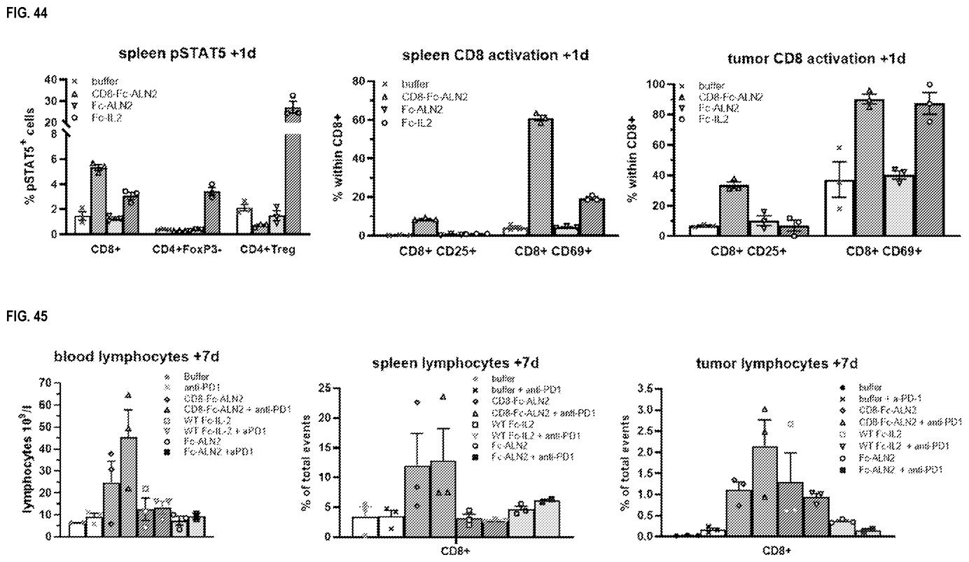

C57BI/6J mice (female, 9 weeks old) were injected subcutaneously (s.c.) with 6E+5 MC38 cells. Seven days later, treatments were started: mice received intravenously (i.v.) either (1) buffer, (2) targeted CD8-Fc-ALN2 (12.5 μg/mouse), (3) an equimolar dose of untargeted Fc-ALN2 (10.7 μg/mouse) or (4) an equimolar dose of wild type Fc-IL2 (10.7 μg/mouse). One day later, spleens and tumors were removed for intracellular analysis of STAT5 phosphorylation (documenting IL-2 receptor signaling), as well as membrane CD69 and CD25 expression (documenting activation) in CD8− or CD4-positive T cells. The results plotted in FIG. 44 show that while untargeted Fc-ALN2 did not induce pSTAT5, CD69, or CD25 in CD8-positive T cells, the CD8-Fc-ALN2 construct did so very robustly and surprisingly in general even more efficiently than wild type Fc-ALN2. In contrast, wild type Fc-IL2 was very efficient to induce STAT5 phosphorylation in CD4 and FoxP3-positive regulatory T cells (Treg) and other (FoxP3-negative) CD4-positive T cells. Importantly, CD8-Fc-ALN2 did not have any effect on the FoxP3-positive regulatory T cells (Treg) or even reduced activation of these cells as evidenced by the pSTAT5 data.

To evaluate T cell proliferation, lymphocytes were counted in circulation (using a Vetscan HMS, Zoetis), as well as in spleen and tumor (via flow cytometry) 14 days after tumor inoculation, which is 1 week after the start of the treatment, i.e. after 2 treatments on days 7 and 10. The results plotted in FIG. 45 show that both in blood and in spleen, targeted CD8-Fc-ALN2 was the only treatment that efficiently caused lymphocyte proliferation (in contrast to untargeted Fc-ALN2 and wild type Fc-IL2, injected in equimolar amount). In tumor tissue, wild type Fc-IL2 was capable of increasing CD8-positive T cell numbers. Adding anti-PD-1 Ab treatment further augmented CD8-Fc-ALN2 induced lymphocyte proliferation in circulation and in tumor tissue but surprisingly did not do so with Fc-IL2.

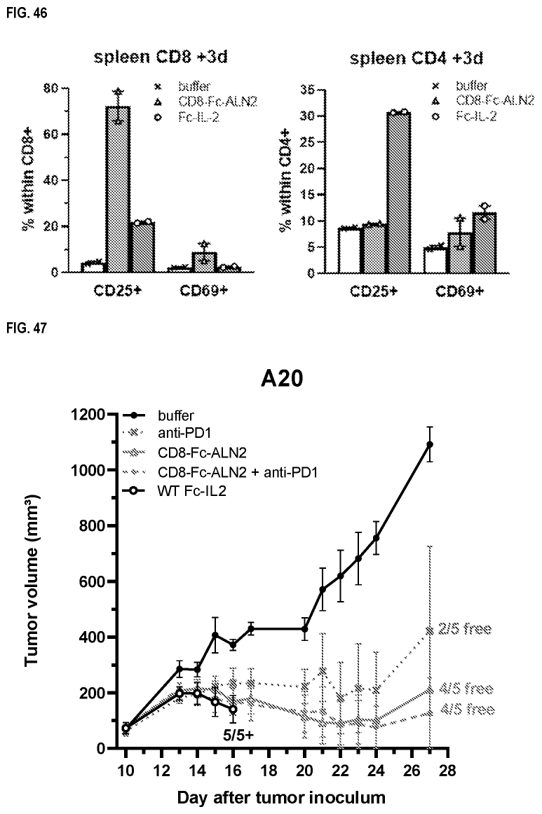

Furthermore, in spleens collected 3 days after treatment, 25 μg CD8-Fc-ALN2 was much better than the equimolar 21.7 μg wild type Fc-IL2 to enhance CD25 expression (and thus activation) in CD8-positive T cells (70% vs 20%, respectively), while only wild type Fc-IL2 induced CD25 expression in CD4-positive T cells (see FIG. 46).

The following amino acid sequences were used in the Example:
mCD8 VHH-hFc3 (SEQ ID NO: 495)
hFc4-hIL-2_R38A_F42K_N88G_C125A (SEQ ID NO: 335)
hFc3 (SEQ ID NO: 369)
hFc4-hIL-2_C125A (SEQ ID NO: 482)

### Example 18: Efficacy of CD8-Fc-ALN2 in A20, B16, and Panc02 Tumor Models

The anti-tumor efficacy of the mouse CD8-targeted ALN2 variant (CD8-Fc-ALN2) with combined alpha:beta mutations R38A_F42K_N88G was previously shown to be very successful in two syngeneic murine models for colon cancer, MC38 and CT26. In addition, the anti-tumoral efficacy was analyzed in 3 other tumor models: lymphoma A20, melanoma B16F10, and pancreatic adenocarcinoma Panc02. For this experiment, Balb/c mice (female, 9 weeks old) were inoculated subcutaneously (s.c.) with 6E+5 A20 cells and treatment started on day 10, or C57BI/6J (female, 9 weeks old) with 6E+5 B16F10 or Panc02 cells and treatment started on day 6.

Firstly, A20 tumor-bearing mice were treated intravenously (i.v.) with either (1) buffer, (2) targeted CD8-Fc-ALN2 (12.5 μg/mouse) as a monotherapy, (3) or targeted CD8-Fc-ALN2 (12.5 μg/mouse) in combination with 200 μg/mouse anti-PD-1 Ab, or (4) untargeted wild type Fc-IL2 (equimolar 10.7 μg/mouse), on days 10, 14 and 17 after tumor inoculation. Tumor sizes (means+/−SEM; n=5 per group) were plotted in FIG. 47. FIG. 47 shows that both untargeted wild type Fc-IL-2 and targeted CD8-Fc-ALN2 substantially inhibited tumor growth, but the wild-type Fc-IL-2 treated mice all succumbed after the second treatment (indicated in FIG. 47 as “5/5+” or all animals died). This result contrasts with the CD8-Fc-ALN2 treated mice, documenting the extreme susceptibility of Balb/c mice for wild type IL-2 toxic side effects, but not for CD8-Fc-ALN2. At the end of the experiment, 4 out of 5 CD8-Fc-ALN2 treated animals were completely tumor-free. Extra treatment with anti-PD-1 could initially not increase CD8-Fc-ALN2 efficacy; also, in this group 4 out of 5 mice were tumor-free but this changed after an additional two months follow up. At this later time point, six mice (2 from the group treated with CD8-Fc-ALN2 and 4 from the group treated with CD8-Fc-ALN2+anti-PD1) were still tumor-free and re-challenged with A20 tumor cells on the other flank. All 6 mice remained tumor-free and did not develop a new tumor, in contrast to naïve mice inoculated with the same A20 cells on the same day.

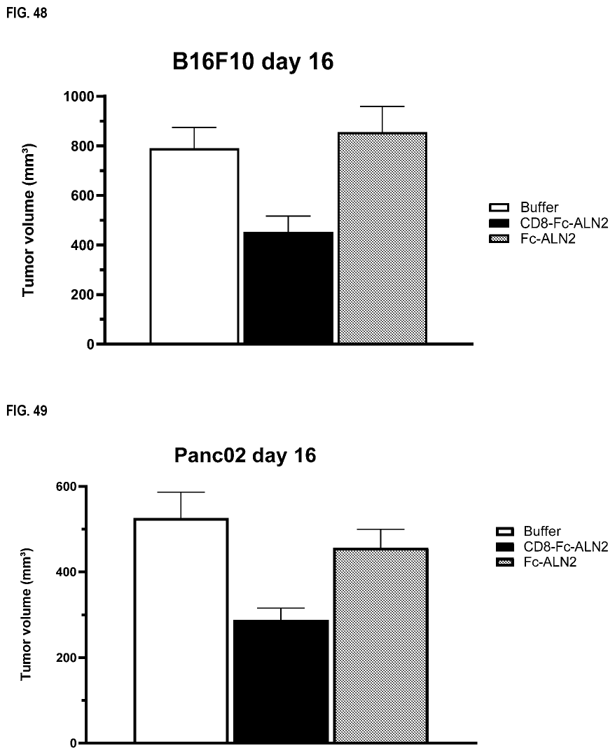

Secondly, B16F10 tumor-bearing mice were treated i.v. with either (1) buffer, (2) targeted CD8-Fc-ALN2 (12.5 μg/mouse) or (3) untargeted Fc-ALN2 (equimolar 10.7 μg/mouse) on days 6, 9 and 13 after tumor inoculation. Tumor sizes on day 16 after inoculation (means+/−SEM; n=4-5 per group) were plotted in FIG. 48. CD8-Fc-ALN2 substantially slowed down tumor growth while the untargeted ALN2 had no effect.

Finally, Panc02 tumor-bearing mice were treated i.v. with either (1) buffer, (2) targeted CD8-Fc-ALN2 (12.5 μg/mouse) or (3) untargeted Fc-ALN2 (equimolar 10.7 μg/mouse) on days 6 and 13 after tumor inoculation. Tumor sizes on day 16 after inoculation (means+/−SEM; n=5 per group) were plotted in FIG. 49. CD8-Fc-ALN2 substantially slowed down tumor growth which was not observed with the untargeted ALN2.

The following amino acid sequences were used in the Example:
mCD8 VHH-hFc3 (SEQ ID NO: 495)
hFc4-hIL-2_R38A_F42K_N88G_C125A (SEQ ID NO: 335)
hFc3 (SEQ ID NO: 369)

### Example 19: Tumor Targeting of ALN2

In this example the potential of targeting ALN2 activity to tumor (or other immune cells) using a PD-L1 VHH was evaluated. PD-L1-Fc-ALN2 protein was made by transfection of ExpiCHO cells with following plasmid combination and purified as described earlier:
PD-L1-Fc-IL-2_R38A_F42K_N88G: mPD-L1 VHH-hFc3+hFc4-IL-2_R38A_F42K_N88G

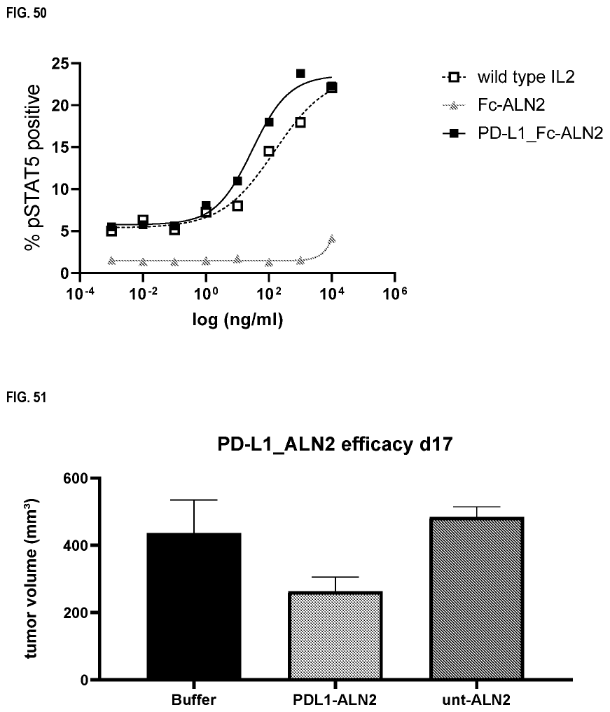

The potency and specificity of the PD-L1 ALN2 was verified on mouse splenocytes by analyzing phosphorylation of STAT5 as described in Example 13. FIG. 50 shows that an untargeted Fc-ALN2 does not induce pSTAT5 in mouse splenocytes up to 10 μg/ml, while PD-L1 targeting is able to induce a pSTAT5 signal similar as WT IL-2.

To test efficacy the MC38 tumor-model, which is a PD-L1 expressing tumor, PD-L1 targeted ALN2 was selected. C57BI/6J mice (female, 9 weeks old) were injected s.c. with 6E+5 MC38 cells. Six days later, treatments were started: mice received intravenously (i.v.) either (1) buffer, (2) targeted Fc-ALN2 (with IL-2 mutations R38A_F42K_N88G; 12.5 μg/mouse), or (3) untargeted Fc-ALN2, three times (on days 6, 9 and 13). Tumor sizes on day 17 after inoculation (means+/−SEM; n=4-6 per group) were plotted in FIG. 51 showing a specific tumor growth inhibition by the PD-L1 targeted ALN2 only.

In addition, an ALN2 targeted to the extracellular tumor matrix (based on a TenascinC-A1 specific VHH) in a cancer context was generated. This TNC-Fc-ALN2 protein was made by transfection of ExpiCHO cells with the following plasmid combinations and purified as described above:
TNC-Fc-IL-2_R38A_F42K_N88G: mTNC VHH-hFc3+hFc4-IL-2_R38A_F42K_N88G

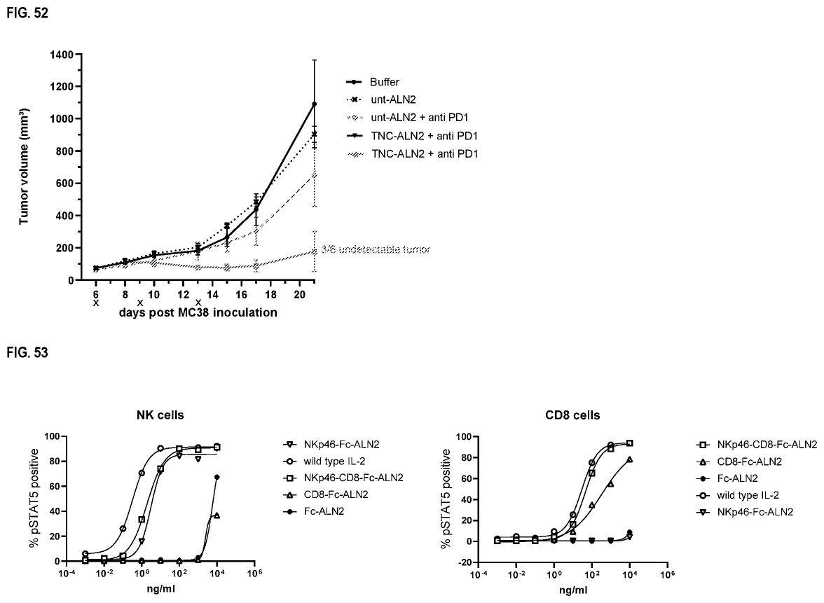

To test efficacy in the MC38 tumor-model, C57BI/6J mice (female, 9 weeks old) were injected s.c. with 6E+5 MC38 cells. Six days later, treatments were started: mice received intravenously (i.v.) either (1) buffer, (2) untargeted Fc-ALN2, or (3) TNCA1 targeted Fc-ALN2 (with IL-2 mutations R38A_F42K_N88G; 12.5 μg/mouse), three times (on days 6, 9 and 13). Tumor sizes were measured three times a week and means+/−SEM (n=4-6) were plotted in FIG. 52. When mentioned, the ALN2 treatment was supplemented by intraperitoneal injection (i.p.) anti-PD-1 Ab treatments (200 μg/mouse) on the same days as the i.v. treatments. As can be appreciated, in combination with anti-PD-1 Ab therapy and in contrast to untargeted Fc-ALN2, TNC-targeted Fc-ALN2 treatments clearly and substantially inhibited tumor growth. Surprisingly, at day 21, only for TNC-Fc-ALN2+PD-L1 treated group a number of mice without detectable tumor were identified, i.e. in three out of 6 animals.

### Example 20: Bispecific Targeting of ALN2

A FAP targeted ALN2, NKp46 targeted ALN2, and the following bi-specific ALN2s were generated and purified from ExpiCHO supernatant as described earlier:
NKp46-Fc-ALN2 (combination of NKp46 VHH-hFc3 (sequence below)+hFc4-hIL-2_R38A_F42K_N88G). This construct targets ALN2 to NK cells.
NKp46-CD8-Fc-ALN2 (combination of NKp46 VHH-hFc3+CD8 VHH-hFc4-hIL-2_R38A_F42K_N88G; sequences below). This construct targets ALN2 to both NK and CD8 cells.
FAP-Fc-ALN2 (combination of FAP VHH-hFc3 (sequence below)+hFc4-hIL-2_R38A_F42K_N88G). This construct targets ALN2 to the tumor or cancer-associated fibroblasts via the FAP specific VHH.
FAP-CD8-Fc-ALN2 (combination of FAP VHH-hFc3+CD8 VHH-hFc4-hIL-2_R38A_F42K_N88G; sequences below). This construct targets ALN2 to the tumor or cancer-associated fibroblasts via the FAP specific VHH and to CD8 cells.

The potency and specificity of the NKp46-CD8-ALN2 was verified on mouse splenocytes by analyzing phosphorylation of STAT5 as described in Example 13. FIG. 53 shows that an untargeted Fc-ALN2 does not induce pSTAT5 in CD8 cells up to 10 μg/ml and only marginally in NK cells with an EC50 estimated (extrapolated data as max signal not reached at 10 μg/ml) at about 5 μg/mL. CD8 and NK cells can be simultaneously stimulated by the bispecific construct with EC50s of respectively about 46 (>200-fold window vs untargeted) and 2 ng/ml (about 2500-fold window vs untargeted) which is at least as similar as the CD8 or NKp46 monospecific ALN2s.

As a follow-on to above experiment, 2 more bi-specific constructs were generated and purified from ExpiCHO supernatant as described earlier:
CD8_PD-1_Fc-ALN2 (combination of PD-1 VHH-hFc3+CD8 VHH-hFc4-hIL-2_R38A_F42K_N88G).
CD8_PD-L1_Fc-ALN2 (combination of PD-L1 VHH-hFc3+CD8 VHH-hFc4-hIL-2_R38A_F42K_N88G)

These constructs are evaluated for their anti-tumoral effect in mice bearing an MC38 tumor. These mice are treated once i.v. when tumor size is about 100-200 mm³ or repeatedly with a 3-4 day interval and tumor size is assessed every 2 to 3 days.

Sequences used in this experiment:
mNKp46 VHH-hFc3 (SEQ ID NO: 525)
mFAP VHH-hFc3 (SEQ ID NO: 526)
mPD-L1 VHH-hFc3 (SEQ ID NO: 527)
mTNC VHH-hFc3 (SEQ ID NO: 528)
mCD8 VHH-hFc4-hIL-2_R38A_F42K_N88G_C125A (SEQ ID NO: 529)
mPD-1 VHH-hFc3 (SEQ ID NO: 530)

### Example 21: Activation of CD8 Cells with D20E and N88G CD8-Fc-ALN2 Variants

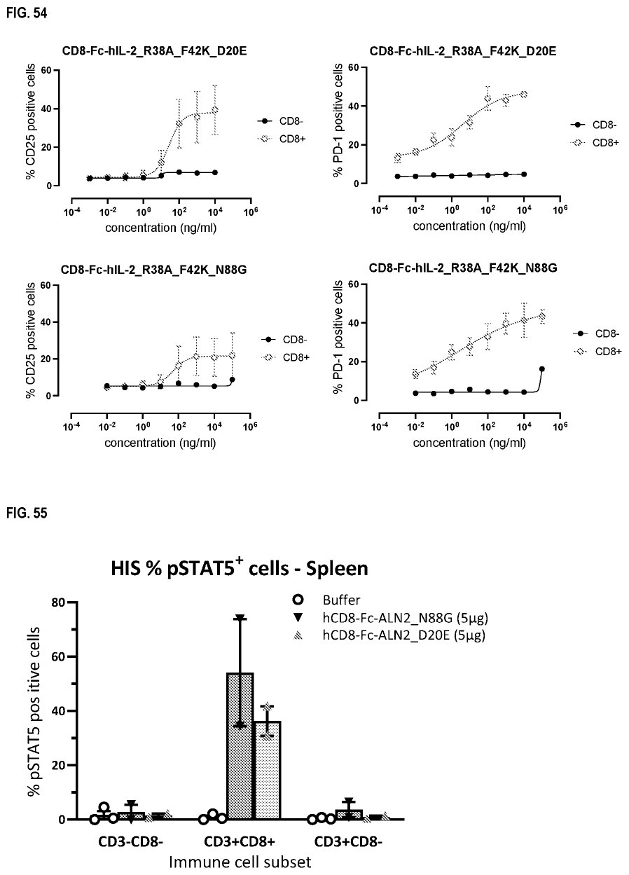

Human CD8 targeted IL-2 variants with a combination of “no_alpha” mutations that ablate activity at the IL-2R alpha chain (R38A_F42K) and a recoverable loss-of-function beta mutation hCD8-Fc-ALN2_N88G or hCD8-Fc-ALN2_D20E) were evaluated for their selective activation by analyzing the cell-surface expression of the activation-markers CD25 (IL-2R alpha chain), and PD-1 in FACS. Expression was monitored after five days in three PBMC donors. Data (average of the three donors) are plotted in FIG. 54 and clearly show that CD8 targeting of these mutants allows selective up-regulation (and thus activation) of these markers in CD8-positive vs. off-target cells.

Also, in immune-deficient mice provided with a human immune system (HIS mice) bearing an MDA-MB-231 tumor, the same human CD8 targeted ALN2 molecules efficiently and specifically induced STAT5 phosphorylation (analyzed 45 minutes after injection) in only CD8-positive T lymphocytes (FIG. 55). Humanized HIS mice were obtained via irradiation and intrahepatic injection of NSG 2-day-old pups with human cord blood CD34-positive stem cells; the tumor-bearing HIS mice were used for intravenous hCD8-Fc-ALN2 injection at the age of 6 months. Similar to the human CD8 targeted variants, mouse CD8-targeted Fc-ALN2 variants were generated with a combination of “no_alpha” mutations with the beta chain recoverable loss-of-function mutations D20E or N88G (which has been used in previous examples) as follows:
mCD8-Fc-IL-2_R38A_F42K_D20E: mCD8 VHH-hFc3+hFc4-IL-2_R38A_F42K_D20E
mCD8-Fc-IL-2_R38A_F42K_N88G: mCD8 VHH-hFc3+hFc4-IL-2_R38A_F42K_N88G

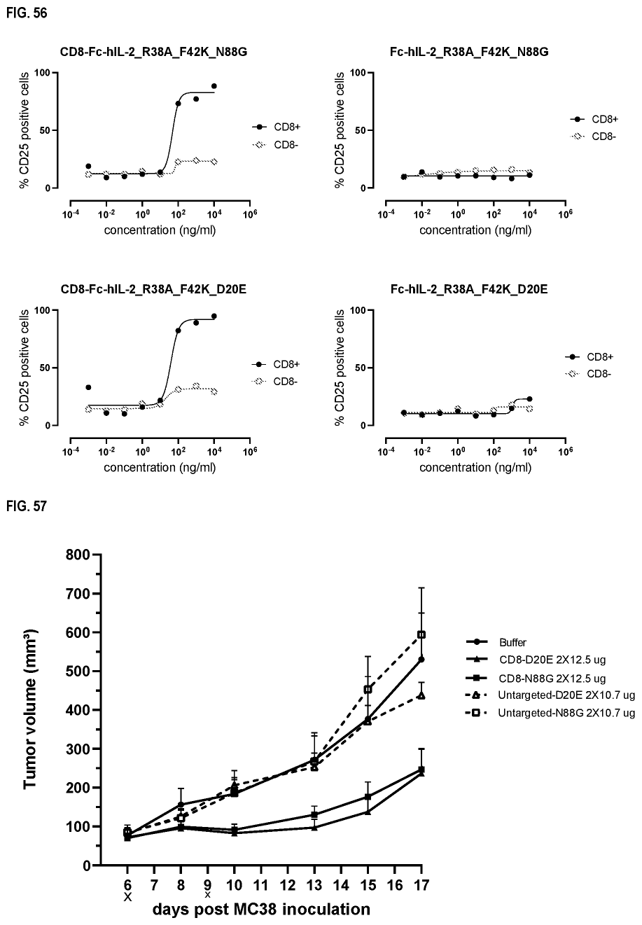

Variants were expressed and purified as described above and tested on mouse splenocytes for the activation of CD8-positive cells by measuring up-regulation of cell surface expression of CD25 after 6 days (FIG. 56). Both R38A_F42K_D20E and R38A_F42K_N88G variants clearly increase expression of this activation marker in a selective and comparable manner, i.e. clear effects in CD8-positive but not in off-target (CD8 negative) cells.

The following amino acid sequences were used in the Example:
mCD8 VHH-hFc3 (SEQ ID NO: 495)
hCD8 VHH-hFc3 (SEQ ID NO: 483)
hFc4-hIL-2_R38A_F42K_N88G_C125A (SEQ ID NO: 335)
hFc4-IL-2_R38A_F42K_D20E (SEQ ID NO: 332)

### Example 22: Antitumoral Effect of D20E and N88G CD8-ALN2 Variants

For evaluation in the MC38 tumor model, C57BI/6J mice (female, 9 weeks old) were injected s.c. with 6E+5 MC38 cells. Six days later, treatments were started: mice received i.v. either (1) buffer, (2) targeted CD8-Fc-ALN2 (12.5 μg/mouse), or (3) equimolar amount of untargeted Fc-ALN2 (10.7 μg/mouse), twice on days 6 and 9.
CD8-Fc-IL-2_R38A_F42K_D20E: mCD8 VHH-hFc3 (SEQ ID NO: 495)+hFc4-IL-2_R38A_F42K_D20E (SEQ ID NO: 333)
CD8-Fc-IL-2_R38A_F42K_N88G: mCD8 VHH-hFc3 (SEQ ID NO: 495)+hFc4-IL-2_R38A_F42K_N88G (SEQ ID NO: 335)
Fc-IL-2_R38A_F42K_N88G: VHH-hFc3 (SEQ ID NO: 369)+hFc4-IL-2_R38A_F42K_N88G (SEQ ID NO: 335)
Fc-IL-2_R38A_F42K_N88G: VHH-hFc3 (SEQ ID NO: 369)+hFc4-IL-2_R38A_F42K_D20E (SEQ ID NO: 332)

Tumor sizes were measured three times a week and average growth curves were plotted in FIG. 57 and indicate that D20E-based CD8-Fc-ALN2 was at least as efficient as N88G-based CD8-Fc-ALN2 in the absence of an effect by both untargeted counterparts.

### Example 23: In Vivo Evaluation of Alpha:Gamma ALN2 in the MC38 Tumor Model

In this example, the anti-tumor efficacy of “no_alpha” mutations in combination with a recoverable loss-of-function gamma mutation was analyzed. The following constructs were expressed, purified, and tested for STAT5 phosphorylation in primary splenocytes as described earlier:
CD8-Fc-IL-2_R38A_F42K_Q126G: mCD8 VHH-hFc3 (SEQ ID NO. 495)+hFc4-IL-2_R38A_F42K_Q126G (SEQ ID NO. 522)
CD8-Fc-IL-2_R38A_F42K_Q1261: mCD8 VHH-hFc3 (SEQ ID NO. 495)+hFc4-IL-2_R38A_F42K_Q126| (SEQ ID NO. 523)
CD8-Fc-IL-2_R38A_F42K_Q126Y: mCD8 VHH-hFc3 (SEQ ID NO. 495)+hFc4-IL-2_R38A_F42K_Q126Y (SEQ ID NO. 524)

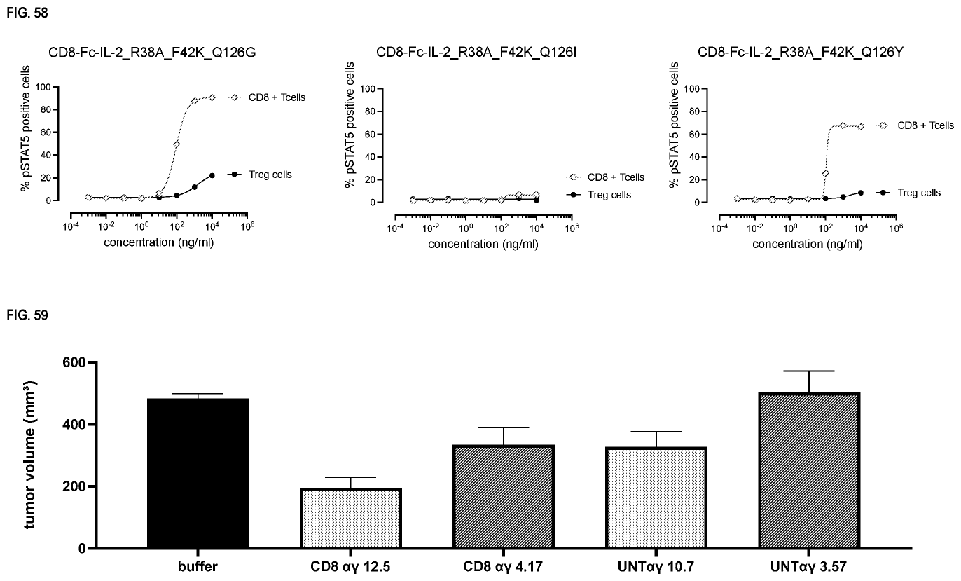

Data in FIG. 58, show that the combination of “no_alpha” mutations and Q126G or Q126Y resulted in ALN2 variants with comparable activity (EC50 of 90 and 110 ng/ml, respectively) and selectivity towards CD8-positive mouse cells (almost no pSTAT5 in Treg cells). The Q1261 mutations cannot be recovered at concentrations at or below 10 μg/mL in a mouse context, resulting in a variant devoid of almost all activity (and this in contrast to the effect on human PBMC's in the earlier example). The variant CD8-Fc-IL-2_R38A_F42K_Q126G was subsequently evaluated in vivo.

For evaluation in vivo, C57BI/6J mice (female, 9 weeks old) were injected s.c. with 6E+5 MC38 cells. Six days later, treatments were started: mice received i.v. (1) buffer, (2) CD8-Fc-ALN2 (12.5 μg/mouse), (3) CD8-Fc-ALN2 (4.17 μg/mouse), (4) equivalent untargeted ALN2 (12.5 μg/mouse), or (4) equivalent untargeted ALN2 (3.57 μg/mouse), on days 6, 9 and 13. Tumor sizes at day 17 (i.e. 4 days after the last treatment) were plotted in FIG. 59 as means+/−SEM (n=7). As can be seen in FIG. 59, the alpha:gamma ALN2 is dose-dependently able to substantially delay tumor growth with superior effects for the CD8 targeted ALN2.

### Example 24: In Vivo Evaluation of the Beta:Gamma Combination

The ALN2 variant CD8-Fc-IL-2_N88G_Q126G (mCD8 VHH-hFc3 (SEQ ID NO: 495)+hFc4-IL-2_N88G_Q126G) was expressed in ExpiCHO and purified from the supernatant as described earlier:
hFc4-IL-2_R38A_F42K_N88G_C125A_Q126G (SEQ ID NO: 531)

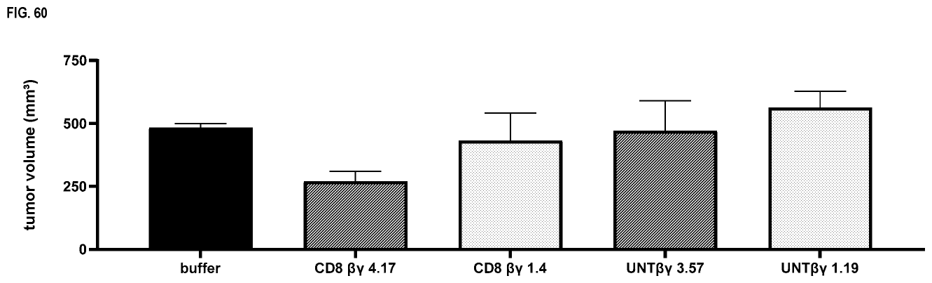

For evaluation in vivo, C57BI/6J mice (female, 9 weeks old) were injected s.c. with 6E+5 MC38 cells. Six days later, treatments were started: mice received i.v. (1) buffer, (2) CD8-Fc-ALN2 (4.17 μg/mouse), (3) CD8-Fc-ALN2 (1.4 μg/mouse), (4) equivalent untargeted ALN2 (3.57 μg/mouse), or (4) equivalent untargeted ALN2 (1.19 μg/mouse), on days 6, 9 and 13. Tumor sizes at day 17 (i.e. 4 days after the last treatment) were plotted in FIG. 60 as means+/−SEM (n=7). As can be seen FIG. 60, the beta:gamma ALN2 is dose-dependently able to substantially delay tumor growth with superior effects for the CD8 targeted ALN2.

### Example 25: Screening of IL-2 Variants to Specifically Target Cells Expressing the IL-2R Alpha Chain

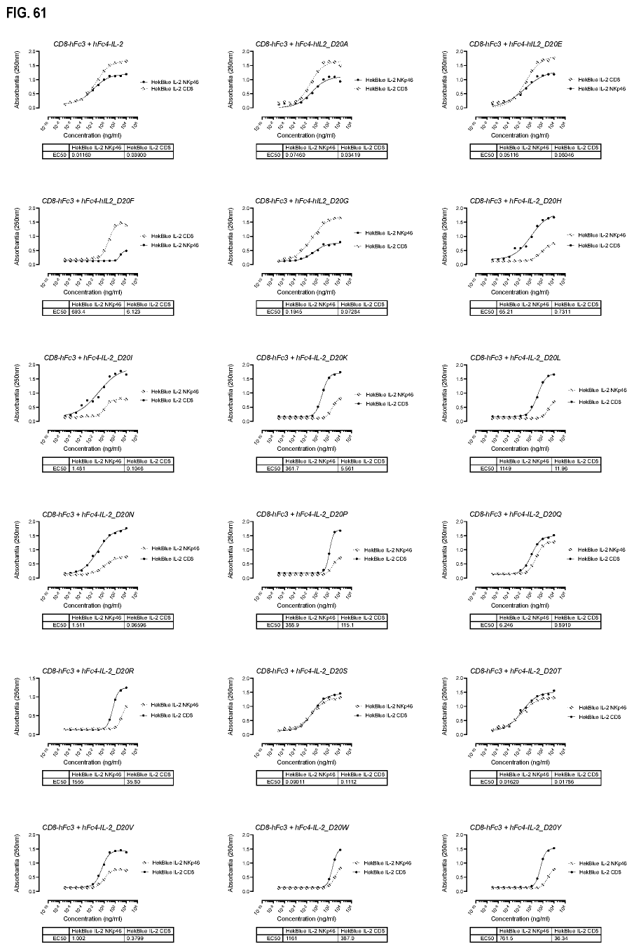

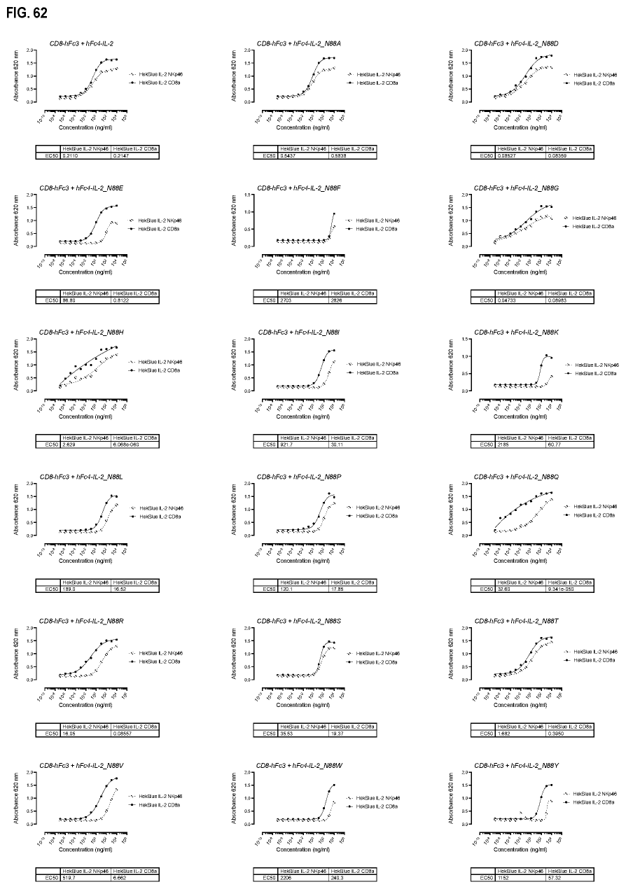

In previous examples, beta (D20 or N88) and gamma (Q126) attenuating mutations were combined to make a beta:gamma ALN2 variant with an intact IL-2R alpha binding-site and CD8 targeting was used to increase selectivity towards activated CD8-positive cells over e.g. Tregs. Unfortunately, such ALN2 variants were expressing poorly. Hence, as an alternative for the combination of a beta and gamma mutation, the beta mutations (D20 and N88) that appeared non-recoverable in Example 3 were rescreened. The initial screening of beta-mutants in Example 3 was done on ‘resting’ or non-activated CD8 positive PBMC's, which are negative for the IL-2R alpha expression. In this example, the D20 and N88 mutants were re-screened on cells expressing a functional trimeric alpha:beta:gamma IL-2 receptor. To this end, signaling in HEK-Blue IL-2 cells (InvivoGen) stably expressing either human CD8 (HEK-Blue CD8) or human NKp46 (HEK-Blue NKp46) was analyzed. Data for the D20 (FIG. 61) and N88 (FIG. 62) variants surprisingly illustrate that for a number of mutants (e.g. the D20F, D20G, D20H, D20I, D20K, D20L, D20N, N88E, N88I, N88K, N88Q, N88R, and N88V) there is a clear attenuation of signaling (compared to CD8-Fc-IL2) and that this loss in activity can be, at least in part, restored upon CD8 targeting (HEK-Blue CD8 vs HEK-Blue NKp46 cells), while this was not the case for these mutants when screened on the functional dimeric beta:gamma IL-2 receptor.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A chimeric protein or protein complex comprising:
(a) a modified IL-2 signaling agent, wherein the modified IL-2 signaling agent has one or more mutations or modifications relative to a wild type signaling agent having SEQ ID NO: 1 that confer improved safety and reduced affinity or bioactivity of the modified IL-2 signaling agent for IL-2Rαβγ and/or IL-2Rβ and/or IL-2Rγ and/or IL-2Rα, relative to the wild type IL-2 signaling agent; and
(b) one or more targeting moieties, said targeting moieties comprising recognition domains which specifically bind to antigens or receptors of interest,
wherein the modified IL-2, and the one or more targeting moieties are optionally connected with one or more linkers,
wherein the modified IL-2 signaling agent comprises:
(i) a mutation selected from D20E, D20F, D20G, D20H, D20I, D20K, D20L, and D20V; or
(ii) a mutation selected from N88G, N88E, N88K, N88Q, and N88V; or
(iii) a mutation selected from Q126G, Q126A, Q126E, Q126F, Q126H, Q126I, Q126K, Q126L, Q126N, Q126P, Q126R, Q126S, Q126T, Q126V, Q126W, and Q126Y; or
(iv) a mutation selected from (i) and (iii) or a mutation selected from (ii) and (iii); or
(v) one or more mutations selected from:
R38A/F42K/N88G/C125A,
F42K/C125A,
D20E/C125A,
D20F/C125A,
D20G/C125A,
D20H/C125A,
D20I/C125A,
D20K/C125A,
D20L/C125A,
D20N/C125A,
D20S/C125A,
D20T/C125A,
D20V/C125A,
N88A/C125A,
N88D/C125A,
N88G/C125A,
N88E/C125A,
N88H/C125A,
N88I/C125A,
N88K/C125A,
N88Q/C125A,
N88R/C125A,
N88T/C125A,
N88V/C125A,
D20E/R38A/F42K,
D20E/R38A/F42K/C125A,
D20V/R38A/F42K,
D20V/R38A/F42K/C125A,
R38A/F42Y/E62A/C125A,
R38A/F42Y/Y45A/E62A,
F42Y/Y45A/L72G,
R38A/F42Y/Y45A/E62A/C125A,
F42Y/Y45A/L72G/C125A,
R38A/F42K/N88G,
R38A/F42K/Q126G,
R38A/F42K/Q126I,
R38A/F42K/Q126Y,
R38A/F42K/Q126G/C125A,
R38A/F42K/Q1261/C125A,
R38A/F42K/Q126Y/C125A,
R38A/F42K/E62A/N88G, and
R38A/F42K/E62A/N88G/C125A.

2. The chimeric protein or protein complex of claim 1, wherein the modified IL-2-signaling agent further comprises a glycosylation mutation, wherein the mutation decreases or eliminates glycosylation of the modified IL-2 signaling agent.

3. The chimeric protein or protein complex of claim 2, wherein the glycosylation mutation is a T3 substitution, wherein the mutation is one of T3A, T3F, T3H, T3L, T3V, and T3Y.

4. The chimeric protein or protein complex of claim 2, wherein the glysosylation mutation is a deletion of the first 3, 4, 5, 6, 7, or 8 residues of the modified IL-2.

5. A chimeric protein or protein complex comprising:
(a) a modified IL-2 signaling agent, wherein the modified IL-2 signaling agent has one or more mutations or modifications relative to a wild type signaling agent having SEQ ID NO: 1 that confer improved safety and reduced affinity or bioactivity of the modified IL-2 signaling agent for IL-2Rαβγ and/or IL-2Rβ and/or IL-2Rγ and/or IL-2Rα, relative to the wild type IL-2 signaling agent; and
(b) one or more targeting moieties, said targeting moieties comprising recognition domains which specifically bind to antigens or receptors of interest,
wherein the modified IL-2, and the one or more targeting moieties are optionally connected with one or more linkers,
wherein the modified IL-2 signaling agent comprises a glycosylation mutation, wherein the mutation decreases or eliminates glycosylation of the modified IL-2 signaling agent.

6. The chimeric protein or protein complex of claim 5, wherein the glycosylation mutation is a T3 substitution, optionally wherein the mutation is one of T3A, T3F, T3H, T3L, T3V, and T3Y.

7. The chimeric protein or protein complex of claim 5, wherein the glysosylation mutation is a deletion of the first 3, 4, 5, 6, 7, or 8 residues of the modified IL-2.

8. A chimeric protein or protein complex comprising:
(a) a modified IL-2 signaling agent, wherein the modified IL-2 signaling agent has one or more mutations or modifications relative to a neoleukin signaling agent having SEQ ID NO: 2 that confer improved safety and reduced affinity or bioactivity of the modified IL-2 signaling agent for IL-2Rαβγ and/or IL-2Rβ and/or IL-2Rγ and/or IL-2Rα, relative to the neoleukin signaling agent; and
(b) one or more targeting moieties, said targeting moieties comprising recognition domains which specifically bind to antigens or receptors of interest,
wherein the modified IL-2 signaling agent, and the one or more targeting moieties are optionally connected with one or more linkers,
wherein the modified IL-2 signaling agent comprises one or more mutations at a position selected from D15 and N40, optionally wherein the mutation is selected from D15T, D15H, N401, N40G, and N40R.

9. The chimeric protein or protein complex of any one of claims 1 to 8, wherein the modified IL-2 comprises one or more mutations that confer reduced affinity for an IL-2 receptor-chain.

10. The chimeric protein or protein complex of any one of claims 1 to 9, wherein the modified IL-2 exhibits reduced affinity for IL-2Rαβγ.

11. The chimeric protein or protein complex of any one of claims 1 to 9, wherein the modified IL-2 exhibits reduced affinity for IL-2Rβ, IL-2Rγ, or a combination thereof.

12. The chimeric protein or protein complex of any one of claims 1 to 9, wherein the modified IL-2 exhibits ablated affinity for IL-2Rα, IL-2Rβ, IL2Rγ or a combination thereof.

13. The chimeric protein or protein complex of claim 10, wherein the modified IL-2 exhibits reduced affinity for:
IL-2Rα and IL2Rβ, optionally wherein the modified IL-2 comprises one or more mutations selected from D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, and R38A/F42K/E62A/N88G/C125A; or
IL-2Rα and IL-2Rγ, optionally wherein the modified IL-2 comprises one or more mutations selected from R38A/F42K/Q126G, R38A/F42K/Q126I, and R38A/F42K/Q126Y; or
IL-2Rβ, optionally wherein the modified IL-2 comprises one or more mutations selected from D20E, D20F, D20G, D20H, D20I, D20K, D20L, D20V, N88G, N88E, N88K, N88Q, N88V, D20E/C125A, D20F/C125A, D20G/C125A, D20H/C125A, D20I/C125A, D20L/C125A, D20K/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, and N88V/C125A; or
IL-2Rγ, optionally wherein the modified IL-2 comprises one or more mutations selected from Q126A, Q126E, Q126F, Q126G, Q126H, Q126I, Q126K, Q126L, Q126N, Q126P, Q126R, Q126S, Q126T, Q126V, Q126W, and Q126Y, or a combination thereof.

14. The chimeric protein or protein complex of any one of claims 1 to 9, wherein the modified IL-2 exhibits reduced affinity for IL-2Rβγ, optionally wherein the modified IL-2 comprises one or more mutations selected from D20E/Q126A, D20E/Q126D, D20E/Q126E, D20E/Q126F, D20E/Q126G, D20E/Q126H, D20E/Q126I, D20E/Q126K, D20E/Q126L, D20E/Q126M, D20E/Q126N, D20E/Q126P, D20E/Q126R, D20E/Q126S, D20E/Q126T, D20E/Q126V, D20E/Q126W, D20E/Q126Y, D20V/Q126A, D20V/Q126D, D20V/Q126E, D20V/Q126F, D20V/Q126G, D20V/Q126H, D20V/Q126I, D20V/Q126K, D20V/Q126L, D20V/Q126M, D20V/Q126N, D20V/Q126P, D20V/Q126R, D20V/Q 126S, D20V/Q126T, D20V/Q126V, D20V/Q126W, D20V/Q126Y, N88A/Q126A, N88A/Q126D, N88A/Q126E, N88A/Q126F, N88A/Q126G, N88A/Q126H, N88A/Q126I, N88A/Q126K, N88A/Q126L, N88A/Q126M, N88A/Q126N, N88A/Q126P, N88A/Q126R, N88A/Q126S, N88A/Q126T, N88A/Q126V, N88A/Q126W, N88A/Q126Y, N88G/Q126A, N88G/Q126D, N88G/Q126E, N88G/Q126F, N88G/Q126G, N88G/Q126H, N88G/Q126I, N88G/Q126K, N88G/Q126L, N88G/Q126M, N88G/Q126N, N88G/Q126P, N88G/Q126R, N88G/Q126S, N88G/Q126T, N88G/Q126V, N88G/Q126W, and N88G/Q126Y.

15. The chimeric protein or protein complex of any one of claims 1 to 14, wherein the one or more mutations in the IL-2 signaling agent confers reduced affinity or bioactivity that is restorable by attachment to one or more targeting moieties.

16. The chimeric protein or protein complex of any one of the above claims, wherein the one or more targeting moieties are directed against a tumor cell, endothelial cell, epithelial cell, mesenchymal cell, tumor stroma or stromal cell, and/or ECM.

17. The chimeric protein or protein complex of any one of the above claims, wherein the one or more targeting moieties are directed against an immune cell and/or organ cells, and/or tissue cells.

18. The chimeric protein or protein complex of claim 17, wherein the immune cell is selected from a T cell, a Treg, a cytotoxic T lymphocyte, a T helper cell, a B cell, a dendritic cell, an anti-tumor or tumor-associated macrophage (e.g., a M1 or M2 macrophage), a neutrophil, myeloid derived suppressor cell, a natural killer (NK) cell, and a natural killer T (NKT) cell, optionally, the immune cell is a T cell and the targeting moiety targets CD8, or optionally wherein the immune cell is a Treg and the targeting moiety targets CTLA4, or optionally wherein the immune cell is an NK cell and the targeting moiety is NKp46.

19. The chimeric protein or protein complex of any one of the above claims, wherein the targeting moiety comprises a recognition domain that is a full-length antibody, a single-domain antibody, a recombinant heavy-chain-only antibody (VHH), a single-chain antibody (scFv), a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

20. The chimeric protein or protein complex of any one of the above claims, wherein the recognition domain is a single-domain antibody.

21. The chimeric protein or protein complex of any one of the above claims, wherein the recognition domain is a VHH or humanized VHH.

22. The chimeric protein or protein complex of any one of the above claims, wherein the recognition domain functionally modulates the antigen or receptor of interest.

23. The chimeric protein or protein complex of any one of the above claims, wherein the recognition domain binds but does not functionally modulate the antigen or receptor of interest.

24. The chimeric protein or protein complex of any one of the above claims, comprising two or more targeting moieties.

25. The chimeric protein or protein complex of any one of the above claims, wherein the targeting moieties binds to one or more of the following targets: CD8, CTLA4, CD3, CD4, DNAM-1, Nrp1 (neurophilin), TNFR1, TNFR2, GITR, ICOS, CD20, CD70, Clec9a, NKp46, PD-1, PD-L1, PD-L2, SIRP1a, FAP, XCR1, tenascin, or ECM proteins.

26. The chimeric protein or protein complex of any one of the above claims, further comprising one or more additional modified signaling agents.

27. The chimeric protein or protein complex of any one of the above claims, wherein the chimeric protein comprises two signaling agents and one targeting moiety or two targeting moieties and one signaling agent or two of both.

28. The chimeric protein or protein complex of any one of the above claims, wherein the chimeric protein comprises three signaling agents or three targeting moieties or three of both.

29. The chimeric protein or protein complex of claim 26, wherein the additional modified signaling agent comprises one or more mutations conferring reduced affinity or activity for a receptor relative to an unmutated signaling agent.

30. The chimeric protein or protein complex of claim 29, wherein the one or more mutations allow for attenuation of activity.

31. The chimeric protein or protein complex of claim 30, wherein agonistic or antagonistic activity is attenuated.

32. The chimeric protein or protein complex of any one of the above claims, wherein the chimeric protein is suitable for use in a patient having one or more of: cancer, infections, immune disorders, autoimmune diseases, cardiovascular diseases, wound, ischemia-related diseases, neurodegenerative diseases, and/or metabolic diseases.

33. A recombinant nucleic acid composition encoding one or more chimeric proteins or protein complexes of any one of the above claims.

34. A host cell comprising the recombinant nucleic acid of claim 33.

35. A method for treating cancer, comprising administering an effective amount of the chimeric protein or protein complex of any one of claims 1-32 to a patient in need thereof.

36. The method of claim 35, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

37. Use of a chimeric protein or protein complex of any one of claims 1-32 in the manufacture of a medicament.

38. Use of a chimeric protein or protein complex of any one of claims 1-31 in the treatment of cancer or autoimmune disease in a subject in need thereof.

39. A Fc-based chimeric protein complex comprising:
(a) a modified IL-2 signaling agent, wherein the modified IL-2 signaling agent has one or more mutations or modifications relative to a wild type signaling agent having SEQ ID NO: 1 that confer improved safety and reduced affinity or bioactivity of the modified IL-2 signaling agent for IL-2Rαβγ and/or IL-2Rβ and/or IL-2Rγ and/or IL-2Rα, relative to the wild type IL-2 signaling agent; and
(b) one or more targeting moieties, said targeting moieties comprising recognition domains which specifically bind to antigens or receptors of interest; and
(c) a Fc domain, the Fc domain optionally having one or more mutations that reduces or eliminates one or more effector functions of the Fc domain, promotes Fc chain pairing in the Fc domain, and/or stabilizes a hinge region in the Fc domain,
wherein the modified IL-2 signaling agent comprises:
(i) a mutation selected from D20E, D20F, D20G, D20H, D20I, D20K, D20L, and D20V; or
(ii) a mutation selected from N88G, N88E, N88K, N88Q, and N88V; or
(iii) a mutation selected from Q126G, Q126A, Q126E, Q126F, Q126H, Q126I, Q126K, Q126L, Q126N, Q126P, Q126R, Q126S, Q126T, Q126V, Q126W, and Q126Y; or
(iv) a mutation selected from (i) and (iii) or a mutation selected from (ii) and (iii); or
(v) one or more mutations selected from:
R38A/F42K/N88G/C125A,
F42K/C125A,
D20E/C125A,
D20F/C125A,
D20G/C125A,
D20H/C125A,
D20I/C125A,
D20K/C125A,
D20L/C125A,
D20N/C125A,
D20S/C125A,
D20T/C125A,
D20V/C125A,
N88A/C125A,
N88D/C125A,
N88G/C125A,
N88E/C125A,
N88H/C125A,
N88I/C125A,
N88K/C125A,
N88Q/C125A,
N88R/C125A,
N88T/C125A,
N88V/C125A,
D20E/R38A/F42K,
D20E/R38A/F42K/C125A,
D20V/R38A/F42K,
D20V/R38A/F42K/C125A,
R38A/F42Y/E62A/C125A,
R38A/F42Y/Y45A/E62A,
F42Y/Y45A/L72G,
R38A/F42Y/Y45A/E62A/C125A,
F42Y/Y45A/L72G/C125A,
R38A/F42K/N88G,
R38A/F42K/Q126G,
R38A/F42K/Q126I,
R38A/F42K/Q126Y,
R38A/F42K/Q126G/C125A,
R38A/F42K/Q1261/C125A,
R38A/F42K/Q126Y/C125A,
R38A/F42K/E62A/N88G, and
R38A/F42K/E62A/N88G/C125A.

40. The Fc-based chimeric protein complex of claim 39, wherein the modified IL-2-signaling agent further comprises a glycosylation mutation, wherein the mutation decreases or eliminates glycosylation of the modified IL-2 signaling agent.

41. The Fc-based chimeric protein complex of claim 40, wherein the glycosylation mutation is a T3 substitution, wherein the mutation is one of T3A, T3F, T3H, T3L, T3V, and T3Y.

42. The Fc-based chimeric protein complex of claim 40, wherein the glysosylation mutation is a deletion of the first 3, 4, 5, 6, 7, or 8 residues of the modified IL-2.

43. A Fc-based chimeric protein complex comprising:
(a) a modified IL-2 signaling agent, wherein the modified IL-2 signaling agent has one or more mutations or modifications relative to a wild type signaling agent having SEQ ID NO: 1 that confer improved safety and reduced affinity or bioactivity of the modified IL-2 signaling agent for IL-2Rαβγ and/or IL-2Rβ and/or IL-2Rγ and/or IL-2Rα, relative to the wild type IL-2 signaling agent; and
(b) one or more targeting moieties, said targeting moieties comprising recognition domains which specifically bind to antigens or receptors of interest; and
(c) a Fc domain, the Fc domain optionally having one or more mutations that reduces or eliminates one or more effector functions of the Fc domain, promotes Fc chain pairing in the Fc domain, and/or stabilizes a hinge region in the Fc domain,
wherein the modified IL-2 signaling agent comprises a glycosylation mutation, wherein the mutation decreases or eliminates glycosylation of the modified IL-2 signaling agent.

44. The Fc-based chimeric protein complex of claim 43, wherein the glycosylation mutation is a T3 substitution, optionally wherein the mutation is one of T3A, T3F, T3H, T3L, T3V, and T3Y.

45. The Fc-based chimeric protein complex of claim 43, wherein the glysosylation mutation is a deletion of the first 3, 4, 5, 6, 7, or 8 residues of the modified IL-2.

46. A Fc-based chimeric protein complex comprising:
(a) a modified IL-2 signaling agent, wherein the modified IL-2 signaling agent has one or more mutations or modifications relative to a neoleukin signaling agent having SEQ ID NO: 2 that confer improved safety and reduced affinity or bioactivity of the modified IL-2 signaling agent for IL-2Rαβγ and/or IL-2Rβ and/or IL-2Rγ and/or IL-2Rα, relative to the neoleukin signaling agent; and
(b) one or more targeting moieties, said targeting moieties comprising recognition domains which specifically bind to antigens or receptors of interest,
wherein the modified IL-2 signaling agent, and the one or more targeting moieties are optionally connected with one or more linkers,
wherein the modified IL-2 signaling agent comprises one or more mutations at a position selected from D15 and N40, optionally wherein the mutation is selected from D15T, D15H, N401, N40G, and N40R.

47. The Fc-based chimeric protein complex of any one of claims 39-46, wherein the modified IL-2 comprises one or more mutations that confer reduced affinity for an IL-2 receptor.

48. The Fc-based chimeric protein complex of any one of claims 39-47, wherein the modified IL-2 exhibits reduced affinity for IL-2Rαβγ.

49. The Fc-based chimeric protein complex of any one of claims 39-47, wherein the modified IL-2 exhibits reduced affinity for IL-2Rβ, IL-2Rγ, or a combination thereof.

50. The Fc-based chimeric protein complex of any one of claims 39-47, wherein the modified IL-2 exhibits ablated affinity for IL-2Rα, IL-2Rαβ, IL-2Rγ, or a combination thereof.

51. The Fc-based chimeric protein complex of claim 48, wherein the modified IL-2 exhibits reduced affinity for:
IL-2Rα and IL2Rβ, optionally wherein the modified IL-2 comprises one or more mutations selected from D20E/R38A/F42K, D20E/R38A/F42K/C125A, D20V/R38A/F42K, D20V/R38A/F42K/C125A, D20V/R38A/F42K/E62A, D20V/R38A/F42K/E62A/C125A, R38A/F42K/N88G, R38A/F42K/N88G/C125A, R38A/F42K/E62A/N88G, and R38A/F42K/E62A/N88G/C125A; or
IL-2Rα and IL-2Rγ, optionally wherein the modified IL-2 comprises one or more mutations selected from R38A/F42K/Q126G, R38A/F42K/Q126I, and R38A/F42K/Q126Y; or
IL-2Rβ, optionally wherein the modified IL-2 comprises one or more mutations selected from D20E, D20F, D20G, D20H, D20I, D20K, D20L, D20V, N88G, N88E, N88K, N88Q, N88V, D20E/C125A, D20F/C125A, D20G/C125A, D20H/C125A, D20I/C125A, D20L/C125A, D20K/C125A, D20N/C125A, D20S/C125A, D20T/C125A, D20V/C125A, N88A/C125A, N88D/C125A, N88G/C125A, N88E/C125A, N88H/C125A, N88I/C125A, N88K/C125A, N88Q/C125A, N88R/C125A, N88T/C125A, and N88V/C125A; or
IL-2Rγ, optionally wherein the modified IL-2 comprises one or more mutations selected from Q126A, Q126E, Q126F, Q126G, Q126H, Q126I, Q126K, Q126L, Q126N, Q126P, Q126R, Q126S, Q126T, Q126V, Q126W, and Q126Y, or a combination thereof.

52. The Fc-based chimeric protein complex of any one of claims 39-47, wherein the modified IL-2 exhibits reduced affinity for IL-2Rβγ, optionally wherein the modified IL-2 comprises one or more mutations selected from D20E/Q126A, D20E/Q126D, D20E/Q126E, D20E/Q126F, D20E/Q126G, D20E/Q126H, D20E/Q126I, D20E/Q126K, D20E/Q126L, D20E/Q126M, D20E/Q126N, D20E/Q126P, D20E/Q126R, D20E/Q126S, D20E/Q126T, D20E/Q126V, D20E/Q126W, D20E/Q126Y, D20V/Q126A, D20V/Q126D, D20V/Q126E, D20V/Q126F, D20V/Q126G, D20V/Q126H, D20V/Q126I, D20V/Q126K, D20V/Q126L, D20V/Q126M, D20V/Q126N, D20V/Q126P, D20V/Q126R, D20V/Q126S, D20V/Q126T, D20V/Q126V, D20V/Q126W, D20V/Q126Y, N88A/Q126A, N88A/Q126D, N88A/Q126E, N88A/Q126F, N88A/Q126G, N88A/Q126H, N88A/Q126I, N88A/Q126K, N88A/Q126L, N88A/Q126M, N88A/Q126N, N88A/Q126P, N88A/Q126R, N88A/Q126S, N88A/Q126T, N88A/Q126V, N88A/Q126W, N88A/Q126Y, N88G/Q126A, N88G/Q126D, N88G/Q126E, N88G/Q126F, N88G/Q126G, N88G/Q126H, N88G/Q126I, N88G/Q126K, N88G/Q126L, N88G/Q126M, N88G/Q126N, N88G/Q126P, N88G/Q126R, N88G/Q126S, N88G/Q126T, N88G/Q126V, N88G/Q126W, and N88G/Q126Y.

53. The Fc-based chimeric protein complex of any one of claims 39-52, wherein the one or more mutations in the IL-2 signaling agent confers reduced affinity or bioactivity that is restorable by attachment to one or more targeting moieties or upon inclusion in the Fc-based chimeric protein complex.

54. The Fc-based chimeric protein complex of any one of claims 39-53, wherein the one or more targeting moieties are directed against a tumor cell, endothelial cell, epithelial cell, mesenchymal cell, tumor stroma or stromal cell, and/or ECM.

55. The Fc-based chimeric protein complex of any one of claims 39-54, wherein the one or more targeting moieties are directed against an immune cell and/or organ cells, and/or tissue cells.

56. The Fc-based chimeric protein complex of claim 55, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof, optionally, wherein the immune cell is a T cell and the targeting moiety targets CD8, or optionally wherein the immune cell is a Treg and the targeting moiety targets CTLA4, or optionally wherein the immune cell is an NK cell and the targeting moiety is NKp46.

57. The Fc-based chimeric protein complex of any one of claims 39-56, wherein the targeting moiety comprises a recognition domain that is a full-length antibody, a single-domain antibody, a recombinant heavy-chain-only antibody (VHH), a single-chain antibody (scFv), a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

58. The Fc-based chimeric protein complex of any one of claims 39-57, wherein the recognition domain is a single-domain antibody.

59. The Fc-based chimeric protein complex of any one of claims 39-58, wherein the recognition domain is a V_{HH} or humanized V_{HH}.

60. The Fc-based chimeric protein complex of any one of claims 39-59, wherein the recognition domain functionally modulates the antigen or receptor of interest.

61. The Fc-based chimeric protein complex of any one of claims 39-60, wherein the recognition domain binds but does not functionally modulate the antigen or receptor of interest.

62. The Fc-based chimeric protein complex of any one of claims 39-61, comprising two or more targeting moieties.

63. The Fc-based chimeric protein complex of any one of claims 39-62, wherein the targeting moieties binds to one or more of the following targets: CD8, CTLA4, CD3, CD4, DNAM-1, Nrp1 (neurophilin), TNFR1, TNFR2, GITR, ICOS, CD20, CD70, Clec9a, NKp46, PD-1, PD-L1, PD-L2, SIRP1a, FAP, XCR1, tenascin, or ECM proteins.

64. The Fc-based chimeric protein complex of any one of claims 39-63, further comprising one or more additional modified signaling agents.

65. The Fc-based chimeric protein complex of any one of claims 39-64, wherein the chimeric protein comprises two signaling agents or two targeting moieties or two of both.

66. The Fc-based chimeric protein complex of any one of claims 39-65, wherein the chimeric protein comprises three signaling agents or three targeting moieties or three of both.

67. The Fc-based chimeric protein complex of claim 64, wherein the additional modified signaling agent comprises one or more mutations conferring reduced affinity or activity for a receptor relative to an unmutated signaling agent.

68. The Fc-based chimeric protein complex of claim 67, wherein the one or more mutations allow for attenuation of activity.

69. The Fc-based chimeric protein complex of claim 68, wherein agonistic or antagonistic activity is attenuated.

70. The Fc-based chimeric protein complex of any one of claims 39-69, wherein the chimeric protein is suitable for use in a patient having one or more of: cancer, infections, immune disorders, autoimmune diseases, cardiovascular diseases, wounds, ischemia-related diseases, neurodegenerative diseases, and/or metabolic diseases.

71. A recombinant nucleic acid composition encoding one or more Fc-based chimeric protein complexes of any one of claims 39-70, or a constituent polypeptide thereof.

72. A host cell comprising the recombinant nucleic acid of claim 71.

73. A method for treating cancer, comprising administering an effective amount of the (i) Fc-based chimeric protein complex of any one of claims 39-69 to a patient in need thereof; ii) the recombinant nucleic acid of claim 71 to a patient in need thereof; or iii) the host cell of claim 72 to a patient in need thereof.

74. The method of claim 73, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

75. Use of a Fc-based chimeric protein complex of any one of claims 39-70 in the manufacture of a medicament.

76. Use of a Fc-based chimeric protein complex of any one of claims 39-69 in the treatment of cancer or autoimmune disease in a subject in need thereof.

77. The Fc-based chimeric protein complex of any one of claims 39-70, wherein the Fc domain is from IgG, IgA, IgD, IgM or IgE.

78. The Fc-based chimeric protein complex of claim 77, wherein the IgG is selected from IgG1, IgG2, IgG3, or IgG4.

79. The Fc-based chimeric protein complex of any one of claims 39-70, wherein the Fc domain is from human IgG, IgA, IgD, IgM or IgE.

80. The Fc-based chimeric protein complex of claim 79, wherein the human IgG is selected from human IgG1, IgG2, IgG3, or IgG4.

81. The Fc-based chimeric protein complex of any one of claims 39-70 and 77-80, wherein the Fc chain pairing is promoted by ionic pairing and/or a knob-in-hole pairing.

82. The Fc-based chimeric protein complex of any one of claims 39-70 and 77-81, wherein the one or more mutations to the Fc domain results in an ionic pairing between the Fc chains in the Fc domain.

83. The Fc-based chimeric protein complex of any one of claims 39-70 and 77-82, wherein the one or more mutations to the Fc domain results in a knob-in-hole pairing in the Fc domain.

84. The Fc-based chimeric protein complex of any one of claims 39-70 and 77-83, wherein the one or more mutations to the Fc domain results in the reduction or elimination of an effector function of the Fc domain.

85. The Fc based chimeric protein complex of any one of claims 39-70 and 77-84, wherein the Fc-based chimeric protein complex is a heterodimer and has a trans orientation/configuration, as relates to any targeting moiety and signaling agent, relative to each other, or any targeting moieties relative to each other, or any signaling agents relative to each other.

86. The Fc based chimeric protein complex of any one of claims 39-70 and 77-85, wherein the Fc-based chimeric protein complex is a heterodimer and has a cis orientation, as relates to any targeting moiety and signaling agent, relative to each other, or any targeting moieties relative to each other, or any signaling agents relative to each other.

87. The Fc based chimeric protein complex of any one of claims 39-70 and 77-86, wherein the Fc comprises L234A, L235A, and K322Q substitutions in human IgG1 (according to EU numbering).

88. The Fc-based chimeric protein complex of any one of claims 39-70 and 77-87, wherein the Fc is human IgG1, and optionally contains one or more mutations of L234, L235, K322, D265, P329, and P331 (according to EU numbering).

89. The Fc-based chimeric protein complex of any one of claims 39-70 and 77-88, wherein the Fc-based chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 31, and 32.

90. The Fc-based chimeric protein complex of any one of claims 39-70 and 77-89, wherein the Fc-based chimeric protein complex comprises a polypeptide having an amino acid sequence having at least 90%, or at least 95%, or at least 96%, or at least 97%, or at least 98%, or at least 99% identity with any one of SEQ ID NOs: 292, 293, 294, 299, 301, 310, 311, 312, 315, 316, 319, 320, 325, 328, 332, 333, and 335.

91. The chimeric protein of any one of claims 1-32 or the Fc-based chimeric protein complex of any one of claim 39-70 or 77-89, wherein the chimeric protein or Fc-based chimeric protein complex comprises a polypeptide having an amino acid sequence having at least 90%, or at least 95%, or at least 96%, or at least 97%, or at least 98%, or at least 99% identity with any one of SEQ ID NOs: 290-449, 478-495, or 501-531.

92. A chimeric protein or protein complex comprising:
(a) a modified IL-2 signaling agent, wherein the modified IL-2 signaling agent has a single mutation relative to a wild type signaling agent having SEQ ID NO: 1 that confers improved safety and reduced affinity or bioactivity of the modified IL-2 signaling agent for IL-2Rβ, relative to the wild type IL-2 signaling agent; and
(b) one or more targeting moieties, said targeting moieties comprising recognition domains which specifically bind to antigens or receptors of interest,
wherein the modified IL-2, and the one or more targeting moieties are optionally connected with one or more linkers,
wherein the single mutation is selected from D20E, D20F, D20G, D20H, D20I, D20K, D20L, D20N, D20V, N88G, N88E, N88I, N88K, N88Q, N88R and N88V, and
wherein the modified IL-2 signaling agent does not comprise a mutation that confers reduced affinity or bioactivity for IL-2Rα.

93. The chimeric protein or protein complex of claim 92, wherein the modified IL-2-signaling agent further comprises a glycosylation mutation, wherein the mutation decreases or eliminates glycosylation of the modified IL-2 signaling agent.

94. The chimeric protein or protein complex of claim 93, wherein the glycosylation mutation is a T3 substitution, wherein the mutation is one of T3A, T3F, T3H, T3L, T3V, and T3Y.

95. The chimeric protein or protein complex of claim 93, wherein the glysosylation mutation is a deletion of the first 3, 4, 5, 6, 7, or 8 residues of the modified IL-2.

96. The chimeric protein or protein complex of any one of claims 92 to 95, wherein the one or more mutations in the IL-2 signaling agent confers reduced affinity or bioactivity that is restorable by attachment to one or more targeting moieties.

97. The chimeric protein or protein complex of any one of claims 92 to 96, wherein the one or more targeting moieties are directed against a tumor cell, endothelial cell, epithelial cell, mesenchymal cell, tumor stroma or stromal cell, and/or ECM.

98. The chimeric protein or protein complex of any one of claims 92 to 97, wherein the one or more targeting moieties are directed against an immune cell and/or organ cells, and/or tissue cells.

99. The chimeric protein or protein complex of claim 98, wherein the immune cell is selected from a T cell, a Treg, a cytotoxic T lymphocyte, a T helper cell, a B cell, a dendritic cell, an anti-tumor or tumor-associated macrophage (e.g., a M1 or M2 macrophage), a neutrophil, myeloid derived suppressor cell, a natural killer (NK) cell, and a natural killer T (NKT) cell, optionally, the immune cell is a T cell and the targeting moiety targets CD8, or optionally wherein the immune cell is a Treg and the targeting moiety targets CTLA4, or optionally wherein the immune cell is an NK cell and the targeting moiety is NKp46.

100. The chimeric protein or protein complex of any one of claims 92 to 99, wherein the targeting moiety comprises a recognition domain that is a full-length antibody, a single-domain antibody, a recombinant heavy-chain-only antibody (VHH), a single-chain antibody (scFv), a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

101. The chimeric protein or protein complex of any one of claims 92 to 100, wherein the recognition domain is a single-domain antibody.

102. The chimeric protein or protein complex of any one of claims 92 to 101, wherein the recognition domain is a VHH or humanized VHH.

103. The chimeric protein or protein complex of any one of claims 92 to 102, wherein the recognition domain functionally modulates the antigen or receptor of interest.

104. The chimeric protein or protein complex of any one of claims 92 to 103, wherein the recognition domain binds but does not functionally modulate the antigen or receptor of interest.

105. The chimeric protein or protein complex of any one of claims 92 to 104, comprising two or more targeting moieties.

106. The chimeric protein or protein complex of any one of claims 92 to 105, wherein the targeting moieties binds to one or more of the following targets: CD8, CTLA4, CD3, CD4, DNAM-1, Nrp1 (neurophilin), TNFR1, TNFR2, GITR, ICOS, CD20, CD70, Clec9a, NKp46, PD-1, PD-L1, PD-L2, SIRP1a, FAP, XCR1, tenascin, or ECM proteins.

107. The chimeric protein or protein complex of any one of claims 92 to 106, further comprising one or more additional modified signaling agents.

108. The chimeric protein or protein complex of any one of claims 92 to 107, wherein the chimeric protein comprises two signaling agents and one targeting moiety or two targeting moieties and one signaling agent or two of both.

109. The chimeric protein or protein complex of any one of the claims 92 to 108, wherein the chimeric protein comprises three signaling agents or three targeting moieties or three of both.

110. The chimeric protein or protein complex of claim 107, wherein the additional modified signaling agent comprises one or more mutations conferring reduced affinity or activity for a receptor relative to an unmutated signaling agent.

111. The chimeric protein or protein complex of claim 110, wherein the one or more mutations allow for attenuation of activity.

112. The chimeric protein or protein complex of claim 111, wherein agonistic or antagonistic activity is attenuated.

113. The chimeric protein or protein complex of any one of claims 92 to 112, wherein the chimeric protein is suitable for use in a patient having one or more of: cancer, infections, immune disorders, autoimmune diseases, cardiovascular diseases, wound, ischemia-related diseases, neurodegenerative diseases, and/or metabolic diseases.

114. A recombinant nucleic acid composition encoding one or more chimeric proteins or protein complexes of any one of claims 92 to 112.

115. A host cell comprising the recombinant nucleic acid of claim 114.

116. A method for treating cancer, comprising administering an effective amount of the chimeric protein or protein complex of any one of claims 92 to 112 to a patient in need thereof.

117. The method of claim 116, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

118. Use of a chimeric protein or protein complex of any one of claims 92 to 112 in the manufacture of a medicament.

119. Use of a chimeric protein or protein complex of any one of claims 92 to 112 in the treatment of cancer or autoimmune disease in a subject in need thereof.

120. A Fc-based chimeric protein complex comprising:
(a) a modified IL-2 signaling agent, wherein the modified IL-2 signaling agent has a single mutation relative to a wild type signaling agent having SEQ ID NO: 1 that confers improved safety and reduced affinity or bioactivity of the modified IL-2 signaling agent for IL-2Rβ, relative to the wild type IL-2 signaling agent; and
(b) one or more targeting moieties, said targeting moieties comprising recognition domains which specifically bind to antigens or receptors of interest; and
(c) a Fc domain, the Fc domain optionally having one or more mutations that reduces or eliminates one or more effector functions of the Fc domain, promotes Fc chain pairing in the Fc domain, and/or stabilizes a hinge region in the Fc domain,
wherein the single mutation is selected from D20E, D20F, D20G, D20H, D20I, D20K, D20L, D20N, D20V, N88G, N88E, N88I, N88K, N88Q, N88R and N88V, and
wherein the modified IL-2 signaling agent does not comprise a mutation that confers reduced affinity or bioactivity for IL-2Rα.

121. The Fc-based chimeric protein complex of claim 120, wherein the modified IL-2-signaling agent further comprises a glycosylation mutation, wherein the mutation decreases or eliminates glycosylation of the modified IL-2 signaling agent.

122. The Fc-based chimeric protein complex of claim 121, wherein the glycosylation mutation is a T3 substitution, wherein the mutation is one of T3A, T3F, T3H, T3L, T3V, and T3Y.

123. The Fc-based chimeric protein complex of claim 121, wherein the glysosylation mutation is a deletion of the first 3, 4, 5, 6, 7, or 8 residues of the modified IL-2.

124. The Fc-based chimeric protein complex of any one of claims 120 to 123, wherein the one or more mutations in the IL-2 signaling agent confers reduced affinity or bioactivity that is restorable by attachment to one or more targeting moieties or upon inclusion in the Fc-based chimeric protein complex.

125. The Fc-based chimeric protein complex of any one of claims 120 to 124, wherein the one or more targeting moieties are directed against a tumor cell, endothelial cell, epithelial cell, mesenchymal cell, tumor stroma or stromal cell, and/or ECM.

126. The Fc-based chimeric protein complex of any one of claims 120 to 125, wherein the one or more targeting moieties are directed against an immune cell and/or organ cells, and/or tissue cells.

127. The Fc-based chimeric protein complex of claim 126, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof, optionally, wherein the immune cell is a T cell and the targeting moiety targets CD8, or optionally wherein the immune cell is a Treg and the targeting moiety targets CTLA4, or optionally wherein the immune cell is an NK cell and the targeting moiety is NKp46.

128. The Fc-based chimeric protein complex of any one of claims 120 to 127, wherein the targeting moiety comprises a recognition domain that is a full-length antibody, a single-domain antibody, a recombinant heavy-chain-only antibody (VHH), a single-chain antibody (scFv), a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

129. The Fc-based chimeric protein complex of any one of claims 120 to 128, wherein the recognition domain is a single-domain antibody.

130. The Fc-based chimeric protein complex of any one of claims 120 to 129, wherein the recognition domain is a VHH or humanized VHH.

131. The Fc-based chimeric protein complex of any one of claims 120 to 130, wherein the recognition domain functionally modulates the antigen or receptor of interest.

132. The Fc-based chimeric protein complex of any one of claims 120 to 131, wherein the recognition domain binds but does not functionally modulate the antigen or receptor of interest.

133. The Fc-based chimeric protein complex of any one of claims 120 to 132, comprising two or more targeting moieties.

134. The Fc-based chimeric protein complex of any one of claims 120 to 133, wherein the targeting moieties binds to one or more of the following targets: CD8, CTLA4, CD3, CD4, DNAM-1, Nrp1 (neurophilin), TNFR1, TNFR2, GITR, ICOS, CD20, CD70, Clec9a, NKp46, PD-1, PD-L1, PD-L2, SIRP1a, FAP, XCR1, tenascin, or ECM proteins.

135. The Fc-based chimeric protein complex of any one of claims 120 to 134, further comprising one or more additional modified signaling agents.

136. The Fc-based chimeric protein complex of any one of claims 120 to 135, wherein the chimeric protein comprises two signaling agents or two targeting moieties or two of both.

137. The Fc-based chimeric protein complex of any one of claims 120 to 136, wherein the chimeric protein comprises three signaling agents or three targeting moieties or three of both.

138. The Fc-based chimeric protein complex of claim 135, wherein the additional modified signaling agent comprises one or more mutations conferring reduced affinity or activity for a receptor relative to an unmutated signaling agent.

139. The Fc-based chimeric protein complex of claim 138, wherein the one or more mutations allow for attenuation of activity.

140. The Fc-based chimeric protein complex of claim 139, wherein agonistic or antagonistic activity is attenuated.

141. The Fc-based chimeric protein complex of any one of claims 120 to 140, wherein the chimeric protein is suitable for use in a patient having one or more of: cancer, infections, immune disorders, autoimmune diseases, cardiovascular diseases, wounds, ischemia-related diseases, neurodegenerative diseases, and/or metabolic diseases.

142. A recombinant nucleic acid composition encoding one or more Fc-based chimeric protein complexes of any one of claims 120 to 140, or a constituent polypeptide thereof.

143. A host cell comprising the recombinant nucleic acid of claim 142.

144. A method for treating cancer, comprising administering an effective amount of the (i) Fc-based chimeric protein complex of any one of claims 120 to 140 to a patient in need thereof; ii) the recombinant nucleic acid of claim 142 to a patient in need thereof; or iii) the host cell of claim 143 to a patient in need thereof.

145. The method of claim 144, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL;
mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

146. Use of a Fc-based chimeric protein complex of any one of claims 120 to 140 in the manufacture of a medicament.

147. Use of a Fc-based chimeric protein complex of any one of claims 120 to 140 in the treatment of cancer or autoimmune disease in a subject in need thereof.

148. The Fc-based chimeric protein complex of any one of claims 120 to 140, wherein the Fc domain is from IgG, IgA, IgD, IgM or IgE.

149. The Fc-based chimeric protein complex of claim 148, wherein the IgG is selected from IgG1, IgG2, IgG3, or IgG4.

150. The Fc-based chimeric protein complex of any one of claims 120 to 140, wherein the Fc domain is from human IgG, IgA, IgD, IgM or IgE.

151. The Fc-based chimeric protein complex of claim 150, wherein the human IgG is selected from human IgG1, IgG2, IgG3, or IgG4.

152. The Fc-based chimeric protein complex of any one of claims 120 to 140 and 148 to 151, wherein the Fc chain pairing is promoted by ionic pairing and/or a knob-in-hole pairing.

153. The Fc-based chimeric protein complex of any one of claims 120 to 140 and 148 to 152, wherein the one or more mutations to the Fc domain results in an ionic pairing between the Fc chains in the Fc domain.

154. The Fc-based chimeric protein complex of any one of claims 120 to 140 and 148 to 153, wherein the one or more mutations to the Fc domain results in a knob-in-hole pairing in the Fc domain.

155. The Fc-based chimeric protein complex of any one of claims 120 to 140 and 148 to 154, wherein the one or more mutations to the Fc domain results in the reduction or elimination of an effector function of the Fc domain.

156. The Fc based chimeric protein complex of any one of claims 120 to 140 and 148 to 155, wherein the Fc-based chimeric protein complex is a heterodimer and has a trans orientation/configuration, as relates to any targeting moiety and signaling agent, relative to each other, or any targeting moieties relative to each other, or any signaling agents relative to each other.

157. The Fc based chimeric protein complex of any one of claims 120 to 140 and 148 to 156, wherein the Fc-based chimeric protein complex is a heterodimer and has a cis orientation, as relates to any targeting moiety and signaling agent, relative to each other, or any targeting moieties relative to each other, or any signaling agents relative to each other.

158. The Fc based chimeric protein complex of any one of claims 120 to 140 and 148 to 157, wherein the Fc comprises L234A, L235A, and K322Q substitutions in human IgG1 (according to EU numbering).

159. The Fc-based chimeric protein complex of any one of claims 120 to 140 and 148 to 158, wherein the Fc is human IgG1, and optionally contains one or more mutations of L234, L235, K322, D265, P329, and P331 (according to EU numbering).

160. The Fc-based chimeric protein complex of any one of claims 120 to 140 and 148 to 159, wherein the Fc-based chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 31, and 32.

161. The Fc-based chimeric protein complex of any one of claims 120 to 140 and 148 to 160, wherein the Fc-based chimeric protein complex comprises a polypeptide having an amino acid sequence having at least 90%, or at least 95%, or at least 96%, or at least 97%, or at least 98%, or at least 99% identity with any one of SEQ ID NOs: 292, 293, 294, 299, 301, 310, 311, 312, 315, 316, 319, 320, 325, 328, 332, 333, and 335.

162. The chimeric protein of any one of claims 92 to 112 or the Fc-based chimeric protein complex of any one of claims 120 to 140 or 148 to 160, wherein the chimeric protein or Fc-based chimeric protein complex comprises a polypeptide having an amino acid sequence having at least 90%, or at least 95%, or at least 96%, or at least 97%, or at least 98%, or at least 99% identity with any one of SEQ ID NOs: 290-449, 478-495, or 501-531.
